SEQUENCE LISTING (5 sequences)

SEQ ID NO: 1 - protein, 184 aa, Mycobacterium smegmatis
GLDNELSLVDGQDRTLTVQQWDTFLNGVFPLDRNRLTREWFHSGRAKYIVAGPGADEFEGTLELGYQIGFPWSLGVGINFSYTTPNILIDDGDITAPPFGLNSVITPNLFPGVSISADLGNGPGIQEVATFSVDVSGAEGGVAVSNAHGTVTGAAGGVLLRPFARLIASTGDSVTTYGEPWNMN

SEQ ID NO: 2 - protein, 206 aa, synthetic construct
GLDNELSLVDGQDRTLTVQQWDTFLNGVFPLDRNRLTREWFHSGRAKYIVAGPGADEFEGTLELGYQIGFPWSLGVGINFSYTTPNILINNGNITAPPFGLNSVITPNLFPGVSISARLGNGPGIQEVATFSVRVSGAKGGVAVSNAHGTVTGAAGGVLLRPFARLIASTGDSVTTYGEPWNMNDDDDDDDDDDDDDDDDHHHHHH

SEQ ID NO: 3 - protein, 190 aa, synthetic construct
GLDNELSLVDGQDRTLTVQQWDTFLNGVFPLDRNRLTREWFHSGRAKYIVAGPGADEFEGTLELGYQIGFPWSLGVGINFSYTTPNILICNGNITAPPFGLNSVITPNLFPGVSISARLGNGPGIQEVATFSVRVSGAKGGVAVSNAHGTVTGAAGGVLLRPFARLIASTGDSVTTYGEPWNMNHHHHHH

SEQ ID NO: 4 - RNA, 22 nt, synthetic construct
  source 1..22: mol_type = other RNA
  modified_base 9: mod_base = m5c
tagcttatcagactgatgttga

SEQ ID NO: 5 - RNA, 23 nt, synthetic construct
  source 1..23: mol_type = other RNA
  modified_base 13: mod_base = m6a
caaagtgcttacagtgcaggtag

(19) United States
(12) Patent Application Publication
(10) Pub. No.: US 2024/0418701 A1
(43) Pub. Date: Dec. 19, 2024

(54) SINGLE MOLECULE IDENTIFICATION WITH A REACTIVE HETERO-NANOPORE

(71) Applicant: Nanjing University, Nanjing, Jiangsu (CN)

(72) Inventors: Shuo HUANG, Nanjing (CN); Shan Yu ZHANG, Nanjing (CN); Kefan WANG, Nanjing (CN); Yuqin WANG, Nanjing (CN); Yao LIU, Nanjing (CN)

(21) Appl. No.: 18/698,631

(22) PCT Filed: Oct. 9, 2022
(86) PCT No.: PCT/CN2022/124008

§ 371 Date: Apr. 4, 2024

(30) Foreign Application Priority Data
Oct. 9, 2021 (WO) PCT/CN2021/122891
Jul. 8, 2022 (WO) PCT/CN2022/104728

(51) Int. Cl. G01N 33/487; G01N 33/84

(52) U.S. Cl. CPC G01N 33/48721; G01N 33/84

(57) ABSTRACT

A protein nanopore comprising one or more sensing module and a method for characterizing a target molecule using the protein nanopore.

### FIELD OF THE INVENTION

The present invention relates to a system and a method for identifying an analyte using nanopore.

### BACKGROUND OF THE INVENTION

Though saccharide sequence or structure is known to be investigated by (micro) arrays, capillary electrophoresis (CE), liquid chromatography (LC), nuclear magnetic resonance (NMR) or mass spectrometry (MS), characterization performed by any single method can offer only an incomplete picture of the glycan analyte. Specifically, MS is blind to stereochemical information of monosaccharides and fails to discriminate between isomers. Saccharide characterizations by these means are generally expensive and time-consuming.

Analysis of RNA modifications can be performed by thin layer chromatography (TLC), high performance liquid chromatography coupled with UV spectrophotometry (HPLC-UV) or high performance liquid chromatography coupled to mass spectrometry (HPLC-MS). These methods enable simultaneous measurement of a large number of RNA modifications, but they fail to provide any sequence information. The strand sequencing strategy, which is limited by the spatial resolution equivalent to an average reading of ˜5-nucleotides, still suffers from discrimination between all epigenetic modifications by sequencing. This situation is even more serious when the modified nucleotides are close neighbours.

The analysis and detection of alditols are necessary in the medical and food industries, but the similarities in their chemical structures pose significant technical challenges to the design of sensing strategies.

The analysis and detection of natural amino acids by a nanopore are critical to achieve nanopore sequencing of peptide or protein. However, there is still no nanopore method that can simultaneously discriminate between all 20 natural amino acids and their post translational chemical modifications.

There still is a need for a new detection method.

### SUMMARY OF THE INVENTION

The first aspect of the present invention provides a protein nanopore comprising at least one sensing moiety, wherein the sensing moiety is a metal ion which is attached to a reactive amino acid residue in the nanopore and is capable of interacting with a target analyte.

In some embodiments, the metal ion is attached to the reactive amino acid residue via a ligand, and the metal ion and the ligand form a coordination complex.

In some embodiments, the ligand is nitrilotriacetic acid (NTA).

In some embodiments, the metal ion is selected from Ni²⁺, Cu²⁺, Co²⁺, Zn²⁺, Cd²⁺, Ag²⁺ Pb²⁺, Fe²⁺ or Fe³⁺.

In some embodiments, the reactive amino acid residue is selected from the group consisting of cysteine, methionine and lysine.

In some embodiments, the protein nanopore is a heterogeneous protein nanopore in which one or more but not all monomers comprise the sensing moiety and the other monomers do not comprise the sensing moiety.

In some embodiments, the heterogeneous protein nanopore is a variant of the nanopore selected from the group consisting of MspA, α-HL, Aerolysin, ClyA, FhuA, FraC, PlyA/B, CsgG and Phi 29 connector.

In some embodiments, the heterogeneous protein nanopore is a variant of MspA.

In some embodiments, the protein nanopore is a heterogeneous MspA nanopore that comprises Ni²⁺ attached to the reactive amino acid residue via a ligand.

In some embodiments, Ni²⁺ is attached to the reactive amino acid residue via NTA.

In some embodiments, the reactive amino acid residue is located at a position selected from 83-111, preferably 90, 91, 92 and 93.

In some embodiments, the heterogeneous protein nanopore has a mutation of N90C, N90M or N91C on one or more monomers compared to M2 MspA.

The second aspect of the present invention provides a protein nanopore comprising at least one sensing module, wherein the protein nanopore is a heterogeneous MspA in which one or more but not all monomers comprise the sensing module and the other monomers do not comprise the sensing module, wherein the sensing module is capable of interacting with a target analyte.

In some embodiments, the sensing module consists of one or more reactive amino acid residues that are comprised in one or more monomers of the heterogeneous MspA.

In some embodiments, the reactive amino acid residue is selected from methionine, histidine, cysteine or lysine or their combination thereof.

In some embodiments, the sensing module consists of one or more sensing moieties that are attached to one or more reactive amino acid residues comprised in one or more monomers of the heterogeneous protein nanopore, and the other monomers of the heterogeneous protein nanopore do not comprise the reactive amino acid residue.

In some embodiments, the reactive amino acid residue is selected from the group consisting of cysteine, methionine, lysine.

In some embodiments, the sensing moiety is a moiety comprising boronic acid.

In some embodiments, the moiety comprising boronic acid is phenylboronic acid (PBA).

In some embodiments, the reactive amino acid residue is located at one or more positions selected from 83-111, preferably 90, 91, 92 and/or 93.

In some embodiments, the heterogeneous protein nanopore has a mutation of N90C, N90M and/or N91C on one or more monomers compared to M2 MspA.

The third aspect of the present invention provides a method for characterizing a target analyte, comprising:
(i) providing any one of the above protein nanopores;
(ii) applying a voltage between the two sides of the protein nanopore reactor;
(iii) allowing the target analyte to pass through the nanopore; and
(iv) measuring an ionic current through the nanopore to provide a current pattern, and characterizing the target analyte based on the current pattern.

In some embodiments of the third aspect, the target analyte is in a sample, and step (iii) comprises allowing the sample to pass through the nanopore.

In some embodiments of the third aspect, the sample is selected from fruit juice, drink, tea and extract of herbal medicine.

The fourth aspect of the present invention provides use of any one of the above protein nanopores in characterizing a target analyte.

In some embodiments of the fourth aspect, the target analyte is in a sample.

In some embodiments of the fourth aspect, the sample is selected from fruit juice, drink, tea and extract of herbal medicine.

In some embodiments of the third or the fourth aspect, the target analyte can interact with boronic acid, metal ion, methionine, histidine, cysteine, lysine or any combination thereof.

In some embodiments of the third or the fourth aspect:
the analyte that can interact with boronic acid is selected from a chemical compound comprising 1,2-diol or 1,3-diol, an ion comprising metal element, hydrogen peroxide and any combination thereof;
the analyte that can interact with metal ion is a molecule that can interact with the metal ion by coordination; and
the analyte that can interact with methionine, histidine, cysteine or lysine is an ion comprising metal element.

In some embodiments of the third or the fourth aspect:
the ion comprising metal element is selected from alkaline-earth metal ion, transition metal ion and any combination thereof, preferably selected from AuCl₄⁻, Mg²⁺, Ca²⁺, Ba²⁺, Ni²⁺, Cu²⁺, Co²⁺, Zn²⁺, Cd²⁺, Ag²⁺, Pb²⁺ and any combination thereof.

In some embodiments of the third or the fourth aspect:
the chemical compound comprising 1,2-diol or 1,3-diol is selected from saccharide or a derivative thereof, α-hydroxy acid, a chemical compound comprising a ribose, nucleotide sugar, alditol, polyphenol, catecholamine or catecholamine derivative, tris(hydroxymethyl)methyl aminomethane (Tris), protocatechualdehyde, protocatechuic acid, caffeic acid, rosmarinic acid, lithospermic acid, salvianic acid A, salvianolic acid B and any combination thereof;

In some embodiments of the third or the fourth aspect:
the saccharide is selected from monosaccharide, oligosaccharide, polysaccharide and any combination thereof;
the derivative of saccharide is selected from N-acetylneuraminic acid (sialic acid), N-Acetyl-D-Galactosamine and any combination thereof;
α-hydroxy acid is selected from tartaric acid, malic acid, citric acid, isocitric acid and any combination thereof;
the chemical compound comprising a ribose is selected from nucleotide or modified nucleotide, derivative of nucleotide or modified nucleotide, nucleoside or nucleoside analogue, and any combination thereof;
the nucleotide sugar is selected from uridine diphosphate glucose (UDPG), uridine diphosphate N-acetylglucosamine, uridine diphosphate glucuronic acid, adenosine diphosphate glucose, uridine diphosphate galactose, uridine diphosphate xylose, guanosine diphospbate mannose, guanosine diphosphate fucose, cytidine monophosphate N-acetylneuraminic acid, uridine diphosphate N-acetylgalactosamine and any combination thereof;
the alditol is selected from glycerin, propanetriol, tetritol, pentitol, hexitol, erythritol, threitol, arabitol, xylitol, adonitol, fucitol, sorbitol such as L-sorbitol or D-sorbitol, mannitol, dulcitol, iditol, talitol, allitol, maltitol, lactitol, isomalt and any combination thereof;
the polyphenol is selected from catechin, neochlorogenic acid, anthocyanin, proanthocyanidin, catechol or derivative thereof, such as catechol, 3-fluorocatechol, 3-chlorocatechol, 3-bromocatechol, 4-fluorocatechol, 4-chlorocatechol, 4-bromocatechol, 3-methylcatechol, 4-methylcatechol, 3-methoxycatechol, 3-propylcatechol, 3-isopropylcatechol, 3,6-dibromocatechol, 4,5-dibromocatechol, 3,6-dichlorocatechol, and any combination thereof; and
the catecholamine or catecholamine derivative is selected from epinephrine, norepinephrine, isoprenaline and any combination thereof.

In some embodiments of the third or the fourth aspect:
the monosaccharide is selected from D-glyceraldehyde, D-erythrose, D-ribose, 2′-deoxy-D-ribose, D-xylose, L-arabinose, D-lyxose, D-glucose, D-galactose, D-mannose, D-fructose, L-sorbose, L-fucose, D-allose, D-tagatose, L-rhamnose, D-galactose and any combination thereof;
the oligosaccharide is selected from disaccharide (such as sucrose, isomaltulose, maltulose, turanose, leucrose, trehalulose, lactulose, maltose), trisaccharide (such as raffinose), tetrasccharide (such as stachyose) and complex oligosaccharide (such as acarbose) and any combination thereof;
the polysaccharide is selected from pentasaccharide, such as verbascose;
the nucleotide is selected from adenine nucleotide, cytosine nucleotide, uracil nucleotide, guanine nucleotide and any combination thereof;
the modified nucleotide is selected from a nucleotide containing 5-methylcytidine (m⁵C), N6-methyladenosine (m⁶A), pseudouridine (Ψ), inosine (I), N7-methylguanosine (m⁷G), N1-methyladenosine (m¹A), dihydrouridine (D), N2-methylguanosine (m²G), N2,N2-dimethylguanosine (m₂²G), wybutosine (Y), 5-methyluridine (T), N-acetylcytidine (ac4C) and any combination thereof;
the derivative of nucleotide or modified nucleotide is selected from monophosphate derivative, diphosphate derivative, triphosphate derivative and tetraphosphate derivative of a nucleotide or a modified nucleotide and any combination thereof, such as ADP, UDP, GDP, CDP, ATP, UTP, GTP, CTP and any combination thereof; and
the nucleoside analogue is selected from galidesvir, ribavirin, molnupiravir, remdesivir, loxoribine, mizoribine, 5-azacytidine, capecitabine, doxifluridine, 5-fluorouridine, forodesine, clitocine, pyrazofurin, sangivamycin, pseudouridimycin and any combination thereof.

In some embodiments of the third or the fourth aspect:
the molecule that can interact with the metal ion by coordination contains nitrogen, oxygen, sulfur, phosphorus or carbon atom that can coordinate with the metal ion.

In some embodiments of the third or the fourth aspect:
the molecule that can interact with the metal ion by coordination is a compound contains at least one carboxylic acid group or at least one amine group, an amino acid, modified amino acid, polymer of amino acids or modified amino acids, a chemical compound comprising guanine, adenine, thymine, cytosine or uracil, and any combination thereof.

In some embodiments of the third or the fourth aspect:
the amino acid is selected from alanine, cysteine, aspartic acid, glutamic acid, phenylalanine, glycine, histidine, isoleucine, lysine, leucine, methionine, asparagine, proline, glutamine, arginine, serine, threonine, valine, tryptophan, tyrosine, pyrolysine, selenocysteine and any combination thereof;
the modified amino acid is selected from phosphorylate amino acid, glycosylated amino acid, acetylated amino acid, methylated amino acid and any combination thereof, such as O-phospho-serine (p-S), N4-(β-N-acetyl-D-glucosaminyl)-asparagine (GlcNAc-N), O-acetyl-threonine (Ac-T), Nω,N′ω-dimethyl-arginine (SDMA) and any combination thereof; and
the chemical compound comprising guanine, adenine, thymine, cytosine or uracil is selected from guanine, adenine, thymine, cytosine or uracil, or a nucleoside comprising any one of them, or a nucleotide comprising any one of them, wherein the nucleotide is a ribonucleotide or a deoxyribonucleotide.

### BRIEF DESCRIPTION OF THE DRAWINGS

FIG. 1: The preparation of a boronated MspA for saccharide sensing. (a) The structure of (N90C)₁(M2)₇. (N90C)₁(M2)₇ is a heterogeneously assembled MspA octamer composed of seven units of M2 MspA-D16H6 (grey) and one unit of N90C MspA-H6 (red). A sole thiol group exists in the pore lumen of (N90C)₁(M2)₇. (b) The gel electrophoresis result demonstrating different types of heterogeneously assembled MspA octamers. Gel electrophoresis was performed on a 10% SDS-PAGE gel. Left lane: the homo-octameric M2 MspA-D16H6. Right lane: the homo-octameric N90C MspA-H6. Middle lane: all possible combinations of heterogeneously assembled MspA octamers when M2 MspA-D16H6 and N90C MspA-H6 were co-expressed and assembled. The cartoons on the right denote the corresponding type of hetero-octameric MspA. A grey dot represents an M2 MspA-D16H6 monomer and a red dot stands for a N90C MspA-H6. (N90C)₁(M2)₇, the desired MspA hetero-octamer, appeared as the top 2^{nd} band in the middle lane (red rectangle). (c) Single molecule demonstration of MPBA [3-(maleimide) phenylboronic acid] modification to a (N90C)₁(M2)₇. Single channel recording was performed with a single (N90C)₁(M2)₇ MspA pore, as described in Methods in Example 1. A +100 mV bias was continuously applied. Prior to MPBA conjugation, additional shot noises were also observed. When a MPBA was conjugated to the pore, an irreversible drop of current, measuring ˜53 pA was observed. I₀ stands for the open pore current of (N90C)₁(M2)₇ and Iₚ stands for the open pore current of the MPBA modified (N90C)₁(M2)₇. (d) The mechanism of saccharide sensing. By taking L-Sorbose as a representative saccharide, reversible binding/dissociation of L-Sorbose to the phenylboronic acid forms the basis of sensing. (e) A representative trace of L-Sorbose sensing. The trace was acquired when a +160 mV bias was continuously applied and L-Sorbose was added to cis with a 10 mM final concentration. A 1.5 M KCl, 10 mM MOPS, pH 7.0 buffer was used. Iₛ stands for the blockage level when a saccharide was bound to the pore. A ˜102 pA current drop was monitored when an L-Sorbose was sensed. (f) A representative event of L-Sorbose. The corresponding all-point histogram plot of the blockage level is placed to the right. (g) The scatter plot of the ΔI vs the standard deviation (S.D.) when L-Sorbose was sensed as the sole analyte. ΔI=Iₚ−Iₛ. 910 events (n=910) were included in the plot. The scatter plot was color coded (ggplot 2, R) according to the local event density around each data point. (h) The plot of the reciprocals of the mean inter-event intervals (1/τₒₙ) and the reciprocals of the mean residence time (1/τ_{off}) of L-Sorbose sensing events versus the L-Sorbose concentration in cis. Three independent measurements (N=3) were performed for each condition.

FIG. 2: Single molecule identification of D-Fructose, D-Galactose, D-Mannose and D-Glucose. The measurements were performed as described in Methods in Example 1, in a 1.5 M KCl, 10 mM MOPS, pH 7.0 buffer. Different saccharides were respectively added to cis to reach the desired final concentration. (a, d, g, j) The chemical structure of D-Fructose (Fru, a), D-Galactose (Gal, d), D-Mannose (Man, g) or D-Glucose (Glc, j). All mentioned monosaccharides measure 180.16 in the molecular weight (MW). (b, e, h, k) Representative event types of D-Fructose (20 mM, b), D-Galactose (20 mM, e), D-Mannose (20 mM, h) or D-Glucose (60 mM, k). All mentioned monosaccharides report more than one type of events, respectively denoted with roman numerals. (c, f, i, l) Scatter plots of ΔI/Iₚ versus S.D. for different monosaccharides. To assist recognition of different event populations, the scatter plots were color coded (ggplot 2, R) according to the local event density around each data point. Different event types, as marked with roman numerals, were respectively denoted on the scatter plot, consistent with that demonstrated in (b, e, h, k).

FIG. 3: Discrimination between five monosaccharides by machine learning. (a) The workflow of machine learning. Five classes of events, respectively from D-Fructose, D-Galactose, D-Mannose, D-Glucose or L-Sorbose sensing, were collected to form a database. Nine event features were extracted to form a feature matrix. Then 1000 samples were randomly selected from each class to form the labeled data set. The labeled data set was randomly split into a training set (80%) and a testing set (20%) for model training and testing. A 10-fold cross validation was performed to evaluate the models. The validation accuracy is defined as the ratio of events being correctly recognized in the whole dataset. Six models were separately employed and the Random Forest model has reported the highest accuracy score: 0.974. Random Forest was then further tuned and an improved accuracy score: 0.975 was achieved. (b) The feature importance of the Random Forest model trained. (c) The confusion matrix result generated by the testing set. (d) The learning curve with varying training set sizes. When the size of the training set exceeds 508, the validation accuracy reaches 0.95. (e-f) A representative trace containing a mixture of saccharide sensing events. The events in the trace was automatically predicted as D-Fructose (green pentagon), D-Galactose (yellow circle), D-Mannose (green circle), D-Glucose (blue circle) and L-Sorbose (orange pentagon) by the machine learning algorithm. The measurement was performed in a 1.5 M KCl, 10 mM MOPS, pH 7.0 buffer, as described in Methods in Example 1. D-Fructose (5 mM), D-Galactose (10 mM), D-Mannose (10 mM), D-Glucose (60 mM), L-Sorbose (1 mM) were added to cis to form a mixture.

FIG. 4: Single molecule identification of D-Ribose, D-Xylose, L-Rhamnose and N-Acetyl-D-Galactosamine. The measurements were performed as described in Methods in Example 1, in a 1.5 M KCl, 10 mM MOPS, pH 7.0 buffer. Different saccharides were respectively added to cis to reach the desired final concentration. (a, d, g, j) The chemical structure of D-Ribose (Rib, a), D-Xylose (Xyl, d), L-Rhamnose (L-Rha, g) and N-Acetyl-D-Galactosamine (GalNAc, j). (b, e, h, k) Representative event types of D-Ribose (20 mM, b), D-Xylose (20 mM, e), L-Rhamnose (40 mM, h) or N-Acetyl-D-Galactosamine (20 mM, k). All mentioned monosaccharides report more than one type of events, respectively denoted with roman numerals. (c, f, i, l) Scatter plots of ΔI/Iₚ versus S.D. for different monosaccharides. To assist recognition of different event populations, the scatter plots were color coded (ggplot 2, R) according to the local event density around each data point. Different event types, as marked with roman numerals, were respectively denoted on the scatter plot, consistent with that demonstrated in (b, e, h, k).

FIG. 5: Discrimination between nine monosaccharides by machine learning. (a) The schematic diagram. Nine types of monosaccharides were tested by MspA-PBA. (b) The confusion matrix result generated by the testing set. (c) The scatter plot demonstration of results acquired from a mixture of nine monosaccharides. The label of each saccharide type was predicted by machine learning. (d-f) A representative trace containing events when nine saccharide types were sensed in a mixture. The events in the trace was automatically predicted as D-Fructose (green pentagon), D-Galactose (yellow circle), D-Mannose (green circle), D-Glucose (blue circle), L-Sorbose (orange pentagon), D-Ribose (pink star), D-Xylose (orange star), L-Rhamnose (green triangle) and N-Acetyl-D-Galactosamine (yellow square) by machine learning. The measurement was performed with MspA-PBA in a 1.5 M KCl, 10 mM MOPS, pH 7.0, as described in Methods in Example 1. D-Fructose (2.5 mM), D-Galactose (5 mM), D-Mannose (5 mM), D-Glucose (30 mM), L-Sorbose (0.5 mM), D-Ribose (5 mM), D-Xylose (5 mM), L-Rhamnose (10 mM) and N-Acetyl-D-Galactosamine (2.5 mM) were simultaneously added to cis to form a mixture.

FIG. 6: The co-expression vector map. Two target genes, N90C MspA-H6 and M2 MspA-D16H6, were custom synthesized and simultaneously constructed in the co-expression vector pETDuet-1 by Genscript (New Jersey). Specifically, the gene coding for N90C MspA-H6 was constructed at the first multiple cloning site between the restriction site of Nco I and Hind III. The gene coding for M2 MspA-D16H6 was constructed at the second multiple cloning site between the restriction site of Nde I and Blp I.

FIG. 7: The preparation of heterogeneously assembled MspA. (a) A schematic diagram demonstrating heterogeneously assembled MspA octamers. The (N90C)₁(M2)₇ octameric assembly, which contains a sole cysteine in the pore lumen, is the desired hetero-MspA type. (b) The UV absorbance spectrum during column elution. The marked fractions were further characterized by gel electrophoresis. (c, d) Gel electrophoresis results of different elution fractions. Gel electrophoresis was performed on a 4-15% gradient SDS-polyacrylamide gel. Lanes: M, precision plus protein standards (Bio-Rad); 1, the supernatant of the bacteria lysate; 2, the eluent collected from the nickel affinity column immediately after loading of the bacteria lysate; 3,5-8, 10-16, the corresponding elution fractions, as described in (b); 4,9, the purified octameric M2 MspA as a standard. According to the electrophoresis results, the fractions of 10-16 contain major hetero-MspAs. These fractions were collected for further characterizations and purifications (FIG. 1b).

FIG. 8: Single molecule characterization of (N90C)₁(M2)₇ before and after chemical modification. (a) The reaction mechanism of (N90C)₁(M2)₇ modification. Briefly, the cysteine thiol at site 90 of the pore lumen reacts irreversibly with 3-(maleimide) phenylboronic acid (MPBA) by Michael addition^{1,2}. (b) Spontaneous insertions of (N90C)₁(M2)₇ into the membrane. (c) Spontaneous insertions of MPBA modified (N90C)₁(M2)₇ (MspA-PBA) into the membrane. (d) The histogram of open pore currents acquired with (N90C)₁(M2)₇ (red) and MspA-PBA (black). The measurements (b-d) were performed in a 1.5 M KCl buffer (1.5 M KCl, 10 mM MOPS, pH 7.0) and a +100 mV voltage was continually applied. The histogram plot is overlaid with the corresponding Gaussian fitting results. Pore modification by a sole MPBA results in a current drop from I₀ (295.23±0.32 pA) to Iₚ (241.95±0.10 pA) at this condition (N=58). (e) The I-V (Current-Voltage) curve of (N90C)₁(M2)₇ MspA (red) and (N90C)₁(M2)₇ MspA-MPBA (black) (N=3). The I-V curve was acquired in an aqueous buffer of 1.5 M KCl and 10 mM MOPS at pH=7.0. A voltage ramp between −150 mV and +150 mV was applied. (N=3 for each condition)

FIG. 9: The concentration dependence of L-Sorbose sensing. Single-channel recordings were performed with MspA-PBA in a 1.5 M KCl, 10 mM MOPS pH=7.0 buffer. A +160 mV bias was continually applied. L-Sorbose was added to cis with a 0 to 2 mM final concentration. Generally, no sensing events were observed in the absence of L-Sorbose. The rate of event appearance is proportional to the final concentration of L-Sorbose.

FIG. 10: Definition of event parameters. A representative trace containing successive L-Sorbose sensing events is demonstrated. The trace was acquired with MspA-PBA as described in FIG. 1. The open pore current (Iₚ), the blockage level (Iₛ), the dwell time (τ_{off}), the inter-event duration (τₒₙ), the mean and the standard deviation (S.D.) are marked on the trace. The mean dwell time (τ_{off}) or the mean inter-event interval (τₒₙ) were respectively derived by performing exponential fitting to the histograms of τₒₙ or τ_{off}³.

FIG. 11: L-Sorbose sensing by an octameric M2 MspA-D16H6. L-Sorbose sensing was performed with an octameric M2 MspA-D16H6 in a 1.5 M KCl, 10 mM MOPS, pH7.0 buffer. A +160 mV bias was continually applied. The addition of L-Sorbose to cis to a 50 mM final concentration failed to produce any L-Sorbose sensing events.

FIG. 12: L-Sorbose sensing at different voltages. (a) Plot of the reciprocals of the mean inter-event intervals (1/τₒₙ) and the reciprocals of the mean residence time (1/tour) for L-Sorbose sensing at different voltages. (b) Plot of the mean blockage depth for L-Sorbose sensing at different voltages. The concentration of L-Sorbose in cis was set at 5 mM. All results were derived from measurements performed with MspA-PBA in a 1.5 M KCl, 10 mM MOPS, pH 7.0 buffer.

FIG. 13: The event scatter plot of ΔI/Iₚ versus S.D. for L-Sorbose. (a) The chemical structure of L-Sorbose. (b-d) Scatter plots of ΔI/Iₚ versus S.D. for L-Sorbose. Events in each plot were from a 50 min continually recorded trace, which was acquired as described in FIG. 1. Nanopore sensing of L-Sorbose results, which appear as separate event populations in the scatter plot. The event populations were marked with Roman numerals. For demonstration purposes, each scatter point is color coded according to the local density around each dot. The scatter plot was generated using the ggplot2 package of R.

FIG. 14: Different types of D-Fructose events. The measurements were performed with MspA-PBA (Methods in Example 1). A +160 mV bias was continually applied. D-Fructose was added to cis with a 20 mM final concentration (a) A representative trace containing D-Fructose sensing events. Different types of D-Fructose events were marked respectively with Roman numerals, I-IV. Repeated appearances of the same type of events are marked with Arabic numerals immediately following the Roman numerals. (b-f) Zoomed-in demonstration of each type of events.

FIG. 15: The event scatter plot of ΔI/Iₚ versus S.D. for D-Fructose. (a) The chemical structure of D-Fructose. (b-d) Scatter plots of ΔI/Iₚ versus S.D. for D-Fructose. Events in each plot were from a 30 min continually recorded trace, which was acquired as described in FIG. 14. Nanopore sensing of D-Fructose results in different types of sensing events, which appear as separate event populations in the scatter plot. Different event populations are marked with Roman numerals, consistent with that defined in FIG. 14. For demonstration purposes, each scatter point is color coded according to the local density around each dot. The scatter plot was generated using the ggplot2 package of R.

FIG. 16: Different types of D-Galactose events. The measurements were performed with MspA-PBA (Methods in Example 1). A +160 m V bias was continually applied D-Galactose was added to cis with a 20 mM final concentration. (a) A representative trace containing D-Galactose sensing events. Different types of D-Galactose events are respectively marked with Roman numerals, I-IV. Repeated appearances of the same type of events were mark with Arabic numerals immediately following the Roman numerals. (b-f) Zoomed-in demonstration of each type of events.

FIG. 17: The event scatter plot of ΔI/Iₚ versus S.D. for D-Galactose. (a) The chemical structure of D-Galactose. (b-d) Scatter plots of ΔI/Iₚ versus S.D. for D-Galactose. Events in each plot were from a 30 min continually recorded trace, which was acquired as described in FIG. 16. Nanopore sensing of D-Galactose results in different types of sensing events, which appear as separate event populations in the scatter plot. Different event populations are marked with Roman numerals, consistent with that defined in FIG. 16. For demonstration purpose, each scatter point is color coded according to the local density around each dot. The scatter plot was generated using the ggplot2 package of R.

FIG. 18: Different types of D-Mannose events. The measurements were performed with MspA-PBA (Methods in Example 1). A +160 m V bias was continually applied. D-Mannose was added to cis with a 20 mM final concentration. (a) A representative trace containing D-Mannose sensing events. Different types of D-Mannose events are respectively marked with Roman numerals, I-III. Repeated appearances of the same type of events are marked with Arabic numerals immediately following the Roman numerals. (b-f) Zoomed-in demonstration of each type of event.

FIG. 19: The event scatter plot of ΔI/Iₚ versus S.D. for D-Mannose. (a) The chemical structure of D-Mannose. (b-d) Scatter plots of ΔI/Iₚ versus S.D. for D-Mannose. Events in each plot were from a 30 min continually recorded trace, which was acquired as described in FIG. 18. Nanopore sensing of D-Mannose results in different types of sensing events, which appear as separate event populations in the scatter plot. Different event populations are marked with Roman numerals, consistent with that defined in FIG. 18. For demonstration purposes, each scatter point is color coded according to the local density around each dot. The scatter plot was generated using the ggplot2 package of R.

FIG. 20: Different types of D-Glucose events. The measurements were performed with MspA-PBA (Methods in Example 1). A+160 mV bias was continually applied. D-Glucose was added to cis with a 60 mM final concentration. (a) A representative trace containing D-Glucose sensing events. Different types of D-Glucose events are respectively marked with Roman numerals, I-IV. Repeated appearances of the same type of events are marked with Arabic numerals immediately following the Roman numerals. (b-f) Zoomed-in demonstration of each type of events.

FIG. 21: The event scatter plot of ΔI/Iₚ versus S.D. for D-Glucose. (a) The chemical structure of D-Glucose. (b-d) Scatter plots of ΔI/Iₚ versus S.D. for D-Glucose. Events in each plot were from a 50 min continually recorded trace, which was acquired as described in FIG. 20. Nanopore sensing of D-Glucose results in different types of sensing events, which appear as separate event populations in the scatter plot. Different event populations are marked with Roman numerals, consistent with that defined in FIG. 20. For demonstration purpose, each scatter point is color coded according to the local density around each dot. The scatter plot was generated using the ggplot2 package of R.

FIG. 22: Labeling monosaccharide sensing events by machine learning. (a) A scatter plot of ΔI/Iₚ versus S.D. for events acquired from a mixture of D-Fructose (5 mM), D-Galactose (10 mM), D-Mannose (10 mM), D-Glucose (60 mM) and L-Sorbose (1 mM). (b) Events labelled by machine learning. The distribution of each monosaccharide type is consistent with the corresponding monosaccharide sensing events when acquired separately.

FIG. 23: Different types of D-Ribose events. The measurements were performed with MspA-PBA (Methods in Example 1). A +160 m V bias was continually applied. D-Ribose was added to cis with a 20 mM final concentration. (a) A representative trace containing D-Ribose sensing events. Different types of D-Ribose events are respectively marked with Roman numerals, I-IV. Repeated appearances of the same type of events are marked with Arabic numerals immediately following the Roman numerals. (b-f) Zoomed-in demonstration of each type of events.

FIG. 24: The event scatter plot of ΔI/Iₚ versus S.D. for D-Ribose. (a) The chemical structure of D-Ribose. (b-d) Scatter plots of ΔI/Iₚ versus S.D. for D-Ribose. Events in each plot were from a 30 min continually recorded trace, which was acquired as described in FIG. 23. Nanopore sensing of D-Ribose results in different types of sensing events, which appear as separate event populations in the scatter plot. Different event populations are marked with Roman numerals, consistent with that defined in FIG. 23. For demonstration purpose, each scatter point is color coded according to the local density around each dot. The scatter plot was generated using the ggplot2 package of R.

FIG. 25: Different types of D-Xylose events. The measurements were performed with MspA-PBA (Methods in Example 1). A +160 mV bias was continually applied. D-Xylose was added to cis with a 20 mM final concentration. (a) A representative trace containing D-Xylose sensing events. Different types of D-Xylose events are respectively marked with Roman numerals, I-IV. Repeated appearances of the same type of events are marked with Arabic numerals immediately following the Roman numerals. (b-f) Zoomed-in demonstration of each type of events.

FIG. 26: The event scatter plot of ΔI/Iₚ versus S.D. for D-Xylose. (a) The chemical structure of D-Xylose. (b-d) Scatter plots of ΔI/Iₚ versus S.D. for D-Xylose. Events in each plot were from a 50 min continually recorded trace, which was acquired as described in FIG. 25. Nanopore sensing of D-Xylose results in different types of sensing events, which appear as separate event populations in the scatter plot. Different event populations are marked with Roman numerals, consistent with that defined in FIG. 25. For demonstration purpose, each scatter point is color coded according to the local density around each dot. The scatter plot was generated using the ggplot2 package of R.

FIG. 27: Different types of L-Rhamnose events. The measurements were performed with MspA-PBA (Methods in Example 1). A +160 m V bias was continually applied. L-Rhamnose was added to cis with a 40 mM final concentration. (a) A representative trace containing L-Rhamnose sensing events. Different types of L-Rhamnose events are respectively marked with Roman numerals, I-II. Repeated appearances of the same type of events are marked with Arabic numerals immediately following the Roman numerals. (b-f) Zoomed-in demonstration of each type of events.

FIG. 28: The event scatter plot of ΔI/Iₚ versus S.D. for L-Rhamnose. (a) The chemical structure of L-Rhamnose. (b-d) Scatter plots of ΔI/Iₚ versus S.D. for L-Rhamnose. Events in each plot were from a 30 min continually recorded trace, which was acquired as described in FIG. 27. Nanopore sensing of L-Rhamnose results in different types of sensing events, which appear as separate event populations in the scatter plot. Different event populations are marked with Roman numerals, consistent with that defined in FIG. 27. For demonstration purpose, each scatter point is color coded according to the local density around each dot. The scatter plot was generated using the ggplot2 package of R.

FIG. 29: Different types of N-Acetyl-D-Galactosamine events. The measurements were performed with MspA-PBA (Methods in Example 1). A +160 m V bias was continually applied. N-Acetyl-D-Galactosamine was added to cis with a 20 mM final concentration (a) A representative trace containing N-Acetyl-D-Galactosamine sensing events. Different types of N-Acetyl-D-Galactosamine events are respectively marked with Roman numerals, I-II. Repeated appearances of the same type of events are marked with Arabic numerals immediately following the Roman numerals. (b-f) Zoomed-in demonstration of each type of events.

FIG. 30: The event scatter plot of ΔI/Iₚ versus S.D. for N-Acetyl-D-Galactosamine. (a) The chemical structure of N-Acetyl-D-Galactosamine. (b-d) Scatter plots of ΔI/Iₚ versus S.D. for N-Acetyl-D-Galactosamine. Events in each plot were from a 50 min continually recorded trace, which was acquired as described in FIG. 29. Nanopore sensing of N-Acetyl-D-Galactosamine results in different types of sensing events, which appear as separate event populations in the scatter plot. Different event populations are marked with Roman numerals, consistent with that defined in FIG. 29. For demonstration purpose, each scatter point is color coded according to the local density around each dot. The scatter plot was generated using the ggplot2 package of R.

FIG. 31: Model evaluation for the classifier trained to discriminate nine monosaccharides. The model was trained as described in FIG. 5. (a) Classification performance of the different machine learning algorithms based on nine saccharides. Validation accuracy was calculated with 10-fold cross validation. The Random Forest model has outperformed all other models by reporting the highest validation accuracy (red marked). It was selected for further hyperparametrically-tuning and model evaluation. (b) Feature importance of the trained model based on the Random Forest model. (c) The learning curve with varying training sample number. According to the result in the image inset, a 0.92 validation accuracy was achieved when the size of the training set has exceeded 720.

FIG. 32: Machine learning predictions performed on results of nine monosaccharides in a mixture. The measurement was performed with a mixture of nine saccharides (FIG. 5), including D-Fructose (2.5 mM), D-Galactose (5 mM), D-Mannose (5 mM), D-Glucose (30 mM), L-Sorbose (0.5 mM), D-Ribose (5 mM), D-Xylose (5 mM), L-Rhamnose (10 mM) and N-Acetyl-D-Galactosamine (2.5 mM). (a) A scatter plot of ΔI/Iₚ versus S.D. generated from the extracted events. The label for each event is unknown at this stage. (b) The scatter plot of ΔI/Iₚ versus S.D. with machine learning predicted label. The distribution of each monosaccharide in the scatter plot is consistent with results separately acquired.

FIG. 33: Discrimination of canonical NMPs using a PBA modified MspA. (a) The structure of (N90C)₁(M2)₇. (N90C)₁(M2)₇ is a hetero-octameric MspA composed of seven units of M2 MspA-D16H6 (grey) and one unit of N90C MspA-H6 (pink). (N90C)₁(M2)₇ contains a sole cystine (blue), ready for subsequent modifications. Square box: the top view of a (N90C)₁(M2)₇. (b) The mechanism of NMP identification. A phenylboronic acid (PBA) was introduced to the pore constriction by modifying the sole cysteine thiol with a 3-(maleimide) phenylboronic acid (MPBA) via Michael addition. NMPs, when electrophoretically driven to the pore constriction, can reversibly react with PBA, generating stochastic sensing events. (c) Single channel observation of MPBA modification. After the addition of MPBA, a current drop of about 100 pA was observed, indicating the success of MPBA modification. With the subsequent addition of AMP, successive binding events immediately appear. To minimize bilayer rupture, the applied bias is switched to +20 m V whenever the Faraday cage is opened. The large noise is introduced during opening of the Faraday cage to perform MPBA or AMP addition. The open pore current (Iₚ) of MspA-PBA and the blockage level (I_{b}) are also marked. (d) The NMPs and their corresponding events. Top: The chemical structures of CMP (C), UMP (U), AMP (A) and GMP (G), of which the nucleobases were clearly demonstrated. Bottom: Representative sensing events corresponding to the NMPs described in the top panel. The measurements were carried out as described in Methods in Example 2 in a 1.5 M KCl buffer (1.5 M KCl, 10 mM MOPS, pH 7.0). A +200 m V bias was continually applied. NMPs were added to cis with a final concentration of 300 μM for each analyte. Iₚ marked with a grey dashed line. The blockage levels are marked with colour bands. (e) Top: A scatter plot of % I_{b} versus S.D. from results acquired with four types of NMPs. Bottom: Corresponding event histogram of % I_{b}. Events were acquired from four individual measurements, in which four types of NMP were separately added to cis with a final concentration of 300 μM. 500 successive events of each NMP were employed to generate the statistics. (f) A representative trace when four types of NMPs were simultaneously sensed. NMPs were simultaneously added to cis with a final concentration of 300 μM for each analyte. Events of different NMPs were identified according to its characteristic blockage depth and were respectively marked with C, U, A and G.

FIG. 34: Epigenetic NMPs identified by MspA-PBA. (a) Top: The epigenetic NMPs investigated in this paper. Seven types of epigenetic NMPs, including monophosphates of 5-methylcytidine (m⁵C), N6-methyladenosine (m⁶A), pseudouridine (Ψ), dihydrouridine (D), inosine (I), N7-methylguanosine (m⁷G) and N1-methyladenosine (m¹A) were investigated. For ease of display, only the nucleobases are shown and all modifications are highlighted in red. Bottom: Representative events of corresponding NMPs. From left to right, the representative events were respectively from m⁵C, m⁶A, Ψ, D, I, m⁷G and m¹A. The measurements were carried out as described in Methods in Example 2 in a 1.5 M KCl buffer (1.5 M KCl, 10 mM MOPS, pH 7.0). A transmembrane potential of +200 mV was continually applied. Epigenetic NMPs were added to cis with a final concentration of 300 μM for each analyte. The open pore current (Iₚ) of MspA-PBA is marked with a dashed line. The blockage levels are marked with colour bands. Noticeable differences in % I_{b} and S.D. were observed. (b) The violin plot of % I_{b} of different NMPs. Solely by analysing % I_{b}, different NMPs are generally distinguishable except for U and m⁵C (c) The scatter plot of % I_{b} versus S.D. for canonical and epigenetic NMPs. Events from eleven types of NMPs are clearly distinguishable when both % I_{b} and S.D. were considered. Events in (b) and (c) were acquired from eleven independent measurements, in which eleven types of NMP were separately added to cis as the sole analyte with a final concentration of 300 μM. 500 successive events of each NMP were employed to generate the statistics in (b) and (c).

FIG. 35: Distinguishing canonical and epigenetic NMPs. (a) A representative trace acquired from simultaneous sensing of CMP and m⁵C. Events of m⁵C show a deeper blockage amplitude and a larger noise than that of CMP. (b) The corresponding scatter plot of % I_{b} versus S.D. from results of a. 365 successive events were employed to generate the statistics. (c) A representative trace during simultaneous sensing of GMP and m⁷G. Events of m⁷G are significantly distinct from GMP events. (d) The corresponding scatter plot of % I_{b} versus S.D. from results of c. 865 successive events were employed to generate the statistics. (e) A representative trace acquired from simultaneous sensing of AMP, m⁶A and m¹A. Events of m¹A demonstrate a much deeper blockage depth than that of AMP and m⁶A. (f) The corresponding scatter plot of % I_{b} versus S.D. from results of e. 2230 successive events were employed to generate the statistics. (g) A representative acquired from simultaneous sensing of AMP and I. Events of I demonstrate a slightly deeper blockage than that of AMP. (h) The corresponding scatter plot of % I_{b} versus S.D. from results of g. 558 successive events were employed to generate the statistics. (i) A representative trace acquired from simultaneous sensing of UMP and ψ. Events of ψ are generally deeper in the blockage depth than that of UMP. (j) The corresponding scatter plot of % I_{b} versus S.D. from results of i. 710 successive events were employed to generate the statistics. (k) A representative trace acquired from simultaneous sensing of UMP and D. Events of D are generally deeper in the blockage depth than that of UMP. (l) The corresponding scatter plot of % I_{b} versus S.D. from results of k. 735 successive events were employed to generate the statistics. The NMP sensing events are labelled according to their characteristic event features. All measurements were performed as described in Methods in Example 2. The chambers were filled with a 1.5 M KCl buffer (1.5 M KCl, 10 mM MOPS, pH 7.0). A potential of +200 mV was continually applied. NMPs were simultaneously added to cis with a final concentration of 300 μM for each analyte.

FIG. 36: Machine learning assisted NMP identification. (a) The flow diagram of the training process. Eleven classes of events, including C, U, A, G, m⁵C, m⁶A, Ψ, D, I, m⁷G and m¹A were applied as the input dataset. Each class is composed of 400 events, randomly selected from a pool of events respectively acquired with each analyte type. The mean and the standard deviation of each event were extracted to form a feature matrix. Results in the matrix were further randomly split into a training subset for model training and a validation subset for model validation, with which a 10-fold cross validation was performed. All classifiers in the Classification Learner toolbox of MATLAB were evaluated to screen the best performing model. The SVM and the Naïve Bayes model have demonstrated the highest accuracy score of 0.996. The SVM was selected for all further investigations. (b) The confusion matrix of NMP classification generated using the SVM model. 100 events from each NMP class were treated as the testing set. (c) The decision boundary produced by the SVM model. Each coloured region represents the area in which a corresponding NMP event is to be predicted. The scatter plot of % I_{b} versus S.D. generated using the testing data is superimposed on the decision boundary for a demonstration. (d) A representative trace acquired by simultaneous sensing of eleven types of NMP. The measurements were carried out as described in Methods in Example 2. The chambers were filled with a 1.5 M KCl buffer (1.5 M KCl, 10 mM MOPS, pH 7.0). A transmembrane potential of +200 m V was continually applied. NMPs were simultaneously added to cis with a final concentration of 100 μM for each analyte. Characteristic events from different NMPs were automatically predicted by the trained SVM models and labelled with different colour dots (CMP: red; UMP: blue; AMP: green; GMP: purple; m⁵C: yellow, m⁶A: orchid, Ψ: orange, I: lime, D: cyan, m⁷G: teal, m¹A: pink).

FIG. 37: Detection of epigenetic modifications from RNA. (a) The schematic diagram of NMP identification from RNA using MspA-PBA. S1 Nuclease (green), an endonuclease insensitive to epigenetic modifications, was employed to decompose target RNAs into NMPs. The generated NMPs were then characterized using MspA-PBA, enabling profiling of RNA modifications in a quantitative manner. (b) The sequence of hsa-miR-21 and the corresponding trace of nanopore sensing of the digested products. Hsa-miR-21 was reported to contain a m⁵C at position 9. Characteristic events from C, U, A, G and m⁵C were clearly detected in the trace, which are respectively marked. The blockage level of m⁵C was marked with a yellow dashed line. Although the blockage level of m⁵C is close to that of U, the noise of m⁵C is significantly larger. (c) The scatter plot of % I_{b} versus S.D. for hsa-miR-21 digestion products. Events demonstrated were acquired from a 60 min continuous recording. The NMP identity is predicted by the SVM model. Five populations respectively from events of C, U, A, G and m⁵C were detected. (d) The sequence of hsa-miR-17 and the corresponding trace of nanopore sensing of the digested products. Hsa-miR-21 was reported to contain a m⁶A at position 13. Characteristic events of C, U, A, G and m⁶A are clearly detected, and are marked with the corresponding labels. The blockage level of m⁶A was marked with an orchid dashed line. (e) The scatter plot of % I_{b} versus S.D. derived from sensing events of hsa-miR-17 digestion products. Events demonstrated were acquired from a 60 min continuous recording. The NMP identity was predicted by the SVM model. Five populations including C, U, A, G and m⁶A were detected. All measurements were carried out as described in Methods in Example 2 in a 1.5 M KCl buffer (1.5 M KCl, 10 mM MOPS, pH 7.0). A transmembrane potential of +200 mV was continually applied. MicroRNA digestion product was added to cis with a final concentration of 100 ng/μl.

FIG. 38: Quantitative detection of epigenetic modifications of yeast tRNA^{phe}. (a) The sequence and the modifications of a yeast tRNA^{phe} Positions of modifications, including m²G=N2-methylguanosine, D=dihydrouridine, m₂²G=N2,N2-dimethylguanosine, Cₘ=2′-O-methylcytidine, Gₘ=2′-O-methylguanosine, Y=wybutosine, ψ=pseudouridine, T=5-methyluridine, m⁵C=5-methylcytidine, m⁷G=7-methylguanosine, and m¹A=1-methyladenosine, are shaded. These modifications can be divided into three categories. D, ψ, m⁵C, m⁷G and m¹A (shaded with red circles), which have known event features, are identifiable. m²G, m₂²G, T and Y (shaded with blue circles) are in principle detectable by MspA-PBA but are not identifiable. Due to a lack of cis-diol, Cₘ and Gₘ (shaded with grey circles) are in principle not detectable by MspA-PBA. (b) Gel electrophoresis results. Lane 1: Low range ssRNA ladder (New England Biolabs). Lane 2: yeast tRNA^{phe}; Lane 3: yeast tRNA^{phe} treated with S1 nuclease. The gel result shows that the yeast tRNA^{phe} were completely digested by S1 nuclease treatment. Operations of yeast tRNA^{phe} digestion are detailed in Methods in Example 2. (c) The scatter plot of % I_{b} versus S.D. for tRNA^{phe} digestion products. Events demonstrated were acquired from a 240 min continuous recording. The NMP identity is predicted by the Linear SVM model. Nine main populations respectively corresponding to events of C, U, A, G, m⁵C, ψ, D, m⁷G and m′A were identified. Four populations of events, which doesn't belong to any previously identified event type, were also detected by the unsupervised learning method DBSCAN (FIG. 74). (d) Comparison of the yeast tRNA^{phe} composition between that derived from measurements and the true value. The measured values were determined and calibrated according to Methods in Example 2. (e) Representative traces acquired during nanopore sensing of the tRNA^{phe} digestion products. Characteristic events of canonical and epigenetic NMPs are clearly detected, and are marked with the corresponding labels. UT stands for unidentified events. All measurements were carried out as described in Methods in Example 2 in a 1.5 M KCl buffer (1.5 M KCl, 10 mM MOPS, pH 7.0). A transmembrane potential of +200 m V was continually applied. Yeast tRNA^{phe} digestion product was added to cis with a final concentration of 100 ng/μl.

FIG. 39: The construction of the co-expression vector. The vector pETDuet-1 was employed to co-express both target genes (N90C MspA-H6 and M2 MspA-D16H6) in the same host cells. Specifically, the gene coding for N90C MspA-H6 was inserted between the restriction site of Nco I and Hind III. The gene coding for M2 MspA-D16H6 was inserted between the restriction site of Nde I and Blp I.

FIG. 40: The preparation of hetero-octameric MspA. (a) The UV absorbance spectrum during the gradient elution of the nickel column. Two major peaks around the 12^{th} and the 26^{th} fractions were observed in the spectrum. Their identities were confirmed by subsequent gel electrophoresis. (b-c) Hetero-octameric MspA characterized using SDS-polyacrylamide gel electrophoresis (4-20% gradient gel) Gel electrophoresis was continually run for 30 min with a +200 V applied potential. Lane M: precision plus protein standards (Bio-Rad). Lane 1: the supernatant of the bacterial lysate. Lane 2: the eluent of the bacterial lysate after column loading. Lanes 3-15: the eluted fractions as described in (a). The index of the fraction is respectively marked with red characters on the gel. The gel results show that the first peak (fraction 12) corresponds to proteins from the host cells. The second peak (fractions 21-33) corresponds to the eluted hetero-octameric MspA, consisting of different combinations of M2 MspA-D16H6 and N90C MspA-H6 monomers. The fractions containing hetero-octameric MspA were collected to be further separated on a 10% SDS-PAGE gel (FIG. 41).

FIG. 41: Gel electrophoresis results. All hetero-octameric MspAs were further characterized a 10% SDS-PAGE gel to separate the desired pore assembly type. Gel electrophoresis was run continually for 16 h with a +160 V applied potential. Left: results obtained from a 10% SDS-PAGE gel. Lane 1: the homo-octameric M2 MspA-D16H6. Lane 2: hetero-octameric MspAs prepared using the co-expression plasmid (FIG. 39). Lane 3: the homo-octameric N90C MspA-H6. (N90C)₁(M2)₇ which contains one fraction of N90C MspA-H6 and seven fractions of M2 MspA, appeared as the top 2^{nd} band in Lane 2 (marked with a dashed pink box). Right: cartoon illustrations of corresponding hetero-octameric MspA assemblies. A red dot stands for a N90C MspA-H6 monomer and a grey dot stands for an M2 MspA-D16H6 monomer.

FIG. 42: Single channel characterization of (N90C)₁(M2)₇ and MspA-PBA. (a) A representative trace demonstrating successive insertions of (N90C): (M2)₇. (b) A representative trace demonstrating successive insertions of MspA-PBA. The measurements in (a-b) were performed in a 1.5 M KCl buffer (1.5 M KCl, 10 mM MOPS, pH 7.0) and a bias of +200 mV was continually applied. Nanopores were added to cis to trigger spontaneous pore insertions into the membrane. (c) I-V curves of (N90C)₁(M2)₇ and MspA-PBA. A voltage ramp between −200 mV and +200 mV was applied to obtain the I-V curves. The statistics were based on three independent measurements for each condition (N=3). (d) Histogram of open pore currents of (N90C)₁(M2)₇ and MspA-PBA measured at +200 m V. The statistics was based on 50 events for each type of the pore (N=50). A sole MPBA modification to the pore results in a significant current drop from 623±13 (mean±FWHM) pA to 510±14 (mean±FWHM) pA according to the Gaussian fitting results (red and black lines). The relative frequency stands for the relative frequency of event counts in the histogram plot.

FIG. 43: The conductance of (N90C)₁(M2)₇ and MspA-PBA. The conductance of (N90C)₁(M2)₇ and MspA-PBA were evaluated at different KCl concentrations. (a) I-V curves of (N90C)₁(M2)₇ measured at different concentrations of KCl. (b) The plot of conductance of (N90C)₁(M2)₇ versus the KCl concentration. (c) I-V curves of MspA-PBA measured at different concentrations of KCL. (d) The plot of conductance of MspA-PBA versus the KCl concentration. The measurements were respectively performed in a 0.15 M, 0.5 M, 1.0 M, 1.5 M and 2.0 M KCl buffer. A voltage ramp between −150 mV and +150 mV was applied at each condition. The conductance is derived according to the slope of the I-V curve. The statistics were based on three independent measurements for each condition (N=3).

FIG. 44: NMP sensing performed with M2 MspA. (a-d) Representative traces of (a) CMP, (b) UMP, (c) AMP, (d) GMP sensing performed with M2 MspA. All measurements were carried out as described in Methods in Example 2 in a 1.5 M KCl buffer (1.5 M KCl, 10 mM MOPS, pH 7.0). A +200 mV bias was continually applied. NMPs were added to cis with a final concentration of 1 mM for each analyte. No binding events were observed, concluding that NMPs cannot be directly detected by M2 MspA, which lacks a phenylboronic acid chemical modification.

FIG. 45: Single molecule sensing of AMP and dAMP with MspA-PBA. (a-b) The chemical structure of (a) adenine ribonucleotide (AMP) and (b) adenine deoxyribonucleotide (dAMP). AMP and dAMP differ only in the sugar subunit (marked red). (c-d) Representative traces acquired with MspA-PBA when (c) AMP or (d) dAMP respectively was added as the sole analyte. Successive appearance of AMP sensing events was observed in (c). However, no dAMP sensing events were observed in (d). The results indicate that PBA only has an affinity for cis-diol and the deoxyribonucleotide is in principle not detectable by MspA-PBA. The measurement was carried out as described in Methods in Example 2 in a 1.5 M KCl buffer (1.5 M KCl, 10 mM MOPS, pH 7.0). A +200 mV bias was continually applied. AMP and dAMP were respectively added to cis with a final concentration of 1 mM.

FIG. 46: Definition of event parameters. A representative trace containing AMP sensing events is shown as a demonstration. The measurement was carried out as described in Methods in Example 2. The chambers were filled with a 1.5 M KCl buffer (1.5 M KCl, 10 mM MOPS, pH 7.0). A transmembrane potential of +200 mV was continually applied. AMP was added to cis with a final concentration of 300 μM. The open pore current (Iₚ), the residual current (I_{b}), the dwell time (τ_{off}) and the inter-event duration (τₒₙ) are defined as marked on the trace. The percentage blockage (% I_{b}) is defined as (Iₚ−I_{b})/Iₚ. The noise amplitude (S.D.) is defined as the standard deviation of the blockage level.

FIG. 47: The binding kinetics of CMP. (a-e) Representative traces acquired with varying CMP concentrations. The measurements were carried out as described in Methods in Example 2 in a buffer of 1.5 M KCl, 10 mM MOPS, pH 7.0. A potential of +200 m V was continually applied. CMP was added to cis with a final concentration of 100-500 μM. (f) Plot of 1/τ_{off} versus the CMP concentration. (g) Plot of 1/τₒₙ versus the CMP concentration. Three independent measurements were performed to generate the statistics. Generally, 1/τ_{off} remains almost constant with varying CMP concentrations. Whereas 1/τₒₙ increases when the CMP concentration is increased. For each condition, events acquired from a 5 min recording were employed to generate the statistics in (f) and (g).

FIG. 48: The binding kinetics of UMP. (a-e) Representative traces acquired with varying UMP concentrations. The measurements were carried out as described in Methods in a buffer of 1.5 M KCl, 10 mM MOPS, pH 7.0. A potential of +200 mV was continually applied. UMP was added to cis with a final concentration of 100-500 μM. (f) Plot of 1/τ_{off} versus the UMP concentration. (g) Plot of 1/τₒₙ versus the UMP concentration. Three independent measurements were performed to gather the statistics. Generally, 1/τ_{off} stays almost constant with varying UMP concentrations. Whereas 1/τₒₙ increases when the UMP concentration is increased. For each condition, events acquired from a 5 min recording were employed to generate the statistics in (f) and (g).

FIG. 49: The binding kinetics of AMP. (a-e) Representative traces acquired with varying AMP concentrations. The measurements were carried out as described in Methods in Example 2 in a buffer of 1.5 M KCl, 10 mM MOPS, pH 7.0. A potential of +200 mV was continually applied. AMP was added to cis with a final concentration of 100-500 μM. (f) Plot of 1/τ_{off} versus the AMP concentration. (g) Plot of 1/τₒₙ versus the AMP concentration. Three independent measurements were performed to form the statistics. Generally, 1/τ_{off} stays almost constant with varying AMP concentrations. Whereas 1/τₒₙ increases when the AMP concentration is increased. For each condition, events acquired from a 5 min recording were employed to generate the statistics in (f) and (g).

FIG. 50: The binding kinetics of GMP. (a-e) Representative traces acquired with varying GMP concentrations. The measurements were carried out as described in Methods in Example 2 in a buffer of 1.5 M KCl, 10 mM MOPS, pH 7.0. A potential of +200 m V was continually applied. GMP was added to cis with a final concentration of 100-500 μM. (f) Plot of 1/τ_{off} versus the GMP concentration. (g) Plot of 1/τₒₙ versus the GMP concentration. Three independent measurements were performed to form the statistics. Generally, 1/τ_{off} remains almost constant with varying GMP concentrations. Whereas, 1/τₒₙ increases when the GMP concentration is increased. For each condition, events acquired from a 5 min recording were employed to generate the statistics in (f) and (g).

FIG. 51: The binding kinetics of AMP at different voltages. (a-e) Representative traces of AMP sensing acquired with varying voltages. The measurements were carried out as described in Methods in Example 2 in a buffer of 1.5 M KCl, 10 mM MOPS, pH 7.0. AMP was added to cis with a final concentration of 500 μM. The applied transmembrane potential ranges from +40 mV to +200 mV, as also marked above each trace. (f) Plot of 1/τ_{off} versus the voltage. (g) Plot of 1/τₒₙ versus the voltage. Three independent measurements were performed to form the statistics. When the voltage is increased, 1/τ_{off} decreases but 1/τₒₙ increases. The result suggests that a higher applied voltage increases the rate of event appearance but the event dwell time is systematically decreased. For each condition, events acquired from a 5 min recording were employed to generate the statistics in (f) and (g).

FIG. 52: Distinguishing of NMPs performed at different voltages. (a) Representative binding events of CMP, UMP, AMP and GMP acquired at +100 mV. (b) Histogram of the % I_{b} derived from results of simultaneous sensing of CMP, UMP, AMP and GMP at +100 mV. Events acquired from a 10 min-recording were employed to generate the statistics. AMP and GMP events are indistinguishable in this condition. (c) Representative binding events of four NMPs measured at +150 mV. (d) Histogram of the % I_{b} derived from results of simultaneous sensing of four NMPs measured at +150 m V. Events acquired from a 10 min-recording were employed to generate the statistics. The blockage amplitude of AMP and GMP events are barely distinguishable in this condition. (e) Representative binding events of four NMPs acquired at +200 mV. (f) Histogram of % I_{b} derived from results of simultaneous sensing of four NMPs measured at +200 mV. Events acquired from a 10 min-recording were employed to generate the statistics. All four NMPs are now completely distinguishable. All measurements were carried out as described in Methods in Example 2. The chambers were filled with a 1.5 M KCl buffer (1.5 M KCl, 10 mM MOPS, pH 7.0). Different types of NMPs (CMP: 600 μM; UMP: 600 μM; AMP: 300 μM; GMP: 300 μM) were simultaneously added to cis. The concentration of CMP and UMP was set higher to balance the rate of event appearance during simultaneous sensing.

FIG. 53: Nanopore sensing of different NMPs. The measurements were carried out as described in Methods. The chambers were filled with a 1.5 M KCl buffer (1.5 M KCl, 10 mM MOPS, pH 7.0). A transmembrane potential of +200 mV was continually applied. NMPs were added to the cis side with a final concentration of 300 μM for each analyte. (a-d) Representative traces acquired with (a) CMP, (b) UMP, (c) AMP or (d) GMP as the sole analyte. (e-h) Event histogram of % I_{b} derived from results of (e) CMP, (f) UMP, (g) AMP and (h) GMP sensing. The blockage amplitudes are well-discriminated between four NMPs.

FIG. 54: Fitting results of NMP sensing events. Four parameters, % I_{b}, S.D., τ_{off}, τₒₙ were evaluated during single molecule sensing of NMPs. (a-d) Histogram of % I_{b}, S.D., τ_{off}, τₒₙ of (a) CMP, (b) UMP, (c) AMP or (d) GMP binding events. Histograms of % I_{b} and S.D. follow Gaussian distributions, from which the mean percentage blockage (%̅ I̅_̅{̅b̅}̅) and the mean noise amplitude (S̅.̅D̅.̅) were derived. Histograms of τ_{off} and τₒₙ were singly exponentially fitted. The mean dwell time (τ_{off}) and the mean inter-event interval (τₒₙ) were derived from corresponding fitting results. The measurements were carried out as described in Methods. The chambers were filled with a 1.5 M KCl buffer (1.5 M KCl, 10 mM MOPS, pH 7.0). A transmembrane potential of +200 mV was continually applied. NMPs were respectively added to cis with a final concentration of 300 μM for each analyte.

FIG. 55: Comparison of characteristic event parameters. (a-d) Comparison of (a) %̅ I̅_̅{̅b̅}̅, (b) S̅.̅D̅.̅, (c) τ_{off} and (d) τₒₙ of CMP, UMP, AMP and GMP. The measurements were carried out as described in Methods. The chambers were filled with a 1.5 M KCl buffer (1.5 M KCl, 10 mM MOPS, pH 7.0). A transmembrane potential of +200 mV was continually applied. NMPs were added to cis with a final concentration of 300 μM for each analyte. Three independent measurements (N=3) were performed to form the statistics.

FIG. 56: Sequential addition of NMPs during nanopore sensing. The measurements were carried out as described in Methods. The chambers were filled with a 1.5 M KCl buffer (1.5 M KCl, 10 mM MOPS, pH 7.0). CMP, UMP, AMP and GMP were sequentially added to cis. The final concentration of each NMP was 300 μM. Single channel recordings were continually performed at +200 mV for 20 minutes immediately after each addition. (a) A representative trace in the presence of CMP. Only CMP events (marked with the letter “C”) were observed. (b) The corresponding histogram of % I_{b}. The % I_{b} of CMP events was Gaussian fit. (c) A representative trace acquired immediately after the addition of UMP. UMP events (marked “U”) were observed afterwards. (d) The corresponding histogram of % I_{b}. The distribution of % I_{b} show two populations which were respectively Gaussian fit. (e) A representative trace acquired immediately after further addition of AMP. AMP events (marked “A”) were then observed in the trace. (f) The corresponding histogram of % I_{b}. Three event distributions can now be clearly seen in the histogram. (g) A representative trace acquired immediately after further addition of GMP. GMP events (marked “G”) demonstrate the deepest blockage amplitude. (h) The corresponding histogram of % I_{b}. Four fully distinguishable Gaussian distributions are clearly observed, respectively corresponding to CMP, UMP, AMP and GMP events.

FIG. 57: ¹H NMR spectrum of pseudouridine-5′-monophosphate (ψ). The preparation and characterization of w were provided by Wuxi AppTec.

FIG. 58: ¹H NMR spectrum of dihydrouridine-5′-monophosphate (D). The preparation and characterization of D were provided by Wuxi AppTec.

FIG. 59: Single molecule sensing of epigenetic NMPs. The measurements were carried out as described in Methods. The chambers were filled with a 1.5 M KCl buffer (1.5 M KCl, 10 mM MOPS, pH 7.0). A transmembrane potential of +200 mV was continually applied. Epigenetically modified NMPs were added to cis with a final concentration of 300 μM for each analyte. (a-g) Representative traces acquired respectively with (a) m⁵C, (b) m⁶A, (c) Ψ, (d) D, (e) I, (f) m⁷G or (g) m¹A as the sole analyte. (h-n) The corresponding event histograms of % I_{b} respectively derived from results of (h) m⁵C, (i) m⁶A, (j) Ψ, (k) D, (l) I, (m) m⁷G or (n) m¹A sensing.

FIG. 60: Demonstration of non-specific events. (a) The representative trace containing successive binding events of w. Few non-specific events (red arrow) were observed, which appeared as deeper blockades than that of ψ (marked with an orange dotted line). (b) The corresponding scatter plot of % I_{b} versus S.D. for ψ. The non-specific events demonstrate only 0.9% of all events being detected. (c) The representative trace containing successive binding events of m⁷G. Few non-specific events (red arrow) were observed, which appeared as shallower blockades than that of m⁷G (marked with a blue dotted line). (d) The corresponding scatter plot of % I_{b} versus S.D. for m⁷G. The non-specific events demonstrate 1.7% of all events being detected. These non-specific events may result from minor impurities in the sample. They don't interfere with the measurements as they produce only an extremely small fraction of events. The measurements were carried out as described in Methods in Example 2. The chambers were filled with a 1.5 M KCl buffer (1.5 M KCl, 10 mM MOPS, pH 7.0). A transmembrane potential of +200 mV was continually applied. ψ and m⁷G were respectively added to cis with a final concentration of 300 μM for each measurement.

FIG. 61: Statistics of sensing events of epigenetically modified NMPs. Four parameters, including % I_{b}, S.D., τ_{off}, τₒₙ were evaluated during single molecule sensing of modified NMPs. (a-g) Histogram of % I_{b}, S.D., τ_{off}, τₒₙ of (a) m⁵C, (b) m⁶A, (c) Ψ, (d) D, (e) I, (f) m⁷G or (g) m¹A binding events. The histograms of % I_{b} and S.D. follow Gaussian distributions, from which the mean percentage blockage (%̅ I̅_̅{̅b̅}̅) and the mean noise amplitude (S̅.̅D̅.̅) were derived. Histograms of τ_{off} and τₒₙ were singly exponentially fitted. The mean dwell time (τ_{off}) and the mean inter-event interval (τₒₙ) were derived from corresponding fitting results. The measurements were carried out as described in Methods in Example 2. The chambers were filled with a 1.5 M KCl buffer (1.5 M KCl, 10 mM MOPS, pH 7.0). A transmembrane potential of +200 mV was continually applied. Modified NMPs were added to cis with a final concentration of 300 μM for each analyte.

FIG. 62: Comparison of characteristic parameters of NMPs. (a-d) Comparison of (a) %̅ I̅_̅{̅b̅}̅, (b) S̅.̅D̅.̅, (c) τ_{off} and (d) τₒₙ of all eleven NMPs. The measurements were carried out as described in Methods. The chambers were filled with a 1.5 M KCl buffer (1.5 M KCl, 10 mM MOPS, pH 7.0). A transmembrane potential of +200 mV was continually applied. NMPs were added to cis with a final concentration of 300 μM for each analyte. Three independent measurements (N=3) were performed for each condition to produce the statistics.

FIG. 63: Sequential addition of epigenetic NMPs. The measurements were carried out as described in Methods in Example 2 with a 1.5 M KCl buffer (1.5 M KCl, 10 mM MOPS, pH 7.0). A +200 mV potential was continually applied during the measurement. In the presence of CMP, UMP, AMP and GMP, m⁵C, m⁶A, I, m⁷G, m¹A, Ψ, and D were sequentially added to cis. The final concentration of each NMP was 100 μM. Single channel recordings were continually performed for 10 minutes after each addition. (a) A representative trace in the presence of CMP, UMP, AMP and GMP. (b-g) Representative traces after the sequential addition of (b) m⁵C, (c) m⁶A, (d) I, (e) m⁷G, (f) m¹A, (g) ψ and (h) D. Each event was identified using the trained linear SVM model and the predicted identity is marked on top of each event with a dot of the corresponding color code (CMP: red; UMP: blue; AMP: green; GMP: purple; m⁵C: yellow, m⁶A: orchid, Ψ: orange, I: lime, D: cyan, m⁷G: teal, m¹A: pink).

FIG. 64: NMP identification by machine learning. The measurements were carried out as described in Methods in a 1.5 M KCl buffer (1.5 M KCl, 10 mM MOPS, pH 7.0). In the presence of CMP, UMP, AMP and GMP, m⁵C, m⁶A, I, m⁷G, m¹A, Ψ and D were sequentially added to cis. The final concentration of each NMP was 100 μM. Single channel recordings were continually performed at +200 mV for 10 minutes immediately after each NMP addition. Identification of NMP was performed with the trained linear SVM model. (a) Left: the scatter plot of % I_{b} versus S.D. in the presence of CMP, UMP, AMP and GMP. Events of CMP, UMP AMP and GMP were demonstrated with red, blue, green and purple dots respectively. Right: the corresponding histogram of event counts. (b-h) Left: the scatter plot of the % I_{b} versus S.D. after the successive addition of (b) m⁵C, (c) m⁶A, (d) I, (e) m⁷G, (f) m¹A, (g) ψ, (h) D. Right: the corresponding histogram of event counts after each NMP addition. With the linear SVM model, each newly added NMPs can be accurately identified. The grey circles in the scatter plots and the black arrows in the histograms indicate the distribution of events corresponding to the newly added NMP.

FIG. 65: The learning curves. Varying amount of training samples were fed into the machine learning model to evaluate the accuracy score of the model. The training score and the validation score were derived from the 10-fold cross-validation results. According to the learning curve, when the sample of the training set exceeds 176, the accuracy of validation has reached 0.990. When it exceeds 3124, the accuracy saturates at ˜0.996. The learning curves respectively produced with the training or the validation data merges with each other, confirming that overfitting of the model is not happening.

FIG. 66: Direct nanopore detection of methylated microRNA. The measurements were carried out with MspA-PBA, as described in Methods in Example 2. The chambers were filled with a 1.5 M KCl buffer (1.5 M KCl, 10 mM MOPS, pH 7.0). A transmembrane potential of +200 mV was applied continually. MicroRNAs were added to cis with a final concentration of 200 nM for each analyte. (a) A representative trace acquired with hsa-miR-21. (b) A representative trace acquired with hsa-miR-17. Only short residing spike events were observed for both analytes.

FIG. 67: MspA-PBA sensing of the stock solution of S1 nuclease and glycerol. The measurements were carried out as described in Methods in Example 2. The chambers were filled with a 1.5 M KCl buffer (1.5 M KCl, 10 mM MOPS, pH 7.0). A transmembrane potential of +200 mV was continually applied. (a) A representative trace acquired with the stock solution of S1 nuclease. The stock solution was added to cis with a final concentration of 1 U/μL. Successive binding events were observed. The events may result from the glycerol in the stock solution, which serves to minimize the damage to the S1 nuclease caused by repeated freezing and thawing. (b) A representative trace acquired with glycerol addition. Identical events appear with the addition of 1 μL 40% glycerol to the cis side. (c) The scatter plot of % I_{b} versus S.D. for the S1 nuclease stock solution and glycerol. The perfect alignment of event parameters as shown in (c) concludes that the observed events in the S1 nuclease stock solution is caused by glycerol. 400 successive events of each analyte were employed to generate the scatter plot.

FIG. 68: Ultrafiltration of the S1 nuclease solution. The measurements were carried out with MspA-PBA as described in Methods in Example 2. The chambers were filled with a 1.5 M KCl buffer (1.5 M KCl, 10 mM MOPS, pH 7.0). A transmembrane potential of +200 mV was continually applied. (a) A representative trace acquired with the S1 nuclease stock solution. The S1 nuclease stock solution was added to cis with a final concentration of 1 U/μL. The glycerol in the solution can report characteristic binding events. To minimize interferences of the glycerol, the S1 nuclease stock solution can be removed by ultrafiltration. (b) A representative trace acquired with the S1 nuclease solution after two runs of ultrafiltration. During each centrifugation operation, the S1 nuclease solution was added to the centrifugal filter with a 10 kDa-MWCO and centrifuged at 8000 rpm for 60 min at 4° C. Then the remaining solution in the filter device which contained S1 nuclease was collected. (Methods in Example 2). After two runs of ultrafiltration, S1 nuclease was added to cis with a final concentration of 1 U/μL. A significant decrease in the glycerol binding events was observed. (c) A representative trace acquired with the S1 nuclease solution after four runs of ultrafiltration. Most glycerol events disappeared. The S1 nuclease stock solution pre-treated with four turns of ultrafiltration was thus used for all subsequent RNA digestion experiments.

FIG. 69: Gel electrophoresis results of microRNA digestion. Briefly, this reaction was performed by mixing 150 μg microRNA (hsa-miR-21 or hsa-miR-17), 21 μL pre-treated S1 nuclease solution (180 U/μL), 6 μL 10× S1 nuclease buffer (300 mM CH₃COONa, 2800 mM NaCl 10 mM ZnSO₄, pH 4.6) and ultrapure water to a final volume of 60 μL. Then the mixture was kept at 23° C. for 4 h before gel electrophoresis. RNA samples were loaded on a 15% urea-PAGE gel. Gel electrophoresis was continually run for 60 min with a +200 V applied potential. The S1 nuclease was pretreated by ultrafiltration to remove glycerol in its stock solution (Methods in Example 2). Lane M: microRNA Marker (New England Biolabs). Lane 1: hsa-miR-21; L2: hsa-miR-21 treated with S1 nuclease; L3: hsa-miR-17; L4: hsa-miR-17 treated with S1 nuclease. The gel shows that both hsa-miR-21 and hsa-miR-17 were completely digested by S1 nuclease treatment. Operations of RNA digestion are detailed in Methods in Example 2.

FIG. 70: Identification of microRNA modification by SVM. (a) The scatter plot of % I_{b} versus S.D. for the digestion products of hsa-miR-21. The events demonstrated were acquired from a 60 min continuous recording. The events were predicted by the trained SVM model. Populations of AMP (red), UMP (blue), CMP (green), GMP (purple) and m° C. (yellow) were successfully detected, consistent with the composition of hsa-miR-21. Furthermore, the events of the remaining glycerol in the S1 nuclease stock solution were also accurately identified by the SVM model. (b) The corresponding histogram of event counts. (c) The scatter plot of % I_{b} versus S.D. for the digestion products of hsa-miR-17. The events demonstrated were acquired from a 60 min recording. An event distribution corresponding to m⁶A is clearly observed. (d) The corresponding histogram of event counts. The measurements were carried out with MspA-PBA as described in Methods in Example 2. The chambers were filled with a 1.5 M KCl buffer (1.5 M KCl, 10 mM MOPS, pH 7.0). A transmembrane potential of +200 mV was continually applied. RNA digestion products were added to cis with a final concentration of 100 ng/μL. The corresponding NMP modifications were detected by the machine learning algorithm.

FIG. 71: MicroRNA composition identification by the SVM model. (a) The sequences of hsa-miR-21 and hsa-miR-17. (b) Comparison of the hsa-miR-21 composition between that derived from measurements and the true value. The measured values were determined and calibrated according to Methods in Example 2. The NMP composition of hsa-miR-21 is calculated to be 2.2 CMP, 6.8 μMP, 6.9 AMP, 4.9 GMP, 1.0 m⁵C The counts of C, G and m⁵C in hsa-miR-21 are generally consistent with the true value. (c) Comparison of the hsa-miR-17 composition between that derived from measurements and the true value. The NMP composition of hsa-miR-17 is calculated to be 4.5 CMP, 4.1 μMP, 6.5 AMP, 6.4 GMP, 1.1 m⁶C. The count of m⁶C. in hsa-miR-21 is also generally consistent with the true value.

FIG. 72: Removal of glycerol events using machine learning. During nanopore sensing of yeast tRNA^{phe} digestion products, both NMPs and residual glycerol in the S1 nuclease stock solution would report binding events. Acknowledging the high resolution of MspA, events of glycerol (FIG. 67) and NMPs are fully distinguishable from each other. The scatter plot on the left demonstrates all events acquired from a 240 min continuous recording of yeast tRNA^{phe} digestion products. Both glycerol and NMP events were observed in this scatter plot. The glycerol events contain a highly characteristic negative going spikes on top of the blockage level. By learning event characteristics of glycerol described by multiple event features, including % I_{b}, S.D., dwell time, skewness and kurtosis of events, glycerol events were automatically recognized and removed, generating a new set of data containing no glycerol events, as shown in the scatter plot on the right. The remaining data were further analyzed for NMP identification. Here, model training was performed using One-Class SVM with 400 glycerol events. Multiple event features including % I_{b}, S.D., dwell time, skewness and kurtosis of events were employed for training. Events acquired with yeast tRNA^{phe} digestion products were predicted by the model. According to the predicting results, events with a judgement score above zero were identified as glycerol events. The glycerol events were removed from the dataset to avoid interference with further data analysis.

FIG. 73: Outlier boundary analysis. In machine learning, the outlier detection, also called anomaly detection, is widely used to detect abnormal events that do not appear to belong to any previously trained data types. One-Class SVM, an anomaly detection algorithm, is trained to learn whether an event belongs to a previously trained group of data or not. If not, this event is labelled as an outlier event. Otherwise, it is labelled as an inlier event. (a) Outlier boundaries of eleven types of NMPs generated by One-Class SVM. Left: The scatter plot of % I_{b} versus S.D. for eleven types of NMPs. The events were acquired from eleven independent measurements, in which eleven types of NMP were respectively added to cis as the sole analyte. Right: Corresponding outlier boundaries. The boundary separating the outliers from the inliers occurs where the contour value is 0. (b) Inlier and outlier detection of events generated by tRNA^{phe} digestion products. Left: The scatter plot of % I_{b} versus S.D. for tRNA^{phe} digestion products. Glycerol events have already been removed by machine learning (FIG. 72). Right: Separation of inliers and outliers. The inliers are marked with black dots, which are events that belong to one of the previously trained NMP types. The outliers, which don't belong to any previously trained NMP types, are marked with grey dots.

FIG. 74: Identification of NMPs using supervised and unsupervised learning. (a) The scatter plot of % I_{b} versus S.D. for NMPs of yeast tRNA^{phe} digestion products. Events of glycerol were automatically removed from the scatter plot by machine learning (FIG. 72). The events are further divided into inlier events (b) and outlier events (c) assisted by outlier boundary analysis (FIG. 73). (b) The scatter plot of % I_{b} versus S.D. for inliers. (d) Identification of inlier events using the Linear SVM model. Populations of AMP (red), UMP (blue), CMP (green), GMP (purple), m⁵C (yellow), ψ (orange), D (cyan), m^{7G} (teal) and m¹A (pink) were successfully identified, consistent with the composition of tRNA^{phe} Few m⁶A (purple) events were also detected, which may from minor background events that coincidently share the same event features of m⁶A or other RNAs. However, the proportion of m⁶A is extremely minor. (c) The scatter plot of % I_{b} versus S.D. for outliers. (e) Cluster analysis of outliers. DBSCAN, an unsupervised machine learning algorithm was employed to identify clustering events in the outlier events. The epsilon was set to 0.12 and the min_samples was set to 18. Four clusters of events were detected, which probably correspond to m²G, m₂²G, T, Y or other epigenetic NMPs in yeast tRNA^{phe}.

FIG. 75: The NMP profile of yeast tRNA^{phe} acquired from three independent trials. (a-c) Left: The scatter plot of % I_{b} versus S.D. acquired from NMP events acquired with enzymatically digested yeast tRNA^{phe}. The tRNA digestion products were added to cis with a final concentration of 100 ng/μl. a) 7925 events, b) 4292 events and c) 5093 events were respectively demonstrated in the scatter plot. Right: Comparison of the yeast tRNA^{phe} composition between that derived from measurements and the true value. Events were predicted by the trained SVM model and DBSCAN clustering (FIG. 74). All measurements were carried out using MspA-PBA as described in Methods in Example 2. The chambers were filled with a 1.5 M KCl buffer (1.5 M KCl, 10 mM MOPS, pH 7.0). A transmembrane potential of +200 mV was continually applied.

FIG. 76: NMP isomers resolved with tandem mass spectrum (MS/MS). MS/MS is a widely used method to identify isomers. It relies on collision induced dissociation (CID) to produce characteristic molecular fragments. But the identification of nucleotide isomers can be challenging, because the CID mass spectra of these isomers yield almost identical nucleobase ions (BH²⁺) from the same molecular ion (MH⁺). (a) Structures of m⁶A and m¹A. They are the isomers of methyladenine nucleotide. (b-c) Fingerprint MS/MS of (b) m⁶A and (c) m¹A. Their molecular ion peaks and fragment ion peaks are exactly the same. (d) Structures of U and ψ. The conversion of U into ψ is “mass-silent”, changing neither the mass of the RNA nor that of the modified residue. (e-f) Fingerprint MS/MS of (e) U and (f) ψ. Their molecular ion peaks are identical. The distinction between fragment ion peaks is insignificant. MS/MS experiments were performed on Agilent Q-TOF 6530B mass spectrometer equipped with an electrospray ionization (ESI) source. CID was employed for MS/MS fragmentation and the collision energy for CID was set as 16 V.

FIG. 77: Identification of alditols using MspA-PBA. (a) The structure of MspA-PBA and the family tree of alditols. The alditol family tree is derived from the aldose family tree. It includes C3-C6 alditols (FIG. 83). The alditols in each branch end of the tree are epimers. MspA-PBA is a hetero-octameric MspA composed of seven units of M2 MspA-D16H6 (grey) and one unit of 3-(maleimide) phenylboronic acid (MPBA) appended N90C MspA-H6 (blue). (b) The Fischer projection of alditols and their corresponding nanopore events. The measurements were carried out as described in Method 1 in Example 3 in a 1.5 M KCl buffer (1.5 M KCl, 10 mM MOPS, pH 7.0). A transmembrane potential of +100 mV was continually applied. Some alditols such as arabitol and the reduced D-lyxose, talitol and altritol have the same structure. Thus, all 15 types of aldoses correspond to 13 types of different alditols. (c) A comparison of the equilibrium binding constant (K_{b}) between PBA and typical alditols with different numbers of hydroxyl groups. K_{b} was derived according to the equation K_{b}=kₒₙ/k_{off}, as detailed in Table 18. (d) The scatter plot of percentage blockage (ratio, %) versus standard deviations of the blocking current (std, pA) from events acquired with 13 independent measurements (n=5129), during which 13 alditols were respectively added to cis (FIGS. 84-86).

FIG. 78: Discrimination of alditol epimers. (a) Fischer projection of glycerol and tetritols. Threitol and erythritol are epimers of each other, possessing opposite configurations at only one stereogenic center, C-2. They both have an extra pair of —H and —OH moieties when compared with glycerol, as colored in blue and red, respectively. (b) A representative trace containing blockage events of glycerol, threitol and erythritol. The analytes were simultaneously added to cis with a final concentration of 8 mM, 4 mM and 4 mM, respectively. Events were identified and marked with dark grey (glycerol), blue (threitol) or red (erythritol) bars below each corresponding trace. (c) The scatter plot of ratio vs. std generated from events as demonstrated in b (n=2147). (d) Fischer projection of pentitols. Arabitol has an opposite configuration at C-4 and C-2, different from that of xylitol. Adonitol and arabitol are epimers of each other, differing only in the stereochemistry at the C-2. (e) A representative trace containing events of adonitol, arabitol and xylitol. The analytes were simultaneously added to cis with a final concentration of 4 mM for each component. Events were identified and marked with royal (adonitol), arabitol (pink) or xylitol (green) bars below each corresponding trace. (f) The scatter plot of ratio vs. std generated from nanopore events as demonstrated in e (n=596). (g) The Fischer projection of hexitols. Allitol and talitol, talitol and dulcitol, D-sorbitol and mannitol, L-sorbitol and iditol are four pairs of epimers. (h) A representative trace containing events of hexitols when hexitols were simultaneously added to cis with a final concentration of 4 mM for each component. Events were identified and marked with dark yellow (allitol), orange (talitol), purple (dulcitol), sky-blue (D-sorbitol), wine (mannitol), brown (L-sorbitol) or dark cyan (iditol) bars below each corresponding trace. The raw continuous trace is shown in FIG. 94. (i) The scatter plot of ratio vs. std generated from blockades as demonstrated in h (n=1387). The measurements were carried out as described in Method 1 in Example 3 in a 1.5 M KCl buffer (1.5 M KCl, 10 mM MOPS, pH 7.0). A transmembrane potential of +100 mV was continually applied.

FIG. 79: Classification model training for alditol identification. (a) The construction of the training dataset. Nanopore events of glycerol (n=898), erythritol (n=559), threitol (n=546), arabitol (n=119), xylitol (n=319), adonitol (n=258), dulcitol (n=264), D-sorbitol (n=278), mannitol (n=134), allitol (n=481), talitol (n=517), iditol (n=403) and L-sorbitol (n=353) were collected to form the training dataset. Seven event features were extracted from the events to form a feature matrix. (b) The parallel coordinate plots. All features play a role during event classification. (c) The Classification Learner toolbox of MATLAB was used to train models for classification. A set of classifiers including decision trees, discriminant analysis, support vector machines (SVM), K nearest neighbors (KNN), naive Bayes, ensemble and neural network classifiers were evaluated. The validation accuracies were evaluated by 10-fold cross-validation. The quadratic SVM model, reporting a validation accuracy of 99.4%, is one of the optimum models. (d) The confusion matrix of alditol classification using a trained quadratic SVM model. The true positive rate (TPR) and the false negative rate (FNR) were also demonstrated on the right. (e) The learning curve with varying sample sizes of the training dataset. When the samples in the training dataset exceed 1450, the accuracy of validation reaches 0.994 and the validation accuracy and the training set re-substitution accuracy barely changed. The learning curve shows the mean accuracy of three independent tests.

FIG. 80: Machine learning-assisted alditol identification. (a) The flow diagram of the predictive process. The unclassified events were extracted from raw traces to form the predicting dataset when different alditols were sensed simultaneously in a mixture. The trained quadratic SVM model was applied to perform the predictions. (b) Representative traces containing simultaneous sensing events when glycerol, tetritols mixture (erythritol and threitol), pentitols mixture (xylitol, adonitol, arabitol) and hexitols mixture (D-/L-sorbitol, talitol, allitol, iditol, dulcitol, mannitol) were added sequentially to cis. The final concentration of glycerol is 6 mM, that of erythritol and threitol are both 4 mM, and that of the each other alditol is 2 mM. The measurements were carried out as described in Method 1 in Example 3 in a 1.5 M KCl buffer (1.5 M KCl, 10 mM MOPS, pH 7.0). A transmembrane potential of +100 mV was continually applied. Characteristic events from different alditols were automatically predicted and labeled with different bars (glycerol: dark gray, erythritol: red, threitol: blue, xylitol: green, adonitol: royal, arabitol: pink, D-sorbitol: sky-blue, dulcitol: purple, mannitol: wine, L-sorbitol: brown, talitol: orange, allitol: dark yellow and iditol: dark cyan). (c) Top: a scatter plot of ratio vs. std corresponding to the predicting dataset (n=2814). Each event was identified using the trained quadratic SVM model and demonstrated with colored dots. Bottom: The proportion of different alditol events determined with the quadratic SVM model. The color code of b and c is consistent with each other.

FIG. 81: Rapid identification of alditols from zero-sugar drinks using MspA-PBA. (a) A flow diagram of zero-sugar drink analysis using MspA-PBA. Four representative zero-sugar drinks (left) were respectively added to cis during independent measurements, each with a volume of 20 μL. All measurements were carried out as described in Method 1 in Example 3 in a 1.5 M KCl buffer (1.5 M KCl, 10 mM MOPS, pH 7.0). A transmembrane potential of +100 mV was continually applied. (b) A diagram of the predictive process. Comparing the event characteristics acquired from zero-sugar drinks and the alditol standards, the type of alditol in the drink could be immediately identified. (c, e, g, i) Representative traces containing the events of soda water (c), fruity water (e), sparkling water (g) and vitamin drink (i). Zoomed-in views of representative events marked with colored arrows are demonstrated on the right. (d, f, h, j) The scatter plots of ratio vs. std derived from sensing events of soda water (d, n=614), fruity water (f, n=950), sparkling water (h, n=781) and vitamin drink (j, n=423). Characteristic events of xylitol were identified in soda water (c). Erythritol was detected in fruity water (e), sparkling water (g) and vitamin drink (i) according to the statistics of events (FIG. 102) and the corresponding machine learning results (FIG. 103).

FIG. 82: Hetero-octameric MspA (N90C)₁(M2)₇ modified with 3-(maleimide)phenylboronic acid (MPBA). Measurements were carried out as described in S1 Methods 1, with a continually applied +100 mV voltage. (a) The structure diagram of a hetero-octameric MspA (N90C)₁(M2)₇. A sole reduced cysteine (orange) is designed for chemical modification to the pore constriction. (b) Single channel characterization of MspA (N90C)₁(M2)₇. The open pore current of a (N90C)₁(M2)₇ measures ˜306 pA. Additional upward noises were observed consistently. (c) The structure diagram of a PBA modified hetero-octameric MspA (MspA-PBA). The phenylboronic acid (PBA, blue) was introduced to the pore constriction via Michael addition. (d) Single channel characterization of MspA-PBA. The open pore current of a MspA-PBA measures ˜230 pA, which was lower than that of (N90C)₁(M2)₇ when measured in the same conditions. The previously observed upward noises also disappeared, confirming the success of MPBA modification. (e-f) I-V curves of MspA (N90C)₁(M2)₇ (e) and MspA-PBA (f). The I-V curves were acquired by applying a voltage ramp between −200 mV and +200 mV.

FIG. 83: The family tree of D-aldose and the corresponding alditols. The carbon atom in the aldehyde group and the corresponding methanol group is defined herein as C-1 in this paper, as shown by the red colored number in a and b. (a) D-Aldoses including triose, tetroses, pentoses and hexoses were shown in Fischer projection formulas. (b) Alditols include glycerol, tetritols, pentitols and hexitols. Some aldoses, such as D-arabinose and D-lyxose, D-altrose and D-talose have the same structure after reduction. Moreover, when glucitol is rotated 180°, it is identical to L-sorbitol.

FIG. 84: Single molecule characterization of glycerol and tetritols. All measurements were carried out as described in SI Methods 1. A transmembrane potential of +100 mV was continually applied. (a) Left: Fischer projection of glycerol; Middle: a representative trace containing blockage events of glycerol. The final concentration of glycerol is 8 mM; Right: a zoomed-in view of a representative event of glycerol. (b) The event scatter plot of the percentage blockage (ratio) versus the standard deviations of the blocking current (std) generated from results as demonstrated in a (n=898). (c) Left: Fischer projection of erythritol. Compared with glycerol, erythritol has an extra pair of —H and —OH as colored in blue and red, respectively. Middle: a representative trace containing blockage events of erythritol. The final concentration of erythritol is 4 mM; Right: a zoomed-in view of a representative event of erythritol. (d) The scatter plot of ratio vs. std generated from blockage events as demonstrated in c (n=559). (e) Left: Fischer projection of threitol. Threitol and erythritol are a pair of epimers which have opposite configuration at only one stereogenic center (C-2) as colored in c and d. Middle: a representative trace containing blockage events of threitol. The final concentration of threitol is 4 mM; Right: a zoomed-in view of a representative event of threitol, which reports unique noise fluctuations. (f) A scatter plot of ratio vs. std generated from blockades as demonstrated in e (n=546).

FIG. 85: Single molecule identification of pentitols. Xylitol, arabitol and adonitol are diastereomeric pentitols. All measurements were carried out as described in Methods 1 in Example 3. A transmembrane potential of +100 mV was continually applied. (a) Left: Fischer projection of xylitol; Middle: a representative trace containing events of xylitol. The final concentration of xylitol is 4 mM; Right: a zoomed-in view of a representative event of xylitol which has unique noise fluctuations. (b) A scatter plot of ratio vs. std generated from blockage events as demonstrated in a (n=319). (c) Left: Fischer projection of arabitol, which has a configuration at C-4 opposite to that of C-2 of xylitol as colored in a and c. Middle: a representative trace containing events of arabitol. The final concentration of arabitol is 4 mM; Right: a zoomed-in view of a representative event of arabitol. (d) A scatter plot of ratio vs. std generated from blockage events as demonstrated in c (n=119). (e) Left: Fischer projection of adonitol. Adonitol and arabitol are epimers which differ only in the stereochemistry at the C-2 position as colored in c and d. Middle: a representative trace containing blockage events of adonitol. The final concentration of adonitol is 4 mM; Right: a zoomed-in view of a representative event of adonitol which has the lowest amplitude fluctuations on the blockade among the three pentitols. (f) A scatter plot of ratio vs. std generated from events as demonstrated in e (n=258).

FIG. 86: Single molecule identification of hexitols. Sorbitol (D-sorbitol), mannitol, dulcitol, talitol, allitol, iditol and glulitol (L-sorbitol) are diastereomeric hexitols. All measurements were carried out as described in Methods 1 in Example 3. A transmembrane potential of +100 mV was continually applied. The final concentration of each hexitol is 4 mM in each independent measurement. (a, c) Left: Fischer projection of D-sorbitol (a), mannitol (c), which are epimers with a different stereochemistry at the C-2 position as colored in a and c; Middle: a representative trace containing blockage events of D-sorbitol (a) and mannitol (c); Right: a zoomed-in view of a representative event of D-sorbitol (a) and mannitol (c). (b, d) A scatter plot of ratio vs. std generated from the blockage events as demonstrated in a and c (n=278 and 134, respectively). (e, g, i) Left: Fischer projection of dulcitol (e), talitol (g) and allitol (i). Dulcitol and talitol are epimers with a different stereochemistry at the C-2 position as colored in e and g. Talitol and allitol are epimers; talitol has an opposite configuration at C-5 than the C-2 of allitol as colored in g and i. Middle: a representative trace containing events of dulcitol (e), talitol (g) and allitol (i); Right: a zoomed-in view of a representative event of dulcitol (e), talitol (g) and allitol (i). (f, h, j) A scatter plot of ratio vs. std generated from blockage events as shown in e (n=264), g (n=517), and i (n=481). (k, m) Left: Fischer projection of iditol (k) and L-sorbitol (m). Iditol and L-sorbitol are a pair of epimers which differ only in the stereochemistry at the C-2 position as colored in k and m. Middle: a representative trace containing blockage events of iditol (k) and L-sorbitol (m); Right: a zoomed-in view of a representative event of iditol (k) and L-sorbitol (m). (1, n) A scatter plot of ratio vs. std generated from blockades as shown in k and m (n=403 and 353, respectively). All above hexitols have unique blockade noise features when characterized by nanopore.

FIG. 87: Alditol sensing using M2 MspA nanopore. Glycerol, erythritol, xylitol and sorbitol were used as the representative alditols with different number of hydroxyl groups. All measurements were carried out as described in Methods 1 in Example 3. Octameric M2 MspA was applied as the nanopore sensor. A transmembrane potential of +100 m V was continually applied. Each alditol was added to cis with a final concentration of 4 mM. No binding events were observed during any above described measurements, confirming that alditols cannot be directly detected by M2 MspA without a boronic acid appendant.

FIG. 88: Definition of event parameters. (a) A representative electrophysiology trace containing nanopore events. Xylitol was treated as the model analyte. I₀ is the open pore current and I_{b} is the residual current caused by alditol blockade. ratio is derived from (I₀−I_{b})/I₀, which is defined as the percentage blockage. τ_{off} represents the dwell time of an event. τₒₙ represents the inter-event interval. std is the standard deviation value of the blockage level. (b-c) The histogram plot of ratio (b) and std (c) acquired from a continually recorded trace. The histograms were Gaussian fitted and the peaks of the fitting results respectively represent the mean value of ratio and the mean std. (d-e) The histogram plots of τ_{off} (d) and τₒₙ (e) acquired from a continually recorded trace. The histogram plots were respectively fit to a single exponential curve, according to the equation y=a*exp (−x/τ), from which the mean event dwell time (τ_{off}) (d) and the mean inter-event interval (τₒₙ) (e) were respectively derived. If not otherwise stated, all results in this manuscript were described by event parameters defined above.

FIG. 89: The binding kinetics of glycerol. (a-d) Representative traces acquired with varied glycerol concentrations. All measurements were carried out as described in Methods 1 in Example 3 in a buffer of 1.5 M KCl, 10 mM MOPS, pH 7.0. A transmembrane potential of +100 mV was continually applied. Glycerol was added to cis with a final concentration of 6-12 mM. (e) Plot of 1̅/̅τ̅ₒ̅ₙ̅ (red) or τ̅_̅{̅o̅f̅f̅}̅ (green) versus the glycerol concentration. Error bars in (e) represent standard deviations derived from independent measurements (N=3). Generally, τ̅_̅{̅o̅f̅f̅}̅ remains almost unchanged with varying glycerol concentrations whereas 1̅/̅τ̅ₒ̅ₙ̅ increases when the glycerol concentration is increased. All results discussed above are detailed in Table S2.

FIG. 90: The binding kinetics of erythritol. (a-d) Representative traces acquired with varying erythritol concentrations. All measurements were carried out as described in Methods 1 in Example 3 in a buffer of 1.5 M KCl, 10 mM MOPS, pH 7.0. A transmembrane potential of +100 mV was continually applied. Erythritol was added to cis with a final concentration of 2-8 mM. (e) Plot of 1̅/̅τ̅ₒ̅ₙ̅ (red) or τ̅_̅{̅o̅f̅f̅}̅ (green) versus the erythritol concentration. Error bars in (e) represent standard deviations between independent measurements (N=3). Generally, τ̅_̅{̅o̅f̅f̅}̅ remains almost unchanged with varying erythritol concentrations, whereas 1̅/̅τ̅ₒ̅ₙ̅ increases when the erythritol concentration is increased. All results discussed above are detailed in Table 18.

FIG. 91: The binding kinetics of xylitol. (a-d) Representative traces acquired with various xylitol concentrations. All measurements were carried out as described in Methods 1 in Example 3 in a buffer of 1.5 M KCl, 10 mM MOPS, pH 7.0. A transmembrane potential of +100 mV was continually applied. Xylitol was added to cis with a final concentration of 2-8 mM. (e) Plot of 1̅/̅τ̅ₒ̅ₙ̅ (red) or τ̅_̅{̅o̅f̅f̅}̅ (green) versus the xylitol concentration. Error bars in (e) represent standard deviations between independent measurements (N=3). Generally, τ̅_̅{̅o̅f̅f̅}̅ remains essentially unchanged with varying xylitol concentrations, whereas 1̅/̅τ̅ₒ̅ₙ̅ increases when the xylitol concentration is increased. All results discussed above are detailed in Table 18.

FIG. 92: The binding kinetics of D-sorbitol. (a-d) Representative traces acquired with various D-sorbitol concentrations. All measurements were carried out as described in Methods 1 in Example 3 in a buffer of 1.5 M KCl, 10 mM MOPS, pH 7.0. A transmembrane potential of +100 mV was continually applied. D-sorbitol was added to cis with a final concentration of 2-8 mM. (e) Plot of 1̅/̅τ̅ₒ̅ₙ̅ (red) or τ̅_̅{̅o̅f̅f̅}̅ (green) versus the D-sorbitol concentration. Error bars in (e) represent standard deviations between independent measurements (N=3). Generally, τ̅_̅{̅o̅f̅f̅}̅ remains almost unchanged with varying D-sorbitol concentrations, whereas 1̅/̅τ̅ₒ̅ₙ̅ increases when the D-sorbitol concentration is increased. All results discussed above are detailed in Table 18.

FIG. 93: The binding kinetics of representative alditols at different voltages. All measurements were carried out as described in Methods 1 in Example 3 in a buffer of 1.5 M KCl, 10 mM MOPS, pH 7.0. The applied potential was increased from 60 to 120 m V. Glycerol was added to cis with a final concentration of 12 mM. Other alditols were added to cis with a final concentration of 8 mM for each component, respectively. The reciprocal of event dwell time (1/τ_{off}) and inter-event interval (1/τₒₙ) of glycerol (a), erythritol (b), xylitol (c) and D-sorbitol (d) were plotted versus the applied voltages. Error bars in a-d represent standard deviations between independent measurements (N=3). The results are also detailed in Table 19. Generally, 1/τ_{off} and 1/τₒₙ stay essentially unchanged at different applied potential.

FIG. 94: (a) The raw trace of FIG. 78h. All nanopore events are marked with dark yellow (allitol), orange (talitol), purple (dulcitol), sky-blue (D-sorbitol), wine (mannitol), brown (L-sorbitol) or dark cyan (iditol) bars on the top of each corresponding trace. (b) A zoomed in view of each hexitol event in a. The digital number of each event is marked above the trace in a and b. Events from different hexitols can be clearly distinguished from each other.

FIG. 95: A workflow of event feature extraction. All nanopore sensing events are first automatically detected with the “single channel research” function in Clampfit 10.7. The event start-time (tₛₜₐᵣₜ) and the event end-time (t_{end}) of each event are recorded as time stamps for each event in a .txt file. The ignored duration is set to 2 ms to preclude events caused by transient collision of the analyte with the pore. The Axon abf file and txt file are imported into MATLAB to extract all event features (Methods 1 in Example 3). Seven event features including percentage blockage (ratio), standard deviation (std), kurtosis (kurt), skewness (skew), dwell time (time), central value of distribution (peak) and noise (FWHM) are extracted to form a feature matrix. The feature matrix was exported as a .xlsx file and is applied to perform machine learning.

FIG. 96: Evaluation of different models. The parameter settings with the best verification accuracy and the lowest cost of each model are demonstrated. All models were trained using the Classification Learner toolbox in MATLAB with the training dataset containing the feature matrix of 13 alditols. The accuracies were derived from 10-fold cross-validation results. The quadratic SVM is the best model which has the highest accuracy and the lowest total cost.

FIG. 97: Glycerol and tetritols identification by machine learning. The measurements were carried out as described in Methods 1 in Example 3 in a buffer of 1.5 M KCl, 10 mM MOPS, pH 7.0. A transmembrane potential of +100 mV was continually applied. Glycerol, erythritol and threitol were sequentially added to cis. (a) A scatter plot of training dataset (ratio vs. std) as a comparison. (b) Left: the scatter plot of ratio vs. std in the presence of glycerol with a final concentration of 8 mM (n=693). Events of glycerol were identified using the trained quadratic SVM model and demonstrated with dark gray dots. Right: the corresponding histogram of the proportion of analytes. (c) Left: the scatter plot of ratio vs. std after successive addition of erythritol with a final concentration of 4 mM (n=1583). Each event was identified using the trained quadratic SVM model and the events of erythritol were demonstrated with red dots. Right: the corresponding histogram of the proportion of analytes. (d) Left: the scatter plot of ratio vs. std after successive addition of threitol with a final concentration of 4 mM (n=2147). Each event was identified using the trained quadratic SVM model and the events of threitol were demonstrated with blue dots. Right: the corresponding histogram of the proportion of analytes. Each time a new tetritol was added, the prediction results would report the appearance of the corresponding tetritol type.

FIG. 98: Pentitol identification by machine learning. The measurements were carried out as described in Methods 1 in Example 3 in a buffer of 1.5 M KCl, 10 mM MOPS, pH 7.0. A transmembrane potential of +100 mV was continually applied. Adonitol, xylitol and arabitol were sequentially added to cis with a final concentration of 4 mM for each component. (a) A scatter plot of training dataset (ratio vs. std) as a reference. (b) Left: the scatter plot of ratio vs. std in the presence of adonitol (n=257). Events of adonitol were identified using the trained quadratic SVM model and demonstrated with royal dots. Right: the corresponding histogram of the proportion of analytes. (c) Left: the scatter plot of ratio vs. std after successive addition of xylitol (n=491). Each event was identified using the trained quadratic SVM model and the events of xylitol were demonstrated with green dots. Right: the corresponding histogram of the proportion of analytes. (d) Left: the scatter plot of ratio vs. std after successive addition of arabitol (n=596). Each event was identified using the trained quadratic SVM model and the events of arabitol were demonstrated with pink dots. Right: the corresponding histogram of the proportion of analytes. Generally, each time when a new type of pentitol was added, the prediction results would report the appearance of the corresponding pentitol type.

FIG. 99: Hexitol identification by machine learning. The measurements were carried out as described in Methods 1 in Example 3 in a buffer of 1.5 M KCl, 10 mM MOPS, pH 7.0. A transmembrane potential of +100 mV was continually applied. D-sorbitol, dulcitol, mannitol, L-sorbitol, talitol, allitol and iditol were sequentially added to cis with a final concentration of 4 mM. (a) A scatter plot of training dataset (ratio vs. std) as a reference. (b-h) Left: the scatter plot of ratio vs. std after each addition of the hexitols (n=316, 490, 720, 688, 797, 941, 1387). Each event was identified using the trained quadratic SVM model and demonstrated with corresponding color dots (D-sorbitol: sky-blue, dulcitol: purple, mannitol: wine, L-sorbitol: brown, talitol: orange, allitol: dark yellow and iditol: dark cyan). Right: the corresponding histogram of the proportion of analytes determined with the quadratic SVM model. Generally, each time when a new pentitol was added, the prediction results report the appearance of the corresponding pentitol type.

FIG. 100: Identification of alditols with different numbers of hydroxyl groups by machine learning. The measurements were carried out as described in Methods 1 in Example 3 in a buffer of 1.5 M KCl, 10 mM MOPS, pH 7.0. A transmembrane potential of +100 mV was continually applied. (a-d) Representative traces acquired when glycerol, tetritols mixture (erythritol and threitol), pentitols mixture (xylitol, adonitol, arabitol) and hexitols mixture (D-/L-sorbitol, talitol, allitol, iditol, dulcitol, mannitol) were sequentially added to cis with a final concentration of 6 mM, 4 mM for each tetritol, 2 mM for each pentitol and 2 mM for each hexitol, respectively. The corresponding nanopore events were marked with colored arrows (glycerol: dark gray, erythritol: red, threitol: blue, xylitol: green, adonitol: royal, arabitol: pink, D-sorbitol: sky-blue, dulcitol: purple, mannitol: wine, L-sorbitol: brown, talitol: orange, allitol: dark yellow and iditol: dark cyan). (e-h) Left: the scatter plot of ratio vs. std after each addition of the alditols (n=1729, 2265, 2230, 2814). Each event was identified using the trained quadratic SVM model and shown with corresponding color dot labels. The color code is consistent with that defined in a-d. Right: the corresponding histogram of the proportion of analytes determined with the quadratic SVM model. Each time alditols with different number of hydroxyl groups was added, the prediction results would report the appearance of the corresponding alditol type.

FIG. 101: Quick analysis of zero-sugar drinks using MspA-PBA. The measurements were carried out as described in Methods 1 in Example 3 and FIG. 81 in a buffer of 1.5 M KCl, 10 mM MOPS, pH 7.0. A transmembrane potential of +100 mV was continually applied. The commercially available zero-sugar drinks including fruity water, soda water, vitamin drink and sparkling water were added to the cis chamber independently with a volume of 20 μL. 3 seconds of magnetic stirring was performed to reach a homogenous analyte distribution in the chamber. Blocking events with characteristic resistive pulses were observed immediately. A 15 min trace containing pore blocking events were sufficient for subsequent data analysis.

FIG. 102: Statistics of sensing events of the zero-sugar drinks. The current percentage blockage (ratio, %) and amplitude std (pA) were evaluated during single molecule sensing of the zero-sugar drinks. The measurements were carried out as described in Methods 1 in Example 3 and FIG. 81 in a buffer of 1.5 M KCl, 10 mM MOPS, pH 7.0. A transmembrane potential of +100 mV was continually applied. The fruity water, soda water, vitamin drink and sparkling water were added to the cis chamber independently. An added volume of 20 μL was applied for each drink sample. (a-h) Histograms of ratio and std of soda water (a, b), fruity water (c, d), sparkling water (e, f) or vitamin drink (g, h). As described in FIG. 84, the histogram was Gaussian fitted and the peak of the fitting results represents the mean value of ratio and the mean std. According to the statistics of alditol standards, it is speculated that the sweetener in fruity water, vitamin drink and sparkling water is erythritol, while the soda water was confirmed to contain xylitol instead.

FIG. 103: Machine learning assisted alditol identification in zero-sugar drinks. (a) A scatter plot of cluster assignments and centroids corresponding to vitamin drink events. The sensing events were clustered using k-means clustering in MATLAB. Events in cluster 1 (red, n=393) which correspond to the major component in vitamin drinks were extracted as predicting datasets of vitamin drink. (b) The corresponding silhouette plot of cluster analysis. It demonstrates a measure of how close each point in one cluster is to data points of neighboring clusters. Most data points in both clusters have a large silhouette value, greater than 0.8, indicating that those points are well-separated from neighboring clusters. (c-f) The histogram of the alditol proportion in soda water (c), fruity drink (d), sparkling water (e) and vitamin drink (f) from the predicting results of Quadratic SVM model. The results (c-f) show that the sweetener in fruity water, vitamin drink and sparkling water is erythritol, while the soda water contains xylitol.

FIG. 104: Single molecule sensing of glycerol by MspA-PBA. (a) The chemical structure of glycerol. (b) A representative trace of glycerol sensing. The trace was acquired when a +200 mV bias was continuously applied and 1 μL glycerol was added to cis compartment. A 1.5 M KCl, 10 mM MOPS, pH 7.0 buffer was used. Iₚ stands for the open pore current of the MPBA modified (N90C)₁(M2)₇ MspA. Iₙ stands for the blockage level when a glycerol was bound to the pore. (c) The scatter plot of the ΔI/Iₚ vs the standard deviation (S.D.) when glycerol was sensed as the sole analyte. ΔI=Iₚ−Iₙ. 493 events (n=493) were included in the plot.

FIG. 105: Single molecule sensing of D-Sorbitol by MspA-PBA. (a) The chemical structure of D-Sorbitol. (b) A representative trace of D-Sorbitol sensing. The trace was acquired when a +100 mV bias was continuously applied and D-Sorbitol was added to cis with a 4 mM final concentration. A 1.5 M KCl, 10 mM MOPS, pH 7.0 buffer was used. Iₚ stands for the open pore current of the MPBA modified (N90C)₁(M2)₇ MspA. Iₐ stands for the blockage level when a D-Sorbitol was bound to the pore. (c) The scatter plot of the ΔI/Iₚ VS the standard deviation (S.D.) when D-Sorbitol was sensed as the sole analyte. ΔI=Iₚ−Iₐ. 263 events (n=263) were included in the plot.

FIG. 106: Single molecule sensing of Leucrose by MspA-PBA. (a) The chemical structure of Leucrose. (b) A representative trace of Leucrose sensing. The trace was acquired when a +160 m V bias was continuously applied and Leucrose was added to cis with a 20 mM final concentration. A 1.5 M KCl, 10 mM MOPS, pH 7.0 buffer was used. Iₚ stands for the open pore current of the MPBA modified (N90C)₁(M2)₇ MspA. Iₘ stands for the blockage level when a Leucrose was bound to the pore. Leucrose report two type of events, respectively denoted with roman numerals. (c) The scatter plot of the ΔI/Iₚ vs the standard deviation (S.D.) when Leucrose was sensed as the sole analyte. ΔI=Iₚ−Iₘ. 788 events (n=788) were included in the plot. Different event types, as marked with roman numerals, were respectively denoted on the scatter plot.

FIG. 107: Single molecule sensing of Acarbose by MspA-PBA. (a) The chemical structure of Acarbose. (b) A representative trace of Acarbose sensing. The trace was acquired when a +160 mV bias was continuously applied and Acarbose was added to cis with a 20 mM final concentration. A 1.5 M KCl, 10 mM MOPS, pH 7.0 buffer was used. Iₚ stands for the open pore current of the MPBA modified (N90C)₁(M2)₇ MspA. Iₘ stands for the blockage level when Acarbose was bound to the pore. Acarbose report two type of events, respectively denoted with roman numerals. (c) The scatter plot of the ΔI/Iₚ vs the standard deviation (S.D.) when Acarbose was sensed as the sole analyte. ΔI=Iₘ−Iₚ. 225 events (n=225) were included in the plot. Different event types, as marked with roman numerals, were respectively denoted on the scatter plot.

FIG. 108: Single molecule sensing of oligosaccharide by MspA-PBA. (a, d g) The chemical structure of oligosaccharide, Raffinose (a), Stachyose (d) and Verbascose (g) (b, e h) A representative trace of oligosaccharide sensing. The trace was acquired when a +160 m V bias was continuously applied and oligosaccharide was added to cis with a 20 mM final concentration. A 1.5 M KCl, 10 mM MOPS, pH 7.0 buffer was used. Iₚ stands for the open pore current of the MPBA modified (N90C)₁(M2)₇ MspA. Iₘ stands for the blockage level when an oligosaccharide was bound to the pore. Raffinose report one type of event (b). Stachyose report two type of events, respectively denoted with roman numerals (e). Verbascose report three type of events, respectively denoted with roman numerals (h). (c, f, i) The scatter plot of the ΔI/Iₚ vs the standard deviation (S.D.) when oligosaccharide was sensed as the sole analyte. ΔI=Iₚ−Iₛ. 597 events (n=597) were included in the plot of Raffinose. 229 events (n=229) were included in the plot of Stachyose. 224 events (n=224) were included in the plot of Verbascose. Different event types, as marked with roman numerals, were respectively denoted on the scatter plot.

FIG. 109: Single molecule sensing of grape juice by MspA-PBA. (a) The cartoon image of grape. (b). Schematic diagram of detecting grape juice contents by MspA-PBA. Squeezed the juice of a fresh grape into a centrifuge tube and sucked the supernatant. 10 μL supernatant was added to cis compartment. (c). A representative trace of grape juice sensing. The trace was acquired when a +160 mV bias was continuously applied. A 1.5 M KCl, 10 mM MOPS, pH 7.0 buffer was used. Iₚ stands for the open pore current of the MPBA modified (N90C)₁(M2)₇ MspA. I_{g} stands for the blockage level when the contents in grape juice (fructose or tartaric acid, etc.) was bound to the pore. The saccharide signal is below Iₚ, and the acid signal is above Iₚ.

FIG. 110: Single nucleoside diphosphate (NDP) discrimination with MspA-PBA. Phenylboronic acid (PBA) is known to rapidly form a cyclic boronate esters with cis-diols of ribose in NDPs. When driven to pass through the pore by the electrical force, NDPs can reversibly bind with PBA, generating characteristic blockade currents related to their nucleobases. (a) The general chemical structure of NDPs. (b) The chemical structures of nucleobases of cytidine diphosphate (CDP), adenosine diphosphate (ADP), uridine diphosphate (UDP) and guanosine diphosphate (GDP). (c) A representative trace during simultaneous sensing of the four types of NDPs. Characteristic events from different NDPs are clearly recognized from the trace. Iₚ stands for the open pore current of the MPBA modified (N90C)₁(M2)₇ MspA. Different NDP stands for the blockage level when CDP, ADP, UDP and GDP were respectively bound to the pore. NDPs were simultaneously added to the cis side with a final concentration of 300 μM for each analyte. The measurements were carried out in a 1.5 M KCl buffer (1.5 M KCl, 10 mM MOPS, pH 7.0). A transmembrane potential of +200 m V was continuously applied. (d) The scatter plot of the ΔI/Iₚ vs the standard deviation (S.D.) when NDPs was sensed at the same time. Four distinct distributions are clearly observed without any overlaps.

FIG. 111: Single nucleoside triphosphate (NTP) discrimination with MspA-PBA. Phenylboronic acid (PBA) is known to rapidly form a cyclic boronate esters with cis-diols of ribose in NTPs. When driven to pass through the pore by the electrical force, NTPs can reversibly bind with PBA, generating characteristic blockade currents related to their nucleobases. (a) The general chemical structure of NTPs. (b) The chemical structures of nucleobases of cytidine triphosphate (CTP), adenosine triphosphate (ATP), uridine triphosphate (UTP) and guanosine triphosphate (GTP). (c) A representative trace during simultaneous sensing of the four types of NTPs. Characteristic events from different NTPs are clearly recognized from the trace. Iₚ stands for the open pore current of the MPBA modified (N90C)₁(M2)₇ MspA. Different NTP stands for the blockage level when CTP, ATP, UTP and GTP were respectively bound to the pore. NTPs were simultaneously added to the cis side with a final concentration of 300 μM for each analyte. The measurements were carried out in a 1.5 M KCl buffer (1.5 M KCl, 10 mM MOPS, pH 7.0). A transmembrane potential of +200 mV was continuously applied. (d) The scatter plot of the ΔI/Iₚ vs the standard deviation (S.D.) when NTPs were sensed at the same time. Four distinct distributions are clearly observed without any overlaps.

FIG. 112: Single molecule sensing of tris(hydroxymethyl)methyl aminomethane by MspA-PBA. (a) The chemical structure of Tris(hydroxymethyl)methyl aminomethane. (b) A representative trace of tris(hydroxymethyl)methyl aminomethane sensing. The trace was acquired when a +160 mV bias was continuously applied and tris(hydroxymethyl)methyl aminomethane was added to cis with a 10 mM final concentration. A 1.5 M KCl, 10 mM MOPS, pH 7.0 buffer was used. Iₚ stands for the open pore current of the MPBA modified (N90C)₁(M2)₇ MspA. I_{T} stands for the blockage level when a tris(hydroxymethyl)methyl aminomethane was bound to the pore. (c) The scatter plot of the ΔI/Iₚ vs the standard deviation (S.D.) when tris(hydroxymethyl)methyl aminomethane was sensed as the sole analyte. ΔI=Iₚ−I_{T}. 2139 events (n=2139) were included in the plot.

FIG. 113: Single molecule sensing of noradrenaline by MspA-PBA. (a) The chemical structure of noradrenaline. (b) A representative trace of noradrenaline sensing. The trace was acquired when a +180 mV bias was continuously applied and noradrenaline was added to cis with a 0.1 mM final concentration. A 0.5 M KCl, 10 mM HEPES, pH 7.0 buffer was used. Iₚ stands for the open pore current of the MPBA modified (N90C)₁(M2)₇ MspA. In stands for the blockage level when a glycerol was bound to the pore. (c) The scatter plot of the ΔI/Iₚ vs the standard deviation (S.D.) when noradrenaline was sensed as the sole analyte. ΔI=Iₚ−Iₙ. 371 events (n=371) were included in the plot.

FIG. 114: Single molecule sensing of Uridine DiphosPhate Glucose (UDPG) by MspA-PBA. (a) The chemical structure of UDPG. (b) A representative trace of UDPG sensing. The trace was acquired when a +100 mV bias was continuously applied and UDPG was added to cis with a 10 mM final concentration. A 1.5 M KCl, 10 mM MOPS, pH 7.0 buffer was used. Iₚ stands for the open pore current of the MPBA modified (N90C)₁(M2)₇ MspA. Iᵤ stands for the blockage level when a UDPG was bound to the pore. (c) The scatter plot of the ΔI/Iₚ vs the standard deviation (S.D.) when UDPG was sensed as the sole analyte. ΔI=Iₚ−Iᵤ. 89 events (n=89) were included in the plot.

FIG. 115: The preparation of a nickel-modified MspA for amino acid sensing (a) The mechanism of constructing nickel-modified MspA. NTA was chemically bonded to the only cysteine of (N90C)₁(M2)₇ MspA by maleimide-thiol coupling. Then nickel ions bind to NTA through coordination. (b) Single molecule demonstration of NTA and nickel modification to a (N90C)₁(M2)₇ MspA. Single channel recording was performed with a single (N90C)₁(M2)₇ MspA pore. A +100 mV bias was continuously applied. When an NTA was conjugated to the pore, an irreversible drop of current was observed. I₀ stands for the open pore current of (N90C)₁(M2)₇ and I_{T} stands for the open pore current of the NTA modified (N90C)₁(M2)₇ MspA. When a nickel was conjugated to the (N90C)₁(M2)₇ MspA-NTA, an irreversible drop of current was observed again. Ix stands for the open pore current of the nickel modified (N90C)₁(M2)₇ MspA-NTA. (c) The chemical structure of glycine (Gly). (d) A representative trace of Gly sensing. The trace was acquired when a +100 mV bias was continuously applied and Gly was added to cis with a 10 mM final concentration. A 1.5 M KCl, 10 mM MOPS, pH 7.0 buffer was used. I_{N} stands for the open pore current of the nickel modified (N90C)₁(M2)₇ MspA-NTA. I_{A} stands for the blockage level when a Gly was bound to the pore. (e) The scatter plot of the ΔI/Iₚ vs the standard deviation (S.D.) when Gly was sensed as the sole analyte. ΔI=I_{A}−I_{N}. 172 events (n=172) were included in the plot.

FIG. 116: Single molecule sensing of glycine (Gly) and lysine (Lys) by MspA-NTA-Ni. (a) The chemical structure of Gly. (b) The chemical structure of Lys. (c) A representative trace during simultaneous sensing of Gly and Lys. Characteristic events from different amino acid are clearly recognized from the trace. I_{N} stands for the open pore current of the nickel modified (N90C)₁(M2)₇ MspA-NTA. Different amino acid stands for the blockage level when Gly (blue) and Lys (green) were respectively bound to the pore. Amino acids were simultaneously added to the cis side with a final concentration of 10 mM for each analyte. The measurements were carried out in a 1.5 M KCl buffer (1.5 M KCl, 10 mM MOPS, pH 7.0). A transmembrane potential of +100 mV was continuously applied. (c) The scatter plot of the ΔI/Iₚ vs the standard deviation (S.D.) when amino acids were sensed at the same time. Two distinct distributions are clearly observed without any overlaps.

FIG. 117: The (N91C) 1 (M2)₇-MspA-PBA for L-Sorbose sensing. In order to expand the versatility of this method, we prepared the heterogeneously assembled MspA octamer with a mutation at position 91 to cysteine. Subsequently, MPBA [3-(maleimide) phenylboronic was modified in (N91C) (M2)₇-MspA and used to sense L-Sorbose. (a) The chemical structure of L-Sorbose (L-Sor). (b) A representative trace of L-Sorbose sensing. The trace was acquired when a −160 mV bias was continuously applied and L-Sorbose was added to cis with a 10 mM final concentration. A 1.5 M KCl, 10 mM MOPS, pH 7.0 buffer was used. Iₚ stands for the open pore current of the MPBA modified (N91C) (M2)₇ MspA. Is stands for the blockage level when a saccharide was bound to the pore. (c) The scatter plot of the ΔI/Iₚ vs the standard deviation (S.D.) when L-Sorbose was sensed as the sole analyte. ΔI=Iₛ−Iₚ. 174 events (n=174) were included in the plot.

FIG. 118: Single molecule sensing of tetrachloroaurate (III) by (N91M): (M2)₇ MspA. (a) The sensing mechanism with [AuCl₄]⁻ ions by (N91M) (M2)₇ MspA. The mutant MspA [(N91M)₁(M2)₇ MspA] possesses only one identical methionine residues at position 91, and capable of binding an [AuCl₄]⁻ ion. Subsequently, [AuCl₄]⁻ oxidizes methionine residues to sulfoxides. (b) A representative trace of tetrachloroaurate (III) sensing. The trace was acquired when a +100 mV bias was continuously applied and tetrachloroaurate (III) was added to cis with a 1 mM final concentration. A 1.5 M KCl, 10 mM HEPES, pH 7.0 buffer was used. Stage 1 for the open pore current of (N91M) (M2)₇ MspA. Stage 2 for the blockage events when an [AuCl₄]⁻ ion was bound to the pore. Stage 3 for the methionine residues were oxidized to sulfoxide in the pore.

FIG. 119: N-acetylcytidine-5-monophosphate (ac4C) sensing with MspA-PBA. (a) The structure of ac4C. ac4C is a modified CMP in which one of the exocyclic amino hydrogens is substituted by an acetyl group. (b) A representative current trace containing successive binding events of ac4C. The blockage level was marked with a dotted line. (c) The scatter plot of % I_{b} versus S.D. for ac4C. The measurements were carried out in a 1.5 M KCl buffer (1.5 M KCl, 10 mM MOPS, pH 7.0). A transmembrane potential of +200 m V was continually applied. Ac4C was added to cis with a final concentration of 300 μM.

FIG. 120: Nucleoside analogs molnupiravir identified by MspA-PBA. a. The chemical structure of molnupiravir. b. Two types of representative events of molnupiravir binding to a PBA. c. The scatter plot of % I_{b} versus τ_{off} for molnupiravir sensing events. The histogram of % I_{b} with corresponding Gaussian fitting results are placed in the right of the scatter plot. The two major populations were marked with type 1 or type 2 according their % I_{b} values. 457 events are included in the scatter plot. The events were extracted from a 25 min continually recorded trace with molnupiravir concentration set at 0.5 mM. d. A representative trace containing molnupiravir binding events. A buffer of 1.5 M KCl, 10 mM MOPS, pH 7.0 was used. A +100 mV potential was continually applied.

FIG. 121. Single molecule sensing of Simple Salvianolic acids by MspA-PBA. (a, d, g, j) The chemical structure of Protocatechualdehyde (a) Protocatechuic Acid (d), Caffeic Acid (g) and Salvianic acid A (j) (b, e, h. k) A representative trace of simple Salvianolic acids sensing. The trace was acquired when a +100 mV bias was continuously applied and Salvianolic acids was added to cis with a 1 mM final concentration. A 1.5 M KCl, 100 mM MOPS, pH 7.0 buffer was used. Iₚ stands for the open pore current of the MPBA modified (N90C)₁(M2)₇ MspA. Iₘ stands for the blockage level when a simple salvianolic acid was bound to the pore. Protocatechualdehyde, Protocatechuic Acid, Caffeic Acid report one type of event (b, e, h). Salvianic acid A report two type of events, respectively denoted with roman numerals (k). (c, f, i, I) The scatter plot of the ΔI/Iₚ vs the standard deviation (S.D.) when simple Salvianolic acids was sensed as the sole analyte. ΔI=Iₚ−Iₛ. 1417 events (n=1417) were included in the plot of Protocatechualdehyde. 503 events (n=503) were included in the plot of Protocatechuic Acid. 944 events (n=944) were included in the plot of Caffeic Acid. 529 events (n=529) were included in the plot of Salvianic acid A. Different event types, as marked with roman numerals, were respectively denoted on the scatter plot.

FIG. 122. Single molecule sensing of Complex Salvianolic acids by MspA-PBA. (a, d, g, j) The chemical structure of Rosmarinic Acid (a) Lithospermic Acid (d), Salvianolic Acid A (g) and Salvianolic Acid B (j) (b, e, h. k) A representative trace of complex Salvianolic acids sensing. The trace was acquired when a +100 mV bias was continuously applied and Salvianolic acids was added to cis with a 1 mM final concentration. A 1.5 M KCl, 100 mM MOPS, pH 7.0 buffer was used. Iₚ stands for the open pore current of the MPBA modified (N90C)₁(M2)₇ MspA. Iₘ stands for the blockage level when an oligosaccharide was bound to the pore. Rosmarinic Acid, Lithospermic Acid report two type of events and Salvianolic Acid A, Salvianolic Acid B report three type of events, all of which respectively denoted with roman numerals (b, e, h, k). (c, f, i, I) The scatter plot of the ΔI/Iₚ vs the standard deviation (S.D.) when the complex Salvianolic acid was sensed as the sole analyte. ΔI=Iₚ−Iₛ. 634 events (n=634) were included in the plot of Rosmarinic Acid. 541 events (n=541) were included in the plot of Lithospermic Acid. 377 events (n=377) were included in the plot of Salvianolic Acid A. 1178 events (n=1178) were included in the plot of Salvianolic Acid B. Different event types, as marked with roman numerals, were respectively denoted on the scatter plot. (m) The scatter plot of the ΔI/Iₚ vs the standard deviation (S.D.) when seven Salvianolic acids were sensed stochastically, including Protocatechualdehyde (PA), Protocatechuic Acid (PCA), Caffeic Acid (CA), Salvianic acid A (SAA), Rosmarinic Acid (RA), Lithospermic Acid (LSA) and Salvianolic Acid B (SalB). Different Salvianolic acids types, as marked with different colors of circles mentioned earlier.

FIG. 123: Single molecule sensing of α-hydroxy acid by MspA-PBA. (a, d, g, j) The chemical structure of α-hydroxy, malic acid (a), tartaric acid (d), citric acid (g) and isocitric acid (j) (b, e,h, k) A representative trace of α-hydroxy sensing. The trace was acquired when a +160 mV bias was continuously applied and α-hydroxy was added to cis with a final concentration. A 1.5 M KCl, 100 mM MOPS, pH 7.0 buffer was used. Iₚ stands for the open pore current of the MPBA modified (N90C)₁(M2)₇ MspA. Iₕ stands for the blockage level when α-hydroxy was bound to the pore. Malic acid was added to 0.2 mM (b). Tartaric acid was added to 0.4 mM (e). Citric acid was added to 6 mM (h). Isocitric acid was added to 1 mM (k). (c, f, I, 1) The scatter plot of the ΔI/Iₚ vs the standard deviation (S.D.) when α-hydroxy was sensed as the sole analyte. ΔI=Iₚ−Iₕ. 500 events (n=500) were included in the plot of malic acid. 500 events (n=500) were included in the plot of tartaric acid. 500 events (n=500) were included in the plot of citric acid. 100 events (n=100) were included in the plot of isocitric acid.

FIG. 124: Single molecule sensing of 1,2-diphenols by MspA-PBA. (a, d) The chemical structure of phenol, catechin (a), neochlorogenic acid (d). (b, e) A representative trace of phenol sensing. The trace was acquired when a +160 mV bias was continuously applied and phenol was added to cis with a final concentration. A 1.5 M KCl, 100 mM MOPS, pH 7.0 buffer was used. Iₚ stands for the open pore current of the MPBA modified (N90C)₁(M2)₇ MspA. Iₕ stands for the blockage level when α-hydroxy was bound to the pore. Catechin was added to 0.8 mM (b). Neochlorogenic acid was added to 0.5 mM (e). (c, f) The scatter plot of the ΔI/Iₚ vs the standard deviation (S.D.) when phenol was sensed as the sole analyte. ΔI=Iₚ−Iₕ. 500 events (n=500) were included in the plot of catechin. 500 events (n=500) were included in the plot of neochlorogenic.

FIG. 125: Single molecule sensing of fruit juice by MspA-PBA. (a, d, g) The cartoon image of fruit, grape (a), prune (d), lemon (g). (b) The scatter plot of the ΔI/Iₚ vs the standard deviation (S.D.) for grape juice. The analyte identity is predicted by machine learning. Four populations respectively from events of malic acid, tartaric acid, glucose and fructose were detected. The above analytes are represented by 1, 2, 3, 4, respectively. (c) The signal proportion of the four analytes in grape juice. (e) The scatter plot of the ΔI/Iₚ vs the standard deviation (S.D.) for prune juice. The analyte identity is predicted by machine learning. Five populations respectively from events of malic acid, glucose, fructose, sorbitol and neochlorogenic acid were detected. The above analytes are represented by 1, 3, 4, 5, 6, respectively. (f) The signal proportion of the five analytes in grape juice. (h) The scatter plot of the ΔI/Iₚ vs the standard deviation (S.D.) for lemon juice. The analyte identity is predicted by machine learning. Five populations respectively from events of malic acid, glucose, fructose, isocitric acid and citric acid were detected. The above analytes are represented by 1, 3, 4, 7, 8, respectively. (i) The signal proportion of the five analytes in lemon juice. All measurements were carried out in a 1.5 M KCl buffer (1.5 M KCl, 100 mM MOPS, pH 7.0). A transmembrane potential of +160 mV was continually applied. 5 μL fruit juice was added to cis, respectively.

FIG. 126: Single-molecule sensing of Glycine by MspA-NTA-Ni. (a) The chemical structure of Glycine. (b) A representative trace of glycine with a final concentration of 1 mM in the cis chamber. I_{N} stands for the open pore current of the nickel modified MspA. I_{G} stands for the blockage level when a glycine was bound to the pore. The measurements were carried out in a 1.5 M KCl buffer (1.5 M KCl, 10 mM CHES, pH 9.0). A transmembrane potential of +100 mV was continuously applied. (c) The scatter plot of the ΔI/I_{N} vs the standard deviation (S.D.) when glycine was sensed as a sole analyte. ΔI is defined as ΔI=I_{G}−I_{N}. 130 events (n=130) were included in the plot.

FIG. 127: Single amino acid discrimination with MspA-NTA-Ni. (a) The chemical structure of twenty proteinogenic amino acids (top) and their corresponding events (bottom). The measurements were carried out in a 1.5 M KCl buffer (1.5 M KCl, 10 mM CHES, pH 9.0). A transmembrane potential of +100 mV was continually applied. (b) The scatter plot of ΔI/I_{N} vs the standard deviation (S.D.) from events acquired with 20 individual nanopore measurements (n=5118), during which the twenty proteinogenic amino acids were respectively added to cis as a sole analyte. Events from twenty amino acids are clearly distinguishable when both ΔI/I_{N} and S.D. are considered.

FIG. 128: Single-molecule sensing of amino acids with post-translational modification by MspA-NTA-Ni. (a) Top: The chemical structure of amino acids with post-translational modification investigated in this manuscript. Bottom: The representative nanopore events of the corresponding amino acids. The measurements were carried out in a 1.5 M KCl buffer (1.5 M KCl, 10 mM CHES, pH 9.0). A transmembrane potential of +100 mV was continually applied. (b) The scatter plot of ΔI/I_{N} vs the standard deviation (S.D.) from events acquired with 4 individual nanopore measurements, 230 events were used to generate the plot. Four distributions are clearly distinguishable without any overlaps.

FIG. 129: The construction of copper-modified MspA. (a) The mechanism of constructing copper-modified MspA. A copper ion was strongly chelated by nitrilotriacetic acid (NTA) that was chemically attached to the pore, which could further coordinate with other ligands. Glycine was chosen as a model ligand here. (b) Single-channel observation of copper modification. The measurements were carried out in a 1.5 M KCl buffer (1.5 M KCl, 10 mM CHES, pH 9.0). A transmembrane potential of +100 mV was continuously applied. I_{N} stands for the open pore current of MspA-NTA, with frequent switching between two current levels (i). An irreversible current drop of about 30 pA was observed after 100 μM copper was added in the trans chamber, indicating the successful coordination of copper with NTA. The open pore of MspA-NTA-Cu was marked with I_{C} (ii). The gray rectangle represented noise caused by opening the Faraday cage in order to add copper. Then, glycine was added in cis with a final concentration of 100 μM, which can coordinate with copper in a reversible manner and caused upward blocking events (I_{G}) (iii).

FIG. 130: Single-molecule sensing of Guanine by MspA-NTA-Ni. (a) The chemical structure of Guanine. (b) A representative trace of guanine (dissolved in DMSO) with a final concentration of 20 mM in the cis chamber. Guanine generated two types of events, which are marked with blue and red circles, respectively. The open pore current (I₀), blockage current (I_{G}) and dwell time (τ_{off}) are respectively marked on the trace. The measurements were performed in a 1.5 M KCl buffer (1.5 M KCl, 10 mM CHES, pH 9.0). A transmembrane potential of +50 mV was continuously applied. (c) The scatter plot of log (τ_{off}) vs ΔI/I₀ and the corresponding histogram of ΔI/I₀. ΔI is defined as ΔI=I_{G}−I₀. 605 events (n=605) were included from a 25 min continuously recorded trace as demonstrated in (b). Events with a dwell time (τ_{off}) less than 2 milliseconds were ignored for the statistics.

### DETAILED DESCRIPTION OF THE INVENTION

It should be understood that the specific methods and conditions described in embodiments of the present invention are for the purpose of describing specific embodiments only and are not meant to be limiting, and that any methods and conditions similar or equivalent to those described herein may be used in the practice or testing of the present invention. The explanations of the relevant theories or mechanisms in the present invention are intended only to aid in the understanding of the invention and should not be considered a limitation of the embodiments protected by the present invention.

Unless otherwise noted, terms used in the present invention have the meanings commonly understood in the art and may be understood by reference to standard textbooks, references, and literature known to those skilled in the art.

Unless otherwise stated, the term “comprise”, “include”, “contain” and variations of these terms, such as comprising, comprises and comprised, are not intended to exclude further members, components, integers or steps. These terms also encompass the meaning of “consist of” or “consisting of”. The term “consist of” or “consisting of” is a particular embodiment of the term “comprise”, wherein any other non-stated member, component, integer or step is excluded.

It must be noted that as used herein and in the appended claims, the singular forms “a”, “an” and “the” include plural referents unless the context clearly dictates otherwise. It is further noted that the claims may be drafted to exclude any optional element. As such, this statement is intended to serve as antecedent basis for use of such exclusive terminology as “solely”, “only” and the like in connection with the recitation of claim elements, or use of a “negative” limitation.

The term “at least one” or “one or more” means one, two, three, four, five, six, seven, eight, nine, ten or more.

The term “about” refers to a range equal to the particular value plus or minus ten percent (+/−10%).

The term “and/or” refers to any one, several or all of the elements connected by the term.

Unless otherwise defined, the terms “first” and “second”, when used in conjunction with an element or a feature, are used only to distinguish one element or feature from another and do not imply any particular meaning or any priority in terms of positions or steps.

The term “derivative” of a compound means that the derivative contains a common core chemical structure with the compound, but differs by having at least one structural difference, e.g., by having one or more substituents added and/or removed and or substituted, and/or by having one or more atoms substituted with different atoms.

The term “analogue” refers to a chemical molecule that is similar to another chemical substance in structure and function, differing structurally by one single element or group, or more than one group (e.g., 2, 3, or 4 groups) if it retains the same chemical scaffold and function as the parental chemical.

It should be understood that the method of the present invention may be performed in vivo, in vitro, or ex vivo. The method of the present invention may be not for the purpose of disease treatment, and/or not for the purpose of disease diagnosis.

The term “nanopore”, as used herein, generally refers to a pore, channel or passage which has a very small diameter on the order of nanometers and extends through a membrane. A nanopore may have a characteristic width or diameter on the order of 0.1 nanometers (nm) to about 1000 nm.

The term “protein nanopore” refers to a polypeptide subunit or a multimer of polypeptide subunits (each subunit may be called a monomer of the protein nanopore) that can form a channel through a membrane. The term “protein nanopore” includes wild-type nanopore, such as alpha-hemolysin (α-HL), Mycobacterium smegmatis porin A (MspA). Aerolysin, curli production assembly/transport component (CsgG), outer membrane porin F (OmpF), Cytolysin A (Cly A), ferric hydroxamate uptake component A (FhuA), Fragaceatoxin C (FraC), Pleurotolysin A (Ply A)/Pleurotolysin B (PlyB), Curli production assembly/transport component CsgG (CsgG) or Phi29 connector protein, or a variant of a wild-type nanopore. Sequences of wild type protein nanopore can be found in GenBank on https://www.ncbi.nlm.nih.gov/. A variety of variants of the above protein nanopores have been establish in recent years.

A variant of protein nanopore may have one or more additions, substitutions and/or deletions of amino acids compared to their parental ones, or may have a sequence identity of at least 70%, 75%, 80%, 85%, 90%, 95%, 96%, 97%, 98%, or 99% compared to their parental ones, wherein the parental protein or peptide may be a wild-type one, or homolog or variant thereof, and retains tunnel-forming capability.

The term “sequence identity”, as used herein, refers to the percentage of identical nucleotide or amino acid residues at corresponding positions in two or more sequences when the sequences are aligned to maximize sequence matching, i.e., taking into account gaps and insertions. The alignment of the sequences and the calculation of percentage of the sequence identity can be carried out with suitable computer programs known in the art. Such programs include, but are not limited to, BLAST, ALIGN, ClustalW, EMBOSS Needle, etc. An example of a local alignment program is BLAST (Basic Local Alignment Search Tool), which is available from the webpage of National Center for Biotechnology Information which can currently be found at http://www.ncbi.nlm.nih.gov// and which was firstly described in Altschul et al. (1990) J. Mol. Biol. 215; 403-410. Examples of a global alignment program (which optimizes the alignment over the full-length of the sequences) are EMBOSS Needle and EMBOSS Stretcher programs based on the Needleman-Wunsch algorithm (Needleman, Saul B.; and Wunsch, Christian D. (1970), “A general method applicable to the search for similarities in the amino acid sequence of two proteins”, Journal of Molecular Biology 48 (3): 443-53), which are both available at http://www.ebi.ac.uk/Tools/psa/.

Preferably, the protein nanopore used in the present invention does not gate spontaneously, even at 150 mV-200 mV or more. “To gate” or “gating” refers to the spontaneous change of electrical conductance through the tunnel of the protein that is usually temporary (e.g., lasting for as few as 1-10 milliseconds to up to a second). For some protein nanopore, the probability of gating increases with the application of higher voltages. Typically, the protein becomes less conductive during gating, and conductance may permanently stop (i.e., the tunnel may permanently shut) as a result, such that the process is irreversible. Optionally, gating refers to the conductance through the tunnel of a protein spontaneously changing to less than 75% of its open state current.

### Protein Nanopore

The protein nanopore of the present invention comprises at least one sensing module in a single protein nanopore, wherein the sensing module can interact with an analyte, which allows the protein nanopore to characterize single molecule of an analyte. In a preferred embodiment, a single protein nanopore comprises only one sensing module.

The term “sensing module”, as used herein, refers to a chemical portion that can interact with single molecule of a target analyte. Said chemical portion may comprise one or more chemical molecules or one or more chemical groups. A sensing module may be comprised of one or more (such as two or more) sensing moieties.

The term “moiety”, as used herein, refers to a chemical molecule or any part of a chemical molecule, such as, a functional group. The term “sensing moiety”, as used herein, refers to a moiety which is capable of interacting with single molecule of a target analyte.

The term “interact” or “interaction”, as used herein, may refer to reaction or binding between the sensing moiety and the target analyte, which may be reversible or irreversible. The interaction between the sensing moiety and the target analyte may cause a change in the ionic current across the nanopore, which is measurable.

A sensing module may consist of only one sensing moiety capable of interacting with single molecule of a target analyte alone, wherein the sensing moiety may be called a non-cooperative sensing moiety. In such cases, the sensing module is equal to the non-cooperative sensing moiety.

A sensing module may also consist of two, three, four or more sensing moieties, wherein the two or more sensing moieties together interact with single molecule of a target analyte and each sensing moiety interacts with one or two or more binding sites of the single molecule. The two or more sensing moieties that interact together with single molecule of a target analyte may be called cooperative sensing moieties. Single molecule of some target analytes may comprise two or more binding sites where the sensing moiety interacts with the target analyte. The two or more cooperative sensing moieties in one sensing module may interact with the two or more binding sites in one molecule, respectively. The two or more cooperative sensing moieties in one sensing module may be identical or different from each other, which can be designed according to the binding sites in the target analyte. The analyte molecule can be grasped more easily and strongly by a sensing module consisting of cooperative sensing moieties.

In some embodiments, a protein nanopore that consists of two or more monomers (which can also be called a multimer nanopore) is used. The at least one sensing module may be comprised in one or more monomers. A single sensing module may be comprised in a single monomer, wherein the single monomer may comprise all the sensing moieties of the single sensing module. In the cases that a sensing module consists of two or more sensing moieties, the two or more sensing moieties may be comprised in two or more monomers respectively, wherein each of the monomers may comprise one or more sensing moieties.

In some embodiments, one or more but not all of the monomers of the multimer nanopore comprise one or more sensing modules (which may be called reactive monomer), and none of the remaining monomers (which may be called non-reactive monomer) comprise a sensing module. Such a multimer nanopore may be referred as a heterogeneous protein nanopore in the present invention. In some embodiments, only one monomer of the heterogeneous protein nanopore comprises one or more sensing modules (preferably, only one sensing module or only one sensing moiety), and none of the remaining monomers comprise a sensing module.

The term “heterogeneous protein nanopore” refers to a protein nanopore in which at least one of the multiple monomers has a different structure (e.g., amino acid sequence or amino acid sequence together with its modifications) from the other monomers.

The sensing moiety may be an amino acid residue in the polypeptide of the protein nanopore protein or is attached to an amino acid residue in the polypeptide of the protein nanopore. In some embodiments, a single sensing moiety consists of a single amino acid residue or is attached to a single amino acid residue. Both the amino acid residue that functions as a sensing moiety (the amino acid residue of the first class) and the amino acid residue that is attached to the sensing moiety (the amino acid residue of the first class) are referred to in the present invention as a reactive amino acid residue (which can also be called a reactive site). A single sensing module may consist of one or more reactive amino acid residues in the polypeptide of the nanopore protein or one or more sensing moieties that are attached respectively to one or more reactive amino acid residues in the polypeptide of the nanopore protein. In some embodiments, the protein nanopore of the present invention comprises one or more reactive amino acid residues (either the first class or the second class). In some embodiments, the protein nanopore comprises only one reactive amino acid residue.

In a heterogeneous protein nanopore, the one or more reactive amino acid residues may be located in one or more but not all of the monomers, and none of the remaining monomers comprise a reactive amino acid. In some embodiments, the protein nanopore comprises only one reactive amino acid residue in a single monomer.

The term “amino acid” refers to any organic molecule that contains at least one amino group and at least one carboxyl group. Typically, at least one amino group is at a position relative to a carboxyl group. The term “amino acid” includes natural amino acid, such as proteinogenic amino acids, including 20 conventional amino acids (i.e., alanine, cysteine, aspartic acid, glutamic acid, phenylalanine, glycine, histidine, isoleucine, lysine, leucine, methionine, asparagine, proline, glutamine, arginine, serine, threonine, valine, tryptophan, and tyrosine) and pyrolysine or selenocysteine; and unnatural amino acid, such as modified amino acid. The nanopore protein of the present invention may comprise at least one reactive amino acid residue that functions as a sensing moiety (first class) or is attached to the sensing moiety (second class).

The term “modified” or “modifying”, as used herein, is meant a changed state or structure of a molecule of the invention. Molecules may be modified in many ways, including chemically, structurally, and functionally, for example, by replacement of the original molecule or a group with a different molecule or a group, or by introduction of a molecule or a group by covalent attachment.

The term “reactive” is specific to a particular analyte, a particular sensing moiety and/or a particular linker. If an amino acid residue can interact with a first analyte but cannot interact with a second analyte, it is considered as being reactive to the first analyte and being non-reactive to the second analyte. If an amino acid residue can be attached to a first sensing moiety but cannot be attached to a second sensing moiety, it is considered as being reactive to the first sensing moiety and being non-reactive to the second analyte. If two different amino acid residues are both capable of interacting with the same analyte, they are both considered as being reactive to said analyte. If an amino acid residue can interact with a first linker but cannot interact with a second linker, it is considered as being reactive to the first linker and being non-reactive to the second linker.

The term “attach” or “attachment” refers to connecting or uniting by a bond or force in order to keep two or more components together, which encompasses either direct or indirect attachment such that, for example, a first compound is directly bound to a second compound, and the embodiments wherein one or more intermediate compounds, and in particular groups, are disposed between the first compound and the second compound. In some embodiments, the sensing moiety or the reactive amino acid residue can be attached to each other through a covalent bond.

The reactive amino acid residue that can function as a sensing moiety (the amino acid residue of the first class) may be a natural amino acid. In some embodiments, the amino acid that functions as a sensing molecule may be selected from methionine, histidine, cysteine, lysine and any combination thereof. In some embodiments, methionine, histidine, cysteine or lysine alone can interact with a single molecule of a metal ion and each of them can be used as a sensing module to characterize a metal ion. In some embodiments, two or more of methionine, histidine, cysteine and lysine can interact together with a single molecule of a metal ion and can be used together as a sensing module consisting of cooperative sensing moieties to characterize a metal ion. In some embodiments, the protein nanopore (especially the heterogeneous protein nanopore) of the present invention comprises a single reactive amino acid residue that functions as a sensing moiety, which for example can be selected from methionine, histidine, cysteine and lysine.

A sensing moiety is attached to the reactive amino acid residue of the second class, optionally via a linker. In some embodiments, the reactive amino acid residue is reactive to the linker. The linker can be attached to the reactive amino acid residue and can be linked to a sensing moiety. In some embodiments, the linker and the sensing moiety may be linked by covalent bond or coordination. In some embodiments, the linker may be a ligand. In some embodiments, the linker and the sensing moiety may form a coordinate complex.

The term “coordination” refers to an interaction in which one multi-electron pair donor coordinately bonds, i.e., is “coordinated,” to one metal ion. The term “coordination” refers to an interaction between an electron pair donor and a coordination site on a metal ion resulting in an attractive force between the electron pair donor and the metal ion. A coordinate bond may be formed between the electron pair donor and the metal ion. The electron pair donor may be a nonmetal atom, such as nitrogen, sulfur, phosphorus, carbon or oxygen, etc. A compound containing the electron pair donor may be referred as a ligand. The term “coordination complex” is a complex in which there is a coordinate bond between the metal ion and the electron pair donor, ligand or chelating group. Thus, ligand or chelating group is generally electron pair donor, molecule or molecular ion having unshared electron pairs available for donation to a metal ion.

The sensing moiety or the linker may be attached to the reactive amino acid residue by any suitable approaches, such as a chemical reaction, e.g., a click reaction. Examples of the click reaction may include, but not limited to, a copper(I)-catalyzed alkyne-azide cycloaddition (CuAAC), such as a reaction between azide and alkyne; a copper free alkyne-azide cycloaddition, such as a reaction between azide and difluorinated cyclooctyne; a staudinger ligation, such as a reaction between azide and phosphine; a radical addition, such as between a reaction thiol and alkene; a michael addition, such as a reaction between thiol and maleimide; a nucleophilic substitution, such as a reaction between amine and para-fluoro (Becer, Hoogenboom, and Schubert, Click Chemistry beyond Metal-Catalyzed Cycloaddition, Angewandte Chemie International Edition, 2009, 48:490-4908; Rostovtsev, V. V. et al., 2002, A stepwise Huisgen cycloaddition process: Copper (I)-catalyzed regioselective “ligation” of azides and terminal alkynes. Angew. Chem., Int. Ed. 41, 2596-2599; Torne, C. W. et al., 2002, Peptidotriazoles on solid phase: [1,2,3]-Triazoles by regiospecific copper(I)-catalyzed 1,3-dipolar cycloadditions of terminal alkynes to azides. J. Org. Chem. 67, 3057-3064; Agard, N. J. et al., 2004, A strainpromoted [3+2] azide-alkyne cycloaddition for covalent modification of blomolecules in living systems. J. Am. Chem. Soc. 126, 15046-15047; Kohn, M., and Breinbauer, R., 2004, The Staudinger ligation: A gift to chemical biology. Angew. Chem., Int. Ed. 43, 3106-3116). In some embodiments, the sensing moiety or the linker may be attached to the reactive amino acid residue by a reaction between reactive handle pair, wherein the first reactive handle is comprised in the reactive amino acid residue, and the second reactive handle is comprised in a chemical molecule that also comprises the sensing moiety or the linker. The chemical molecule comprising the first reactive handle can be brought into contact with the reactive amino acid residue, a reaction occurs between the two reactive handles, and the sensing moiety or the linker is attached to the reactive amino acid residue. In some embodiments, the reactive handle may be a click reaction handle.

The reactive amino acid residue may be a natural amino acid residue comprising the first reactive handle. The first reactive handle may also be introduced into the reactive amino acid residue by modification of the amino acid. In some embodiments, the first reactive handle may be thiol or amino group, i.e., ε amino group. In some embodiments, the second reactive handle may be alkene or maleimide. In some embodiments, the sensing moiety or the linker may be attached to the reactive amino acid residue by a reaction between thiol and maleimide. In some embodiments, the reactive amino acid residue of the second class may be selected from the group consisting of cysteine, methionine and lysine. In some embodiments,

The term “reactive handle”, as used herein, is meant a chemical molecule, a chemical moiety or a chemical group that is exposed and can react with another reactive handle. Reactive handle pair is usually composed of a first reactive handle and a second reactive handle, wherein the first reactive handle can react with the second reactive handle. Reactive handle pair are known to the person skilled in the art. Reactive handle pair that can be used in the present invention include, but are not limited to, click reaction handles. The term “click reaction handle” means the chemical molecule, chemical moiety or chemical group that partake a click reaction.

In some embodiments, the sensing moiety may be a moiety comprising boronic acid, such as phenylboronic acid (PBA), which may be used as a non-cooperative sensing moiety and can be attached to the reactive amino acid residue by a chemical reaction, e.g., a click reaction, for example, a reaction between thiol and maleimide. In some embodiments, the protein nanopore (especially the heterogeneous protein nanopore) of the present invention comprises a single moiety comprising boronic acid, such as a single phenylboronic acid (PBA).

In some embodiments, the sensing moiety may be a metal ion (which may be used as a non-cooperative sensing moiety), such as Ni²⁺, Cu²⁺, Co²⁺, Zn²⁺, Cd²⁺, Ag²⁺ Pb²⁺, Fe²⁺ or Fe³⁺. In some embodiments, the metal ion may be attached to the reactive amino acid residue by a linker, such as a ligand.

In some embodiments, the ligand may be a metal chelating agent, such as nitrilotriacetic acid (NTA) or iminodiacetic acid (IDA), which can be attached to the reactive amino acid residue by a chemical reaction, e.g., a click reaction, for example, a reaction between thiol and maleimide.

In some preferred embodiments, the protein nanopore (especially the heterogeneous protein nanopore) of the present invention comprises Ni²⁺ as a sensing module that is attached to a reactive amino acid residue via NTA, wherein NTA and Ni²⁺ forms a coordination complex that can be called NTA-Ni. The protein nanopore comprising NTA-Ni can also be called a protein nanopore modified by NTA-Ni. In some embodiments, the protein nanopore (especially the heterogeneous protein nanopore) of the present invention comprises a single reactive amino acid residue and comprises a single sensing moiety that is attached to the reactive amino acid residue via a single ligand. In more preferred embodiments, the protein nanopore (especially the heterogeneous protein nanopore) of the present invention comprises a single reactive amino acid residue and comprises a single NTA-Ni attached to the single reactive amino acid residue, wherein “single NTA-Ni” refers to a coordination complex consisting of a single NTA and a single Ni²⁺.

It should be understood that in some cases, a protein nanopore inherently comprises suitable reactive amino acid residue defined in the present invention. In other cases, when a protein nanopore does not comprise a suitable reactive site, a suitable reactive site can be obtained by modification of the amino acid of said protein nanopore. The protein nanopore to be modified may be called a parental protein nanopore. The modified protein nanopore may be called a variant of the parental protein nanopore and may be referred to as being derived from the parental protein nanopore. The modification may include insertion, substitution, deletion and/or chemical modification of an amino acid. For example, an amino acid residue (e.g., a non-reactive amino acid residue) in a parental protein nanopore may be replaced with a reactive amino acid residue, which may be achieved by chemically synthesis or genetic recombination. In cases where the parental protein nanopore contains two or more multiple reactive amino acid residues, a suitable reactive amino acid residue can also be obtained by replacing one or more but not all of these reactive amino acid residues with non-reactive amino acid residue. By “chemical modification of an amino acid” means to add or change a group in an amino acid by a chemical method to make it an unnatural amino acid.

The parental protein nanopore may be a wild-type protein nanopore or a variant thereof. A variant of a multimer protein is a protein nanopore in which one or more monomers, or all monomers, are modified compared to the parental protein nanopore.

In some embodiments, the parental protein nanopore may selected from alpha-hemolysin (α-HL), Mycobacterium smegmatis porin A (MspA), Aerolysin, curli production assembly/transport component (CsgG), outer membrane porin F (OmpF). Cytolysin A (Cly A), ferric hydroxamate uptake component A (FhuA), Fragaceatoxin C (FraC), Pleurotolysin A (PlyA)/Pleurotolysin B (PlyB), Curli production assembly/transport component CsgG (CsgG), Phi29 connector protein, and any variant thereof.

In some embodiments, the parental protein nanopore is selected from wild-type MspA, M1 MspA and M2 MspA.

A wild-type MspA, which is also referred as MspA, is an octameric protein nanopore in which each monomer has the following sequence:

(SEQ ID NO: 1)
GLDNELSLVDGQDRTLTVQQWDTFLNGVFPLDRNRLTREWFHSGRAKYIV
AGPGADEFEGTLELGYQIGFPWSLGVGINFSYTTPNILIDDGDITAPPFG
LNSVITPNLFPGVSISADLGNGPGIQEVATFSVDVSGAEGGVAVSNAHGT
VTGAAGGVLLRPFARLIASTGDSVTTYGEPWNMN.

Variants of a MspA include, but are not limited to, an octameric protein nanopore in which each monomer has a mutation of D90N/D91N/D93N (M1 MspA) or D93N/D91N/D90N/D118R/D134R/E139K (M2 MspA) compared to the wild-type one. The expression of the mutation means that the variant comprises simultaneously all of listed mutations compared to the wild-type one, wherein the amino acid numbering is with reference to the wild-type MspA.

The term “heterogeneous protein nanopore” may be regarded as a variant of a parental protein nanopore in which one or more but not all monomers are modified compared to the parental protein nanopore.

The heterogeneous protein nanopore of the present invention can be prepared by providing one or more monomers that comprises one or more reactive amino acid residues (which may be called reactive monomer), and one or more monomers that do not comprise a reactive site (which may be called non-reactive monomer), and subsequently enabling them to assemble into a protein nanopore under appropriate conditions (such as by mixing them together).

The monomer comprising one or more reactive amino acid residues and the monomer not comprising a reactive amino acid residue may be prepared by modification of a monomer of a protein nanopore. The monomer to be modified may be called a parental monomer and modified monomer may be called a variant of the parental monomer and may be referred to as being derived from the parental monomer. The modification may include insertion, substitution, deletion and/or chemical modification of an amino acid. For example, an amino acid residue (e.g., a non-reactive amino acid residue) in a parental monomer may be replaced with a reactive amino acid residue, which may be achieved by chemically synthesis or genetic recombination. In cases where the parental monomer contains two or more multiple reactive amino acid residues, a suitable reactive amino acid residue can also be obtained by replacing one or more but not all of these reactive amino acid residues with non-reactive amino acid residue.

The parental monomer may be from a parental protein nanopore and may be a monomer of a wild-type protein nanopore or a variant thereof. In some embodiments, the parental monomer may be the monomer of a protein nanopore selected from alpha-hemolysin (α-HL), Mycobacterium smegmatis porin A (MspA), Aerolysin, curli production assembly/transport component (CsgG), outer membrane porin F (OmpF), Cytolysin A (Cly A), ferric hydroxamate uptake component A (FhuA), Fragaceatoxin C (FraC), Pleurotolysin A (Ply A)/Pleurotolysin B (PlyB), Curli production assembly/transport component CsgG (CsgG), Phi29 connector protein, and any variant thereof. In some embodiments, the parental monomer may be a monomer of wild-type MspA, M1 MspA or M2 MspA.

When the heterogeneous protein nanopore comprises two or more non-reactive monomers, the two or more non-reactive monomers may be the same with or different from each other.

The reactive amino acid residue (either the first class or the second class) may be located on the surface of the channel. The reactive amino acid residue may be located at any position on the surface of the nanopore channel, such as the constriction zone, which is the narrowest portion of the nanopore channel, or the vestibule, which is at one end of the nanopore channel and has a larger diameter than the constriction zone.

When the protein nanopore is derived from MspA or variant thereof, or the monomer of the protein nanopore is derived from the monomer of MspA or variant thereof, the one or more reactive amino acid residues are located at one or more positions selected from 83-111, preferably 90, 91, 92 and 93, wherein the position of the amino acid residue is with reference to the wild-type MspA. In some embodiments, the reactive amino acid residue is cysteine or methionine located at positions selected from 90, 91, 92 and 93.

In some embodiments, the heterogeneous protein nanopore of the present invention is a variant of MspA which comprises at least one amino acid mutation in one or more monomers compared to MspA or M2 MspA. In some embodiments, the mutation comprises mutation to cysteine, methionine or lysine, preferably at one or more positions selected from 83-113, preferably 90, 91, 92 and 93.

In some embodiments, the heterogeneous protein nanopore of the present invention is a variant of MspA and comprise a single reactive monomer which comprise a single reactive amino acid residue, wherein the single reactive amino acid residue is located at position 90, 91, 92 or 93 and selected from cysteine and methionine. In some embodiments, the heterogeneous protein nanopore of the present invention has a mutation of N90C, N90M and/or N91C in one or more monomers compared to M2 MspA. In some embodiments, the heterogeneous protein nanopore of the present invention has a mutation of D90C, D90M and/or D91C in one or more monomers compared to MspA.

### Characterization of a Target Analyte

The protein nanopore comprising at least one sensing module of the present invention may be used to characterize (or identify) an analyte. The term “analyte” may also be referred to as “target analyte”, is a target molecule detectable by the protein nanopore of the present invention. The target analyte can interact with the sensing module comprised in the protein nanopore, which can cause a measurable change in the ionic current across the nanopore. It should be understood that the target analyte is matched to the sensing module, i.e., the target analyte may be any molecule that can interact with the sensing module, reversibly or irreversibly, when in contact with the sensing module in the channel of the protein nanopore.

In some embodiments, the target analyte can interact with one or more selected from boronic acid, metal ion (such as Ni²⁺, Cu²⁺, Co²⁺, Zn²⁺, Cd²⁺, Ag²⁺ Pb²⁺, Fe²⁺ or Fe³⁺), methionine, histidine, cysteine, lysine and any combination thereof.

The analyte that can interact with boronic acid may be selected from a chemical compound comprising 1,2-diol or 1,3-diol (which may be a cis-diol), an ion comprising metal element, hydrogen peroxide and any combination thereof.

The chemical compound comprising 1,2-diol or 1,3-diol may be selected from polyol, saccharide or a derivative thereof, α-hydroxy acid, a chemical compound comprising a ribose, nucleotide sugar, alditol, polyphenol, catecholamine or catecholamine derivative, tris(hydroxymethyl)methyl aminomethane (Tris), protocatechualdehyde, protocatechuic acid, caffeic acid, rosmarinic acid, lithospermic acid, salvianic acid A, salvianolic acid B and any combination thereof.

The polyol includes alditol, polyphenol, vitamin, catecholamine and nucleotide analogues. The saccharide may be selected from monosaccharide, oligosaccharide, polysaccharide and any combination thereof.

The monosaccharide may be selected from D-glyceraldehyde, D-erythrose, D-ribose, 2′-deoxy-D-ribose, D-xylose, L-arabinose, D-lyxose, D-glucose, D-galactose, D-mannose, D-fructose, L-sorbose, L-fucose, D-allose, D-tagatose, L-rhamnose, D-galactose and any combination thereof.

The oligosaccharide may be selected from disaccharide (such as sucrose, isomaltulose, maltulose, turanose, leucrose, trehalulose, lactulose, maltose), trisaccharide (such as raffinose), tetrasccharide (such as stachyose) and complex oligosaccharide (such as acarbose) and any combination thereof.

The polysaccharide may be selected from pentasaccharide, such as verbascose.

The derivative of saccharide may be selected from N-acetylneuraminic acid (sialic acid), N-Acetyl-D-Galactosamine and any combination thereof.

α-hydroxy acid may be selected from tartaric acid, malic acid, citric acid, isocitric acid and any combination thereof.

The chemical compound comprising a ribose may be selected from nucleotide or modified nucleotide, derivative of nucleotide or modified nucleotide, nucleoside or nucleoside analogue, and any combination thereof.

The nucleotide may be selected from adenine nucleotide, cytosine nucleotide, uracil nucleotide, guanine nucleotide and any combination thereof.

The modified nucleotide includes methylated, deaminated, reduced or thiolated nucleotide, and a nucleotide with an isomerization to either the ribose or the nucleobase of nucleotides. The modified nucleotide may be selected from a nucleotide containing 5-methylcytidine (m⁵C), N6-methyladenosine (m⁶A), pseudouridine (Ψ), inosine (I), N7-methylguanosine (m⁷G), N1-methyladenosine (m¹A), dihydrouridine (D), N2-methylguanosine (m²G), N2,N2-dimethylguanosine (m₂²G), wybutosine (Y), 5-methyluridine (T), N-acetylcytidine (ac4C) and any combination thereof.

The derivative of nucleotide or modified nucleotide may be selected from monophosphate derivative, diphosphate derivative, triphosphate derivative and tetraphosphate derivative of a nucleotide or a modified nucleotide and any combination thereof, such as ADP, UDP, GDP, CDP, ATP, UTP, GTP, CTP, a derivative of them and any combination thereof. The monophosphate derivative, diphosphate derivative, triphosphate derivative or tetraphosphate derivative of a nucleotide or a modified nucleotide may also be referred to as monophosphate derivative, diphosphate derivative, triphosphate derivative or tetraphosphate derivative of a nucleoside or a modified nucleoside, which refers to nucleoside monophosphate, modified nucleoside monophosphate, nucleoside diphosphate, modified nucleoside diphosphate, nucleoside triphosphate, modified nucleoside triphosphate, nucleoside tetraphosphate, modified nucleoside tetraphosphate, or derivative thereof.

The nucleoside analogue may be selected from galidesvir, ribavirin, molnupiravir, remdesivir, loxoribine, mizoribine, 5-azacytidine, capecitabine, doxifluridine, 5-fluorouridine, forodesine, clitocine, pyrazofurin, sangivamycin, pseudouridimycin and any combination thereof.

The nucleotide sugar may be selected from uridine diphosphate glucose (UDPG), uridine diphosphate N-acetylglucosamine, uridine diphosphate glucuronic acid, adenosine diphosphate glucose, uridine diphosphate galactose, uridine diphosphate xylose, guanosine diphospbate mannose, guanosine diphosphate fucose, cytidine monophosphate N-acetylneuraminic acid, uridine diphosphate N-acetylgalactosamine and any combination thereof.

The alditol may be selected from glycerin, propanetriol, tetritol, pentitol, hexitol, erythritol, threitol, arabitol, xylitol, adonitol, fucitol, sorbitol (including L-sorbitol and D-sorbitol), mannitol, dulcitol, iditol, talitol, allitol, maltitol, lactitol, isomalt and any combination thereof.

The polyphenol may be selected from catechin, neochlorogenic acid, anthocyanin, proanthocyanidin, catechol or derivative thereof, such as catechol, 3-fluorocatechol, 3-chlorocatechol, 3-bromocatechol, 4-fluorocatechol, 4-chlorocatechol, 4-bromocatechol, 3-methylcatechol, 4-methylcatechol, 3-methoxycatechol, 3-propylcatechol, 3-isopropylcatechol, 3,6-dibromocatechol, 4,5-dibromocatechol, 3,6-dichlorocatechol, and any combination thereof.

The catecholamine or catecholamine derivative may be selected from epinephrine, norepinephrine (or noradrenaline), isoprenaline and any combination thereof.

The ion comprising metal element may be selected from alkaline-earth metal ion, transition metal ion and any combination thereof, preferably selected from AuCl₄⁻, Mg²⁺, Ca²⁺, Ba²⁺, Ni²⁺, Cu²⁺, Co²⁺, Zn²⁺, Cd²⁺, Ag²⁺, Pb²⁺ and any combination thereof.

The analyte that can interact with metal ion (such as Ni²⁺, Cu²⁺, Co²⁺, Zn²⁺, Cd²⁺, Ag²⁺ Pb²⁺, Fe²⁺ or Fe³⁺) may be a compound that can interact with said metal ion by any means, such as coordination, etc. Such a compound may contain a nonmetal atom that can act as an electron donor and coordinate with the metal ion, such as nitrogen, oxygen or carbon atom. Such a compound that contains a suitable chemical group that can coordinate with the metal ion. For example, it may contain at least one carboxylic acid group or at least one amine group. which may be selected from amino acid; modified amino acid; unnatural amino acid; polymer of amino acids or modified amino acids; a chemical compound comprising guanine, adenine, thymine, cytosine or uracil; and any combination thereof.

The amino acid may be selected from alanine, cysteine, aspartic acid, glutamic acid, phenylalanine, glycine, histidine, isoleucine, lysine, leucine, methionine, asparagine, proline, glutamine, arginine, serine, threonine, valine, tryptophan, tyrosine, pyrolysine, selenocysteine and any combination thereof.

The modified amino acid may be selected from phosphorylated amino acid, glycosylated amino acid, acetylated amino acid, methylated amino acid and any combination thereof, such as O-phospho-serine (p-S), N4-(β-N-acetyl-D-glucosaminyl)-asparagine (GlcNAc-N), O-acetyl-threonine (Ac-T), Nω,N′ω-dimethyl-arginine (SDMA) and any combination thereof.

The chemical compound comprising guanine, adenine, thymine, cytosine or uracil may be selected from guanine, adenine, thymine, cytosine or uracil, a nucleoside comprising any one of them, and a nucleotide comprising any one of them, wherein the nucleotide may be a ribonucleotide or a deoxyribonucleotide.

The analyte that can interact with methionine, histidine, cysteine and/or lysine may be an ion comprising metal element, for example, as defined above.

The protein nanopore or the method of the present invention may be used to characterize a carbohydrate-based drugs, polysaccharides/oligosaccharides, small molecule glycosides and glycomimetics, glycopeptides and glycoproteins, which may comprise 1,2-diol or 1,3-diol (which may be cis-diol).

The protein nanopore of the present invention may be disposed in a membrane that separates a first conductive liquid medium from a second conductive liquid medium, which may be called a nanopore system. The channel of the nanopore is the only path for the first conductive liquid medium and the second conductive liquid medium to communicate. Generally, a target analyte is added in at least one of the first conductive liquid medium and the second conductive liquid medium. The membrane can be an organic membrane, such as a lipid bilayer, or a synthetic membrane, such as a membrane formed of a polymeric material. The thickness of the membrane through which the nanopore extends can range from 1 nm to around 10 μm.

The preparation of a nanopore system is well known, for example, for a protein nanopore system, when a porin (such as the protein nanopore of the present invention) is placed in any one of the first conductive liquid medium and the second conductive liquid medium separated by a membrane (such as a lipid bilayer), the porin can insert spontaneously into the membrane to form a nanopore.

The sensing moiety may be attached to the reactive amino acid residue before or after the porin insert in the membrane. For example, a sensing moiety can be attached to the reactive amino acid residue of the porin first, and then the porin comprising the sensing moiety can be inserted into the membrane, wherein the sensing moiety can be attached to the reactive amino acid residue by mix the sensing moiety and the porin together in a condition suitable for the binding of them. For another example, a porin without a sensing moiety can be inserted into the membrane first, and then a molecule comprising a sensing moiety is added in the first conductive liquid medium or the second conductive liquid medium and subsequently comes into contact with the reactive amino acid residue while moving across the nanopore and is thereby attached to the porin.

When the sensing moiety is attached to the reactive amino acid residue via a linker, the linker and the sensing moiety may be attached to the reactive amino acid residue before or after the porin insert in the membrane. For example, the linker and the sensing moiety can be attached to the reactive amino acid residue of the porin first, and then the porin comprising the sensing moiety can be inserted into the membrane to form a nanopore, wherein the linker can be attached to the reactive amino acid residue by mix the sensing moiety and the porin together in a condition suitable for the binding of them, and the sensing moiety can be bound to the linker by mix them together in a condition suitable for the interaction of them. For another example, a porin without a sensing moiety can be inserted into the membrane to form a nanopore first, and then a molecule comprising the linker is added in the first conductive liquid medium or the second conductive liquid medium and subsequently comes into contact with the reactive amino acid residue while moving across the nanopore and is thereby attached to the porin, then a molecule comprising the sensing moiety is added in the first conductive liquid medium or the second conductive liquid medium and subsequently comes into contact with the linker while moving across the nanopore and is thereby bound to the linker. The linker can be attached to the reactive amino acid residue by mix the sensing moiety and the porin together in a condition suitable for the binding of them. The sensing moiety can be bound to the linker by mix them together in a condition suitable for the interaction of them.

The target analyte may be added in either side of the nanopore, i.e., the first conductive liquid medium or the second conductive liquid medium. In some embodiments, the final concentration of the analyte added may range from about 0.01 mM to about 100 mM, e.g., from about 0.1 mM to about 50 mM, e.g., from about 0.1 mM to about 40 mM. For example, the final concentration of the analyte added may be from about 0.1 mM to about 0.2 mM, about 300 μM, about 0.4 mM, about 0.5 mM, about 0.8 mM, about 1 mM, about 2 mM, about 4 mM, about 6 mM, about 10 mM, about 20 mM or about 40 mM. For example, the final concentration of the analyte added may be from about 0.1 mM, about 0.2 mM, about 300 μM, about 0.4 mM, about 0.5 mM, about 0.8 mM, about 1 mM, about 2 mM, about 4 mM, about 6 mM, about 10 mM or about 20 mM to about 40 mM. The appropriate concentration of different analytes may vary and can be determined experimentally.

When an electrical potential difference (also called a voltage or an electric field) is applied between the first conductive liquid medium and the second conductive liquid medium (i.e., an electric field or a voltage is applied across the nanopore), an ionic current is generated through the channel of the nanopore, and the target analyte may be driven into the nanopore from the conductive liquid medium and stretch, e.g., under the action of electrophoretic force and/or diffusion. The electrical potential difference may be no less than 20m V, no less than 40 mV, no less than 60 mV, no less than 80 mV, no less than 100 mV, no less than 120 mV, no less than 140 mV, no less than 160 mV, no less than 180 mV or no less than 200 mV; or range from about 20 mV to 220 mV, range from about 40m V to 200m V, range from about 60m V to 180 mV, range from about 80 mV to 180 mV, range from about 100m V to 180 mV, range from about 120 mV to 180 mV, range from about 140 mV to 180 mV, range from about 160 mV to 180m V.

In some embodiments, the electrical potential difference between the first conductive liquid medium and the second conductive liquid medium varies or remains constant. Process and apparatus for applying an electric field to a nanopore are known to the person skilled in the art. For example, a pair of electrodes may be used to applying an electric field to a nanopore. As will be understood, the voltage range that can be used can depend on the type of nanopore system and the analyte being used.

The target analyte is driven into the nanopore and interacts with the sensing module on the nanopore. This interaction leads to a blockage which is measured to characterize the targe analyte. A system for characterization of a target analyte may further comprise the target analyte. Optionally, in the system, the target analyte may have interacted with the sensing module, or the target analyte may have not interacted with the sensing module.

The target analyte may be driven into the nanopore by an electrophoretic force or a concentration difference (diffusion effect). The target analyte interacts with the sensing module present in the channel of the nanopore and the interaction causes a blockage of the ionic current, which is measurable, for example, by measuring the current after the target analyte enters the nanopore and comparing it with the current when the target analyte has not entered the nanopore. The blockage of the ionic current may be related to the identity of the target analyte, the interaction between the target analyte with an agent (such as the sensing moiety), the binding kinetics of the target analyte, etc.

In general, a “blockage of the ionic current” may also be called a “blockade current”, which is evidenced by a change in ionic current that is clearly distinguishable from noise fluctuations and is usually associated with the presence of an analyte molecule within the nanopore. The strength of the blockade, or change in current, will depend on a characteristic of the analyte. More particularly, “blockage” may refer to an interval where the ionic current drops to a level which is about 5-100% lower than the unblocked current level, remains there for a period of time, and returns spontaneously to the unblocked level. For example, the blockade current level may be about, at least about, or at most about 5%, 10%, 15%, 20%, 25%, 30%, 35%, 40%, 45%, 50%, 55%, 60%, 65%, 70%, 75%, 80%, 85%, 90%, 95%, or 100% lower than the unblocked current level. A blockage may be called a blockade event or an event.

The measurement can be performed at any suitable temperature, such as −4° C.-100° C., e.g., 4° C.-50° C., 5° C.-25° C. or room temperature.

Measurement of the current through a nanopore are well known in the art and may be performed by way of optical signal or electric current signal. For example, one or more measurement electrodes could be used to measure the current through the nanopore. These can be, for example, a patch-clamp amplifier or a data acquisition device.

A “liquid medium” includes aqueous, organic-aqueous, and organic-only liquid media. Organic media include, e g., methanol, ethanol, dimethylsulfoxide, and mixtures thereof. Liquids employable in methods described herein are well-known in the art. Descriptions and examples of such media, including conductive liquid media, are provided in U.S. Pat. No. 7,189,503, for example, which is incorporated herein by reference in its entirety. Salts, detergents, or buffering agents may be added to such media. Such agents may be employed to alter pH or ionic strength of the liquid medium. In some embodiments, the salt may comprise KCl. In some embodiments, the concentration of the salt may be 0.5 M-2.5M. In some embodiments, the concentration of KCl is about 1.5 M. The buffering agent may be HEPES, MOPS, CHES or Tris, etc. The pH of the first conductive liquid medium and/or the second conductive liquid medium may range from about 1.0 to about 13.0, preferably from about 6.0 to about 9.0, preferably from about 6.0 to about 8.0, preferably from about 7.0 to about 7.4, which may depend on the desired charge properties of the target analyte. In some embodiments, the first conductive liquid medium and/or the second conductive liquid medium does not contain Tris. In some embodiments, the first conductive liquid medium and/or the second conductive liquid medium comprises 1.5 M KCl, 10 mM MOPS and has a pH of about 7.0. In some embodiments, the first conductive liquid medium and/or the second conductive liquid medium comprises 1.5 M KCl, 10 mM HEPES and has a pH of about 7.0. In some embodiments, the first conductive liquid medium and/or the second conductive liquid medium comprises 1.5 M KCl, 10 mM CHES and has a pH of about 9.0.

A current pattern and a current trace, as used herein, may be used interchangeably, refer to the ionic current over time. A current pattern may contain one or more types of blockade event, and may contain one or more individual blockade events of the same type. Characteristics about distribution, frequency, amplitude, etc. of the blockade events can be learned from the current pattern.

The term “event”, as used herein, refers to a blockage of the nanopore by a target analyte (i.e., an interval where the ionic current drops to a level which is about 5-100% lower than the first blockade current level, remains there for a period of time, and returns spontaneously to the unblocked current level), and also refers to a current change caused by the blockage of the target analyte. The person skilled in the art know how to determine the occurrence of an event.

A variety of characteristic parameters can be obtained from the current pattern. The characteristic parameters include, but not limit to, open pore current (Iₚ), blockage level (Iₛ), blockage amplitude (ΔI, defined as ΔI=Iₚ−Iₛ), inter-event interval (τₒₙ), event dwell time (τ_{off}), mean dwell time (τ_{off}), mean inter-event interval (τₒₙ), percentage blockage (defined as ΔI/Iₚ) and standard deviation (S.D.) of each event. One or more of these characteristic parameters can be used to characterize (or identify) the analyte.

The characterization (or identification) of the target analyte may include, but is not limited to, determining the identity of the target analyte, determining whether the target analyte is a specific substance, determining the presence or absence of the target analyte, determining the interaction of the target analyte and an agent (for example, the agent may be the sensing moiety, and the system and the method of the present may be used to determine whether there is an interaction between the target analyte and the sensing moiety), or measuring the binding kinetics of the target analyte and an agent (for example, the agent may be the sensing moiety, and the system and the method of the present may be used to determine the binding kinetics of the target analyte and the sensing moiety). The identity may include, but is not limited to, what the analyte is, the structure of the analyte, the protonation state or the deprotonation state of the analyte, the chirality of the analyte, etc.

As an example, to determine the identity of the target analyte, a tested current pattern may be compared with a reference current pattern and the identity of the target analyte is determine.

As an example, to determine whether the target analyte and an agent interact with each other, the agent may be comprised in the protein nanopore of the present invention as a sensing module, and occurrence of an event represent the interaction between the target analyte and the agent.

A tested current pattern, as used herein, refers to the current pattern obtained by using the tested analyte (i.e., the target analyte).

A reference current pattern refers the current pattern used as a reference to determine at least one characteristic of the target analyte. According to the purpose of characterization, different reference current pattern can be used. For example, the reference current pattern can be a current pattern obtained by using a known analyte under the same conditions with the tested current pattern. It can be determined whether the tested analyte is the same with or different from the reference analyte.

In some embodiments, the characterization of the target analyte according to the tested current pattern may be achieved by using machine learning algorithm.

In some embodiments, the tested current pattern may be filtered to obtain a high pass and/or a low pass, and the tested current pattern is provided from the high pass and/or the low pass. In some embodiments, the cut off frequency of the high pass and/or the low pass is about 100 Hz, the cut off frequency of the high pass and/or the low pass is about 100 Hz.

The nanopore and method of the present invention can be used to characterize single molecule of the target analyte of. A large number of analytes can be characterized by the nanopore and method of the present invention, as long as the size of the analyte allows it to enter the channel of the nanopore. If the analyte can interact with one or more moieties, the analyte can be characterized through the nanopore and method of the present invention, where the one or more moieties can be used as the sensing module.

The nanopore and method of the present invention may be used to simultaneously characterize multiple (such as two or more) different target analytes. The multiple different target analytes may interact with the same sensing moiety. In some embodiments, the multiple different target analytes may be driven to enter the channel of the nanopore simultaneously, and interact with the sensing module, respectively. The different interactions between the different analytes and the sensing module may be measured respectively and be distinguished from each other according to their respective current patterns.

The term “different” means that there is a difference in the structures of the multiple target analytes. The multiple different target analytes may have different, similar or the same molecular weight, physical properties, chemical properties, and/or biological properties. The multiple different target analytes may be epimers or isomers of each other.

The nanopore or method of the present invention can be used to discriminate two or more different analyte that have similar structure and/or similar or the same molecular weight, such as a compound and its isomer or epimer, or a nucleotide and its epigenetic counterpart.

The nanopore and method of the present invention may be used to characterize one or more analytes in a sample.

The term “sample” may include blood, serum, plasma, body fluids, cerebrospinal fluid, food, beverages, health products, environmental samples, water samples, etc. The nanopore or method of the present invention can be used to determine the identity of the analyte that is comprised in the sample.

The sample is preferably a liquid, or preferably can be dissolved in a liquid medium, such as water or an organic solvent. The sample can be added directly to the nanopore system or added to the nanopore system after dilution or dissolution to an appropriate concentration.

For example, the sample may be a fruit juice (such as grape juice, prune juice, lemon juice), a sugar-free drink, a tea or an extract of Chinese herb (such as salvia miltiorrhiza). The system and method of the present invention may be used to characterize the saccharide, α-hydroxy acid and/or alditol in the fruit juice, the alditol in the sugar-free drink, a polyphenol in the tea, or protocatechualdehyde, protocatechuic acid, caffeic acid, rosmarinic acid, lithospermic acid, salvianic acid A and/or salvianolic acid B in the extract of Chinese herb (such as salvia miltiorrhiza).

The nanopore and methods of the present invention can be used to characterize nucleotides in RNA (e.g., microRNA or tRNA), including both unmodified and modified nucleotides. RNA can be digested with a nuclease into individual nucleotides, and these nucleotides can then be added as analytes to the nanopore system of the present invention to be characterized.

The present invention also relates to the following solutions.

Solution 1. A heterogeneous protein nanopore comprising two or more monomers, wherein at least one monomer contains a reactive site, and the other monomers do not contain a reactive site.

Solution 2. The heterogeneous protein nanopore according to solution 1, wherein the reactive site is an amino acid that is capable of interacting with a target analyte or is capable of linking to a sensing moiety, wherein the sensing moiety is capable of interacting with a target analyte.

Solution 3. The heterogeneous protein nanopore according to solution 1 or 2, wherein the heterogeneous protein nanopore is a variant of the nanopore selected from the group consisting of MspA, α-HL, Aerolysin, ClyA, FhuA, FraC, PlyA/B, CsgG, Phi 29 connector and a homolog thereof.

Solution 4. The heterogeneous protein nanopore according to any one of solutions 1-3, wherein heterogeneous protein nanopore is a variant of MspA which comprises at least one amino acid mutation on at least one monomer compared to MspA or M2 MspA.

Solution 5. The heterogeneous protein nanopore according to solution 4, comprising one monomer that contains the reactive site, and seven monomers that do not contain the reactive site.

Solution 6. The heterogeneous protein nanopore according to solution 4 or 5, wherein the reactive site is an amino acid located at a position selected from 83-111, preferably 90, 91, 92 and 93.

Solution 7. The heterogeneous protein nanopore according to any one of solutions 1-6, wherein the reactive site is selected from the group consisting of cysteine, methionine, lysine, and unnatural amino acid.

Solution 8. A protein nanopore reactor, comprising the heterogeneous protein nanopore according to any one of solutions 1-7 and optionally a sensing moiety linked to the reactive site.

Solution 9. The protein nanopore reactor according to solution 8, wherein the reactive site or the sensing moiety is capable of interacting with a target analyte.

Solution 10. The protein nanopore reactor according to solution 9, wherein the sensing moiety is phenylboronic acid (PBA).

Solution 11. The protein nanopore reactor according to any one of solutions 8-10, wherein the target analyte is selected from the group consisting of:
ion comprising metal element; preferably ion comprising alkaline-earth metal or transition metal; more preferably, AuCl₄⁻, Mg²⁺, Ca²⁺, Ba²⁺, Ni²⁺, Cu²⁺, Co²⁺, Zn²⁺, Cd²⁺, Ag²⁺ or Pb²⁺;
monosaccharide; preferably D-glyceraldehyde, D-erythrose, D-ribose, 2′-deoxy-D-ribose, D-xylose, L-arabinose, D-lyxose, D-glucose, D-galactose, D-mannose, D-fructose, L-sorbose, L-fucose, D-allose, D-tagatose, L-rhamnose, N-acetylneuraminic acid (sialic acid);
oligosaccharide; preferably disaccharide such as sucrose, isomaltulose, maltulose, turanose, leucrose, trehalulose, lactulose, maltose, trisaccharide such as raffinose or tetrasccharide such as acarbose or stachyose;
polysaccharide such as verbascose;
a compound containing a ribose moiety; preferably, nucleotide or modified nucleotide, or monophosphate derivative, diphosphate derivative, triphosphate derivative or tetraphosphate derivative of nucleotide or modified nucleotide, or nucleoside or nucleoside analogue; preferably, nucleotide comprises adenine nucleotide, cytosine nucleotide, uracil nucleotide or guanine nucleotide; preferably, the modified nucleotide comprises a nucleotide containing 5-methylcytidine (m⁵C), N6-methyladenosine (m⁶A), pseudouridine (Ψ), inosine (I), N7-methylguanosine (m⁷G) or N1-methyladenosine (m¹A); preferably, the nucleoside analogue comprises galidesvir, ribavirin, molnupiravir or remdesivir;
nucleotide sugar, such as uridine diphosphate glucose, uridine diphosphate N-acetylglucosamine, uridine diphosphate glucuronic acid, adenosine diphosphate glucose, uridine diphosphate galactose, uridine diphosphate xylose, guanosine diphosphate mannose, guanosine diphosphate fucose, cytidine monophosphate N-acetylneuraminic acid or uridine diphosphate N-acetylgalactosamine;
alditols, such as erythritol, threitol, arabitol, xylitol, adonitol, fucitol, sorbitol, mannitol, dulcitol, iditol, talitol, allitol, maltitol, lactitol or isomalt;
polyphenol, such as anthocyanin or proanthocyanidin;
catecholamine or catecholamine derivative; preferably, epinephrine, norepinephrine or isoprenaline;
catechol or derivative thereof, such as catechol, 3-fluorocatechol, 3-chlorocatechol, 3-bromocatechol, 4-fluorocatechol, 4-chlorocatechol, 4-bromocatechol, 3-methylcatechol, 4-methylcatechol, 3-methoxycatechol, 3-propylcatechol, 3-isopropylcatechol, 3,6-dibromocatechol, 4,5-dibromocatechol, 3,6-dichlorocatechol;
hydrogen peroxide;
buffer reagent; preferably, tris;
glycerin;
or any combination thereof.

Solution 12. The protein nanopore reactor according to solution 9, wherein the sensing moiety is nickel ions, cobalt ions or copper ions.

Solution 13. The protein nanopore reactor according to solution 9 or 12, wherein the target analyte is selected from the group consisting of natural amino acids, unnatural amino acids and modified amino acids such as selenocysteine.

Solution 14. A method for identifying a target analyte, comprising:
(i) providing the protein nanopore reactor according to any one of solutions 8-13;
(ii) applying a voltage between the two sides of the protein nanopore reactor;
(iii) allowing a target analyte to pass through the nanopore; and
(iv) measuring an ionic current through the nanopore to provide a current pattern, and identifying the target analyte based on the current pattern.

Solution 15. The method according to solution 14, wherein the target analyte is selected from the group consisting of:
ion comprising metal element; preferably ion comprising alkaline-earth metal or transition metal; more preferably, AuCl₄⁻, Mg²⁺, Ca²⁺, Ba²⁺, Ni²⁺, Cu²⁺, Co²⁺, Zn²⁺, Cd²⁺, Ag²⁺ or Pb²⁺;
monosaccharide; preferably D-glyceraldehyde, D-erythrose, D-ribose, 2′-deoxy-D-ribose, D-xylose, L-arabinose, D-lyxose, D-glucose, D-galactose, D-mannose, D-fructose, L-sorbose, L-fucose, D-allose, D-tagatose, L-rhamnose, N-acetylneuraminic acid (sialic acid);
oligosaccharide; preferably disaccharide such as sucrose, isomaltulose, maltulose, turanose, leucrose, trehalulose, lactulose, maltose, trisaccharide such as raffinose or tetrasccharide such as acarbose or stachyose;
polysaccharide such as verbascose;
a compound containing a ribose moiety; preferably, nucleotide or modified nucleotide, or monophosphate derivative, diphosphate derivative, triphosphate derivative or tetraphosphate derivative of nucleotide or modified nucleotide, or nucleoside or nucleoside analogue; preferably, nucleotide comprises adenine nucleotide, cytosine nucleotide, uracil nucleotide or guanine nucleotide; preferably, the modified nucleotide comprises a nucleotide containing 5-methylcytidine (m⁵C), N6-methyladenosine (m⁶A), pseudouridine (Ψ), inosine (I), N7-methylguanosine (m⁷G) or N1-methyladenosine (m¹A); preferably, the nucleoside analogue comprises galidesvir, ribavirin, molnupiravir or remdesivir;
nucleotide sugar, such as uridine diphosphate glucose, uridine diphosphate N-acetylglucosamine, uridine diphosphate glucuronic acid, adenosine diphosphate glucose, uridine diphosphate galactose, uridine diphosphate xylose, guanosine diphosphate mannose, guanosine diphosphate fucose, cytidine monophosphate N-acetylneuraminic acid or uridine diphosphate N-acetylgalactosamine;
alditols, such as erythritol, threitol, arabitol, xylitol, adonitol, fucitol, sorbitol, mannitol, dulcitol, iditol, talitol, allitol, maltitol, lactitol or isomalt;
polyphenol, such as anthocyanin or proanthocyanidin;
catecholamine or catecholamine derivative; preferably, epinephrine, norepinephrine or isoprenaline;
catechol or derivative thereof, such as catechol, 3-fluorocatechol, 3-chlorocatechol, 3-bromocatechol, 4-fluorocatechol, 4-chlorocatechol, 4-bromocatechol, 3-methylcatechol, 4-methylcatechol, 3-methoxycatechol, 3-propylcatechol, 3-isopropylcatechol, 3,6-dibromocatechol, 4,5-dibromocatechol, 3,6-dichlorocatechol;
hydrogen peroxide;
buffer reagent; preferably, tris;
glycerin;
or any combination thereof.

Solution 16. Use of the heterogeneous protein nanopore according to any one of solutions 1-7 or the protein nanopore reactor according to any one of solutions 8-13 in identifying a target analyte.

Solution 17. the use according to solution 16, wherein the target analyte is selected from the group consisting of:
ion comprising metal element; preferably ion comprising alkaline-earth metal or transition metal; more preferably, AuCl₄⁻, Mg²⁺, Ca²⁺, Ba²⁺, Ni²⁺, Cu²⁺, Co²⁺, Zn²⁺, Cd²⁺, Ag²⁺ or Pb²⁺;
monosaccharide; preferably D-glyceraldehyde, D-erythrose, D-ribose, 2′-deoxy-D-ribose, D-xylose, L-arabinose, D-lyxose, D-glucose, D-galactose, D-mannose, D-fructose, L-sorbose, L-fucose, D-allose, D-tagatose, L-rhamnose, N-acetylneuraminic acid (sialic acid);
oligosaccharide; preferably disaccharide such as sucrose, isomaltulose, maltulose, turanose, leucrose, trehalulose, lactulose, maltose, trisaccharide such as raffinose or tetrasccharide such as acarbose or stachyose;
polysaccharide such as verbascose;
a compound containing a ribose moiety; preferably, nucleotide or modified nucleotide, or monophosphate derivative, diphosphate derivative, triphosphate derivative or tetraphosphate derivative of nucleotide or modified nucleotide, or nucleoside or nucleoside analogue; preferably, nucleotide comprises adenine nucleotide, cytosine nucleotide, uracil nucleotide or guanine nucleotide; preferably, the modified nucleotide comprises a nucleotide containing 5-methylcytidine (m⁵C), N6-methyladenosine (m⁶A), pseudouridine (Ψ), inosine (I), N7-methylguanosine (m⁷G) or N1-methyladenosine (m¹A); preferably, the nucleoside analogue comprises galidesvir, ribavirin, molnupiravir or remdesivir;
nucleotide sugar, such as uridine diphosphate glucose, uridine diphosphate N-acetylglucosamine, uridine diphosphate glucuronic acid, adenosine diphosphate glucose, uridine diphosphate galactose, uridine diphosphate xylose, guanosine diphosphate mannose, guanosine diphosphate fucose, cytidine monophosphate N-acetylneuraminic acid or uridine diphosphate N-acetylgalactosamine;
alditols, such as erythritol, threitol, arabitol, xylitol, adonitol, fucitol, sorbitol, mannitol, dulcitol, iditol, talitol, allitol, maltitol, lactitol or isomalt;
polyphenol, such as anthocyanin or proanthocyanidin;
catecholamine or catecholamine derivative; preferably, epinephrine, norepinephrine or isoprenaline;
catechol or derivative thereof, such as catechol, 3-fluorocatechol, 3-chlorocatechol, 3-bromocatechol, 4-fluorocatechol, 4-chlorocatechol, 4-bromocatechol, 3-methylcatechol, 4-methylcatechol, 3-methoxycatechol, 3-propylcatechol, 3-isopropylcatechol, 3,6-dibromocatechol, 4,5-dibromocatechol, 3,6-dichlorocatechol;
hydrogen peroxide;
buffer reagent; preferably, tris;
glycerin;
or any combination thereof.

Solution 18. A method for preparing the heterogeneous protein nanopore according to any one of solutions 1-7, comprising:
(a) expressing the modified monomers and the unmodified monomers in the same host cell, wherein an additional polyamino acid is added to the end of any one of the modified monomer and the unmodified monomer, and the polyamino acid is sufficient to make the monomers with the polyamino acid and the monomers without the polyamino acid have distinguishable molecular weight differences;
(b) allowing the modified monomer and the unmodified monomer to self-assemble;
(c) purifying heterogeneous protein nanopores with a specific number of modified monomers and a specific number of unmodified monomers by the molecular weight difference.

### EXAMPLES

### Example 1: Single Molecule Identification of Monosaccharides with a Mycobacterium smegmatis Porin A Nanopore Modified with Boronic Acid

Saccharides play critical roles in many forms of cellular activities including energy provision, structural constitution and immune recognition. Saccharide structures are however extremely complicated and similar, setting a technical hurdle for direct identification. Nanopores, which are emerging single molecule tools sensitive to minor structural differences between analytes, can be engineered to identity saccharides. A hetero-octameric Mycobacterium smegmatis porin A (MspA) nanopore containing a sole phenylboronic acid (PBA) was prepared, and was able to clearly identify nine monosaccharide types, including D-Fructose, D-Galactose, D-Mannose, D-Glucose, L-Sorbose, D-Ribose, D-Xylose, L-Rhamnose and N-Acetyl-D-Galactosamine. Acknowledging the high resolution provided by the conical structure of MspA, minor structural differences between saccharide epimers can also be distinguished. To assist automatic event classification, a machine learning algorithm was developed, with which a general accuracy score of 0.96 was achieved. This sensing strategy is generally suitable for other saccharide types or even small oligosaccharides and may bring new insights to nanopore saccharide sequencing.

### INTRODUCTION

Saccharides, also known as carbohydrates, are critical biomolecules for almost all living creatures¹. As a core component of food, they provide energy to fuel almost all cellular activities². They also constitute the main building blocks of cellulose and pectin, providing structural integrity to cells³. Glycosylation, the process by which glycans are covalently linked to lipid or protein to form lipopolysaccharides or glycoproteins, is essential for the physiological and pathological functions of cells⁴⁻⁶. The recent discovery of glycoRNA demonstrates that conserved small noncoding RNAs also bear sialylated glycans⁷. The diverse functions of saccharides result from their versatile structures, which can be extremely complicated and whose mechanisms of action are not fully understood^{8,9}. Though investigation of polysaccharide sequence or structure can be performed by (micro) arrays^{10,11} capillary electrophoresis (CE)^{12,13}, liquid chromatography (LC)^{14,15}, nuclear magnetic resonance (NMR)^{16,17} and mass spectrometry (MS)^{18,19}, characterization performed by any single method can offer only an incomplete picture of the glycan analyte²⁰. Specifically, MS is blind to stereochemical information of monosaccharides and fails to discriminate between isomers^{20,21}. The low abundance of ¹⁵N in nature makes use of NMR to determine the amino-modified structure carried on glycans difficult^{20,22}. Saccharide characterizations by these means are generally expensive and time-consuming. A large quantity of input material may be required and the corresponding data interpretation is not straightforward^{23,24.}

Recent developments in nanopore sequencing of nucleic acids²⁵⁻²⁷ or peptides^{28,29} have suggested its potential to sequence polysaccharides in a similar manner. However, due to extremely similar structures of the monosaccharide components³⁰, the need for a nanopore which can fully discriminate between monosaccharides becomes urgent. Though polysaccharide sensing using solid state nanopores was previously performed³¹⁻³⁴, direct identification of monosaccharides using solid state nanopores has never been reported. In an aqueous environment, boronic acid is known to form reversible covalent bonds with 1,2 or 1,3-diols³⁵, including saccharides^{36,37}. However, the design of a boronic acid sensor which selectively reports binding of a specific saccharide type against all others can be extremely sophisticated³⁸ By placing a phenylboronic acid (PBA) adapter in α-hemolysin (α-HL), direct sensing of D-Glucose, D-Fructose and D-Maltose was reported³⁹. However, probably due to its disadvantageous cylindrical lumen geometry which resulted in a low resolution, discrimination between D-Glucose and D-Fructose was not truly achieved and no other types of saccharides were tested by this method.³⁹

Mycobacterium smegmatis porin A (MspA), an octameric pore-forming toxin with an overall conical lumen structure⁴⁰⁻⁴², is the first nanopore that successfully sequenced DNA²⁵. It then demonstrated direct discrimination between epigenetic modifications⁴³ and DNA lesions^{44,45} during nanopore sequencing. Engineering of its pore constriction also enabled MspA to directly monitor chemical reactions at a high resolution^{46,47}. A recent demonstration using a programmable nanopore reactor also showed that a phenylboronic acid can be placed in the pore lumen to report binding of polyols such as epinephrine or Remdesivir⁴⁸. This report suggests installation of a PBA in MspA for saccharide sensing. However, to the best of our knowledge, report of saccharide sensing using engineered MspA has never appeared.

### Results

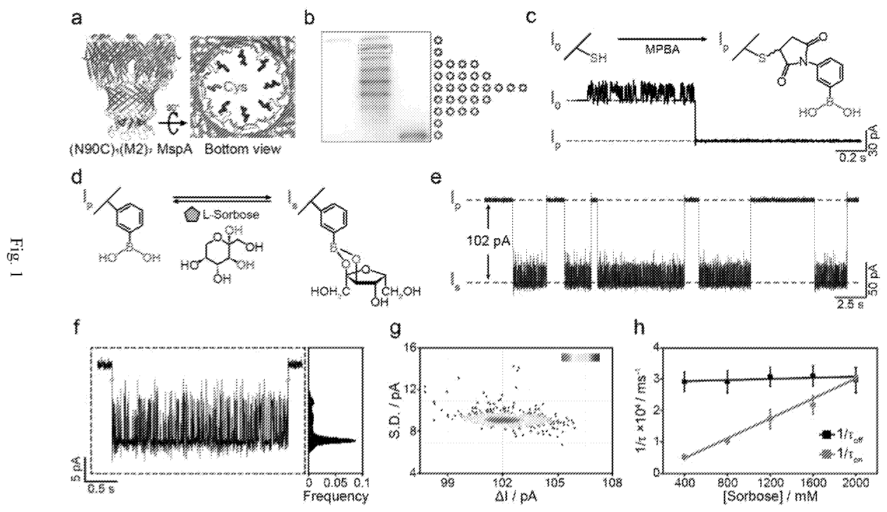

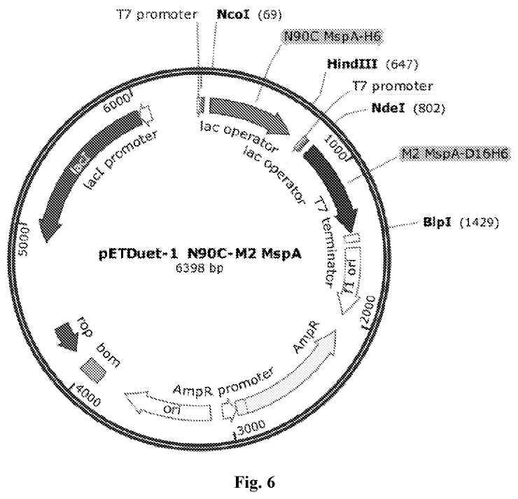

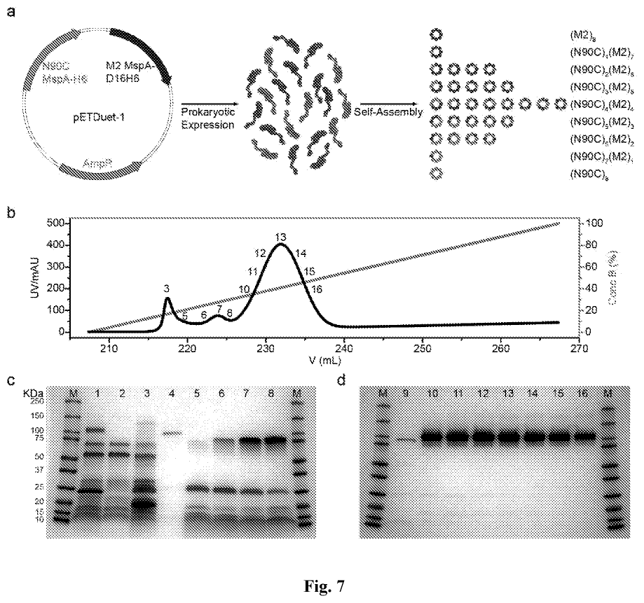

Prior to the placement of a sole PBA to MspA, a hetero-octameric MspA was first prepared. Experimentally, two different genes, coding respectively for M2 MspA-D16H6 (Table 1) and N90C MspA-H6 (Table 1), were custom synthesized and simultaneously inserted in a pETDUET-1 co-expression vector (FIG. 6). After heat shock transformation with this vector, the E. coli BL21 (DE3) pLysS strain was applied to co-express both genes (Methods in Example 1, FIG. 7) and generated octameric MspA assemblies composed of different fractions of both protein types (FIG. 1b). The MspA assembly, which is composed of one unit of N90C MspA-H6 and seven units of M2 MspA-D16H6, is the desired MspA hetero-octamer and is referred to as (N90C)₁(M2)₇ (FIG. 1a). (N90C)₁(M2)₇ contains a single cysteine at site 90 of the N90C MspA-H6 component, at the pore constriction. Experimentally, (N90C)₁(M2)₇ was purified from other types of MspA assemblies by gel separation and was used directly for all downstream measurements (FIG. 1b).

To introduce a phenylboronic acid (PBA) group to (N90C)₁(M2)₇, 3-(maleimide) phenylboronic acid (MPBA) was chemically bonded to the only cysteine of (N90C)₁(M2)₇ by maleimide-thiol coupling (FIG. 1c). A real-time single molecule characterization of this reaction was achieved by performing single channel recording (Methods in Example 1). Briefly, the electrophysiology measurement was performed in a 1.5 M KCl, 10 mM MOPS, pH 7.0 buffer with a single (N90C)₁(M2)₇. With a +100 mV bias continually applied, the open pore current of (N90C)₁(M2)₇ (I₀) measures ˜295 pA. Additional shot noises were also observed at this stage (FIG. 1c). The thiol residue of the cysteine at the pore constriction may contribute to the generation of these noises, a phenomenon also previously reported by engineered α-hemolysin (α-HL) mutants⁴⁹. Subsequently, MPBA was added to cis to a 2 mM final concentration. An irreversible single-step drop of current measuring ˜53 pA was consequently observed. No further drop of current was noted and the previously observed shot noises also simultaneously disappeared. These phenomena proved that an MPBA had been successfully conjugated to the only cysteine of (N90C)₁(M2)₇. This PBA conjugated MspA hetero-octamer is referred to as MspA-PBA, of which the open pore current is defined as Iₚ (FIG. 1c).

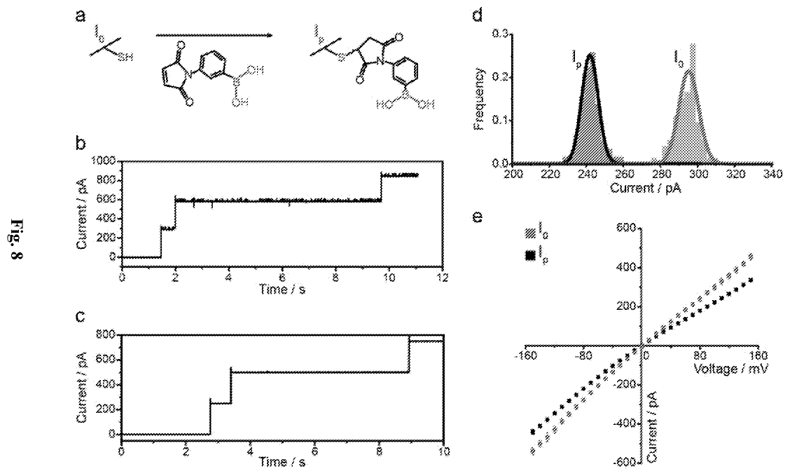

Preparation of MspA-PBA in an ensemble was performed by mixing purified (N90C)₁(M2)₇ with MPBA prior to single channel recording (Methods in Example 1). Further characterization of the open pore current of (N90C)₁(M2)₇ (I₀) and MspA-PBA (Iₚ) demonstrated that a current difference between those measured with (N90C)₁(M2)₇ and those using MspA-PBA was constant, indicating that the prepared MspA-PBA reports a uniform structure and could be easily discriminated from the unmodified form (N90C)₁(M2)₇ (FIG. 8, Tables 2, 3). The I₀ and Iₚ values are also consistent with that previously measured during single channel recording (FIG. 1c), confirming that the ensemble prepared MspA-PBA is identical to that previously characterized during real time pore modification. If not otherwise stated, all subsequent measurements were performed using MspA-PBA prepared in an ensemble.

L-Sorbose is a monosaccharide ketose. It exists in all living species, ranging from bacteria to human⁵⁰. The commercial production of vitamin C (ascorbic acid) often begins with L-Sorbose⁵¹. To further verify the existence of a PBA in the lumen of the pore, MspA-PBA was used to sense L-Sorbose. The measurement was performed with a single MspA-PBA and a 1.5 M KCl, 10 mM MOPS, pH 7.0 buffer with the continuous application of a +160 m V bias. The addition of L-Sorbose to cis to a 10 mM final concentration resulted immediately in the consecutive appearance of long residing resistive pulse events (FIGS. 1d-1f, Supplementary Video 1). However, when a M2 MspA was tested, instead of a MspA-PBA, addition of L-Sorbose to cis to a 50 mM final concentration failed to cause any resistive pulse events (FIG. 11). In this way, it was confirmed that the introduction of a PBA to the pore lumen is critical for the generation of saccharide sensing events.

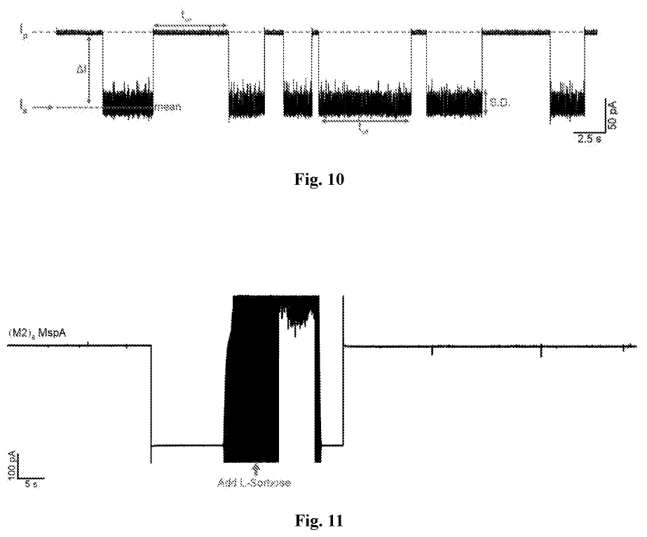

To describe the events quantitatively, parameters such as the event dwell time (τ_{off}), the inter-event interval (τₒₙ), the blockage level (Iₛ), the blockage amplitude (ΔI=Iₚ−Iₛ) and the standard deviation (S.D.) of each event are defined in FIG. 10. Generally, the L-Sorbose sensing events report a large but uniform blockage amplitude (ΔI) measuring ˜100 pA, more than 10-fold larger than that previously reported when a monosaccharide was sensed by an α-HL modified with a boronic acid³⁹. A highly characteristic noise feature was also consistently observed. This has been well described in the scatter plot of ΔI vs. S.D. (FIG. 1g), in which only a single population of events was observed. The normalized scatter plots of ΔI/Iₚ vs. S.D. were also shown with results from three independent measurements (FIG. 13, N=3) and the same conclusion was drawn.

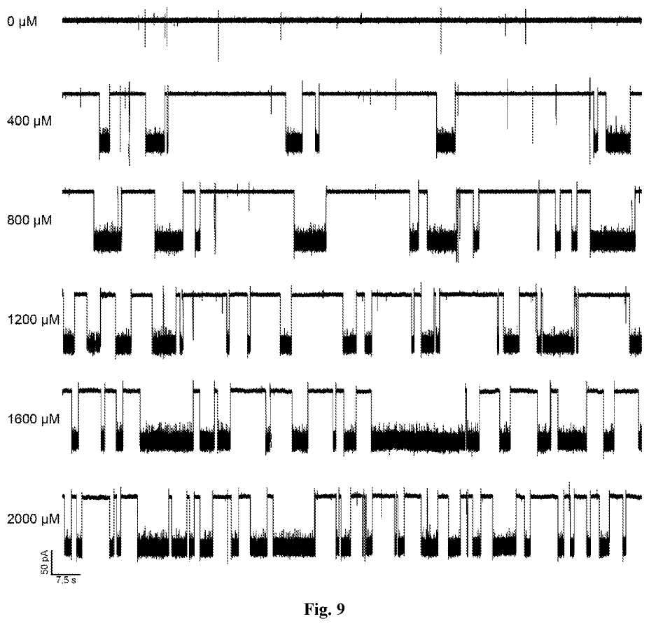

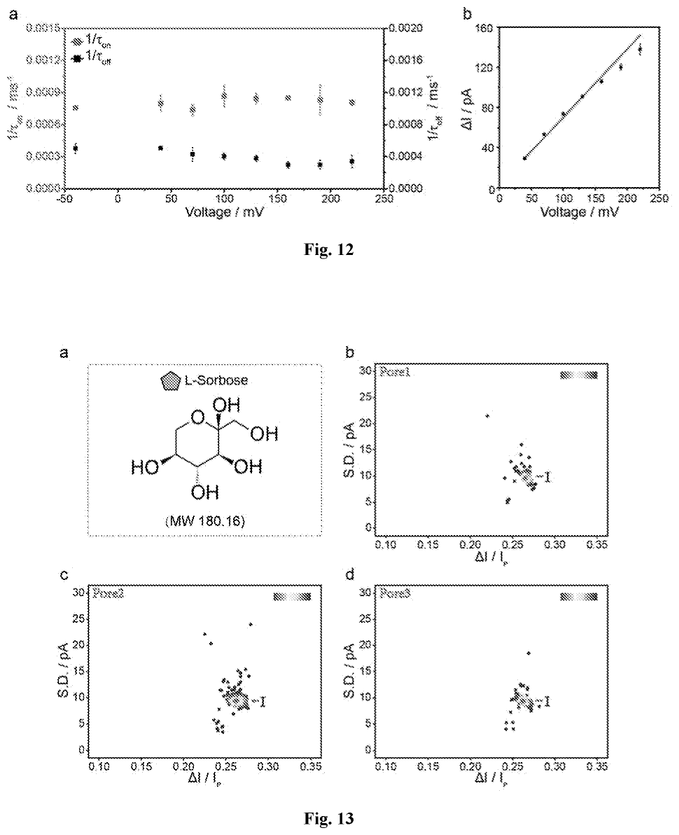

By continually upregulating the L-Sorbose concentration in cis during the measurement, the rate of event appearance was proportionally increased (FIG. 1h, FIG. 9, Table 4). To quantitatively describe the concentration dependence of the binding kinetics, the mean dwell time (τ_{off}) or the mean inter-event interval (τₒₙ) were derived from results of exponential fittings to the histograms of τ_{off} or τₒₙ, respectively. The reciprocal of the mean inter-event interval (1/τₒₙ) increases linearly with the increase in the L-Sorbose concentration, consistent with a bimolecular model. The 1/τ_{off} value however is independent of the L-Sorbose concentration, consistent with a unimolecular dissociation mechanism. This further confirms that the resistive pulse events are the result of reversible binding of individual L-Sorbose molecules to the sole phenylboronic acid reactive site of a single MspA-PBA. The same measurement was also performed with different applied voltages. Though the blockage amplitude (ΔI) increases linearly with the applied voltage, the τₒₙ and τ_{off} stay almost unchanged (FIG. 12, Tables 5, 6). Though geometrically confined in the pore constriction, the reaction between a PBA and a L-Sorbose is not interfered with by the local electric field. Instead, diffusion of the analyte plays a more critical role in the modulation of the rate of event appearance. This is expected because the L-Sorbose is an electrically neutral molecule under the condition being tested. Though not demonstrated in this paper, it is expected that the binding kinetics of charged saccharides would be strongly modulated by the applied voltage. Experimentally, though the measurement at a higher voltage reports a larger event amplitude, spontaneous pore closure is also observed more frequently. A +160 mV bias was thus found to be optimum for continuous and time-extended measurements.

The feasibility of saccharide sensing by MspA-PBA has now been successfully demonstrated with L-Sorbose. The same principle may also be applied to sensing of other saccharide analytes, as long as the analyte can react with the PBA at the pore constriction. The giant event amplitude and a unique fluctuation noise observed from L-Sorbose binding to MspA-PBA suggest that MspA has a resolution which may directly discriminate between different saccharide types solely by nanopore readouts. To approve this speculation, D-Fructose, D-Galactose, D-Mannose and D-Glucose were used as the analyte. These four types of saccharide also represent the most abundant monosaccharide types in nature⁵². Their molecular weights are identical, meaning that direct discrimination between them solely by mass spectrometry is impossible. Specifically, D-Mannose and D-Galactose are respectively the C2 and the C4 epimers of D-Glucose, and possess an extremely minor structural difference.

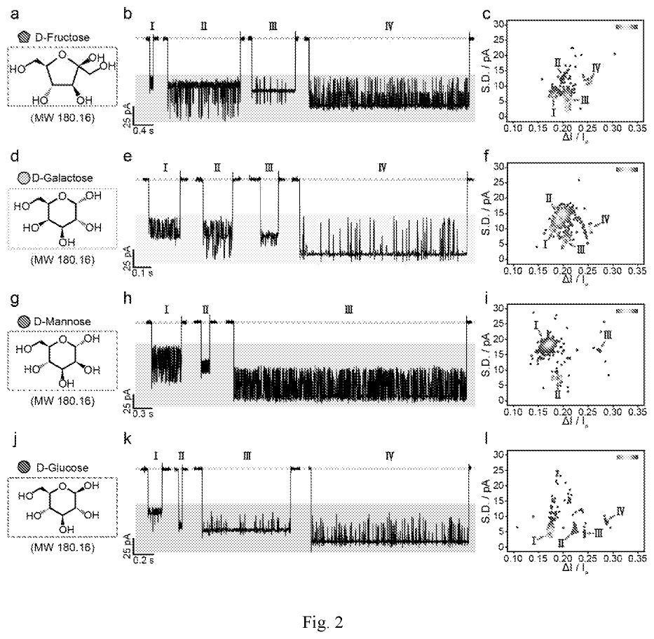

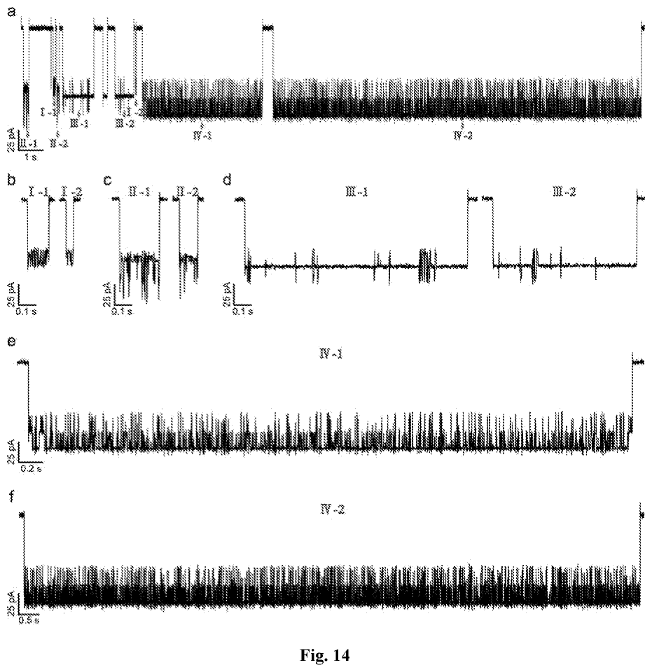

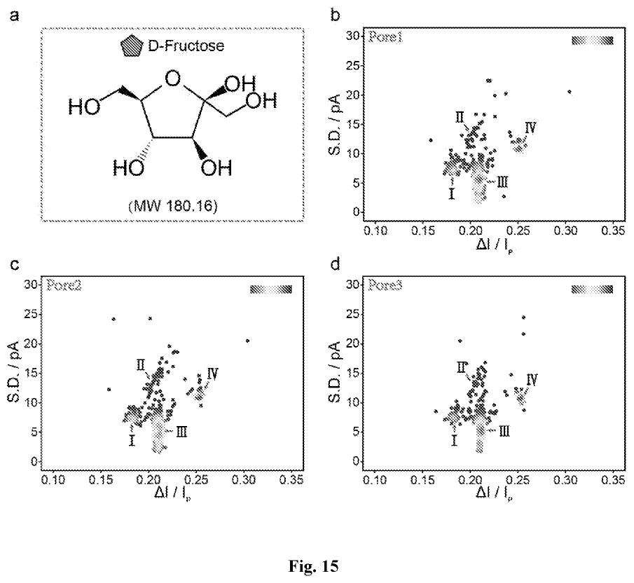

All subsequent measurements were performed with MspA-PBA in a 1.5 M KCl, 10 mM MOPS, pH 7.0 buffer. A +160 mV bias was continually applied. D-Fructose, D-Galactose, D-Mannose or D-Glucose was added to cis to the desired concentration. For D-Fructose (FIG. 2a), nanopore sensing reports more than one type of event. Representative events of each type were summarized in FIG. 2b and FIG. 14. This is expected because in an aqueous environment the D-Fructose exists as a mixture of pyranose and furanose isomers, each with an α- and β-anomer³⁸. Binding between different combinations of hydroxyl groups to a PBA may also contribute to the generation of different types of sensing events³⁶⁻³⁸. However, the events demonstrate highly consistent characteristics, as shown in the event scatter plot of ΔI/Iₚ vs. S.D., The local density of events is color coded to clearly show the event distribution. Independent measurements with D-Fructose (N=3) also show an extremely consistent pattern of event distribution as well (FIG. 15).

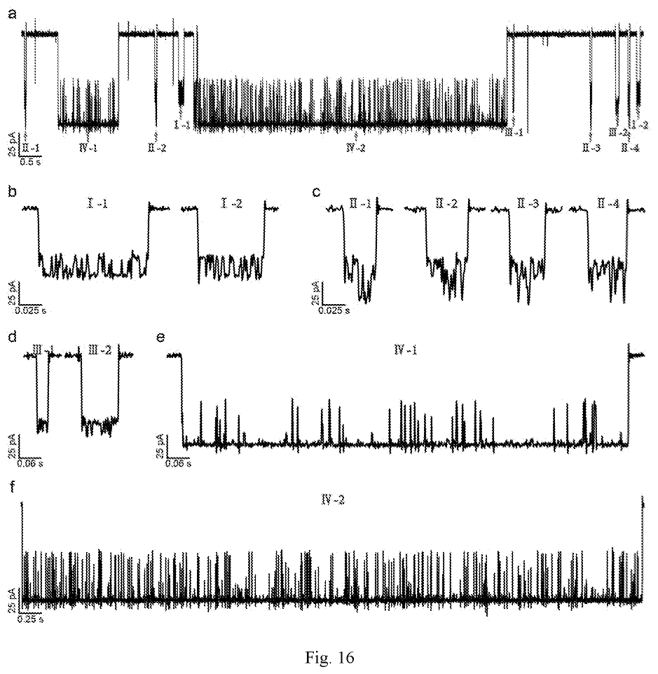

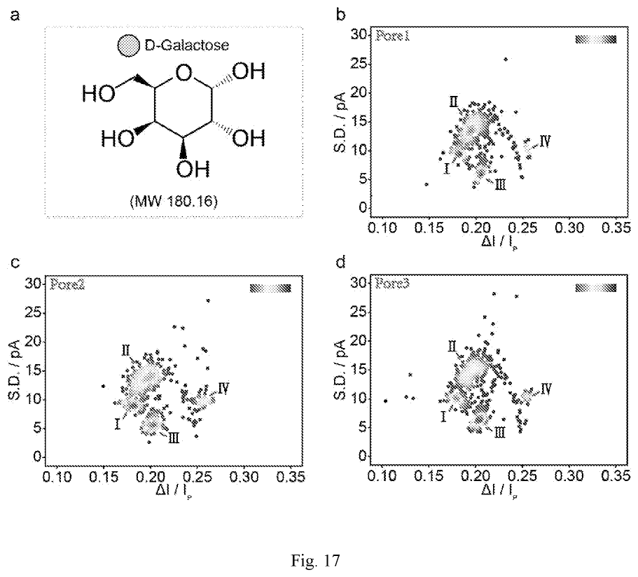

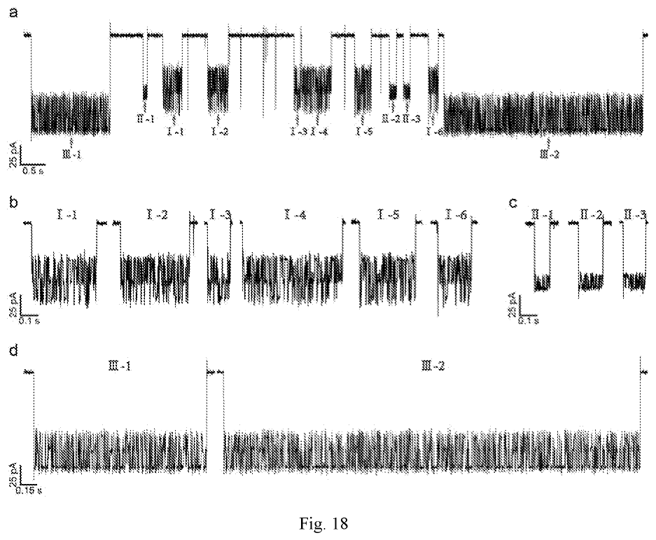

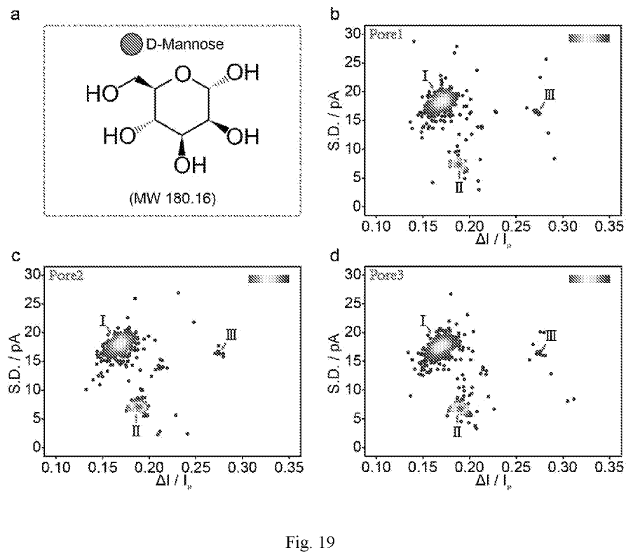

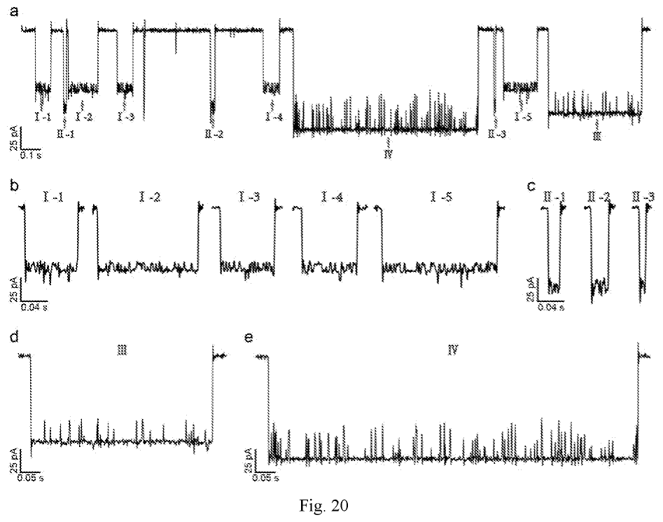

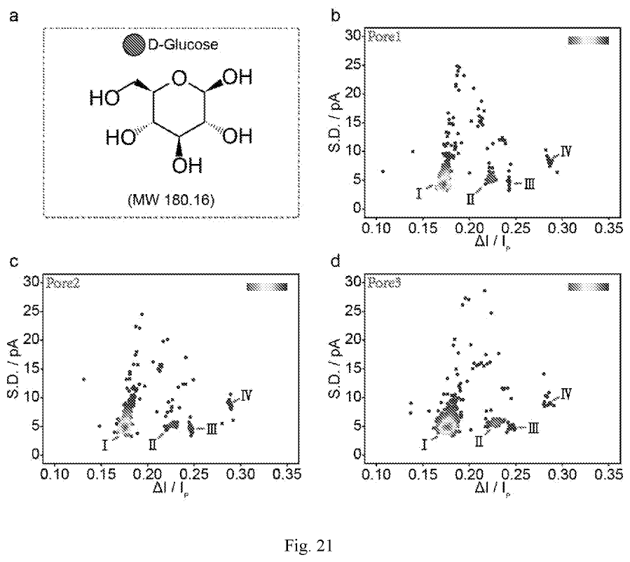

Following the same principle, D-Galactose (FIGS. 2d-2f), D-Mannose (FIGS. 2g-2i, Supplementary Video 2) and D-Glucose (FIGS. 2j-21) were also tested and evaluated. Similar to D-Fructose, either type of saccharide demonstrates more than one type of events when sensed by MspA-PBA (FIGS. 2e, 2h, 2k). These event types demonstrate highly discriminable blockage depth and characteristic event noises, useful in the identification of different saccharide types. The scatter plot results of D-Galactose (FIG. 2f), D-Mannose (FIG. 2i) and D-Glucose (FIG. 21) also show distinct event populations between different saccharide types. Detailed demonstrations of the consistency of event types and the repeatability between different trails for each condition (N=3) are summarized in FIGS. 16-21. Specifically, when the same type of saccharide was tested, the scatter plot results demonstrate a highly consistent pattern of event distribution (FIGS. 15, 17, 19, 21), providing information useful to discriminate between different saccharide types. Though events from different saccharide types are visually identifiable, it is still challenging to describe automatically and quantitatively the differences between saccharide types by only considering ΔI/Iₚ and S.D. of each event. The task can be even more complicated when different saccharides are sensed in a mixture.

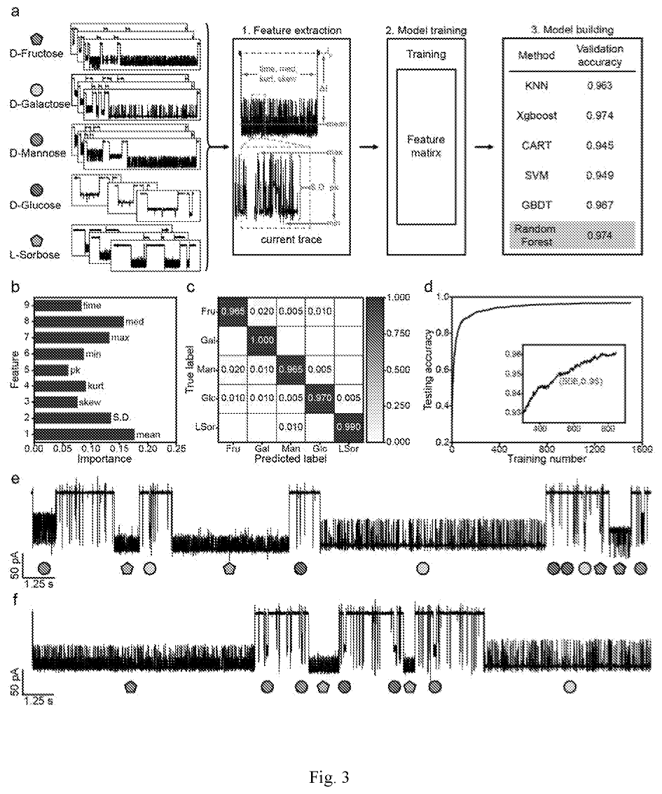

Machine learning, which aims to build computerized algorithms which can learn from data instead of focusing on the programming, is an important branch of artificial intelligence research^{53,54}. Machine learning has also been widely applied in previous reports of nanopore research^{32,33,55-60}. Existing sensing data of D-Fructose, D-Galactose, D-Mannose, D-Glucose and L-Sorbose demonstrate highly discriminable event features between each other and a high consistency when the same saccharide type was tested, forming the basis for automatic event classification by machine learning. The overall training process of machine learning contains feature extraction, model training and model building. First, nanopore measurements with MspA-PBA were separately performed with D-Fructose (FIGS. 2a-2c, FIGS. 14, 15), D-Galactose (FIGS. 2d-2f, FIGS. 16, 17), D-Mannose (FIGS. 2g-2i, FIGS. 18, 19), D-Glucose (FIGS. 2j-21, FIG. 20-21) or L-Sorbose (FIGS. 1e-1h, FIGS. 9, 13). Events from raw current-time traces of saccharide sensing were then extracted to derive the corresponding event features including mean (ΔI/Iₚ), standard deviation (S.D.), skewness (skew), kurtosis (kurt), minimum (min), maximum (max), peak-to-peak value (pk), median (med) and dwell time (time). The label of each event was assigned with the saccharide type being tested (FIG. 3a). Different event types for the same saccharide type being tested were however not differently labeled in machine learning. For each saccharide type, continuous measurements of more than 1 h were performed to collect sufficient events for each class. Events with less than 30 ms in the dwell time were however omitted. Subsequently, a minimum of 5000 events were collected for each class to form the database.

Prior to model training, 1000 events of each saccharide type were randomly sampled from the database to assemble a data set. The data set was then split into a training set (80%) for model training and a testing set (20%) for model testing. Six common machine learning models, including KNN, Xgboost, Regression Tree (CART), SVM, Gradient Boost (GBDT) and Random Forest were evaluated. All model evaluations were carried out with default hyperparameters. To avoid bias, 10-fold cross validation was applied during model training and evaluation, from which the validation accuracies for each model were derived and reported (FIG. 3a). Specifically, the validation accuracy is defined as the proportion of events being correctly recognized in the whole validation set. Generally, all models demonstrate a satisfactory performance by reporting a minimum accuracy score of 0.945. It indicates that the data quality of saccharide sensing is sufficient for saccharide identification. Specifically, the Random Forest model performed the best by reporting the highest validation accuracy of 0.974. This model was thus further tuned by hyperparameter optimization. The hyperparameters “n_estimators” and the “max_depth” were finely-tuned and then the validation accuracy was improved to 0.975. The feature importance of the finely-tuned Random Forest model was demonstrated in FIG. 3b, in which all parameters play an important role. However, the feature of mean (ΔI/I₀), median (med) and standard deviation (S.D.) contributed the most.

The finely-tuned model was further applied on the testing set to produce the confusion matrix (FIG. 3c), in which the accuracy of D-Fructose (Fru), D-Galactose (Gal), D-Mannose (Man), D-Glucose (Glc) and L-Sorbose (L-Sor) are 0.965, 1.000, 0.965, 0.970 and 0.990, respectively. The D-Galactose and the L-Sorbose demonstrate the highest score among all five monosaccharides. To estimate the efficiency of model training, a learning curve was produced, giving the accuracy score against a varying size of the training set. The results indicate that an overall judgement accuracy of 95% was achieved when 508 events, randomly selected from the whole training set, were fed to the program (FIG. 3d).

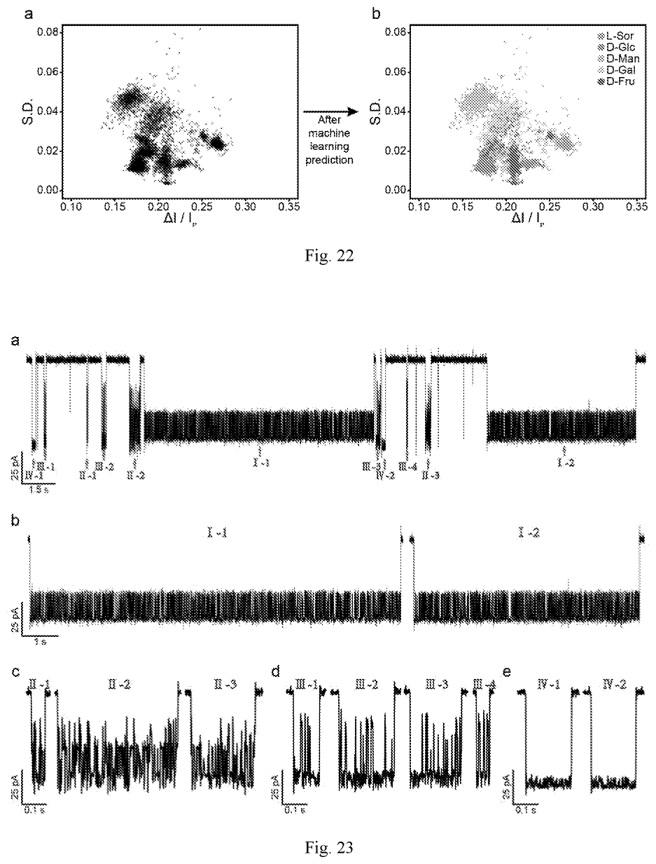

A nanopore measurement was then carried out with a mixture of D-Fructose, D-Galactose, D-Mannose, D-Glucose and L-Sorbose. The previously trained model was employed to predict unlabeled events acquired from this measurement. Representative traces were demonstrated in FIGS. 3e and 3f, on which the labels of events predicted by machine learning are marked. The marked labels are consistent with the event types previously demonstrated when a corresponding saccharide was tested as the sole analyte (FIGS. 1-2). A scatter plot of all events is also shown in FIG. 22. After prediction by machine learning, events resulted from binding of different saccharides were clearly discriminated from each other. The event distribution for each saccharide type in the scatter plot is also consistent with that when tested separately (FIGS. 13, 15, 17, 19 and 21).

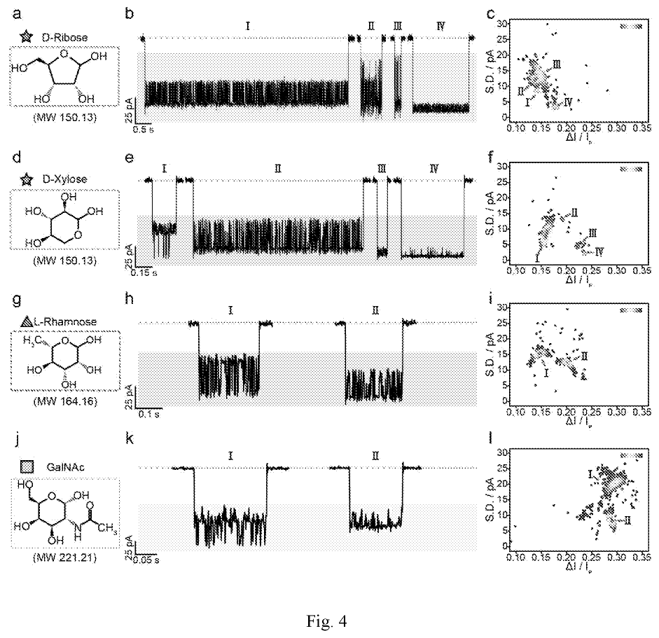

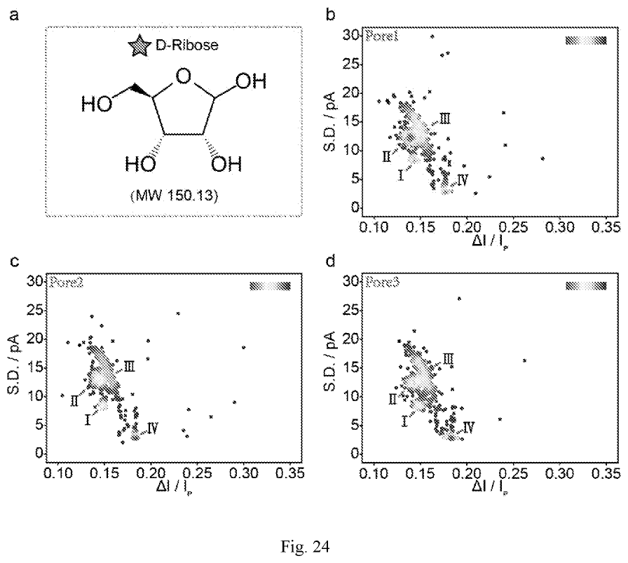

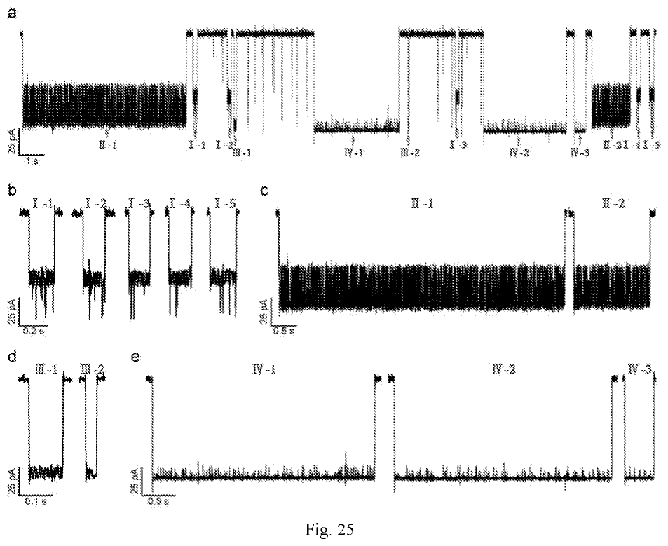

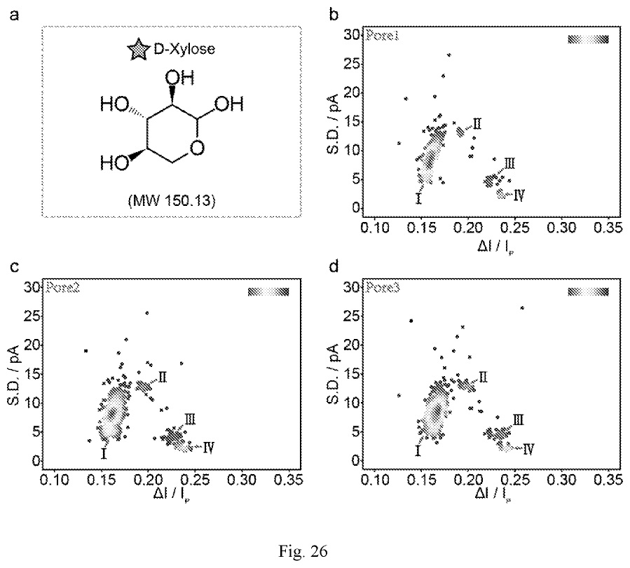

The saccharide types that were tested so far are all six-carbon saccharides. D-Ribose and D-Xylose, which are naturally occurring five-carbon sugars and epimers of each other, are in principle also detectable by MspA-PBA. Experimentally, D-Ribose (FIGS. 4a-c) and D-Xylose (FIGS. 4d-f) both report multiple types of events. Detailed demonstrations of event types and consistency of repetitive measurements are summarized in FIGS. 23-26. Though D-Ribose and D-Xylose have the same molecular weight, the event features and the pattern of event distribution are highly discriminatory.

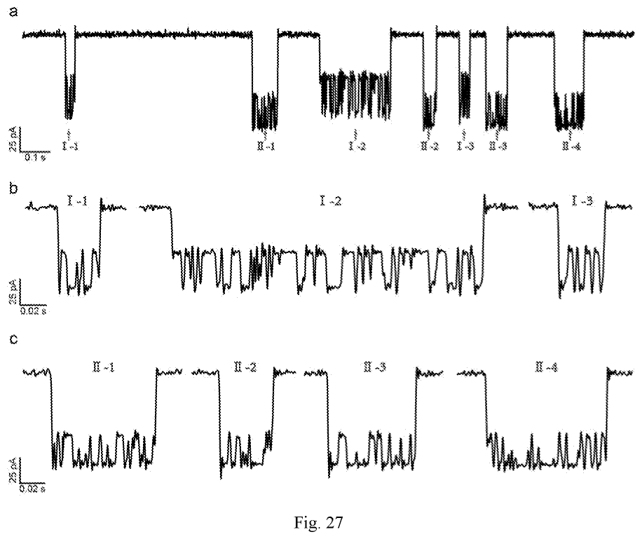

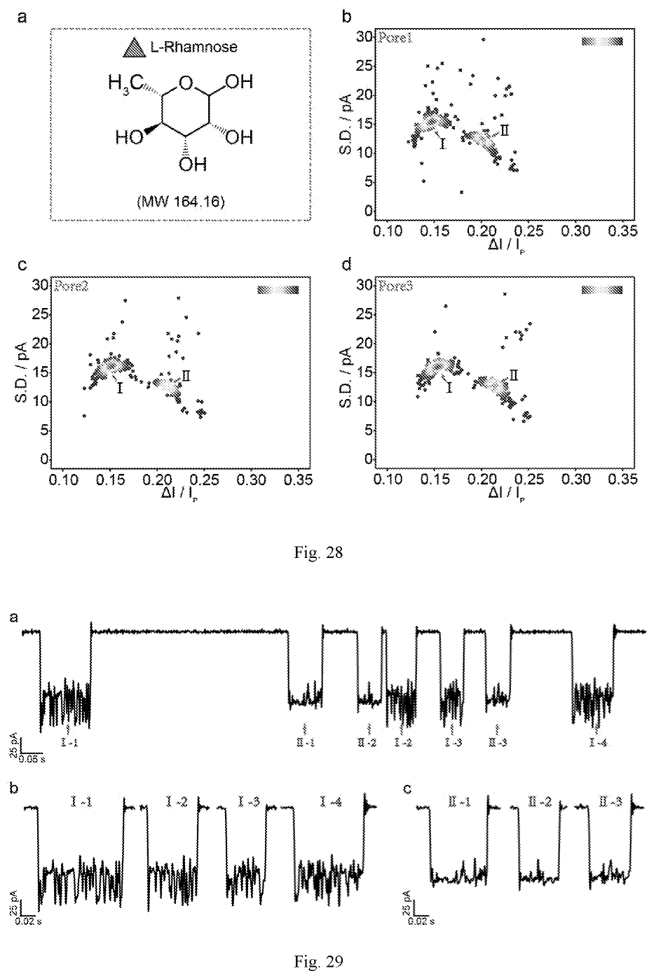

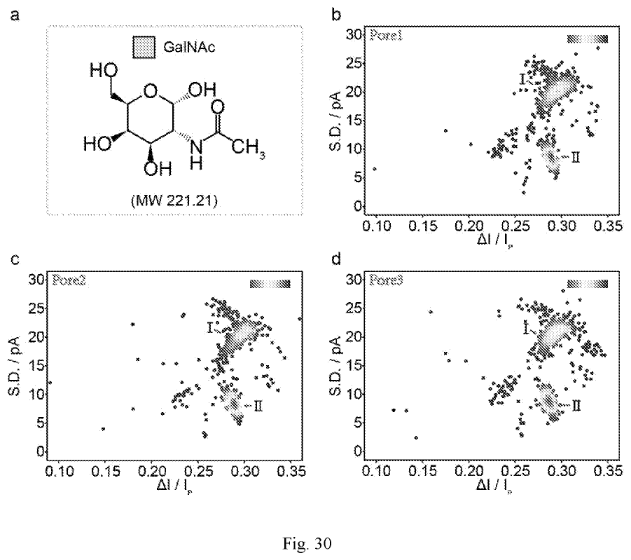

In a subsequent demonstration, L-Rhamnose (L-Rha) serves as a representative deoxysugar and N-Acetyl-D-Galactosamine (GalNAc) serves as a representative amino sugar. Both types of saccharides have a substituted hydroxyl group and the overall structures are significantly different from saccharide types tested so far, suggesting that they may also be easily discriminated by nanopores. The measurements were performed the same as that described above (FIGS. 1-2). L-Rhamnose or N-Acetyl-D-Galactosamine was respectively tested as the analyte when measured with MspA-PBA. L-Rhamnose (FIGS. 4g-4i) and N-Acetyl-D-Galactosamine (FIGS. 4j-41) both report two types of events. Detailed demonstration of event types and the consistency between different trials are summarized in FIGS. 27-32. Specifically, the N-Acetyl-D-Galactosamine reported the largest event amplitude. This presumably resulted from its clearly larger molecular size compared to all other saccharides being tested.

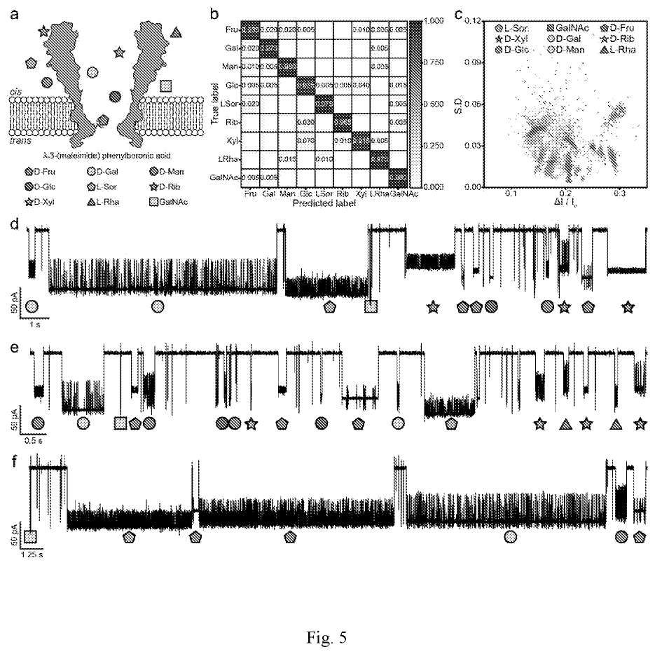

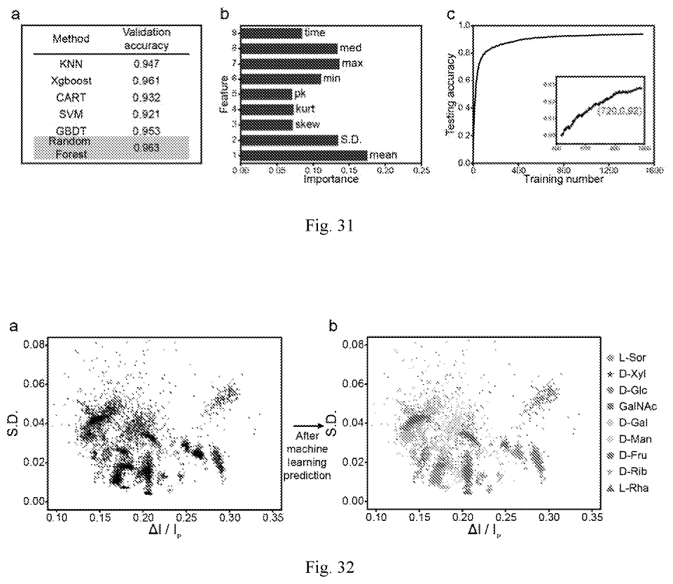

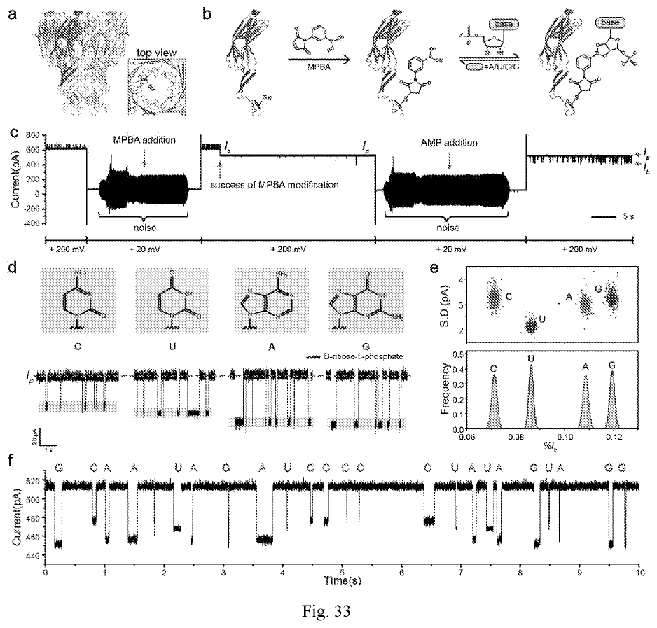

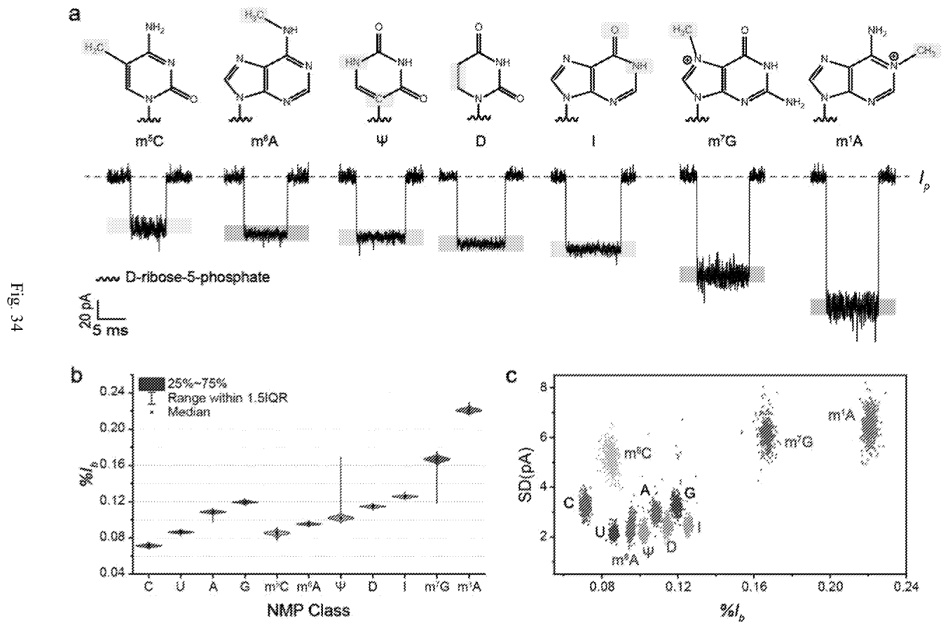

We now have nine classes of input data for machine learning, respectively taken from D-Fructose (Fru), D-Galactose (Gal), D-Mannose (Man), D-Glucose (Glc), L-Sorbose (L-Sor), D-Ribose (Rib), D-Xylose (Xyl), L-Rhamnose (L-Rha) and N-Acetyl-D-Galactosamine (GalNAc) (FIG. 5a). From each class, 1000 events were again randomly selected to form the data sets. The previous six models were evaluated for a second time with a larger database now containing nine classes of events (FIG. 31). The Random Forest model again outperformed all other models and was further finely-tuned. The learning curve and the feature importance of the finely-tuned model are shown in FIG. 31. The confusion matrix results of the testing set are shown in FIG. 5b, in which the accuracies of nine monosaccharides are all above 0.915. Though the general prediction accuracy has slightly decreased upon the inclusion of more saccharide types in the model, the Gal, Man, L-Sor, Rib, L-Rha and GalNAc all demonstrate extremely high accuracy scores, above 0.965. Nanopore measurements with a mixture of all nine saccharide types were performed the same as previously described (FIGS. 1-4). The acquired events from the mixture were collected to generate the corresponding scatter plot of ΔI/Iₚ versus S.D., Event labelling was predicted by the trained machine learning classifier (FIG. 5c, FIG. 32). The labelled scatter plot demonstrates discriminated saccharide sensing events, consistent with the results from separate tests performed with each saccharide type. Representative traces of saccharide sensing in a mixture were also demonstrated in FIGS. 5d-5f, in which the corresponding labels predicted by machine learning are marked.

### Conclusion

In summary, we have demonstrated direct identification of nine types of monosaccharide using a PBA attached hetero-octameric MspA. To the best of our knowledge, a hetero-octameric MspA containing a solely attached chemical reactive group as a nanoreactor has never been reported before. By generating large event amplitudes and rich event features during saccharide sensing, MspA demonstrates a superior performance of saccharide identification in single molecule^{31-34,39}. According to experimental^{46,57,61} and theoretical assessments^{62,63}, the conical lumen geometry of MspA contributes most to this superior resolution. Discrimination between saccharide isomers or epimers was also demonstrated, further confirming that MspA is structurally superior in saccharide identification. The extracted event features were fed into a machine learning based classifier and a 0.96 accuracy was reported. Some specific saccharide types such as GalNAc even report an accuracy score of 0.99. Though only demonstrated with representative monosaccharides, this sensing strategy is in principle suitable for other types of monosaccharides³⁶⁻³⁸, saccharide derivatives³⁶, saccharide medicines or small oligosaccharides^{64,65}, as long as the analyte can interact with the PBA and fits the size of the pore constriction. According to literatures^{35,66} and a recent report⁴⁸ performed using a programmable nanopore reactor, other polyols such as glycerol, vitamins, catechol, catecholamine and nucleotide analogues may also be sensed by MspA-PBA but will be reported in subsequent studies.

### Methods

### 1. Preparation of Homo-Octameric MspAs

The genes coding for M2 MspA-D16H6 and N90C MspA-H6 (Table 1) respectively were custom synthesized by GenScript (New Jersey). These two genes were separately inserted in pET-30a (+) plasmid DNAs between the restriction site of Nde I and Hind III. The constructed plasmids, referred to as pET-M2 MspA and pET-N90C respectively, were separately used in the preparation of homo-octameric M2 MspA-D16H6 and N90C-H6. Homo-octameric M2 MspA-D16H6 and N90C-H6 were applied as the standard during gel electrophoresis (FIG. 1b). Homo-octameric M2 MspA-D16H6 was also applied as a representative nanopore which doesn't contain any reactive site in the pore lumen (FIG. 11).

Experimentally, 1 μL (100 ng/μL) either plasmid DNA was added to 100 μL E. coli BL21 (DE3) pLysS competent cells (Sangon Biotech) in an Eppendorf tube and shaken to reach a homogeneous distribution. The tube was ice incubated for 30 min, incubated at 42° C. for 90 s and ice incubated for another 3 min. Then, 800 μL Luria-Bertani (LB) medium was added to the tube. The medium was then cultured at 37° C. and 175 rpm for 50 min. The medium was then evenly spread on an agar plate with 30 μg/mL kanamycin sulfate and 34 μg/mL chloramphenicol and cultured at 37° C. for 18 h. A single colony was collected and added to a 250 ml conical flask containing 100 mL LB liquid medium with 30 μg/mL kanamycin sulfate and 34 μg/mL chloramphenicol. The conical flask was shaken (175 rpm) at 37° C. until OD₆₀₀=0.7. It was then inducted by addition of isopropyl β-D-thiogalactoside (IPTG) to a 0.5 mM final concentration and shaken (175 rpm) at 16° C. for 16 h. Afterwards, the cells were harvested by centrifugation (4500 rpm, 4° C., 20 min). The bacterial pellet was resuspended in a 40 mL lysis buffer (100 mM Na₂HPO₄/NaH₂PO₄, 0.1 mM EDTA, 150 mM NaCl, 0.5% (w/v) Genapol X-80, pH 6.5) and heated at 60° C. for 10 min. The suspension was first cooled on ice for 10 min and then centrifuged at 4° C. for 40 min at 13,000 rpm to collect the supernatant. The supernatant was then syringe filtration treated and loaded to a nickel affinity column (HisTrap™ HP, GE Healthcare). The column was first eluted with buffer A (0.5 M NaCl, 20 mM HEPES, 5 mM imidazole, 0.5% (w/v) Genapol X-80, pH 8.0) and then eluted with a linear gradient of imidazole (5-500 mM) by mixing buffer A and buffer B (0.5 M NaCl, 20 mM HEPES, 500 mM imidazole, 0.5% (w/v) Genapol X-80, pH 8.0) during elution. When purifying N90C MspA-H6, an additional 2 mM Tris(2-carboxyethyl) phosphine (TCEP) was added to the buffer to prevent the formation of disulfide bonds between cysteine residues in the homo-octameric MspA. The eluted fractions were further characterized by SDS-polyacrylamide gel electrophoresis (PAGE) and the fraction containing the target protein was identified. A 4-15% Mini-PROTEAN TGX Gel (Bio-Rad. Cat #4561083) was used in this step. The identified fraction was immediately used or held at −80° C. for long term storage⁶⁷.

### 2. Preparation of Hetero-Octameric MspA

To prepare hetero-octameric MspAs composed of M2 MspA-D16H6 and N90C MspA-H6, both genes were simultaneously placed in a co-expression vector pETDuet-168. Briefly, the gene coding for N90C MspA-H6 was placed at the first multiple cloning site, between the restriction site Nco I and Hind III. The gene coding for M2 MspA-D16H6 was placed at the second multiple cloning site, between the restriction site Nde I and Blp I. The hexa-histidine tag (H6) at the C-terminus of each gene is designed to assist nickel affinity chromatography-based purification. A tag composed of 16 consecutive aspartic acids (D16), is added to the C-terminus of the gene coding for M2 MspA-D16H6, immediately before the hexa-histidine tag (H6). The D16 tag serves to generate a molecular weight difference between hetero-octameric MspAs composed of different fractions of M2 MspA-D16H6 and N90C MspA-H6. The D16 tag is thus useful in the purification of the desired hetero-oligomerized MspA composed of one N90C MspA-H6 and seven M2 MspA-D16H6, namely the (N90C)₁(M2)₇ (FIG. 1b).

1 μL (100 ng/μL) plasmid DNA was added to 100 μL E. coli BL21 (DE3) pLysS competent cells (Sangon Biotech) in an Eppendorf tube and shaken to reach a homogeneous distribution. The tube was ice incubated for 30 min, incubated at 42° C. for 90 s and ice incubated for another 3 min. Then, 800 μL Luria-Bertani (LB) medium was added to the tube. The medium was then cultured at 37° C. and 175 rpm for 50 min. The medium was then evenly spread on an agar plate with 30 μg/mL kanamycin sulfate and 34 μg/mL chloramphenicol and cultured at 37° C. for 18 h. Subsequently, a single colony was picked up and added to a 50 mL tube containing 10 mL LB liquid medium with 50 μg/mL ampicillin and 34 μg/mL chloramphenicol. The tube was shaken at 37° C. and 175 rpm for 5 h until OD₆₀₀=0.7. The medium was then added to a 1 L system for further cultivation at 37° C. and 175 rpm until OD₆₀₀=0.6. Then IPTG was added to the medium to reach a 0.1 mM final concentration and shaken for 24 h at 16° C. to induce protein overexpression. After that, the cells were harvested by centrifugation (4500 rpm, 20 min, 4° C.).

The collected bacterial pellet was resuspended in a 160 mL lysis buffer (100 mM Na₂HPO₄/NaH₂PO₄, 0.1 mM EDTA, 150 mM NaCl, 0.5% (w/v) Genapol X-80, pH=6.5) and heated at 60° C. for 50 min. The suspension was cooled on ice for 30 min and centrifuged at 4° C. for 60 min at 13,000 rpm to collect the supernatant. The supernatant was syringe filtration treated and loaded to a nickel affinity column (HisTrap™ HP, Cat. 17-5248-01, GE Healthcare). The column was first eluted with buffer A (0.5 M NaCl, 20 mM HEPES, 5 mM imidazole, 2 mM TCEP, 0.5% (w/v) Genapol X-80, pH 8.0) and further eluted with a linear gradient of imidazole (5 mM-500 mM) by mixing buffer A with buffer B (0.5 M NaCl, 20 mM HEPES, 500 mM imidazole, 2 mM TCEP, 0.5% (w/v) Genapol X-80, pH 8.0).

All eluent fractions were characterized by gel electrophoresis on a 4-15% gradient SDS-polyacrylamide gel. The fractions corresponding to all heterogeneously-assembled MspAs were collected for further purifications. To further separate the desired (N90C)₁(M2)₇ pore type from the mixture, gel electrophoresis of the collected fractions was performed on a 10% SDS-polyacrylamide gel with a Tris-Gly buffer. A +160 V bias was continuously applied for 16 h at room temperature (rt) (FIG. 1b). The gel was then stained with coomassie brilliant blue (1.25 g coomassie brilliant blue R250, 225 mL MeOH, 50 mL glacial AcOH, 225 mL ultrapure water) for 4 h. Subsequently, the elution buffer (400 mL MeOH, 100 mL glacial AcOH, replenished with ultrapure water to 1 L) was used to decolorize the gel until the protein bands are clearly visible. The gel was soaked in ultrapure water for 10 min and imaged. The gel fragment containing the band which corresponds to the (N90C)₁(M2)₇ pore type was excised, crushed and rehydrated in the extraction solution (150 mM NaCl, 15 mM Tris-HCl, pH 7.5, 0.2% DDM, 0.5% Genapol X-80, 5 mM TCEP, 10 mM EDTA). The resulting suspension was set at rt for 12 h and then the supernatant was collected. The collected (N90C)₁(M2)₇ was immediately used or stored at −80° C. for long term storage.

3. Chemical Modification of (N90C)₁(M2)₇.

To chemically modify the (N90C)₁(M2)₇, 5 μL of freshly prepared (N90C)₁(M2)₇ and 2.5 μL DMSO solution of 3-(maleimide) phenylboronic acid (500 mM) were mixed and added to a 42.5 L 1.5 M KCl buffer (1.5 M KCl, 10 mM MOPS, pH 7.0). The mixture was set at rt for 10 min. The chemically modified (N90C)₁(M2)₇ was immediately used in all downstream nanopore measurements. For simplicity, if not otherwise stated, the modified hetero-MspA is referred to as MspA-PBA throughout the manuscript.

### 4. Nanopore Measurements and Data Analysis

The measurement device is composed of two custom-made polyformaldehyde chambers separated by a ˜20 μm-thick Teflon film with a drilled aperture (˜100 μm in diameter). Before the measurement, the aperture was first treated with 0.5% (v/v) hexadecane in pentane and set for evaporation of the pentane. Afterwards, 500 μL electrolyte buffers were added to both chambers. The buffer used for all electrical recordings is composed of 1.5 M KCl and 10 mM MOPS at pH 7.0, if not otherwise stated. Two custom made Ag/AgCl electrodes, which were electrically connected to the patch-clamp amplifier, were placed in the chambers, in contact with the buffers. Conventionally, the chamber that is electrically grounded was defined as the cis chamber and the opposing chamber was defined as the trans chamber. After adding 100 μL pentane solution of DPhPC (5 mg/mL) to both chambers, a lipid bilayer would spontaneously form when manually pipetting the electrolyte buffer in either chamber up and down several times. Upon bilayer formation, the acquired current immediately drops to 0 pA, indicating that the aperture has now been electrically sealed. MspA was added to the cis chamber to initiate spontaneous pore insertion. Upon a single nanopore insertion, the buffer in the cis chamber was immediately exchanged to avoid further pore insertions. To avoid interferences from external electromagnetic and vibrational noises, the device was shielded in a custom Faraday cage (34 cm by 23 cm by 15 cm) mounted on a floating optical table (Jiangxi Liansheng Technology). All electrophysiology measurements were performed with an Axonpatch 200B patch-clamp amplifier paired with a Digidata 1550B digitizer (Molecular Devices). Unless otherwise stated, the voltage applied during all measurements is +160 mV. All measurements were carried out at rt (23° C.). All single-channel recordings were sampled at 25 kHz and low-pass filtered with a corner frequency of 1 kHz. Saccharide sensing was performed with a single MspA-PBA pore inserted in the planar lipid bilayer and the saccharide analyte was added to cis prior to single channel recording. All events were detected by the “single channel research” function in Clampfit 10.7. Subsequent analyses, including histogram plotting, scatter plot generation and curve fitting were performed by Origin Pro 2018.

### 5. Event Feature Extraction

For each class, results of three independent measurements were included. From the raw time current trace, the start and the end time of each event was identified by Clampfit 10.7. The star and the end time act as the marker to segment an event from the raw trace and was used to derive the dwell time feature of each event. The segmented event fraction was used to extract other event features, including mean, standard deviation, skewness, kurtosis, peak-to-peak value minimum, maximum and median.

Specifically, the mean current amplitude before the start and after the end of each event was calculated to derive the open pore current of MspA-PBA (Iₚ). The event amplitude was derived from ΔI=Iₚ−Iₛ, in which Iₛ represents the mean blockage level of each event. To avoid deviations between pores, the relative current amplitude (ΔI/Iₚ) was considered as the mean of each event. Events with a ΔI/Iₚ value less than 0.35 were collected for subsequent analysis. The extracted event features form a feature matrix. Only events with a duration beyond 30 ms were selected. For each saccharide type, 1000 events were randomly selected to form a labelled data set for model training and testing. To extract event features for model prediction, the above described process is performed identically except that the event label is not assigned.

### 6. Machine Learning

The input data was randomly split into a training set (80% of the labelled data set) and a testing set (20%) for model training and model testing. The data in the training set was first standardized and was then applied to train six models, including KNN, Xgboost, Regression Tree (CART), SVM, Gradient Boost (GBDT) and Random Forest. According to the 10-fold cross validation accuracy, Random Forest was selected and hyperparametrically-tuned. A confusion matrix was generated using the testing set for model evaluation (FIG. 3c, 5b). The model was saved for predictions of unlabelled data (FIG. 22, 32).

### Author Contributions

S. Y. Z. and S. H. conceived the project. S. Y. Z., Z. Y. C., L. Y. W., and K. F. W. performed the measurements. P. P. F. designed the machine-learning algorithms. S. Y. Z., Y. Q. W, Y. L. and S. H. Y. prepared the MspA nanopores. P. K. Z. set up the instruments. S. H. and S. Y. Z. wrote the paper. S. Y. Z. Y. Q. W. and S. H. Y. prepared the supplementary videos. W. D. J, X. Y. D. and C. Z. H. provided inspiring discussions. S. H. and H. Y. C. supervised the project.

### Data Availability Statement

All data presented in this work can be requested from the corresponding author upon reasonable request.

### Code Availability Statement

The custom machine learning algorithm is submitted as a supplementary material, named as “saccharide classifier”. A brief readme document is also provided.

### Competing Interest Statement

S. H. and S. Y. Z. have filed patents describing the heterogeneous MspA and its applications thereof.

### ACKNOWLEDGMENTS

The authors acknowledge Prof. Hagan Bayley (University of Oxford) for valuable suggestions concerning preparation of the manuscript. The authors acknowledge Prof. Zijian Guo, Prof. Shaolin Zhu, Prof. Congqing Zhu, Prof. Jie Li and Prof. Ran Xie of Nanjing University.

This project was funded by National Natural Science Foundation of China (Grant No. 31972917, No. 91753108, No. 21675083), Supported by the Fundamental Research Funds for the Central Universities (Grant No. 020514380257, No. 020514380261), Programs for high-level entrepreneurial and innovative talents introduction of Jiangsu Province (individual and group program), Natural Science Foundation of Jiangsu Province (Grant No. BK20200009), Excellent Research Program of Nanjing University (Grant No. ZYJH004), Shanghai Municipal Science and Technology Major Project, State Key Laboratory of Analytical Chemistry for Life Science (Grant No. 5431ZZXM1902), Technology innovation fund program of Nanjing University.

### REFERENCES

1 Varki, A. & Kornfeld, S. in Essentials of Glycobiology [Internet]. 3rd edition. Vol. Chapter 1. (eds A. Varki et al.) (Cold Spring Harbor Laboratory Press: 2015-2017, 2017).
2 Dashty, M. A quick look at biochemistry: Carbohydrate metabolism. Clin. Biochem. 46, 1339-1352, doi: https://doi.org/10.1016/j.clinbiochem.2013.04.027 (2013).
3 Zeng. Y., Himmel, M. E. & Ding, S.-Y. Visualizing chemical functionality in plant cell walls. Biotechnol. Biofuels 10, 263, doi: 10.1186/s13068-017-0953-3 (2017).
4 Matsuura, M. Structural Modifications of Bacterial Lipopolysaccharide that Facilitate Gram-Negative Bacteria Evasion of Host Innate Immunity. Front. Immunol. 4, doi: 10.3389/fimmu.2013.00109 (2013).
5. Varki, A. Biological roles of oligosaccharides: all of the theories are correct. Glycobiology 3, 97-130, doi: 10.1093/glycob/3.2.97 (1993).
6 Haltiwanger, R. S. & Lowe, J. B. Role of Glycosylation in Development. Annu. Rev. Biochem. 73, 491-537, doi: 10.1146/annurev.biochem.73.011303.074043 (2004).
7 Flynn, R. A. et al. Small RNAs are modified with N-glycans and displayed on the surface of living cells. Cell 184, 3109-3124.e3122, doi: https://doi.org/10.1016/j.cell.2021.04.023 (2021).
8 Reily, C., Stewart, T. J., Renfrow, M. B. & Novak, J. Glycosylation in health and disease. Nat. Rev. Nephrol. 15, 346-366, doi: 10.1038/s41581-019-0129-4 (2019).
9 Moremen, K. W., Tiemeyer, M. & Nairn, A. V. Vertebrate protein glycosylation: diversity, synthesis and function. Nat. Rev. Mol. Cell Biol. 13, 448-462, doi: 10.1038/nrm3383 (2012).
10 Puvirajesinghe, T. M. & Turnbull, J. E. Glycoarray Technologies: Deciphering Interactions from Proteins to Live Cell Responses. Microarrays 5, doi: 10.3390/microarrays5010003 (2016).
11 Hu, S. & Wong, D. T. Lectin microarray. Proteomics: Clin. Appl. 3, 148-154, doi: 10.1002/prca.200800153 (2009).
12 Mantovani, V., Galeotti, F., Maccari, F. & Volpi, N. Recent advances in capillary electrophoresis separation of monosaccharides, oligosaccharides, and polysaccharides. Electrophoresis 39, 179-189, doi: 10.1002/elps.201700290 (2018).
13 Rovio, S., Simolin, H., Koljonen, K. & Siren, H. Determination of monosaccharide composition in plant fiber materials by capillary zone electrophoresis. J. Chromatogr. A 1185, 139-144, doi: https://doi.org/10.1016/j.chroma.2008.01.031 (2008).
14 Nagy, G., Peng. T. & Pohl, N. L. B. Recent Liquid Chromatographic Approaches and Developments for the Separation and Purification of Carbohydrates. Anal. Methods 9, 3579-3593, doi: 10.1039/C7AY01094J (2017).
Vrecker, G. C. M. & Wuhrer, M. Reversed-phase separation methods for glycan analysis. Anal. Bioanal. Chem. 409, 359-378, doi: 10.1007/s00216-016-0073-0 (2017).
16 Lundborg, M., Fontana, C. & Widmalm, G. Automatic Structure Determination of Regular Polysaccharides Based Solely on NMR Spectroscopy. Biomacromolecules 12, 3851-3855, doi: 10.1021/bm201169y (2011).
17 Fontana, C., Kovacs, H. & Widmalm, G. NMR structure analysis of uniformly 13C-labeled carbohydrates. J. Biomol. NMR 59, 95-110, doi: 10.1007/s10858-014-9830-6 (2014).
18 Veillon, L. et al. Characterization of isomeric glycan structures by LC-MS/MS. Electrophoresis 38, 2100-2114, doi: https://doi.org/10.1002/elps.201700042 (2017).
19 Zhou, S., Veillon, L., Dong, X., Huang, Y. & Mechref, Y. Direct comparison of derivatization strategies for LC-MS/MS analysis of N-glycans. Analyst 142, 4446-4455, doi: 10.1039/c7an01262d (2017).
20 Gray, C. J. et al. Advancing Solutions to the Carbohydrate Sequencing Challenge. J. Am. Chem. Soc. 141, 14463-14479, doi: 10.1021/jacs.9b06406 (2019).
21 Aretz, I. & Meierhofer, D. Advantages and Pitfalls of Mass Spectrometry Based Metabolome Profiling in Systems Biology. Int. J. Mol. Sci. 17, 632, doi: 10.3390/ijms 17050632 (2016).
22 Emwas, A. H. The strengths and weaknesses of NMR spectroscopy and mass spectrometry with focus on metabolomics research. Methods Mol. Biol. 1277, 161-193, particular doi: 10.1007/978-1-4939-2377-9_13 (2015).
23 Morimoto, K. et al. GlycanAnalysis Plug-in: a database search tool for N-glycan structures using mass spectrometry. Bioinformatics 31, 2217-2219, doi: 10.1093/bioinformatics/btv110 (2015).
24 Walsh, I. et al. GlycanAnalyzer: software for automated interpretation of N-glycan profiles after exoglycosidase digestions. Bioinformatics 35, 688-690, doi: 10.1093/bioinformatics/bty681 (2019).
25 Manrao, E. A. et al. Reading DNA at single-nucleotide resolution with a mutant MspA nanopore and phi29 DNA polymerase. Nat. Biotechnol. 30, 349-353, doi: 10.1038/nbt.2171 (2012).
26 Yan, S. et al. Direct sequencing of 2′-deoxy-2′-fluoroarabinonucleic acid (FANA) using nanopore-induced phase-shift sequencing (NIPSS). Chem. Sci. 10, 3110-3117, doi: 10.1039/c8sc05228j (2019).
27 Zhang, J. et al. Direct microRNA Sequencing Using Nanopore-Induced Phase-Shift Sequencing. iScience 23, doi: 10.1016/j.isci.2020.100916 (2020).
28 Yan, S. et al. Single Molecule Ratcheting Motion of Peptides in a Mycobacterium smegmatis Porin A (MspA) Nanopore. Nano Lett. 21, 6703-6710, doi: 10.1021/acs.nanolett. 1c02371 (2021).
29 Brinkerhoff, H., Kang, A. S. W., Liu, J., Aksimentiev, A. & Dekker, C. Infinite re-reading of single proteins at single-amino-acid resolution using nanopore sequencing. bioRxiv, 2021.2007.2013.452225, doi: 10.1101/2021.07.13.452225 (2021).
30 Stylianopoulos, C. in Encyclopedia of Human Nutrition (Third Edition) (ed Benjamin Caballero) 265-271 (Academic Press, 2013).
31 Karawdeniya, B. I., Bandara, Y., Nichols, J. W., Chevalier, R. B. & Dwyer, J. R. Surveying silicon nitride nanopores for glycomics and heparin quality assurance. Nat. Commun. 9, 3278, doi: 10.1038/s41467-018-05751-y (2018).
32 Im, J., Lindsay, S., Wang, X. & Zhang, P. Single Molecule Identification and Quantification of Glycosaminoglycans Using Solid-State Nanopores. ACS Nano 13, 6308-6318, doi: 10.1021/acsnano.9b00618 (2019).
33 Xia, K. et al. Synthetic heparan sulfate standards and machine learning facilitate the development of solid-state nanopore analysis. Proc. Natl. Acad. Sci. U.S.A 118, doi: 10.1073/pnas.2022806118 (2021).
34 Cai, Y. et al. A solid-state nanopore-based single-molecule approach for label-free characterization of plant polysaccharides. Plant Commun. 2, 100106, doi: 10.1016/j.xplc.2020.100106 (2021).
35 Guo, Z., Shin, I. & Yoon, J. Recognition and sensing of various species using boronic acid derivatives. Chem. Commun. (Cambridge, U. K.) 48, 5956-5967, doi: 10.1039/c2cc31985c (2012).
36 Wu, X. et al. Selective sensing of saccharides using simple boronic acids and their aggregates. Chem. Soc. Rev. 42, 8032-8048, doi: 10.1039/c3cs60148j (2013).
37 Peters, J. A. Interactions between boric acid derivatives and saccharides in aqueous media: Structures and stabilities of resulting esters. Coord. Chem. Rev. 268, 1-22, doi: https://doi.org/10.1016/j.ccr.2014.01.016 (2014).
38 van den Berg. R., Peters, J. A. & van Bekkum, H. The structure and (local) stability constants of borate esters of mono- and di-saccharides as studied by 11B and 13C NMR spectroscopy. Carbohydr. Res. 253, 1-12, doi: 10.1016/0008-6215 (94) 80050-2 (1994).
39 Ramsay, W. J. & Bayley, H. Single-Molecule Determination of the Isomers of d-Glucose and d-Fructose that Bind to Boronic Acids. Angew. Chem., Int. Ed. Engl. 57, 2841-2845, doi: 10.1002/anie.201712740 (2018).
40 Butler, T. Z., Pavlenok, M., Derrington, I. M., Niederweis, M. & Gundlach, J. H. Single-molecule DNA detection with an engineered MspA protein nanopore. Proc. Natl. Acad. Sci. U.S.A 105, 20647, doi: 10.1073/pnas.0807514106 (2008).
41 Niederweis, M. et al. Cloning of the mspA gene encoding a porin from Mycobacterium smegmatis. Mol. Microbiol. 33, 933-945, doi: 10.1046/j. 1365-2958.1999.01472.x (1999).
42 Faller, M., Niederweis, M. & Schulz, G. E. The structure of a mycobacterial outer-membrane channel. Science 303, 1189-1192, doi: 10.1126/science. 1094114 (2004).
43 Laszlo, A. H. et al. Detection and mapping of 5-methylcytosine and 5-hydroxymethylcytosine with nanopore MspA. Proc. Natl. Acad. Sci. U.S.A 110, 18904-18909, doi: 10.1073/pnas. 1310240110 (2013).
44 Wang, Y. et al. Nanopore Sequencing Accurately Identifies the Mutagenic DNA Lesion O(6)-Carboxymethyl Guanine and Reveals Its Behavior in Replication. Angew. Chem., Int. Ed. Engl. 58, 8432-8436, doi: 10.1002/anie.201902521 (2019).
45 Ma, F. et al. Nanopore Sequencing Accurately Identifies the Cisplatin Adduct on DNA. ACS Sens. 6, 3082-3092, doi: 10.1021/acssensors. 1c01212 (2021).
46 Cao, J. et al. Giant single molecule chemistry events observed from a tetrachloroaurate (III) embedded Mycobacterium smegmatis porin A nanopore. Nat. Commun. 10, 5668, doi: 10.1038/s41467-019-13677-2 (2019).
47 Wang, S. et al. Single molecule observation of hard-soft-acid-base (HSAB) interaction in engineered Mycobacterium smegmatis porin A (MspA) nanopores. Chem. Sci. 11, 879-887, doi: 10.1039/c9sc05260g (2019).
48 Jia, W. et al. Programmable Nano-Reactors for Stochastic Sensing. Nat. Commun., doi: 10.1038/s41467-021-26054-9 (2021).
49 Choi, L.-S. & Bayley, H. S-Nitrosothiol Chemistry at the Single-Molecule Level. Angew: Chem., Int. Ed. Engl. 51, 7972-7976, doi: https://doi.org/10.1002/anie.201202365 (2012).
50 Lehmacher, A. & Bockemühl, J. 1-Sorbose utilization by virulent Escherichia coli and Shigella: Different metabolic adaptation of pathotypes. Int. J. Med. Microbiol. 297, 245-254, doi: https://doi.org/10.1016/j.ijmm.2007.01.007 (2007).
51 Sugisawa, T., Miyazaki, T. & Hoshino, T. Microbial Production of L-Ascorbic Acid from D-Sorbitol, L-Sorbose, L-Gulose, and L-Sorbosone by Ketogulonicigenium vulgare DSM 4025. Biosci., Biotechnol., Biochem. 69, 659-662, doi: 10.1271/bbb.69.659 (2005).
52 Adair, W. L. in xPharm: The Comprehensive Pharmacology Reference (eds S. J. Enna & David B. Bylund) 1-12 (Elsevier, 2007).
53 Deo, R. C. Machine Learning in Medicine. Circulation 132, 1920-1930, doi: 10.1161/CIRCULATIONAHA.115.001593 (2015).
54 Díaz Carral, A., Ostertag, M. & Fyta, M. Deep learning for nanopore ionic current blockades. J. Chem. Phys. 154, 044111, doi: 10.1063/5.0037938 (2021).
55 Schreiber, J. et al. Error rates for nanopore discrimination among cytosine, methylcytosine, and hydroxymethylcytosine along individual DNA strands. Proc. Natl. Acad. Sci. U.S.A 110, 18910, doi: 10.1073/pnas.1310615110 (2013).
56 Misiunas, K., Ermann, N. & Keyser, U. F. QuipuNet: Convolutional Neural Network for Single-Molecule Nanopore Sensing. Nano Lett. 18, 4040-4045, doi: 10.1021/acs.nanolett. 8b01709 (2018).
57 Wang, Y. et al. Structural-profiling of low molecular weight RNAs by nanopore trapping/translocation using Mycobacterium smegmatis porin A. Nat. Commun. 12, 3368, doi: 10.1038/s41467-021-23764-y (2021).
58 Doroschak. K. et al. Rapid and robust assembly and decoding of molecular tags with DNA-based nanopore signatures. Nat. Commun. 11, 5454-5454. doi: 10.1038/s41467-020-19151-8 (2020).
59 Wei. Z.-X. et al. Learning Shapelets for Improving Single-Molecule Nanopore Sensing. Anal. Chem. 91, 10033-10039. doi: 10.1021/acs.analchem.9b01896 (2019).
60 Sui. X.-J. et al. Acrolysin Nanopore Identification of Single Nucleotides Using the AdaBoost Model. J. Anal. Test. 3, 134-139. doi: 10.1007/s41664-019-00088-x (2019).
61 Liu. Y. et al. Allosteric Switching of Calmodulin in a Mycobacterium smegmatis porin A (MspA) Nanopore-Trap. Angew. Chem. Int. Ed. Engl. n/a. doi:https://doi.org/10.1002/anic.202110545 (2021).
62 Zhou, W., Qiu, H., Guo, Y. & Guo, W. Molecular Insights into Distinct Detection Properties of α-Hemolysin. MspA. CsgG. and Acrolysin Nanopore Sensors. J. Phys. Chem. B 124, 1611-1618. doi: 10.1021/acs.jpcb.9b10702 (2020).
63 Yu. M. et al. Unveiling the Microscopic Mechanism of Current Variation in the Sensing Region of the MspA Nanopore for Lett. DNA Sequencing. J. Phys. Chem. 12, 9132-9141. doi: 10.1021/acs.jpclett. 1c02414 (2021).
64 Ma, Q., Zhao, X., Shi, A. & Wu, J. Bioresponsive Functional Phenylboronic Acid-Based Delivery System as an Emerging Platform for Diabetic Therapy. Int. J. Nanomed. 16, 297-314. doi: 10.2147/IJN.S284357 (2021).
65 Cambre. J. N. & Sumerlin. B. S. Biomedical applications of boronic acid polymers. Polymer 52, 4631-4643. doi: https://doi.org/10.1016/j.polymer.2011.07.057 (2011).
66 Bull. S. D. et al. Exploiting the Reversible Covalent Bonding of Boronic Acids: Recognition. Sensing. and Assembly. Acc. Chem. Res. 46, 312-326. doi: 10.1021/ar300130w (2013).
67 Zhang. J. et al. Mapping Potential Engineering Sites of Mycobacterium smegmatis porin A (MspA) to Form a Nanoreactor. ACS Sens. 6, 2449-2456, doi: 10.1021/acssensors. 1c00792 (2021).
68 Pavlenok. M. & Niederweis. M. Hetero-oligomeric MspA pores in Mycobacterium smegmatis. FEMS Microbiol. Lett. 363, doi: 10.1093/femsle/fnw046 (2016).

### Supplementary Information

### Materials

1,2-diphytanoyl-sn-glycero-3-phosphocholine (DPhPC) was obtained from Avanti Polar Lipids. Pentane, hexadecane, tris(2-carboxyethyl) phosphine hydrochloride (TCEP), ethylenediamine-tetraAcOH (EDTA), Genapol X-80, ammonium persulfate (≥98%), sodium dodecyl sulfate (≥98.5%), N,N,N′,N′-tetramethylethylenediamine (99%) and acrylamide/bis-acrylamide, 30% solution were from Sigma-Aldrich. Potassium chloride, sodium chloride (99.99%), sodium hydroxide (99.9%), sodium hydrogen phosphate and sodium dihydrogen phosphate were from Aladdin (China). Hydrochloric acid (HCl) was from Sinopharm (China). 4-(2-Hydroxyethyl)-1-piperazine ethanesulfonic acid (HEPES) was from Shanghai Yuanye Bio-Technology (China). Dioxane-free isopropyl-β-D-thiogalactopyranoside (IPTG), kanamycin sulfate, imidazole and tris(hydroxymethyl) aminomethane (Tris) were from Solarbio. SDS-PAGE electrophoresis buffer powder was from Beyotime (China). Precision Plus Protein™ Dual color Standards, TGX™ FastCast™ Acylamide Kit (4-15%), stacking gel buffer (0.5M Tris-HCl buffer, pH 6.8) and resolving gel buffer (1.5M Tris-HCl buffer, pH 8.8) were from Bio-rad. LB broth and LB agar were from Hopebio (China). 3-(maleimide) phenylboronic acid (MPBA, Cat. #sc-352346) was from Santa Cruz Biotechnology (Shanghai) Co., Ltd. All the items listed above were used as received.

D-(+)-Mannose (≥99%) was from Sigma-Aldrich. D-(+)-Glucose (99%) was from Damas-beta (China). D-(+)-Galactose (98%), D-(+)-Xylose (98%), L-Rhamnose monohydrate (99%), D-(−)-Ribose (≥99%), N-acetyl-D-Galactosamine (98%) were from Aladdin (China). L-(−)-Sorbose (98%) was from Macklin (China). D-(−)-Fructose (≥98%) was from Shanghai Dibai Bio-Technology (China).

1.5 M KCl buffer (1.5 M KCl, 10 mM MOPS, pH 7.0), lysis buffer (100 mM Na₂HPO₄/NaH₂PO₄, 0.1 mM EDTA, 150 mM NaCl, 0.5% (w/v) Genapol X-80, pH 6.5), buffer A (0.5 M NaCl, 20 mM HEPES, 5 mM Imidazole, 0.5% (w/v) Genapol X-80, pH 8.0) and buffer B (0.5 M NaCl, 20 mM HEPES, 500 mM Imidazole, 0.5% (w/v) Genapol X-80, pH 8.0) were prepared with Milli-Q water and membrane (0.2 μm, Whatman) filtered.

TABLE 1
The protein sequence of M2 MspA-D16H6 and
N90C MspA-H6.

| Name | Protein Sequence |
|---|---|
| M2 | GLDNELSLVDGQDRTLTVQQWDTFLNGVFPLDRNRLT |
| MspA-D16H6 | REWFHSGRAKYIVAGPGADEFEGTLELGYQIGFPWSL |
| | GVGINFSYTTPNILINNGNITAPPFGLNSVITPNLFP |
| | GVSISARLGNGPGIQEVATFSVRVSGAKGGVAVSNAH |
| | GTVTGAAGGVLLRPFARLIASTGDSVTTYGEPWNMND |
| | DDDDDDDDDDDDDDDHHHHHH* |
| | (SEQ ID NO: 2) |
| N90C | GLDNELSLVDGQDRTLTVQQWDTFLNGVFPLDRNRLT |
| MspA-H6 | REWFHSGRAKYIVAGPGADEFEGTLELGYQIGFPWSL |
| | GVGINFSYTTPNILICNGNITAPPFGLNSVITPNLFP |
| | GVSISARLGNGPGIQEVATFSVRVSGAKGGVAVSNAH |
| | GTVTGAAGGVLLRPFARLIASTGDSVTTYGEPWNMNH |
| | HHHHH* |
| | (SEQ ID NO: 3) |

### Footnote:

1. The underlined characters in the sequence mark the core sequence differences between both genes. Specifically, the cysteine in N90C MspA-H6 plays a critical role as an adapter to introduce a phenylboronic acid to the pore restriction (FIG. 6).
2. The hexa-histidine tag (H6) is denoted with bold characters in the sequence.
3. The poly-aspartic acids tag (D16) is denoted with italic characters in the sequence.

TABLE 2
Current-voltage (I-V) curves of (N90C)₁(M2)₇ MspA. All measurements
were performed in a 1.5M KCl, 10 mM MOPS, pH 7.0 buffer. A
voltage ramp between −150 mV and +150 mV was applied
in the acquisition of the I-V curves. Three independent measurements
(N = 3) were performed to form the statistics.

| | Current/pA | | | | |
|---|---|---|---|---|---|
| Voltage/mV | Pore1 | Pore2 | Pore3 | Mean | S.D. |
| −150 | −437.7 | −454.2 | −426.13 | −439.3 | 11.5 |
| −140 | −405.6 | −418.12 | −408.3 | −410.7 | 5.4 |
| −130 | −372.8 | −384.2 | −375.3 | −377.4 | 4.9 |
| −120 | −340.9 | −351.4 | −343.2 | −345.2 | 4.5 |
| −110 | −308.7 | −318.6 | −311.14 | −312.8 | 4.2 |
| −100 | −277.9 | −287.10 | −280.12 | −281.7 | 3.9 |
| −90 | −246.6 | −255.4 | −248.7 | −250.2 | 3.8 |
| −80 | −216.4 | −224.6 | −218.4 | −219.8 | 3.5 |
| −70 | −186.2 | −194.0 | −188.03 | −189.4 | 3.3 |
| −60 | −157.2 | −164.5 | −158.9 | −160.2 | 3.10 |
| −50 | −128.3 | −135.14 | −129.8 | −131.07 | 2.9 |
| −40 | −100.6 | −107.07 | −102.10 | −103.3 | 2.8 |
| −30 | −73.05 | −79.2 | −74.5 | −75.6 | 2.6 |
| −20 | −46.9 | −52.7 | −48.2 | −49.3 | 2.5 |
| −10 | −21.04 | −26.6 | −22.2 | −23.3 | 2.4 |
| 0 | 3.4 | −2.04 | 2.4 | 1.3 | 2.4 |
| 10 | 27.08 | 21.9 | 26.2 | 25.03 | 2.3 |
| 20 | 50.7 | 45.6 | 50.02 | 48.8 | 2.3 |
| 30 | 73.4 | 68.2 | 72.7 | 71.4 | 2.3 |
| 40 | 95.4 | 90.6 | 94.8 | 93.6 | 2.2 |
| 50 | 117.7 | 112.2 | 117.10 | 115.7 | 2.5 |
| 60 | 138.8 | 133.3 | 138.6 | 136.9 | 2.6 |
| 70 | 160.13 | 154.8 | 159.8 | 158.2 | 2.5 |
| 80 | 181.5 | 175.6 | 181.2 | 179.4 | 2.7 |
| 90 | 202.4 | 196.5 | 201.8 | 200.2 | 2.6 |
| 100 | 224.2 | 218.05 | 223.2 | 221.8 | 2.7 |
| 110 | 245.9 | 239.4 | 245.3 | 243.5 | 2.9 |
| 120 | 268.03 | 261.3 | 267.2 | 265.5 | 2.9 |
| 130 | 291.08 | 283.9 | 290.2 | 288.4 | 3.2 |
| 140 | 314.3 | 306.6 | 312.9 | 311.3 | 3.3 |
| 150 | 340.2 | 329.4 | 339.10 | 336.2 | 4.9 |

TABLE 3
Current-voltage (I-V) curves of MspA-PBA. All measurements
were performed in a 1.5M KCl, 10 mM MOPS, pH 7.0 buffer.
A voltage ramp between −150 mV and +150 mV was applied
in the acquisition of the I-V curves. Three independent
measurements (N = 3) were performed to form the statistics.

| | Current/pA | | | | |
|---|---|---|---|---|---|
| Voltage/mV | Pore1 | Pore2 | Pore3 | Mean | S.D. |
| −150 | −521.0 | −543.8 | −553.6 | −539.5 | 16.7 |
| −140 | −483.9 | −504.8 | −514.5 | −501.05 | 15.7 |
| −130 | −445.8 | −466.3 | −491.3 | −467.8 | 22.8 |
| −120 | −410.0 | −441.9 | −436.4 | −429.4 | 17.04 |
| −110 | −372.9 | −388.8 | −398.6 | −386.8 | 12.9 |
| −100 | −338.5 | −350.3 | −360.11 | −349.7 | 10.8 |
| −90 | −302.9 | −312.5 | −322.9 | −312.8 | 10.0 |
| −80 | −267.8 | −276.5 | −285.6 | −276.6 | 8.9 |
| −70 | −233.5 | −238.7 | −248.4 | −240.2 | 7.6 |
| −60 | −199.05 | −201.4 | −214.2 | −204.9 | 8.2 |
| −50 | −164.6 | −167.2 | −177.0 | −169.6 | 6.5 |
| −40 | −132.0 | −131.2 | −141.6 | −134.9 | 5.8 |
| −30 | −98.4 | −97.05 | −106.2 | −100.6 | 4.9 |
| −20 | −66.6 | −61.7 | −72.02 | −66.7 | 5.2 |
| −10 | −34.3 | −28.08 | −40.3 | −34.2 | 6.10 |
| 0 | −3.4 | 4.3 | −5.5 | −1.5 | 5.13 |
| 10 | 26.5 | 37.8 | 25.6 | 30.0 | 6.8 |
| 20 | 57.3 | 70.2 | 60.4 | 62.6 | 6.7 |
| 30 | 87.3 | 101.3 | 90.3 | 93.0 | 7.4 |
| 40 | 117.5 | 132.5 | 121.5 | 123.8 | 7.8 |
| 50 | 146.9 | 162.9 | 155.6 | 155.2 | 8.05 |
| 60 | 173.4 | 192.9 | 183.7 | 183.3 | 9.8 |
| 70 | 203.2 | 221.6 | 211.8 | 212.2 | 9.2 |
| 80 | 228.4 | 252.7 | 239.9 | 240.3 | 12.14 |
| 90 | 260.8 | 281.9 | 270.4 | 271.06 | 10.6 |
| 100 | 287.2 | 311.9 | 300.3 | 299.8 | 12.4 |
| 110 | 313.5 | 346.7 | 328.9 | 329.7 | 16.6 |
| 120 | 343.5 | 371.09 | 359.5 | 358.03 | 13.9 |
| 130 | 372.8 | 402.8 | 389.4 | 388.4 | 15.04 |
| 140 | 407.2 | 432.7 | 421.8 | 420.6 | 12.8 |
| 150 | 437.9 | 465.7 | 465.7 | 456.4 | 16.0 |

TABLE 4
1/τₒₙ and 1/τ_{off} of L-Sorbose measured at various concentrations.
The mean inter-event interval (τₒₙ) and the mean dwell time (τ_{off})
were derived from single-exponential fitting results as described in
FIG. 10. All measurements were performed in a 1.5M KCl, 10 mM
MOPS, pH 7.0 buffer. Means and standard deviations (S.D.) were
derived from results of three independent measurement (N = 3).

| Concentration/ | 1/τₒₙ/ms⁻¹ | | 1/τ_{off}/ms⁻¹ | |
|---|---|---|---|---|
| μM | Mean | S.D. | Mean | S.D. |
| 400 | 5.10E−05 | 9.4E−06 | 2.9E−04 | 3.08E−05 |
| 800 | 1.03E−04 | 1.08E−05 | 2.9E−04 | 3.5E−05 |
| 1200 | 1.7E−04 | 3.07E−05 | 3.08E−04 | 3.07E−05 |
| 1600 | 2.2E−04 | 2.8E−05 | 3.11E−04 | 3.2E−05 |
| 2000 | 3.07E−04 | 1.2E−05 | 2.9E−04 | 4.02E−05 |

TABLE 5
1/τₒₙ and 1/τ_{off} of L-Sorbose sensing performed at different
voltages. The concentration of L-Sorbose added to cis was
5 mM. The applied voltage was changed from −40 mV
to +220 mV. The mean inter-event interval (τₒₙ) and the
mean dwell time (τ_{off}) were derived from
single-exponential fitting results as described in FIG. 12.
All measurements were performed in a 1.5M KCl, 10 mM
MOPS, pH 7.0 buffer. Means and standard deviations
(S.D.) were derived from results of three independent
measurement (N = 3).

| | 1/τₒₙ/ms⁻¹ | | 1/τ_{off}/ms⁻¹ | |
|---|---|---|---|---|
| Voltage/mV | Mean | S.D. | Mean | S.D. |
| −40 | 7.6E−04 | 1.2E−05 | 5.01E−04 | 6.11E−05 |
| 40 | 7.9E−04 | 7.7E−05 | 5.06E−04 | 1.4E−05 |
| 70 | 7.4E−04 | 5.07E−05 | 4.3E−04 | 8.6E−05 |
| 100 | 8.7E−04 | 1.10E−04 | 4.04E−04 | 4.09E−05 |
| 130 | 8.4E−04 | 4.7E−05 | 3.8E−04 | 4.01E−05 |
| 160 | 8.5E−04 | 1.10E−05 | 2.9E−04 | 4.2E−05 |
| 190 | 8.3E−04 | 1.4E−04 | 2.9E−04 | 5.6E−05 |
| 220 | 8.07E−04 | 2.4E−05 | 3.4E−04 | 7.9E−05 |

TABLE 6
Amplitude of L-Sorbose events at different voltages. The concentration
of L-Sorbose added to cis was 5 mM. The applied voltage was changed
from +40 mV to +220 mV. The amplitude was derived from
gauss peak fitting results as described in Methods in Example
1. All measurements were performed in a 1.5M KCl, 10 mM MOPS,
pH 7.0 buffer. Means and standard deviations (S.D.) were derived
from results of three independent measurement (N = 3).

| Volt- | | | | | |
|---|---|---|---|---|---|
| age/ | Current/pA | | | | |
| mV | Pore1 | Pore2 | Pore3 | Mean | S.D. |
| 40 | 29.3 ± 0.0005 | 29.05 ± 0.0005 | 29.2 ± 0.002 | 29.2 | 0.13 |
| 70 | 54.5 ± 0.010 | 52.7 ± 0.0008 | 52.5 ± 0.0008 | 53.08 | 0.8 |
| 100 | 74.6 ± 0.002 | 73.2 ± 0.009 | 73.3 ± 0.002 | 73.7 | 0.8 |
| 130 | 91.6 ± 0.003 | 90.3 ± 0.004 | 90.3 ± 0.004 | 90.8 | 0.7 |
| 160 | 107.7 ± 0.005 | 104.9 ± 0.006 | 105.3 ± 0.006 | 105.9 | 1.5 |
| 190 | 122.9 ± 0.007 | 116.7 ± 0.008 | 120.7 ± 0.010 | 120.13 | 3.2 |
| 220 | 143.6 ± 0.02 | 132.9 ± 0.02 | 137.2 ± 0.013 | 137.9 | 5.4 |

Supplementary Video 1: A representative trace acquired with L-Sorbose. Single-channel recordings were performed with MspA-PBA in a 1.5 M KCl, 10 mM MOPS pH=7.0 buffer. A +160 mV bias was continually applied. L-Sorbose was added to cis to a 0.8 mM final concentration. The events of L-Sorbose were labeled with orange pentagons.

Supplementary Video 2: The continuous representative traces of D-Mannose sensing. Single-channel recordings were performed with MspA-PBA in a 1.5 M KCl, 10 mM MOPS pH=7.0 buffer. A +160 mV bias was continually applied. D-Mannose was added to cis to a 20 mM final concentration. Different types of D-Mannose events were observed and respectively marked with Roman numerals I-III. Detailed discussions were provided in FIGS. 2g-2i and FIGS. 18, 19.

### REFERENCES

1 Ramsay, W. J. & Bayley, H. Single-Molecule Determination of the Isomers of d-Glucose and d-Fructose that Bind to Boronic Acids. Angew. Chem., Int. Ed. Engl. 57, 2841-2845, doi: 10.1002/anie.201712740 (2018).
2 Alcock, L. J., Perkins, M. V. & Chalker, J. M. Chemical methods for mapping cysteine oxidation. Chem. Soc. Rev. 47, 231-268, doi: 10.1039/c7cs00607a (2018).
3 Shin, S. H., Luchian, T., Cheley, S., Braha, O. & Bayley, H. Kinetics of a Reversible Covalent-Bond-Forming Reaction Observed at the Single-Molecule Level. Angew. Chem., Int. Ed. Engl. 41, 3707-3709 (2002).

### Example 2: Identification of Nucleoside Monophosphates and their Epigenetic Modifications Using an Engineered Nanopore

Chemical modifications of RNA play critical roles in the regulation of various biological processes and are associated with many human diseases. Direct identification of RNA modifications by sequencing however, remains challenging. Nanopore sequencing may offer a promising solution by directly probing sequence modifications, but the currently available strand sequencing strategy still is complicated by sequence decoding. Alternatively, sequential nanopore identification of enzymatically cleaved nucleoside monophosphates (NMP) may simultaneously provide accurate sequence and modification information. In preparation for that, a hetero-octameric Mycobacterium smegmatis porin A (MspA) modified with phenylboronic acid (PBA) has been prepared, with which direct distinguishing between all four canonical NMPs, 5-methylcytidine (m⁵C), N6-methyladenosine (m⁶A), N7-methylguanosine (m⁷G), N1-methyladenosine (m¹A), inosine (I), pseudouridine (Ψ) and dihydrouridine (D) was achieved. A custom machine learning algorithm was also developed and was found to deliver a general accuracy score of 0.996. This method was applied to the quantitative analysis of base modifications in microRNA and tRNA. It is generally suitable for sensing of a large variety of nucleoside or nucleotide derivatives and may bring new insights to epigenetic RNA sequencing.

### INTRODUCTION

Many RNA modifications are enzymatically driven chemical modifications such as methylation, deamination, reduction and thiolation, or isomerization to either the ribose or the nucleobase of nucleotides. The modifications are carried out by special writer proteins during the post-transcription stage. According to the MODOMICS database, approximately 170 types of RNA modifications are known¹ and are essential for various biological processes such as genetic recoding², pre-mRNA splicing³, mRNA exporting⁴, RNA folding⁵ and chromatin state regulation⁶. Accumulating evidences indicate that a large number of RNA modifications are associated with cancers^{7,8}, neurological disorders⁹ and other human diseases¹⁰, and may thus be treated as either diagnostic markers or therapeutic targets. Recent reports also indicate that RNA modifications are also associated with the yield of grains¹¹. However, there is an unmet but urgent need to map diverse RNA modifications accurately, and this is complicated by the similarity in their chemical structures¹².

Analysis of RNA modifications can be performed by thin layer chromatography (TLC)¹³, high performance liquid chromatography coupled with UV spectrophotometry (HPLC-UV)¹⁴ or high performance liquid chromatography coupled to mass spectrometry (HPLC-MS)¹⁵. These methods enable simultaneous measurement of a large number of RNA modifications, but they fail to provide any sequence information. Methods based on next-generation sequencing (NGS) allow for mapping of transcriptome-wide RNA modifications¹⁶, but they rely on either antibodies to immune-precipitate modified RNA fragments¹⁷ or chemical treatments to alter RNA modifications as mutations or truncations in the preparation of cDNA¹⁸. These methods are typically tailored to only one specific modification, and due to the lack of antibodies or chemical reagents that can deal with all RNA modifications, only a limited type of modifications can be detected by sequencing. These include 419, m⁶A^{20, 21}, m⁵C²², m¹A²³, m⁷G²⁴, 5-hydroxymethylcytosine (5hmC)²⁵, N6,2′-O-dimethyladenosine (m⁶Am)¹⁷, N4-acetylcytidine (ac4C)²⁶ and A-to-I editing²⁷. Third-generation sequencing techniques, including methods developed by Pacific Biosciences (PacBio) or Oxford Nanopore Technologies (ONT), may overcome these shortcomings by performing direct RNA sequencing²⁸. In PacBio sequencing, RNA modifications are identified by the observation of time variation between base incorporations²⁹. On the other hand, nanopore sequencing provided by ONT reports RNA modifications by identifying variations in the ionic current^{30, 31} or the event dwell time³². However, the strand sequencing strategy³³, which is limited by the spatial resolution equivalent to an average reading of ˜5-nucleotides³⁴, still suffers from discrimination between all epigenetic modifications by sequencing. This situation is even more serious when the modified nucleotides are close neighbours³⁵.

Sequencing RNA in an exo-sequencing manner, is a different strategy with which exonuclease-decomposed nucleotides can be sequentially read by a nanopore sensor. This however requires the existence of a high resolution nanopore that can unambiguously recognize all nucleotides and their major modifications. A cyclodextrin embedded α-haemolysin (α-HL)^{36, 37} was previously reported to perform this task, but the results indicate an insufficient resolution which fails to allow true discrimination between for example, cytidine diphosphate (CDP) and uridine diphosphate (UDP). Identification of RNA modifications was also not demonstrated³⁶. This low resolution of sensing should result from the cylindrical lumen geometry of α-HL³⁸. Instead, Mycobacterium smegmatis porin A (MspA)³⁹, which is a conically shaped pore widely applied in nanopore sequencing⁴⁰, single molecule chemistry⁴¹ and structure profiling of biomacromolecule^{42, 43}, may be more advantageous. Phenylboronic acid (PBA) is known to form covalent bonds reversibly with 1,2 or 1,3-diols⁴⁴. Previously, the introduction of PBA to the nanopore lumen was successfully applied to the detection of various cis-diol-containing analytes such as saccharides⁴⁵, epinephrine and Remdesivir⁴⁶. However, a hetero-octameric MspA nanopore containing a single PBA adapter has not been reported previously and nanopore identification of a large variety of epigenetic modified NMPs have also never been reported.

### Nucleoside Monophosphate (NMP) Identification Using a PBA Modified MspA

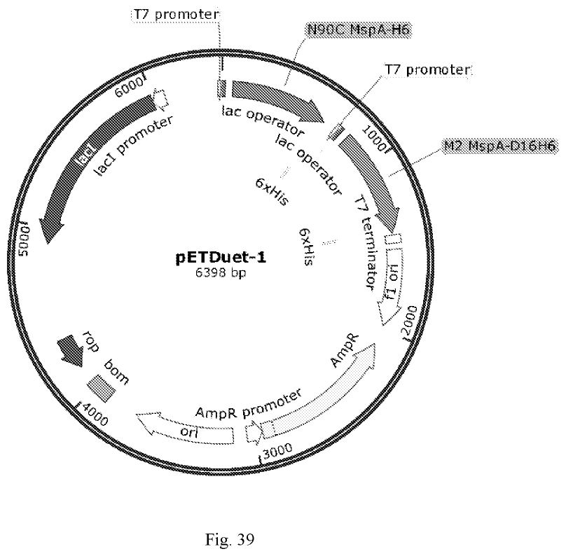

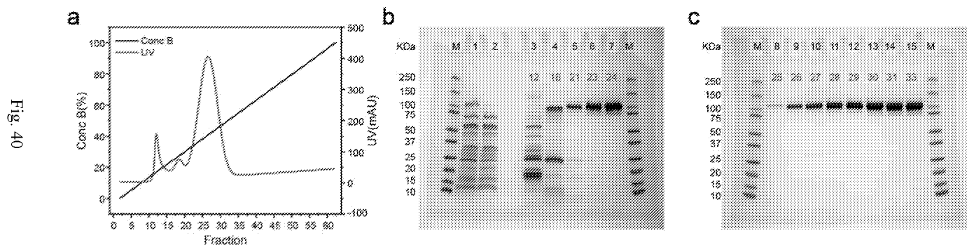

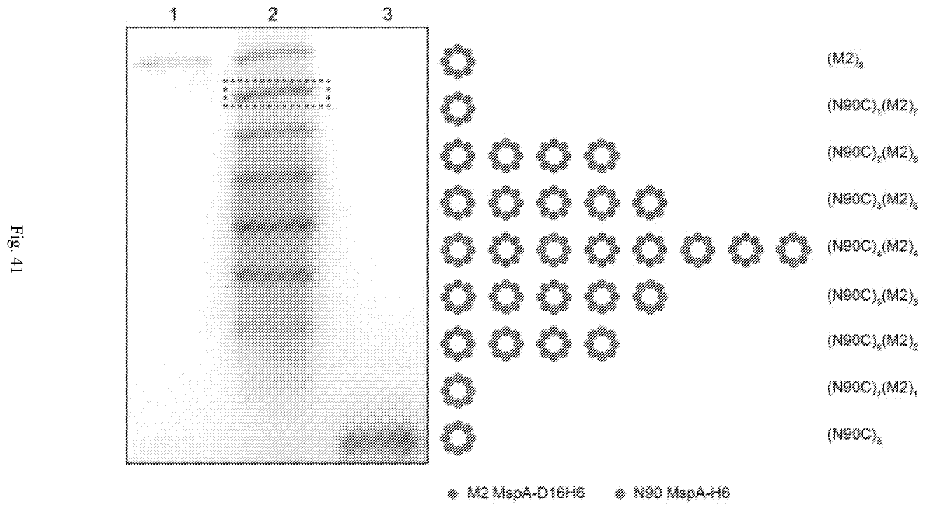

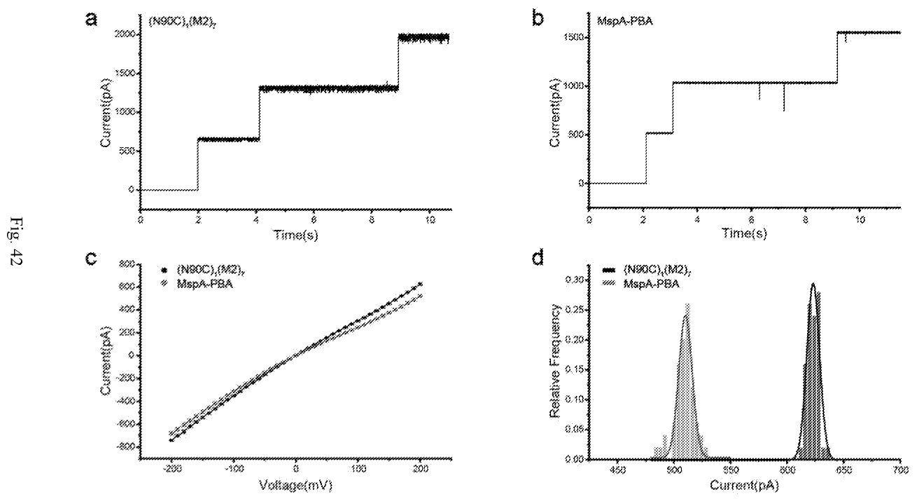

To build a hetero-octameric MspA, two different genes coding respectively for N90C-MspA-H6 and M2 MspA-D16H6 (Table 7) were custom synthesized and simultaneously inserted into a pETDuet-1 co-expression vector (Methods in Example 2). Specifically, the N90C-MspA-H6 codes for an MspA monomer, at the pore constriction in which a sole cysteine is placed. Whereas, the M2 MspA-D16H6 codes for the monomer that doesn't contain any cysteine. Hetero-octameric MspAs composed of different fractions of both gene expression products were generated by prokaryotic co-expression (FIG. 39) and were characterized by gel electrophoresis (FIGS. 40-41). The hetero-octameric MspA consisting of one unit of N90C-MspA-H6 and seven units of M2 MspA-D16H6 is the only desired MspA assembly and is referred to as (N90C)₁(M2)₇ (FIG. 33a). (N90C)₁(M2)₇ was separated from other MspA hetero-octamers by high resolution gel electrophoresis followed with gel extraction (Methods in Example 2, FIG. 40-41). Subsequently, 3-(maleimide) phenylboronic acid (MPBA) was allowed to react with the sole cysteine of (N90C)₁(M2)₇ (FIG. 33b). A real-time observation of this reaction at the level of a single molecule was carried out by single channel recording in a 1.5 M KCl, 10 mM MOPS, pH 7.0 buffer (FIG. 33c, Methods in Example 2). With a single (N90C)₁(M2)₇ inserted in the membrane and a continually applied +200 mV bias, the open pore current of (N90C)₁(M2)₇ (10) measures ˜620 pA. Upward noises, which result from the cysteine residue at the pore constriction as previously reported⁴⁷, were also observed at this stage. With the addition of MPBA to cis at a final concentration of 1 mM, a single current drop measuring ˜100 pA was immediately observed. The previously observed upward noises also disappeared simultaneously, suggesting that the cysteine residue has been occupied and the PBA modification to the pore constriction was successful. The reaction, which is triggered by diffusion of MPBA to the pore constriction, can be accelerated by the addition of a higher concentration of MPBA. For simplicity, this PBA modified MspA is referred to as MspA-PBA. At the same condition, the open pore current of MspA-PBA (Iₚ) measures ˜520 pA (FIG. 33c).

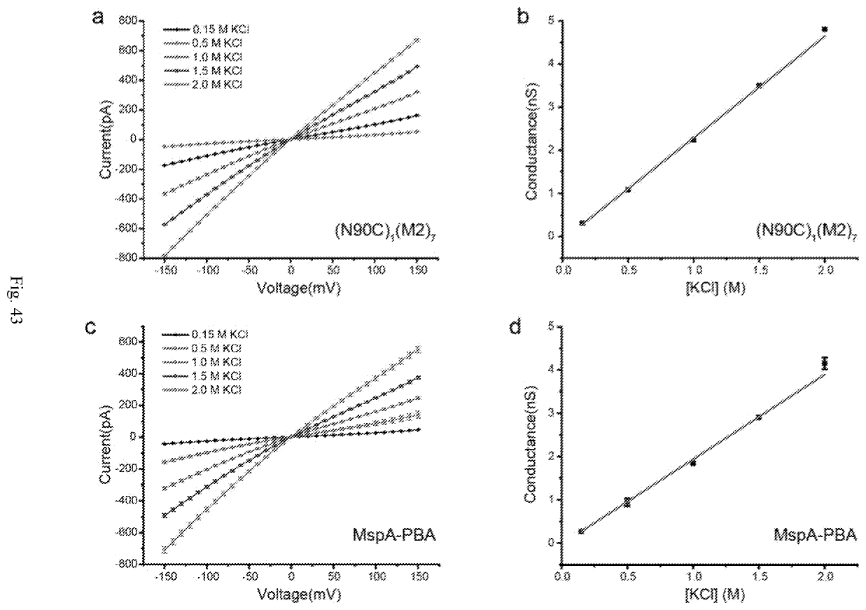

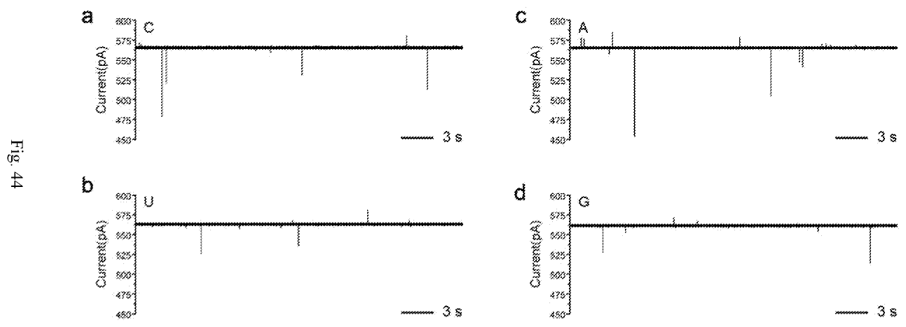

MspA-PBA can also be prepared in ensemble by mixing (N90C)₁(M2)₇ with MPBA (Methods in Example 2). If not otherwise stated, all subsequent measurements were carried out using ensemble-prepared MspA-PBA. After the addition of the ensemble-prepared MspA-PBA to cis, spontaneous pore insertion was observed, confirming that the high pore-forming activity of MspA-PBA is fully retained (FIG. 42). Statistical results of the open pore current of (N90C)₁(M2)₇ and MspA-PBA are measured at 623±13 (mean±FWHM) pA and 510±14 (mean±FWHM) pA (FIG. 42), consistent with the open pore currents previously measured during single channel recording (FIG. 1c). I-V curves of (N90C)₁(M2)₇ and MspA-PBA acquired with varying concentrations of KCl (0.15 M to 2M KCl) were also demonstrated in FIG. 43. According to the slope of the I-V curves, the conductance of MspA-PBA measured with a 1.5M KCl buffer was derived to be ˜2.91 nS, which is satisfyingly large.

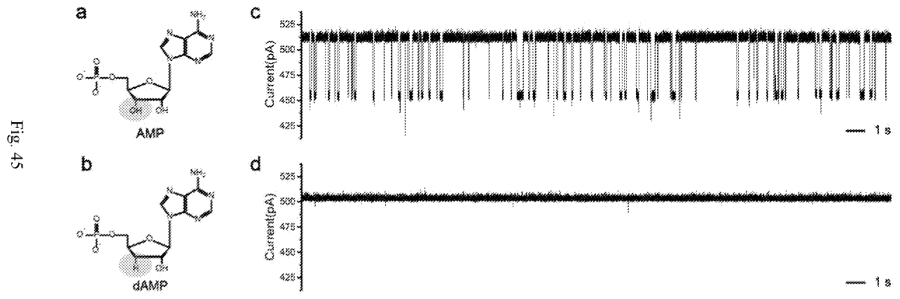

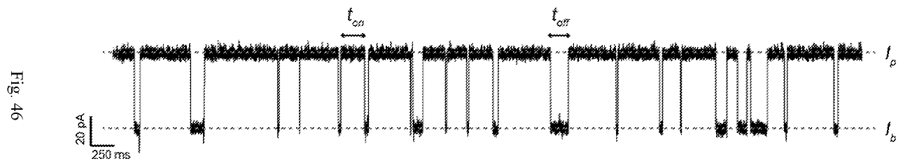

NMPs consist of a ribose, a phosphate group and a nucleobase, serving as monomeric units of RNA. Due to the presence of a cis-diol in the ribose, NMPs possess an affinity to PBA⁴⁸ and may be directly detected by MspA-PBA. Experimentally, single channel recording was performed using MspA-PBA in a 1.5 M KCl buffer (1.5 M KCl, 10 mM MOPS, pH 7.0) (Methods in Example 2). A transmembrane potential of +200 mV was continually applied. Four canonical NMPs, adenine mononucleotide (AMP), guanine mononucleotide (GMP), cytosine mononucleotide (CMP) and uracil mononucleotide (UMP) were tested as analytes (FIG. 33d). Either type of NMP was added to cis at a final concentration of 300 μM. Subsequently, successive resistive pulses caused by either type of NMP were immediately observed (FIG. 33d). However, no events were observed when M2 MspA was tested, confirming that the PBA located at the pore constriction is critical in the generation of NMP sensing events (FIG. 44). At this condition, ˜1800 events/h were obtained from each pore. The pore could normally withstand a few hours of continuous measurement. Deoxyribonucleoside monophosphate (dNMP) fails to report any event when sensed by MspA-PBA (FIG. 45). This is expected because dNMPs have no cis-diol structure and can't form the boronate ester which is necessary for sensing. The molecular mechanism of sensing which results from reversible covalent bond formation between an NMP and the PBA at the pore constriction is thus confirmed. It also suggests that this sensing strategy won't be interfered with dNMPs in a real measurement scenario.

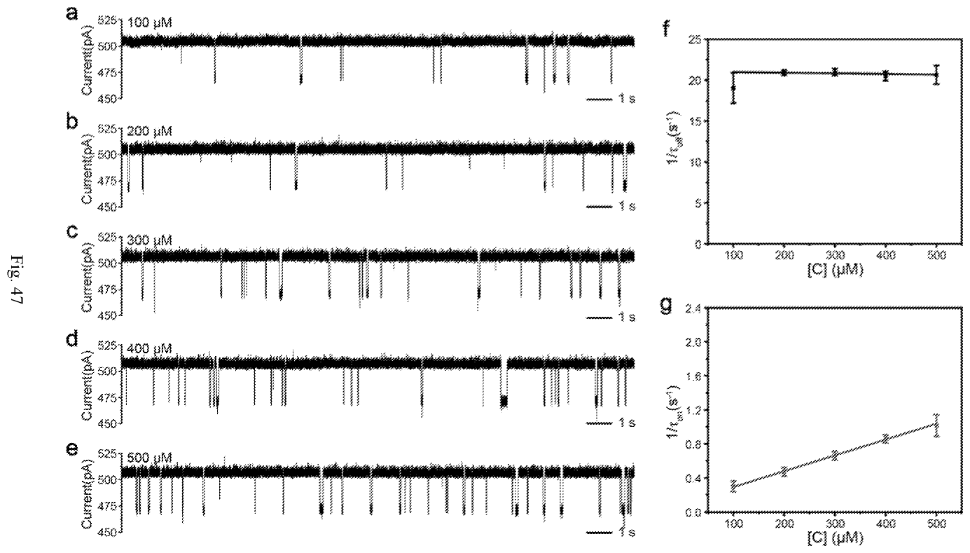

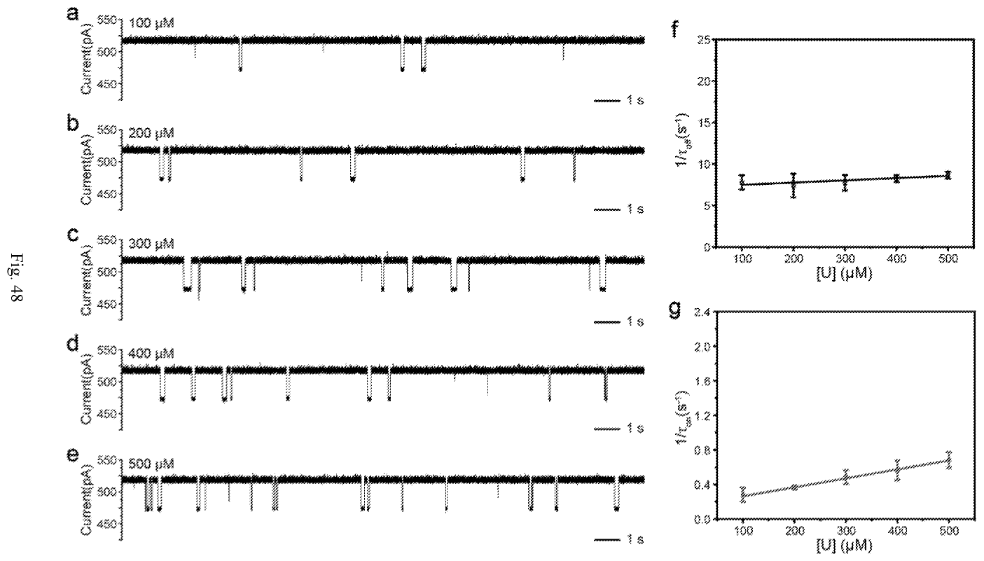

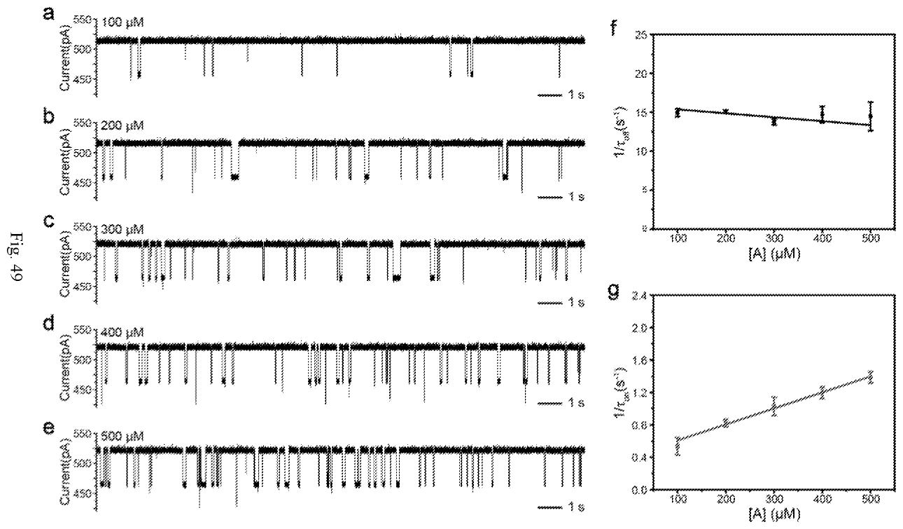

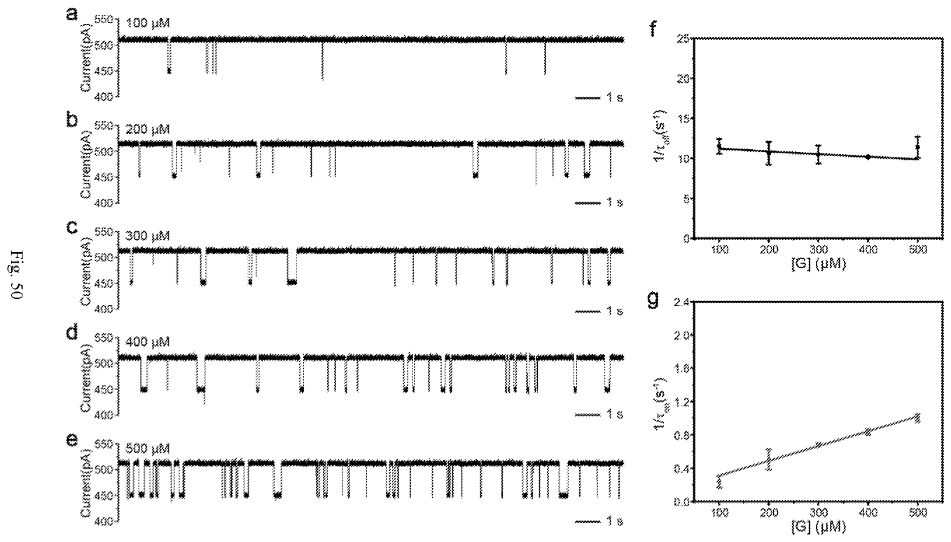

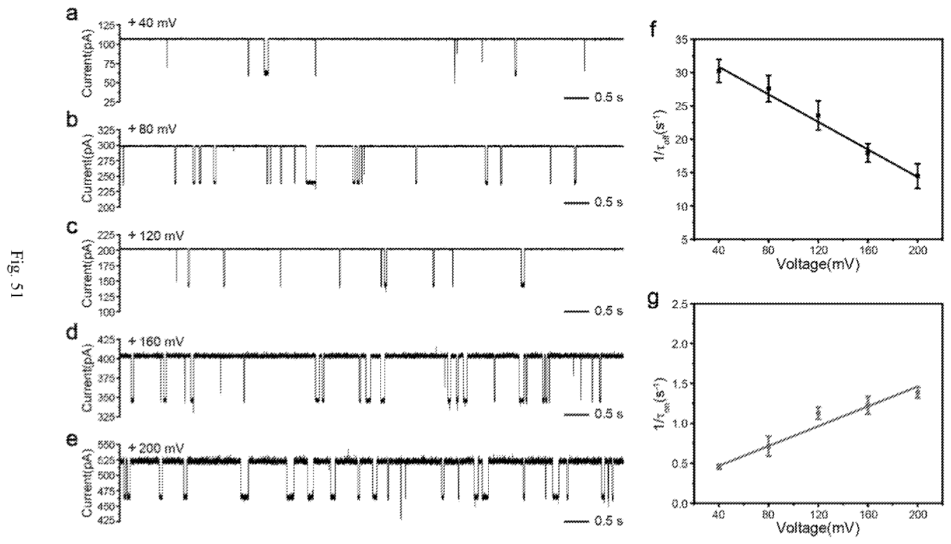

To describe NMP sensing events quantitatively, the event dwell time (τ_{off}), the inter-event interval (τₒₙ), the percentage blockage (% I_{b}=(Iₚ−I_{b})/Iₚ) and the noise amplitude (S.D.) were derived as described in FIG. 46. Generally, the histograms of τ_{off} and τₒₙ show an exponential distribution, and could be fitted to derive the mean time constants τ_{off} or τₒₙ, respectively. The histograms of % I_{b} and S.D. show a Gaussian distribution, which could be fitted to derive the mean percentage blockage % I_{b} and S.D., respectively. During NMP sensing, by varying the NMP concentrations in cis, the reciprocal of dwell time (1/τ_{off}) remains constant, consistent with a unimolecular dissociation mechanism. Whereas the reciprocal of the inter-event interval (1/τₒₙ) linearly correlates with the NMP concentration in cis (Tables 8-11, FIG. 47-50), consistent with a bimolecular model. The dependence of the applied voltage during NMP sensing was investigated using AMP as a representative analyte. Generally, when the voltage is upregulated, the 1/τ_{off} decreases and the 1/τₒₙ increases (Table 12, FIG. 51). This is expected because in a pH 7.0 buffer, the phosphate group of NMP is negatively charged and the electrophoretic force applied on the NMP analyte can strongly regulate the rate of event appearance and the event dwell time.

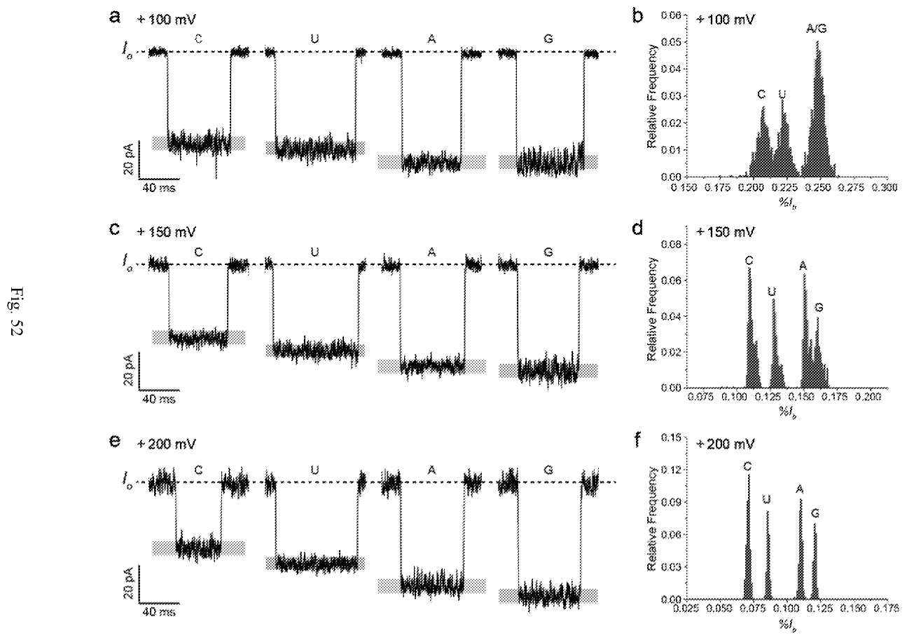

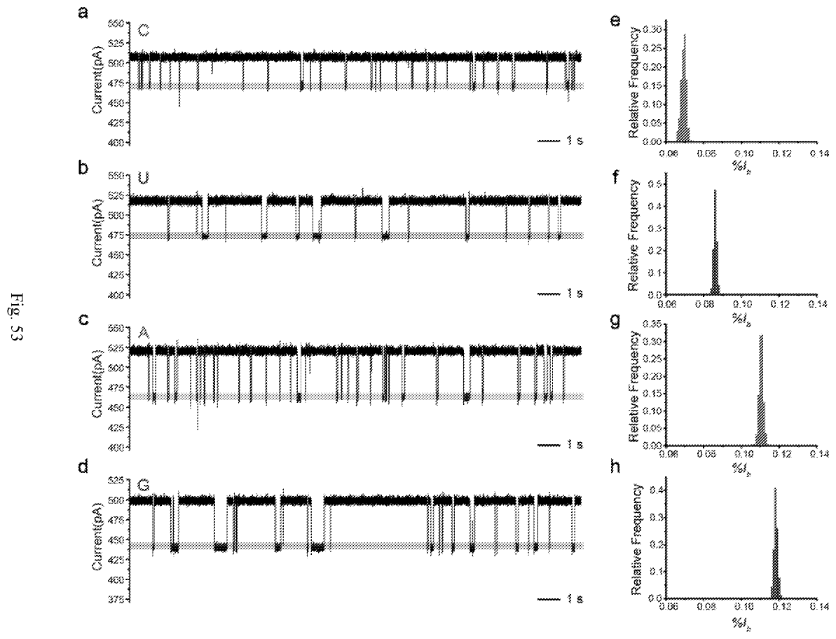

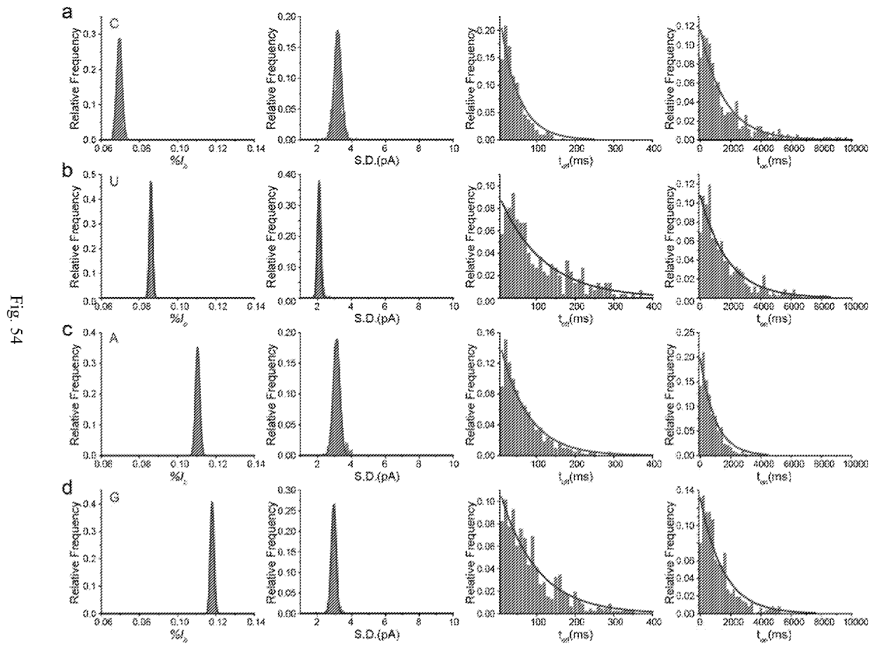

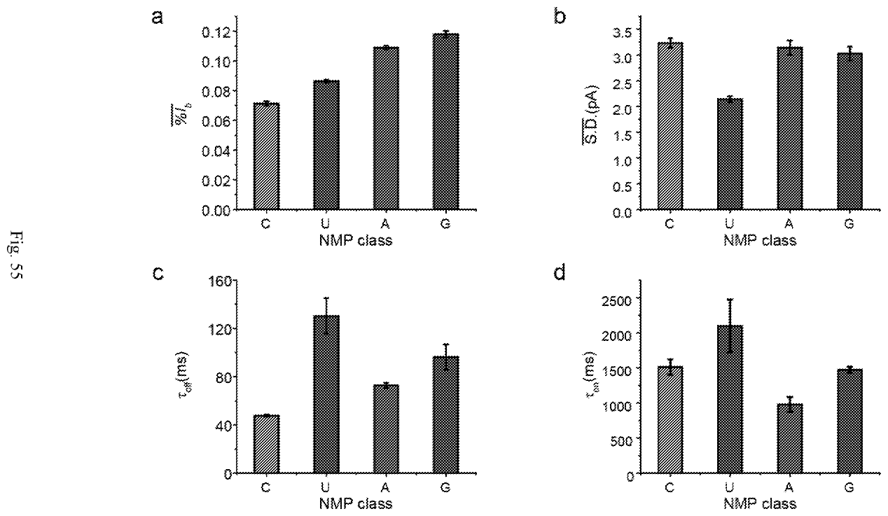

The conical lumen structure of MspA provides excellent resolution with which to distinguish between analytes with minor structural differences⁴¹. Although NMPs differ only in their nucleobase components, bindings of different NMPs to MspA-PBA result in highly distinguishable event features (FIG. 33d). This difference is more amplified at a higher applied voltage (FIG. 52). However, to avoid bilayer rupture while maintaining the high resolution in order to discriminate between different NMPs, all subsequent measurements were carried out at a voltage of +200 mV, if not otherwise stated. In this condition, events generated by different NMPs form highly distinguishable populations in the scatter plot of % I_{b} vs S.D. (FIG. 33e). The histograms of % I_{b} of different NMP events also show fully separated Gaussian distributions (FIG. 33e, FIG. 53), in which CMP (%̅ I̅_̅{̅b̅}̅=7.1±0.2%, N=3), UMP (%̅ I̅_̅{̅b̅}̅=8.64±0.09%, N=3), AMP (%̅ I̅_̅{̅b̅}̅=10.89±0.14%, N=3) and GMP (%̅ I̅_̅{̅b̅}̅=11.8±0.2%, N=3) are fully resolved without any ambiguity (Table 13, FIGS. 54 and 55). The event dwell times for different NMPs are widely distributed, producing events with varying pulse widths. However, the mean event dwell time (τ̅_̅{̅o̅f̅f̅}̅) for different NMPs are generally similar. More details of NMP binding kinetics are also summarized in Table 13 and FIG. 55.

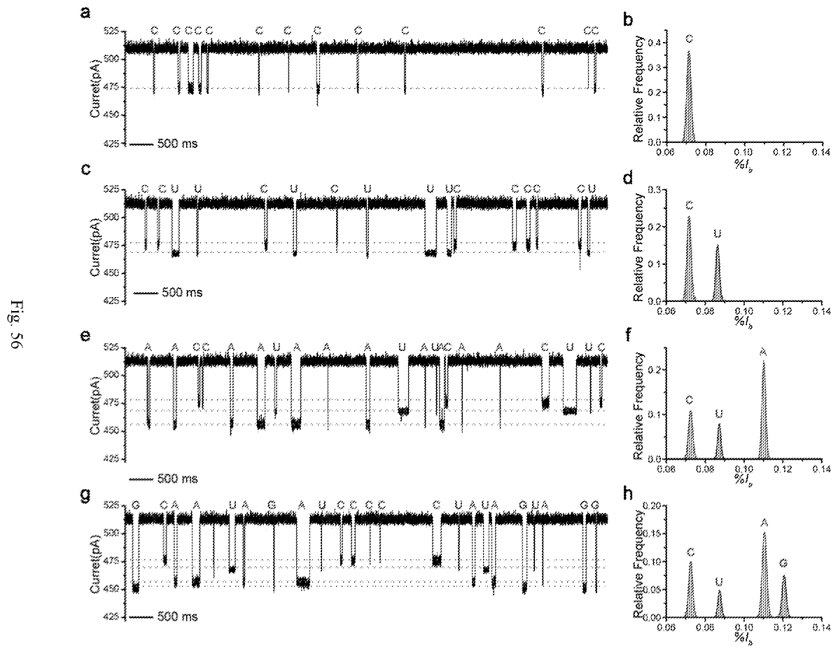

Simultaneous sensing of CMP, UMP, AMP and GMP using MspA-PBA was also performed (FIG. 33f, FIG. 56), from which different NMP identities can be directly called based on their distinct blockage characteristics. The event dwell time, which distributes widely (FIG. 56), is however not considered as a parameter in event identification. To the best of our knowledge, nanopore discrimination between canonical NMPs without any overlaps in the event distribution has never been previously reported.

### Distinguishing of Epigenetic NMPs

The above described method is in principle suitable to detect any nucleoside monophosphate as long as the cis-diol structure of ribose is retained. According to the literature, ˜170 epigenetic NMPs have been previously discovered¹. They are generated post-transcriptionally and play critical roles in many biological activities including cell differentiation, gene expression and disease processes². However, these epigenetic NMPs have extremely minor structural differences and pose a great challenge for direct identification. Acknowledging the high resolution of MspA, this challenge may be solved by directly monitoring event features of nanopore readouts when epigenetic NMPs are bound to the pore constriction.

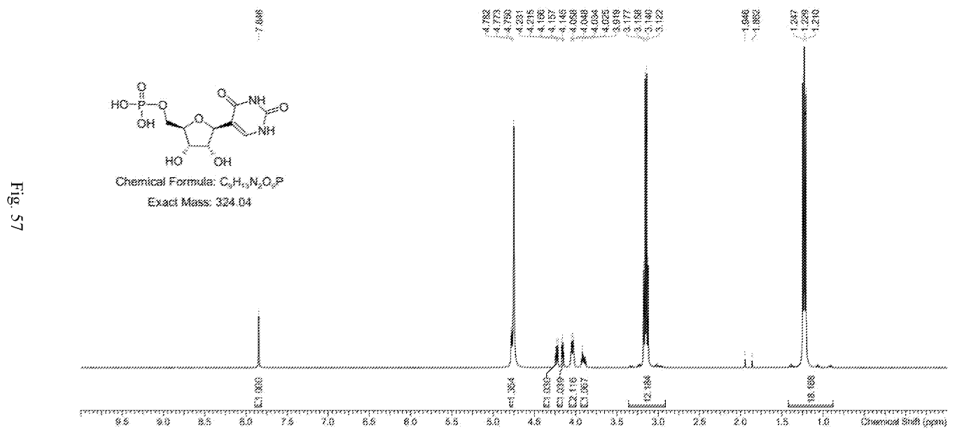

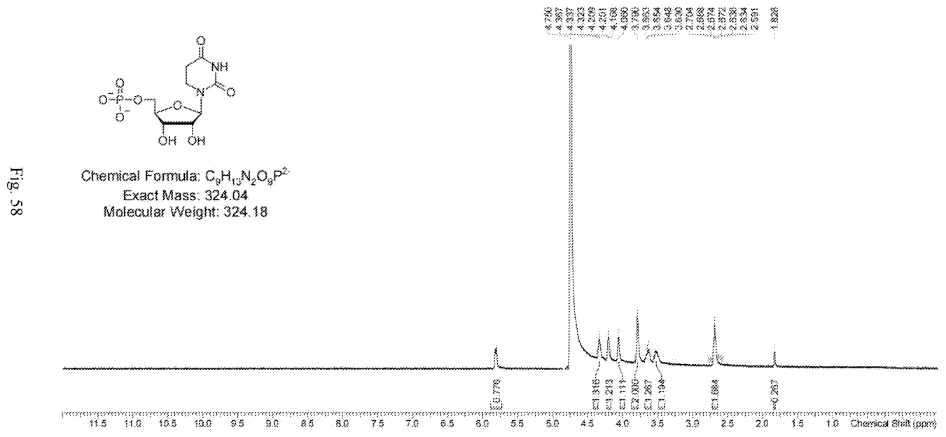

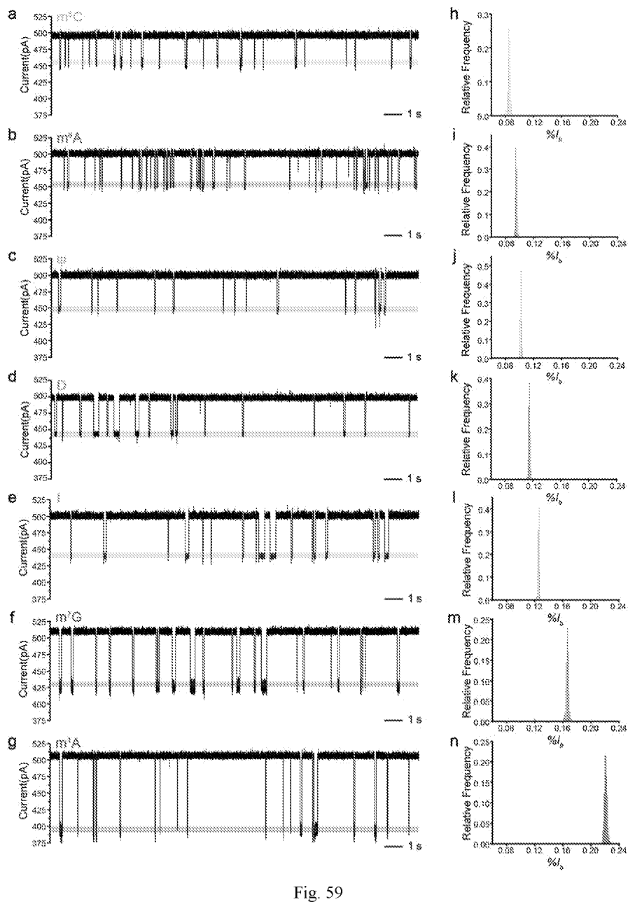

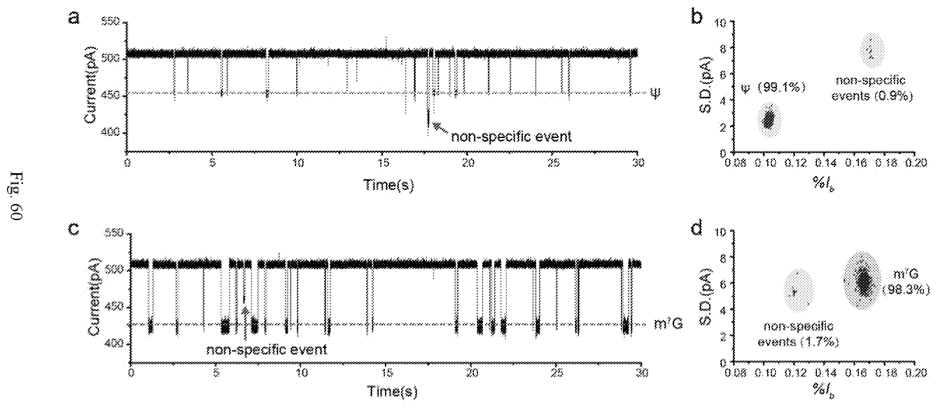

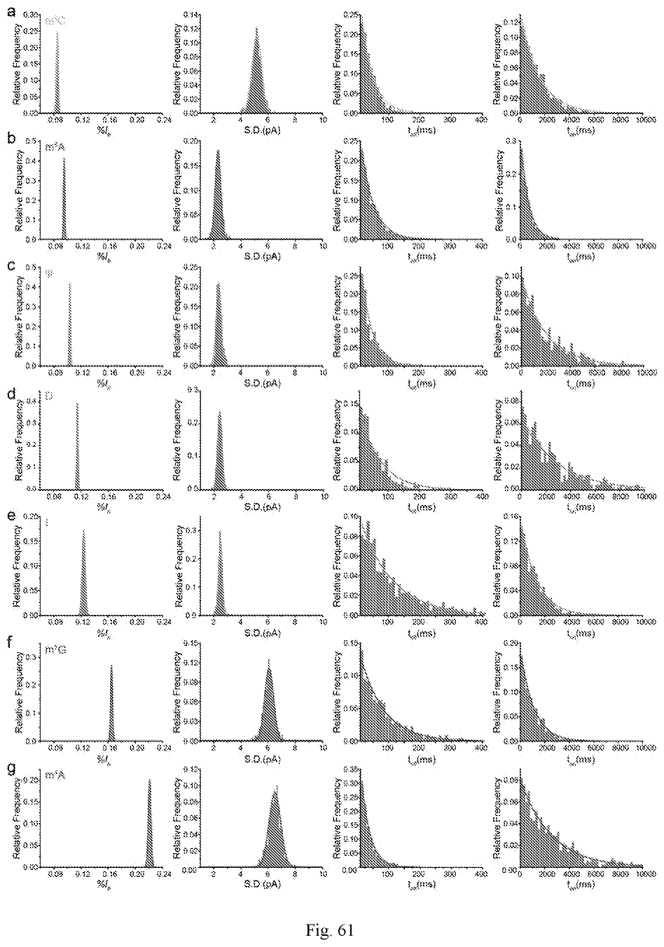

To testify this hypothesis, the same measurements were carried out by taking monophosphates of m⁵C, m⁶A, m⁷G, m¹A, I, Ψ and D as the analyte. Due to a lack of commercially available model compounds, Ψ (FIG. 57) and D (FIG. 58) were custom synthesized and characterized by WuXi AppTec. These epigenetic NMPs have covered the common types of modification occurring with canonical NMPs such as methylation, deamination, isomerization and reduction. To show their chemical structures more clearly, their nucleobase components are demonstrated in FIG. 34a. Nanopore measurements were carried out in a 1.5 M KCl buffer (1.5 M KCl, 10 mM MOPS, pH 7.0) (Methods in Example 2). A transmembrane potential of +200 mV was continually applied. Each epigenetic NMP was added to cis at a final concentration of 300 μM. As shown in FIG. 34a and FIG. 59, events of epigenetic NMPs have significantly different blockage amplitudes. To demonstrate a full comparison between all NMPs being tested to date, the % I_{b} distribution for each NMP were demonstrated in a violin plot, demonstrating that almost all NMPs are already distinguishable solely by analysis of their % I_{b}, though the event distributions of UMP and m⁵C still have some overlaps (FIG. 34b). The big variations in ψ and m⁷G result from the detection of non-specific events away from the main population of events. They may result from impurities introduced during synthesis of the compound. However, these non-specific events only contribute to 0.9% and 1.7% of all events being detected respectively (FIG. 60). The noise characteristics of NMPs may also be included in event analysis to improve the discrimination performance (Table 13, FIGS. 61-62). By plotting a scatter plot containing % I_{b} vs. S.D. of NMP sensing events acquired from eleven different analytes, eleven fully resolved event populations were generated, respectively corresponding to each NMP being sensed (FIG. 34c). This confirms that this sensing configuration is generally compatible with epigenetic NMPs and their events are fully distinguishable. Direct discrimination between these eleven types of NMPs using nanopores has however, never been reported, to the best of our knowledge.

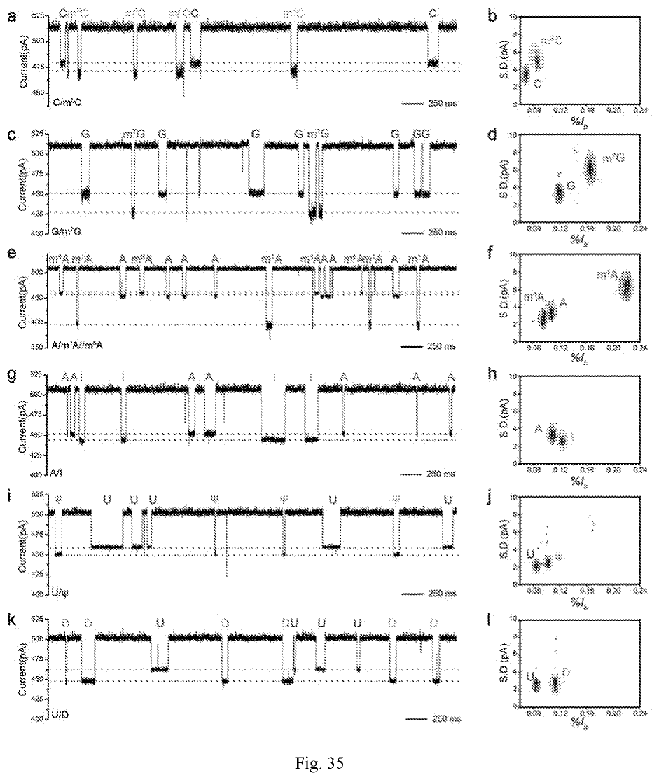

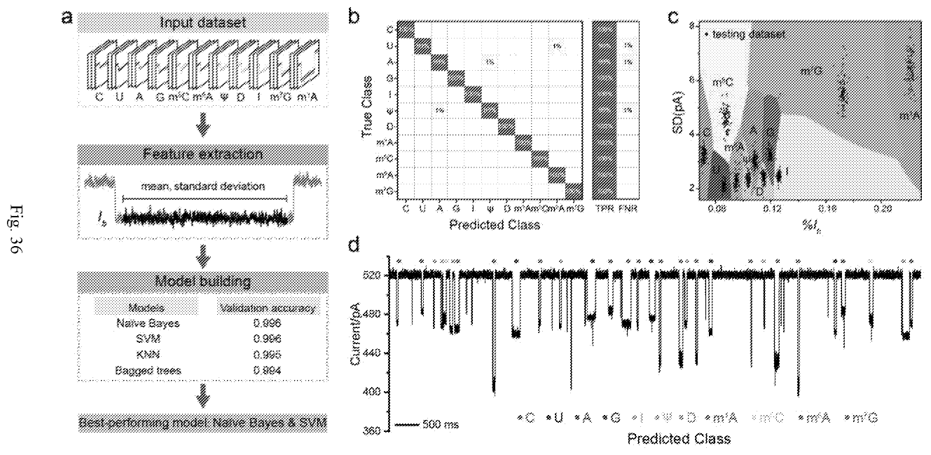

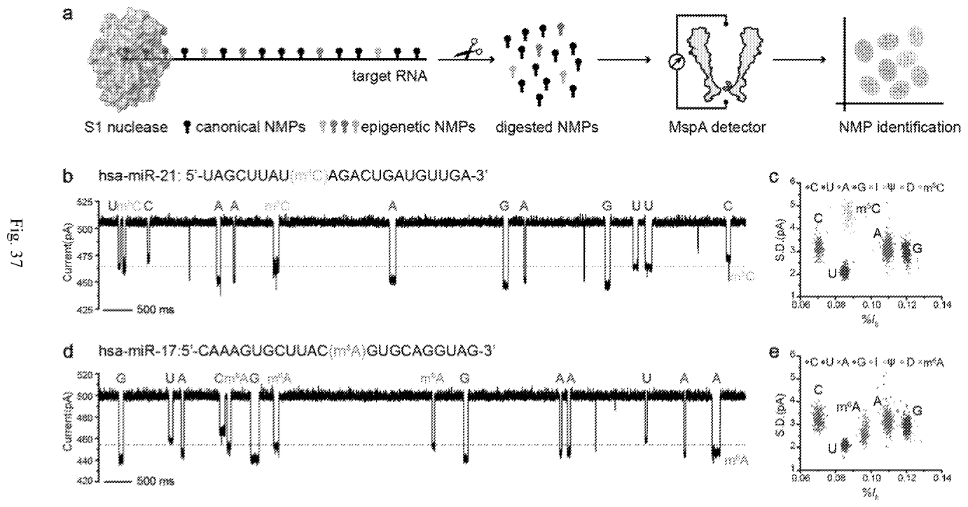

To further demonstrate the discrimination between epigenetic NMPs and their corresponding canonical counterparts, nanopore sensing between epigenetic and canonical NMPs was performed in separate groups (FIG. 35). Methylated RNA nucleotides are ubiquitous in all species of organisms, and two-thirds of RNA modifications involve the addition of methyl groups⁴⁹. From simultaneous sensing of CMP and m⁵C, GMP and m⁷G, AMP, m¹A and m⁶A, it was discovered that methylations in heterocyclic rings generate an obviously enhanced current blockage and noise (FIGS. 35a-35f), whereas methylation to other sites reports an opposite effect (e.g. m⁶A, FIGS. 35e, 35f). Simultaneous sensing of AMP and I show that deamination can decrease the blockage current (˜7.5 pA) and noise (˜31.2 pA) (FIGS. 35g, 35h). This finding is also corroborated by the characteristic events of CMP and UMP. For isomerization of U to y, a current increase of ˜8.0 pA is observed although U and w have identical molecular weights (FIGS. 35i, 35j), which can't be directly distinguished solely by mass spectroscopy. For reduction of U to D, a current increase of ˜14.0 pA is observed (FIGS. 35k, 35l). These findings might provide clues to predict other modification signals. However, these changes in event characteristics are simultaneously determined by molecular volume, net charge and other factors, which may require the assistance of molecular dynamics simulations for further in-depth investigations.

### NMP Identification by Machine Learning

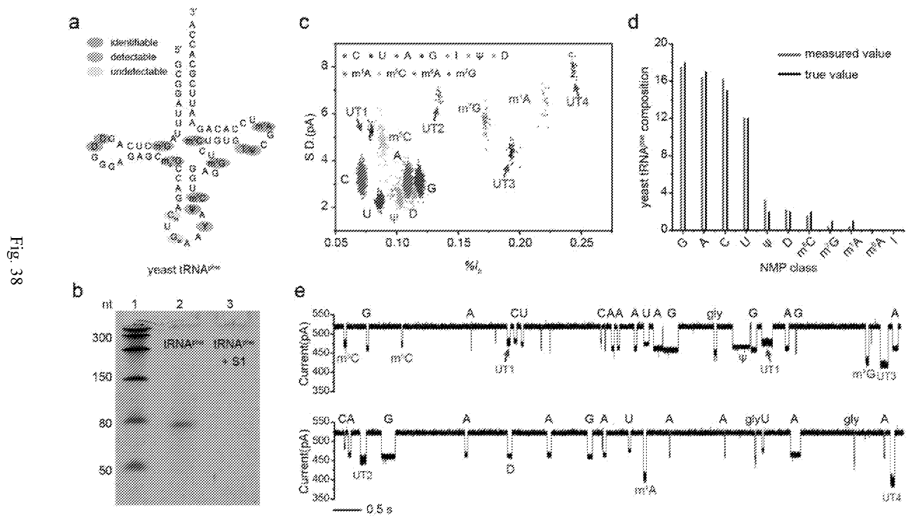

A machine learning algorithm was established to automatically identify NMPs. The overall training process includes dataset input, feature extraction and model building (FIG. 36a, Methods in Example 2). Specifically, 500 representative events acquired from each NMP were used to form a dataset. All events in the dataset have known labels since they were acquired during measurements with a sole NMP with a known identity. The dataset was then split into a training set (80%) for model training and a testing set (20%) for model testing. The % I_{b} and S.D. of each event were automatically extracted using MATLAB to form a feature matrix. A 10-fold cross-validation was performed to randomly split the training data into a training subset for model training and a validation subset for model validation. The process of model training was carried out with the Classification Learner toolbox of MATLAB. Mainstream classifiers including Decision Trees, Discriminant Analysis, Naïve Bayes, Support Vector Machine (SVM), K Nearest Neighbor (KNN), Ensemble and Neural Network were estimated with default settings of parameters. The same dataset was repetitively used in the model evaluation. Most models demonstrated satisfactory validation accuracies, indicating that the input data is of a high quality. Specifically, the Kernel Naïve Bayes model and Linear SVM model reported the highest accuracy score of 0.996 (Table 14). The trained models were further evaluated using the testing set, by which the Linear SVM model performed slightly better (Table 14) and was therefore selected as the optimum model for further evaluation and predictions. The confusion matrix results based on model testing using the Linear SVM model are demonstrated in FIG. 36b, in which most NMP sensing results report an either 99% or 100% accuracy, confirming that there is no significant bias in the identification of different NMPs. In FIG. 36c, a decision boundary plot generated by the Linear SVM model is also demonstrated. To visually demonstrate event recognition, it is placed above the scatter plot of the testing data.

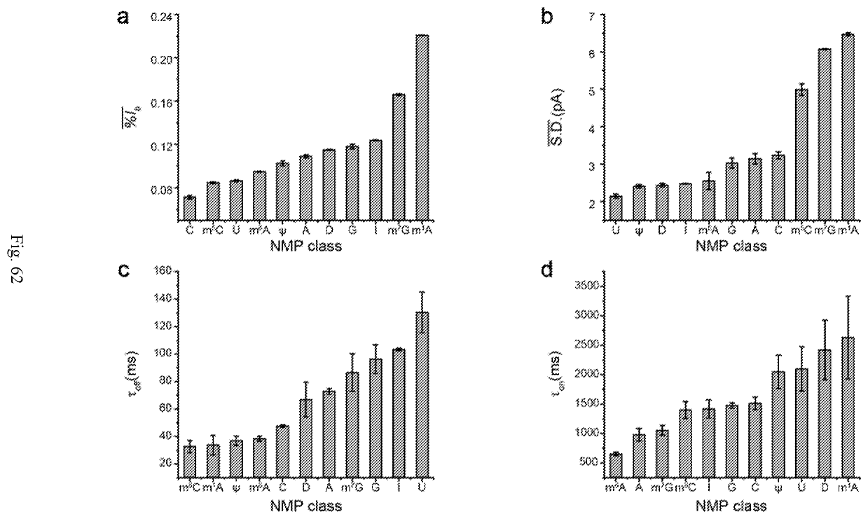

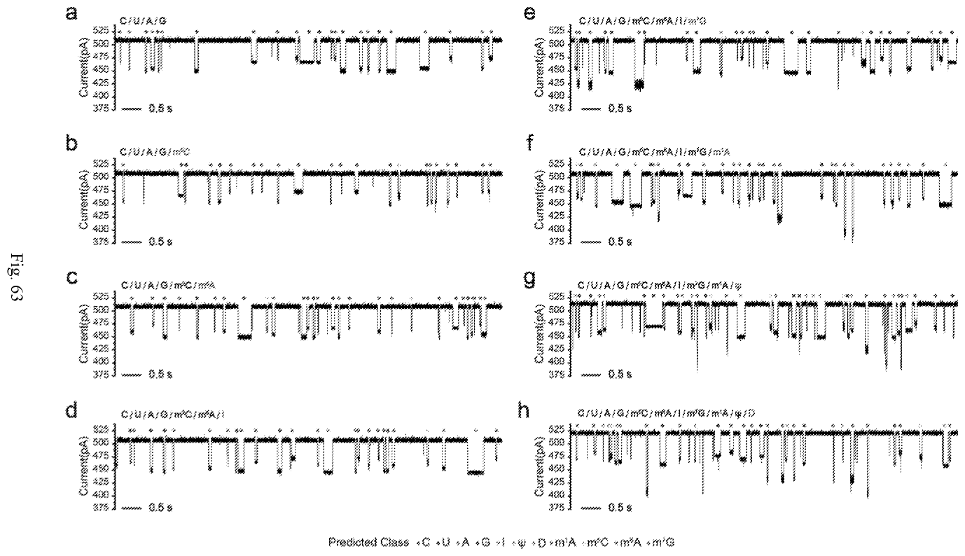

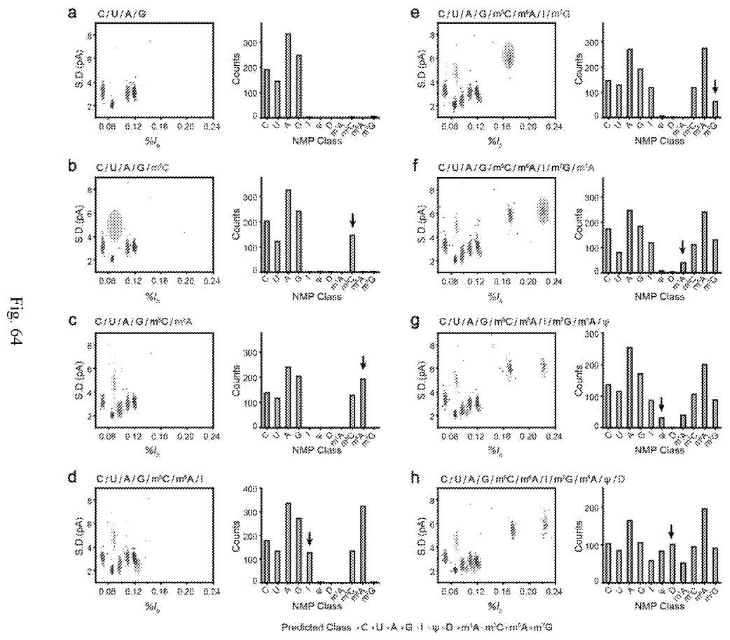

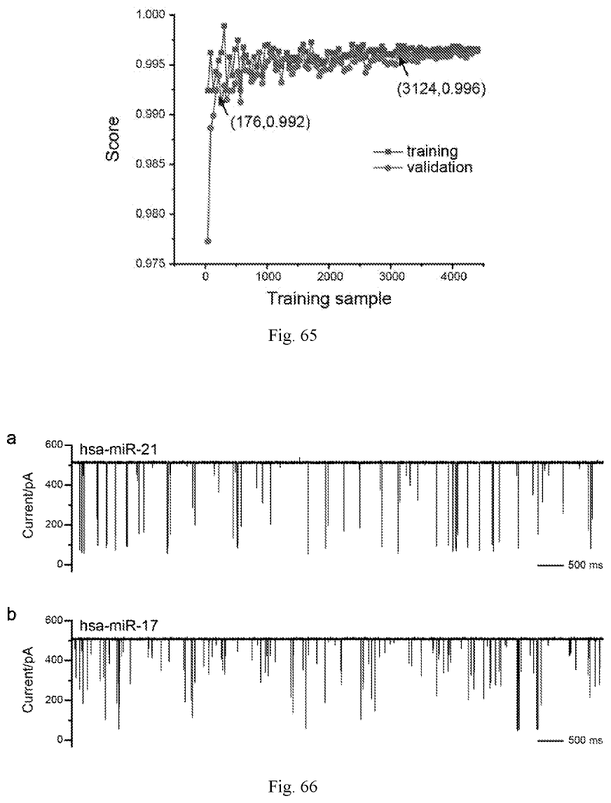

The previously trained Linear SVM model was employed to predict events with unknown identities. The measurements were carried out as described in Methods in Example 2. Modified NMPs were added to the cis side in the order of m⁵C, m⁶A, I, m⁷G, m¹A, Ψ and D with CMP, UMP, AMP and GMP already placed in cis. The final concentration of each NMP in cis was 100 μM. With the Linear SVM model, newly added NMPs can be accurately identified (FIGS. 63 and 64). To evaluate the training efficiency of the model, learning curves were generated respectively with training or validation data (FIG. 65), from which it is conclusive that 176 events were required for the model to reach a 0.990 accuracy. When the dataset size exceeds 3124, the accuracy saturates at ˜0.996. According to the learning curve result, overfitting of the model is also not happening. To show event identification from a mixture, a representative trace containing events from eleven different NMPs is demonstrated in FIG. 36d and Supplementary Movie 1. Different NMP types can be recognized and the corresponding labels predicted by machine learning are marked above the trace. This efficiently assists automatic nanopore sensing of different NMPs in a real measurement scenario in which different NMPs exist as a mixture.

Sensing of Epigenetic NMPs from Methylated microRNA

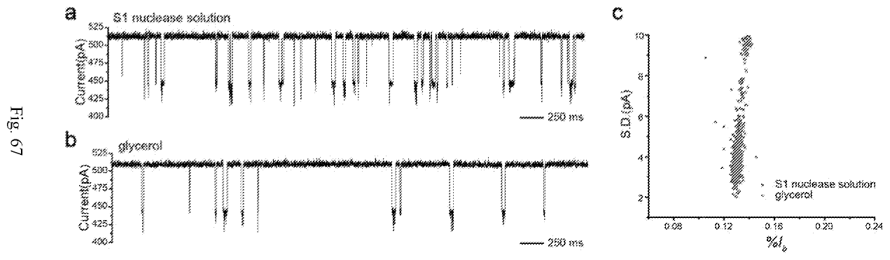

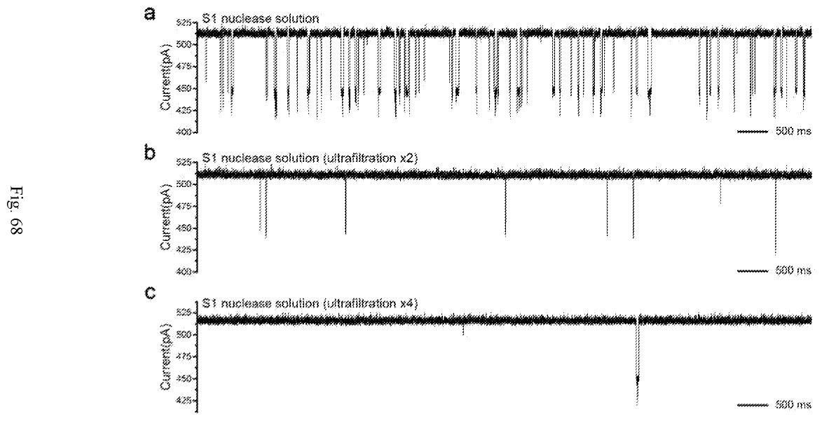

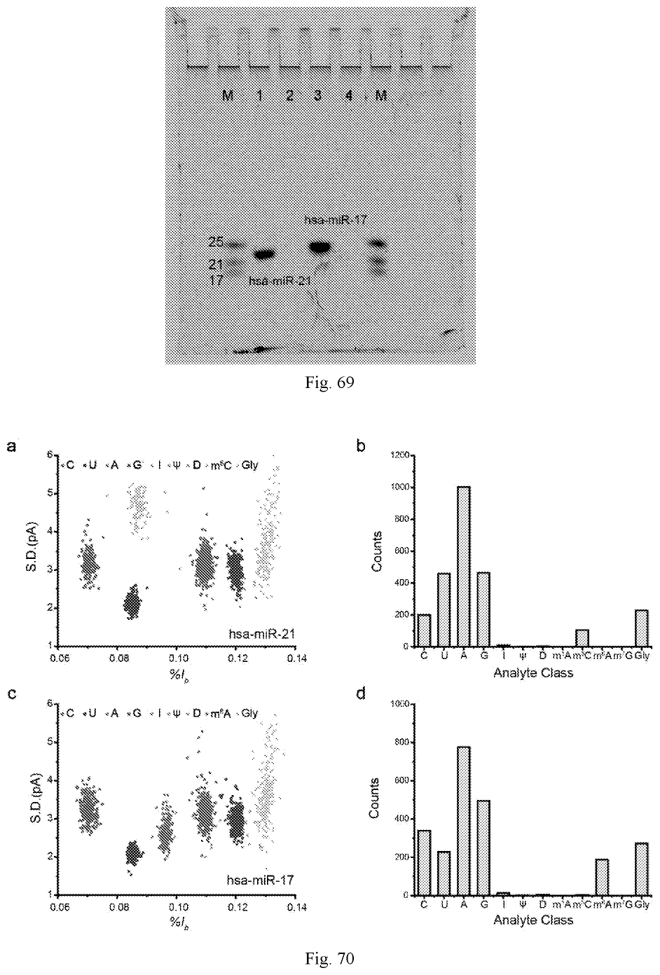

We further sought to demonstrate direct sensing of epigenetic NMPs in RNAs. The measurement diagram is demonstrated in FIG. 37a. Briefly, RNA is first enzymatically decomposed into NMPs by treatment with S1 nuclease. The generated NMPs were then sensed by MspA-PBA. The observed nanopore events were identified by the previously trained machine learning model, which reported the RNA composition including epigenetic modifications. To experimentally demonstrate its feasibility, two microRNAs including hsa-miR-21 and hsa-miR-17 with known methylated sites⁵⁰ were applied. Specifically, the hsa-miR-21 contains a m⁵C at position 9 and the hsa-miR-17 contains a m⁵C at position 13 (Table 15). First, without any enzymatic treatment, hsa-miR-21 and hsa-miR-17 were sensed by MspA-PBA. However, only short-residing spiky events with undefined event amplitudes were observed (FIG. 66), indicating that this sensing configuration is insensitive to the template RNAs itself. To minimize interferences of glycerol in the stock solution of S1 nuclease (FIG. 67), the S1 nuclease was pre-treated by ultrafiltrations to remove glycerol to improve the detection efficiency (Method in Example 2, FIG. 68). The pre-treated S1 nuclease was then employed to digest the microRNAs at 23° C. for 4 h. From the gel electrophoresis results, both microRNAs were thoroughly decomposed (Method in Example 2, FIG. 69). The enzymatic treatment product was then subjected to ultra-filtration to remove the S1 enzyme prior to nanopore measurements (Method in Example 2). Nanopore measurements were carried out with MspA-PBA (Method in Example 2) in a 1.5 M KCl buffer (1.5 M KCl, 10 mM MOPS, pH 7.0). A transmembrane potential of +200 m V was continually applied. The hsa-miR-21 digestion product was added to cis with a final concentration of 100 ng/μl. A representative trace is shown in FIG. 37b, in which many NMP binding events were observed, suggesting that the generated NMPs are well detected by MspA-PBA. Though thoroughly minimized by previous ultrafiltration treatment, events caused by glycerol binding are still noticeable during nanopore sensing. The identities of NMPs were called by the algorithm and the glycerol events were also recognized, which are highly discriminable from the demonstrated NMP events (FIG. 70).

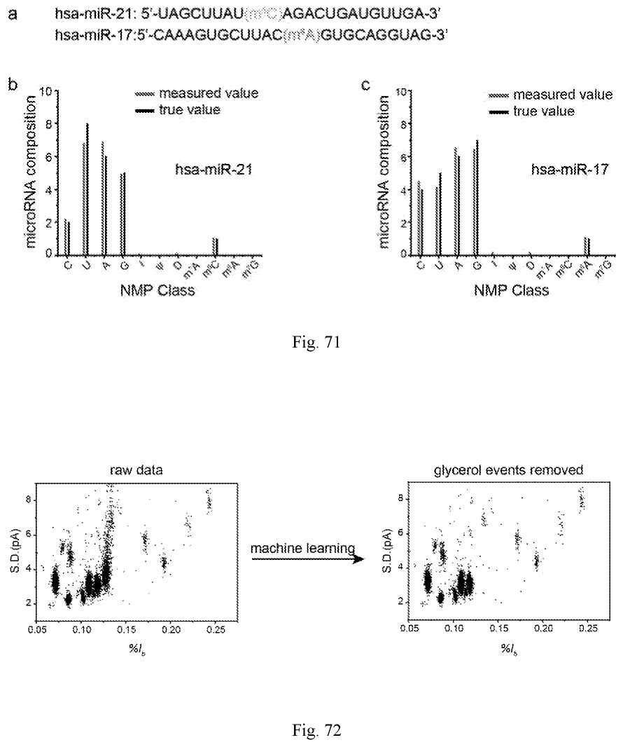

According to the results acquired with hsa-miR-21, five types of NMPs were detected, including CMP, UMP, AMP, GMP and m⁵C (FIGS. 37b and 37c), consistent with the hsa-miR-21 sequence composition (Table 15). The abundance of each NMP type in has-miR-21 was also evaluated based on the rate of event appearance followed with a calibration (Method in Example 2, Table 16). The relative NMP composition in hsa-miR-21 was estimated to be 2.17 CMP, 6.81 UMP, 6.88 AMP, 4.92 GMP, 1.03 m⁵C, 0.06 I, 0.01 ψ and 0.10 D (FIG. 71), generally consistent with the true values. The misjudgement of I, ψ and D result from the minor distribution overlap between AMP, ψ, GMP and I. However, the proportion of misjudgement is negligible. The feasibility of epigenetic NMP identification by nanopore sensing is thus approved. To test its generality, hsa-miR-17, which is another microRNA containing a different epigenetic NMP in the sequence, was tested identically to that shown with hsa-miR-21. A representative trace containing nanopore sensing events of the digestion products of hsa-miR-17 is demonstrated in FIG. 37d. The scatter plot results demonstrate five dominant populations of NMP events, respectively corresponding to CMP, UMP, AMP, GMP and m⁶A (FIG. 37e), consistent with the sequence component of hsa-miR-17 (Table 15). Quantitative analysis also shows that the relative count of m⁶A site is 1.08, indicating that only one m⁶A site was present in the hsa-miR-17 (FIG. 71), also consistent with expectations.

Detection of Epigenetic NMPs from Brewer's Yeast tRNA^{phe}

Transfer RNA (tRNA) is a type of low molecular weight RNA serving to link the messenger RNA sequence into the amino acid sequence of protein. Mature tRNAs also contain rich chemical modifications. As reported, more than 90 types of modifications have been discovered in tRNA⁵¹. It is thus an ideal RNA to evaluate the performance of MspA-PBA in the identification of epigenetic modifications of natural samples. The brewer's yeast phenylalanine specific tRNA (yeast tRNA^{phe})^{42, 52} is applied as a model RNA to test its feasibility. As reported, a mature yeast tRNA^{Phe} contains 14 epigenetically modified sites originated from 11 types of modifications including m²G=N2-methylguanosine, D=dihydrouridine, m₂²G=N2,N2-dimethylguanosine, Cₘ=2′-O-methylcytidine, Gₘ=2′-O-methylguanosine, Y=wybutosine, ψ=pseudouridine, m⁵C=5-methylcytidine, m⁷G=7-methylguanosine, T=5-methyluridine and m¹A=1-methyladenosine (FIG. 38a)^{53,54}. When the yeast tRNA^{phe} is enzymatically decomposed into NMPs, monophosphates of D, ψ, m⁵C, m⁷G, m¹A, m²G, m₂²G, T and Y are in principle detectable by MspA-PBA because their cis-diol structures remain unmodified. The event parameters of D, ψ, m⁵C, m⁷G, m¹A have been previously acquired and used for model training (FIG. 34a, FIG. 36) so that their events are identifiable by the machine learning algorithm. The monophosphates of m²G, m₂²G, T and Y are in principle detectable by MspA-PBA and new clusters of events are expected to be observed. However, due to a lack of corresponding pure compounds to produce events for training, the corresponding nanopore events are detectable but not identifiable. Cₘ and Gₘ, which lack a cis-diol, are in principle undetectable by MspA-PBA.

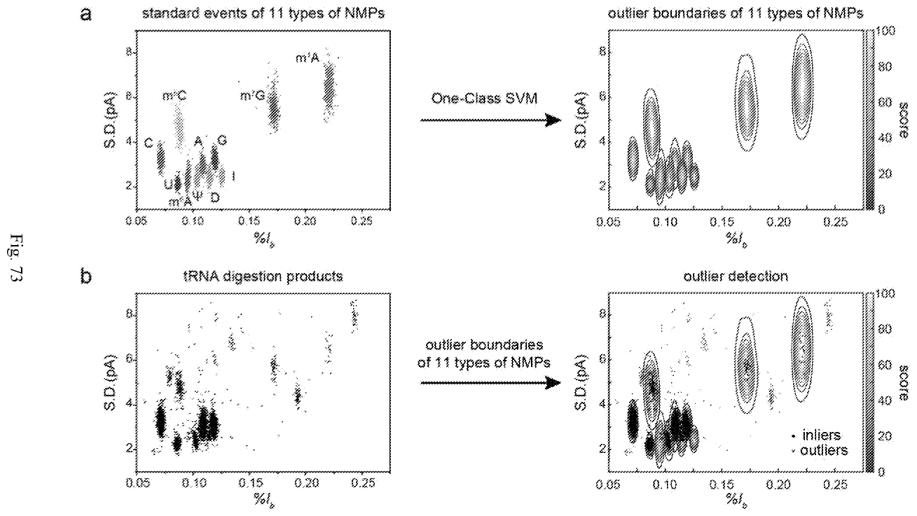

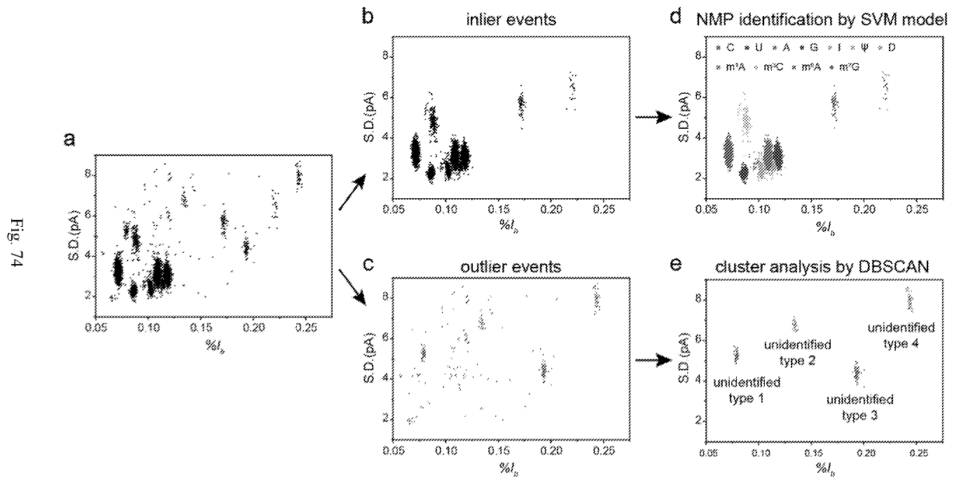

tRNA^{phe} was first enzymatically treated with S1 nuclease at 23° C. for 15 h to produce NMPs (Methods in Example 2). According to the gel electrophoresis result, it is confirmed that the tRNA^{phe} has been thoroughly decomposed (FIG. 38b). The enzymatic treatment product was then ultra-filtrated to remove the S1 nuclease and used in subsequent nanopore measurements (Methods in Example 2). Nanopore measurements were carried out with MspA-PBA (Method in Example 2) in a 1.5 M KCl buffer (1.5 M KCl, 10 mM MOPS, pH 7.0). A transmembrane potential of +200 mV was continually applied. The yeast tRNA^{phe} digestion product was added to cis with a final concentration of 100 ng/μl. The acquired raw events were shown in a scatter plot (FIG. 72). Glycerol events, which were introduced by the stock solution of the S1 nuclease, were further removed from the dataset by recognizing its highly characteristic event features using machine learning (FIG. 72). To cope with unknown epigenetic modifications in yeast tRNA^{phe}, we combined supervised and unsupervised learning algorithms to identify the remaining events of digested NMPs. Here, One-Class SVM was employed to recognize events that don't belong to any previously trained event types. These events are considered as outliers. On the contrary, events that match the previously trained event types are considered as inliers (FIG. 73), which are further identified by the trained Linear SVM model. The outlier events are however analysed with the Density-based spatial clustering of applications with noise (DBSCAN) model to detect events appearing as clusters (FIG. 74). The non-clustered events, which randomly distributed in the scatter plot, are considered as background events and are removed from the data set without further analysis.

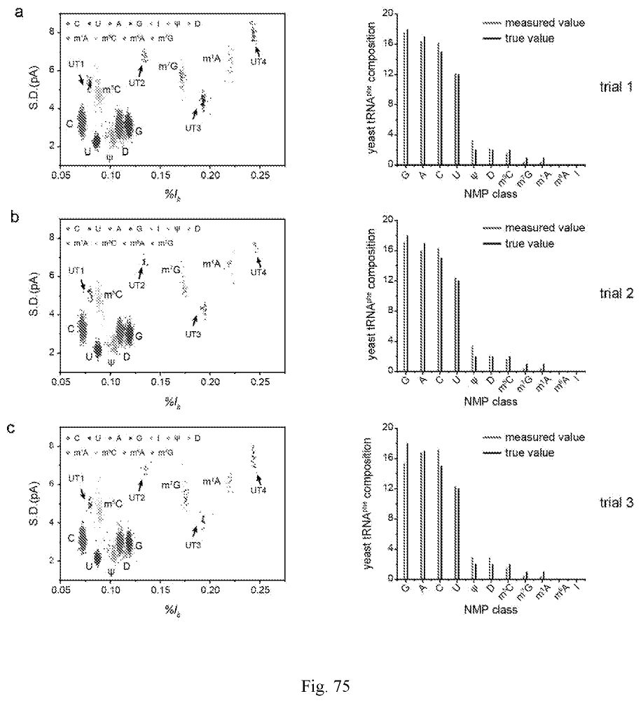

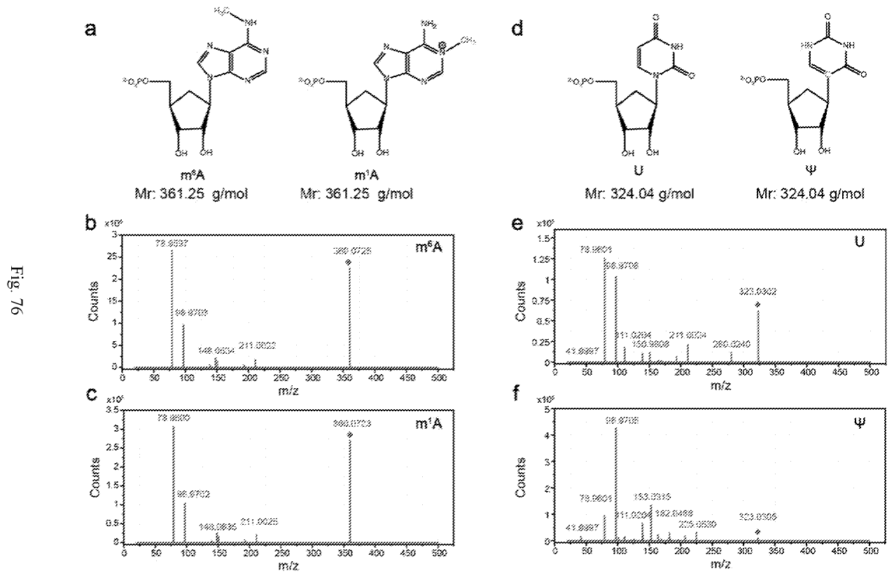

The result of the modification profile of yeast tRNA^{phe} is shown in FIG. 38c. D, y, m⁵C, m⁷G and m¹A were successfully detected, consistent with the previous training results and literatures^{53, 54}. Few m¹A events were observed, which may be from background events which coincidently share a similar event feature of m⁶A or other types of RNA mixed in the sample. Four new clusters of events, which demonstrated event features different from all NMP types that were previously applied for training, were also observed. These new clusters of events are likely from the m²G, m₂²G, T, Y or other unknown modifications in yeast tRNA^{phe} Quantitative analysis shows that the relative NMP composition in yeast tRNA^{phe} is 17.53 GMP, 16.36 AMP, 1619 CMP, 12.06 UMP, 3.24, 2.17 D, 1.53 m⁵C, 0.39 m⁷G, 0.37 m¹A, 0.11 m⁶A and 0.04 I, generally in accordance with the calculated true values (FIG. 38d)^{53, 54}. A total of three independent trials were also performed (FIG. 75) and the same conclusion is drawn, confirming the repeatability of this technique. Representative traces containing events of the yeast tRNA^{phe} digestion products are also presented in FIG. 38e. For demonstration, the identities of each event were automatically labelled by machine learning. With above results, the capacity of MspA-PBA to measure NMPs and their epigenetic modifications from natural RNAs have been well approved.

### Conclusion

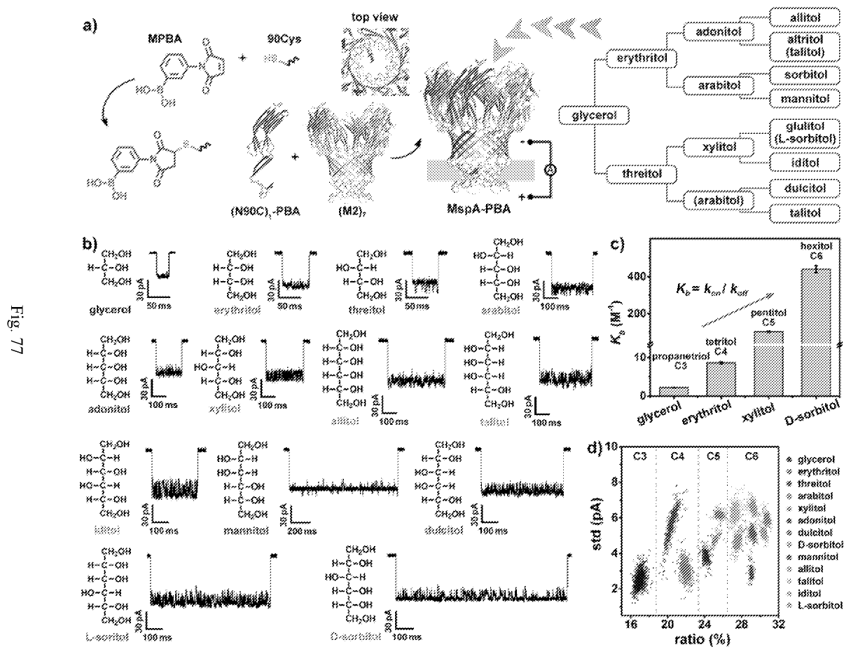

In summary, a hetero-octameric MspA containing a sole PBA adapter is reported. During nanopore sensing, it serves to reversibly react with the cis-diol of NMP to report their identities. Acknowledging the high resolution provided by the conical geometry of the pore lumen, eleven types of NMPs, including CMP, UMP, AMP, GMP, m⁵C, m⁶A, m⁷G, m¹A, I, ψ and D are fully distinguished. The sensing performance also outperforms those demonstrated by other nanopore types such as α-HL^{36, 37} or solid-state nanopores⁵⁵⁻⁵⁸. A custom machine learning algorithm was built, with which the general accuracy score of NMP identification was 0.996. The machine learning algorithm is useful by providing rapid, objective and automatic data analysis without any human interferences. With a dataset containing thousands of events, the training and prediction process only take couple seconds to finish when operated on a personal computer. The automatically generated confusion matrix, learning curves and decision boundary are also useful to evaluate the model performance and are great for data visualization. The algorithm can also automatically remove interfering or background events based on their unique event features, permitting simultaneous sensing of target analyte in a mixture. For events of natural NMPs that were not previously applied for training, anomaly detection and unsupervised machine learning are applied in data analysis. To the best of our knowledge, a PBA conjugated hetero-octameric MspA has not been reported previously. This work also reports the largest number of NMP types that can be fully distinguished. In future prospects, more NMP model compounds may be tested to produce more types of training data to reinforce the machine learning model. The only limitation is that the current sensing strategy fails to detect ribose modified NMPs, such as 2′-O-methylcytidine and 2′-O-methylguanosine. However, they only represent a minor proportion of all known RNA modifications^{1,59}. Machine learning using multiple event features may also be applied for new NMP types that were however difficult to be identified by the current model which relies on only two event features. Compared with mass spectrometry (MS), the gold standard platform for post-transcriptional modification identification, our method offers a higher resolution, especially in distinguishing RNA positional isomers (FIG. 76). It is also more suitable for RNA modification detections from mixed and native samples, without coupled with any chromatographic separation technology and complex data interpretation. This sensing strategy was also applied to identification of enzymatically cleaved NMPs from native RNA samples, with which microRNA and tRNA were tested and their sequence composition were successfully quantified, suggesting the feasibility of exo-sequencing using enzyme conjugated MspA-PBA in follow up studies. Although not demonstrated, this strategy is also in principle suitable for sensing nucleoside diphosphates (NDP), nucleoside triphosphates (NTP), other nucleotide modifications, nucleotide sugars⁶⁰ and nucleoside drugs⁶¹, as long as the cis-diol of the ribose is still retained.

### REFERENCE

1. Boccaletto, P. et al. MODOMICS: a database of RNA modification pathways. 2017 update. Nucleic Acids Research 46, D303-D307 (2018).
2. Roundtree, I. A., Evans, M. E., Pan, T. & He, C. Dynamic RNA Modifications in Gene Expression Regulation. Cell 169, 1187-1200 (2017).
3. Haussmann, I. U. et al. m⁶A potentiates Sxl alternative pre-mRNA splicing for robust Drosophila sex determination. Nature 540, 301-304 (2016).
4. Yang, X. et al. 5-methylcytosine promotes mRNA export-NSUN2 as the methyltransferase and ALYREF as an m⁵C reader. Cell Research 27, 606-625 (2017).
5. Helm, M. Post-transcriptional nucleotide modification and alternative folding of RNA. Nucleic Acids Research 34, 721-733 (2006).
6. Liu, J. et al. N 6-methyladenosine of chromosome-associated regulatory RNA regulates chromatin state and transcription. Science 367, 580-586 (2020).
7. Haruehanroengra, P., Zheng, Y. Y., Zhou, Y., Huang, Y. & Sheng, J. RNA modifications and cancer. RNA biology 17, 1560-1575 (2020).
8. Barbieri, I. & Kouzarides, T. Role of RNA modifications in cancer. Nature reviews Cancer 20, 303-322 (2020).
9. Bednářová, A. et al. Lost in Translation: Defects in Transfer RNA Modifications and Neurological Disorders. Frontiers in Molecular Neuroscience 10, 135 (2017).
10. Jonkhout, N. et al. The RNA modification landscape in human disease. Rna 23, 1754-1769 (2017).
11. Yu, Q. et al. RNA demethylation increases the yield and biomass of rice and potato plants in field trials. Nature Biotechnology 39, 1581-1588 (2021).
12. Ontiveros, R. J., Stoute, J. & Liu, K. F. The chemical diversity of RNA modifications. Biochemical Journal 476, 1227-1245 (2019).
13. Keith, G. Mobilities of modified ribonucleotides on two-dimensional cellulose thin-layer chromatography. Biochimie 77, 142-144 (1995).
14. Xu, J., Gu, A. Y., Thumati, N. R. & Wong, J. M. Y. Quantification of Pseudouridine Levels in Cellular RNA Pools with a Modified HPLC-UV Assay. Genes (Basel) 8, 219 (2017).
15. Wetzel, C. & Limbach, P. A. Mass spectrometry of modified RNAs: recent developments. Analyst 141, 16-23 (2016).
16. Li, X., Xiong, X. & Yi, C. Epitranscriptome sequencing technologies: decoding RNA modifications. Nature methods 14, 23-31 (2017).
17. Linder, B. et al. Single-nucleotide-resolution mapping of m⁶A and m⁶Am throughout the transcriptome. Nature Methods 12, 767-772 (2015).
18. Schaefer, M., Pollex, T., Hanna, K. & Lyko, F. RNA cytosine methylation analysis by bisulfite sequencing. Nucleic Acids Research 37, e12-e12 (2009).
19. Carlile, T. M. et al. Pseudouridine profiling reveals regulated mRNA pseudouridylation in yeast and human cells. Nature 515, 143-146 (2014).
20. Dominissini, D. et al. Topology of the human and mouse m⁶A RNA methylomes revealed by m⁶A-seq. Nature 485, 201-206 (2012).
21. Hu, L. et al. m⁶A RNA modifications are measured at single-base resolution across the mammalian transcriptome. Nature Biotechnology (2022).
22. Edelheit, S., Schwartz, S., Mumbach, M. R., Wurtzel, O. & Sorek, R. Transcriptome-wide mapping of 5-methylcytidine RNA modifications in bacteria, archaea, and yeast reveals m⁵C within archaeal mRNAs. PLOS genetics 9, e1003602 (2013).
23. Dominissini, D. et al. The dynamic N1-methyladenosine methylome in eukaryotic messenger RNA. Nature 530, 441-446 (2016).
24. Enroth, C. et al. Detection of internal N7-methylguanosine (m⁷G) RNA modifications by mutational profiling sequencing. Nucleic Acids Research 47, e126-e126 (2019).
25. Delatte, B. et al. Transcriptome-wide distribution and function of RNA hydroxymethylcytosine. Science 351, 282-285 (2016).
26. Arango, D. et al. Acetylation of Cytidine in mRNA Promotes Translation Efficiency. Cell 175, 1872-1886.e1824 (2018).
27. Okada, S., Ueda, H., Noda, Y. & Suzuki, T. Transcriptome-wide identification of A-to-I RNA editing sites using ICE-seq. Methods 156, 66-78 (2019).
28. Zhao, L. et al. Analysis of Transcriptome and Epitranscriptome in Plants Using PacBio Iso-Seq and Nanopore-Based Direct RNA Sequencing. Frontiers in Genetics 10, 253 (2019).
29. Vilfan, I. D. et al. Analysis of RNA base modification and structural rearrangement by single-molecule real-time detection of reverse transcription. Journal of Nanobiotechnology 11, 8 (2013).
30. Smith, A. M., Jain, M., Mulroney, L., Garalde, D. R. & Akeson, M. Reading canonical and modified nucleobases in 16S ribosomal RNA using nanopore native RNA sequencing. PloS one 14, e0216709 (2019).
31. Stephenson, W. et al. Direct detection of RNA modifications and structure using single molecule nanopore sequencing. bioRxiv (2020).
32. Fleming, A. M., Mathewson, N. J., Howpay Manage, S. A. & Burrows, C. J. Nanopore dwell time analysis permits sequencing and conformational assignment of pseudouridine in SARS-CoV-2. ACS Central Science (2021).
33. Workman, R. E. et al. Nanopore native RNA sequencing of a human poly(A) transcriptome. Nature Methods 16, 1297-1305 (2019).
34. Goodwin, S. et al. Oxford Nanopore sequencing, hybrid error correction, and de novo assembly of a eukaryotic genome. Genome research 25, 1750-1756 (2015).
35. Begik, O. et al. Quantitative profiling of pseudouridylation dynamics in native RNAs with nanopore sequencing. Nature Biotechnology 39, 1278-1291 (2021).
36. Ayub, M., Hardwick, S. W., Luisi, B. F. & Bayley, H. Nanopore-based identification of individual nucleotides for direct RNA sequencing. Nano letters 13, 6144-6150 (2013).
37. Clarke, J. et al. Continuous base identification for single-molecule nanopore DNA sequencing. Nature nanotechnology 4, 265-270 (2009).
38. Song, L. et al. Structure of staphylococcal α-hemolysin, a heptameric transmembrane pore. Science 274, 1859-1865 (1996).
39. Faller, M., Niederweis, M. & Schulz, G. E. The structure of a mycobacterial outer-membrane channel. Science 303, 1189-1192 (2004).
40. Manrao, E. A. et al. Reading DNA at single-nucleotide resolution with a mutant MspA nanopore and phi29 DNA polymerase. Nature Biotechnology 30, 349-353 (2012).
41. Cao, J. et al. Giant single molecule chemistry events observed from a tetrachloroaurate(III) embedded Mycobacterium smegmatis porin A nanopore. Nature Communications 10, 5668 (2019).
42. Wang, Y. et al. Structural-profiling of low molecular weight RNAs by nanopore trapping/translocation using Mycobacterium smegmatis porin A. Nature Communications 12, 3368 (2021).
43. Liu, Y. et al. Allosteric Switching of Calmodulin in a Mycobacterium smegmatis porin A (MspA) Nanopore-Trap. Angewandte Chemie International Edition 60, 23863 (2021).
44. Springsteen, G. & Wang, B. A detailed examination of boronic acid-diol complexation. Tetrahedron 58, 5291-5300 (2002).
45. Ramsay, W. J. & Bayley, H. Single-Molecule Determination of the Isomers of d-Glucose and d-Fructose that Bind to Boronic Acids. Angewandte Chemie 130, 2891-2895 (2018).
46. Jia, W. et al. Programmable nano-reactors for stochastic sensing. Nature Communications 12, 5811 (2021).
47. Choi, L. S. & Bayley, H. S-Nitrosothiol Chemistry at the Single-Molecule Level. Angewandte Chemie International Edition 51, 7972-7976 (2012).
48. Yurkevich, A. M. et al. The reaction of phenylboronic acid with nucleosides and mononucleotides. Tetrahedron 25, 477-484 (1969).
49. Chen, X. et al. RNA methylation and diseases: experimental results, databases, Web servers and computational models. Briefings in Bioinformatics 20, 896-917 (2019).
50. Konno, M. et al. Distinct methylation levels of mature microRNAs in gastrointestinal cancers. Nature communications 10, 3888 (2019).
51. Hori, H. Methylated nucleosides in tRNA and tRNA methyltransferases. Frontiers in genetics 5, 144 (2014).
52. Shi, H. & Moore, P. B. The crystal structure of yeast phenylalanine tRNA at 1.93 Å resolution: a classic structure revisited. Rna 6, 1091-1105 (2000).
53. Barraud, P. et al. Time-resolved NMR monitoring of tRNA maturation. Nature communications 10, 3373 (2019).
54. Hingerty, B., Brown, R. & Jack, A. Further refinement of the structure of yeast tRNAPhe. Journal of molecular biology 124, 523-534 (1978).
55. Jeong, K.-B. et al. Alpha-Hederin nanopore for single nucleotide discrimination. ACS nano 13, 1719-1727 (2019).
56. Yang, H. et al. Identification of single nucleotides by a tiny charged solid-state nanopore. The Journal of Physical Chemistry B 122, 7929-7935 (2018).
57. Feng, J. et al. Identification of single nucleotides in MoS2 nanopores. Nature Nanotechnology 10, 1070-1076 (2015).
58. Sen, P. & Gupta, M. Single nucleotide detection using bilayer MoS2 nanopores with high efficiency. RSC Advances 11, 6114-6123 (2021).
59. Smith, H. C. RNA binding to APOBEC deaminases; Not simply a substrate for C to U editing. RNA biology 14, 1153-1165 (2017).
60. Mikkola, S. Nucleotide sugars in chemistry and biology. Molecules 25, 5755 (2020).
61. Damaraju, V. L. et al. Nucleoside anticancer drugs: the role of nucleoside transporters in resistance to cancer chemotherapy. Oncogene 22, 7524-7536 (2003).

### Supplementary Information (S1)

### Materials

Hexadecane, pentane, ethylenediamine tetraacetic acid (EDTA), Genapol X-80, ammonium persulfate, sodium dodecyl sulfate, N,N,N′,N′-tetramethylethylenediamine and tris (2-carboxyethyl) phosphine hydrochloride (TCEP), 30% acrylamide/bis-acrylamide solution and yeast RNA^{phe} were from Sigma-Aldrich. Potassium chloride, sodium chloride, 3-morpholine propionic acid (MOPS), sodium hydrogen phosphate, sodium dihydrogen phosphate and Coomassie blue fast staining solution were from Aladdin. 4-(2-hydroxyethyl)-1-piperazine ethanesulfonic acid (HEPES) was from Shanghai Yuanye Bio-Technology. 1,2-diphytanoyl-sn-glycero-3-phosphocholine (DPhPC) was from Avanti Polar Lipids. S1 Nuclease and RNase-free water were from Takara. RNA Loading Dye and microRNA marker were from New England Biolabs. Chelex 100 Resin, 4-20% Mini-PROTEAN TGX Gel, Precision Plus Protein™ Dual Xtra Standards, stacking gel buffer (0.5M Tris-HCl buffer, pH 6.8) and resolving gel buffer (1.5M Tris-HCl buffer, pH 8.8) were from Bio-Rad. Luria Broth (LB) and LB Agar were from Hopebio. SDS-PAGE sample loading buffer was from Beyotime. Dioxane-free isopropyl-β-D-thiogalactopyranoside (IPTG), kanamycin sulfate, imidazole and tris (hydroxymethyl)aminomethane (Tris) were from Solarbio. E. coli strain BL21 (DE3) plysS and chloramphenicol was from Sangon Biotech. 3-(maleimide) phenylboronic acid (MPBA) was from Santa Cruz Biotechnology (Shanghai). High-performance liquid chromatography-purified hsa-miR-21 and has-miR-17 were custom synthesized by GenScript (New Jersey, USA). The plasmid DNAs encoding for M2 MspA-D16H6 or M2 MspA-N90C-H6 were custom prepared by GenScript (New Jersey, USA).

Cytidine-5′-monophosphate (CMP), uridine-5′-monophosphate (UMP), adenosine-5′-monophosphate (AMP), guanosine-5′-monophosphate (GMP), inosine-5′-phosphate (I) and 2′-deoxyadenosine-5′-phosphate (dAMP) were from Aladdin. N1-methyladenosine-5′-monophosphate (m¹A) and N7-methylguanosine-5′-monophosphate (m⁷G) were from Jena Bioscience. N6-methyladenosine-5′-monophosphate (m⁶A) and 5-Methylcytidine-5′-monophosphate (m⁵C) were from Carbosynth. Pseudouridine-5′-monophosphate(ψ) and dihydrouridine-5′-monophosphate were synthesised by Wuxi AppTec (FIGS. 57 and 58).

0.15-2.0 M KCl buffer (0.15-2.0 M KCl, 10 mM MOPS, pH 7.0), lysis buffer (100 mM Na₂HPO₄/NaH₂PO₄, 0.1 mM EDTA, 150 mM NaCl, 0.5% (v/v) Genapol X-80, pH 6.5), buffer A (0.5 M NaCl, 20 mM HEPES, 5 mM imidazole, 0.5% (v/v) Genapol X-80, pH 8.0) and buffer B (0.5 M NaCl, 20 mM HEPES, 500 mM imidazole, 0.5% (v/v) Genapol X-80, pH 8.0) were prepared as described by the manufacturer. All buffers were membrane-filtered (0.2 μm cellulose acetate; Nalgene) prior to use. The KCl buffer was treated with Chelex 100 resin (Bio-Rad) overnight and adjusted to pH 7.0 prior to use.

TABLE 7
The protein sequence.

| Source | |
|---|---|
| Plasmid | Protein Sequence |
| M2 | MGLDNELSLVDGQDRTLTVQQWDTFLNGVFPLDRNRLT |
| MspA-D16H6 | REWFHSGRAKYIVAGPGADEFEGTLELGYQIGFPWSLG |
| | VGINFSYTTPNILINNGNITAPPFGLNSVITPNLFPGV |
| | SISARLGNGPGIQEVATFSVRVSGAKGGVAVSNAHGTV |
| | TGAAGGVLLRPFARLIASTGDSVTTYGEPWNMNDDDDD |
| | DDDDDDDDDDDHHHHHH* |
| | (SEQ ID NO: 2) |
| N90C | MGLDNELSLVDGQDRTLTVQQWDTFLNGVFPLDRNRLT |
| MspA-H6 | REWFHSGRAKYIVAGPGADEFEGTLELGYQIGFPWSLG |
| | VGINFSYTTPNILICNGNITAPPFGLNSVITPNLFPGV |
| | SISARLGNGPGIQEVATFSVRVSGAKGGVAVSNAHGTV |
| | TGAAGGVLLRPFARLIASTGDSVTTYGEPWNMNHHHHH |
| | H* |
| | (SEQ ID NO: 3) |

Footnotes:
1. The underlined characters stand for the amino acid identity at site 90 of each gene expression product.
2. The HIS-tag is marked with bold characters in the sequence.
3. The poly-aspartic acids tag (D16) is marked with italic characters in the sequence.

TABLE 8
Statistics of τ_{off} and τₒₙ measured with different CMP concentrations.
All measurements were performed as described in Methods in Example
2. CMP was added to the cis chamber with a desired concentration.
A +200 mV voltage was continually applied during the measurements.
1̅/̅τ̅_̅{̅o̅f̅f̅}̅ and 1̅/̅τ̅ₒ̅ₙ̅ were mean values of 1/τ_{off} and 1/τₒₙ from three
independent measurements, respectively.

| Concentration(μM) | 1̅/̅τ̅_̅{̅o̅f̅f̅}̅(s⁻¹) | 1̅/̅τ̅ₒ̅ₙ̅(s⁻¹) |
|---|---|---|
| 100 | 19.0 ± 1.9 | 0.30 ± 0.06 |
| 200 | 20.9 ± 0.3 | 0.47 ± 0.05 |
| 300 | 21.0 ± 0.4 | 0.66 ± 0.05 |
| 400 | 20.5 ± 0.6 | 0.86 ± 0.05 |
| 500 | 20.6 ± 1.1 | 1.01 ± 0.13 |

TABLE 9
Statistics of τ_{off} and τₒₙ measured with different UMP concentrations.
All measurements were performed as described in Methods in Example
2. UMP was added to the cis chamber with a desired concentration.
A +200 mV voltage was continually applied during the measurements.
1̅/̅τ̅_̅{̅o̅f̅f̅}̅ and 1̅/̅τ̅ₒ̅ₙ̅ were mean values of 1/τ_{off} and 1/τₒₙ from three
independent measurements, respectively.

| Concentration(μM) | 1̅/̅τ̅_̅{̅o̅f̅f̅}̅(s⁻¹) | 1̅/̅τ̅ₒ̅ₙ̅(s⁻¹) |
|---|---|---|
| 100 | 7.8 ± 0.9 | 0.28 ± 0.08 |
| 200 | 7.4 ± 1.4 | 0.36 ± 0.02 |
| 300 | 7.7 ± 0.9 | 0.49 ± 0.08 |
| 400 | 8.2 ± 0.4 | 0.56 ± 0.11 |
| 500 | 8.7 ± 0.4 | 0.68 ± 0.09 |

TABLE 10
Statistics of τ_{off} and τₒₙ measured with different AMP concentrations.
All measurements were performed as described in Methods in Example
2. AMP was added to the cis chamber with desired concentration.
A +200 mV voltage was continually applied during the measurements.
1̅/̅τ̅_̅{̅o̅f̅f̅}̅ and 1̅/̅τ̅ₒ̅ₙ̅ were mean values of 1/τ_{off} and 1/τₒₙ from three
independent measurements, respectively.

| Concentration(μM) | 1̅/̅τ̅_̅{̅o̅f̅f̅}̅(s⁻¹) | 1̅/̅τ̅ₒ̅ₙ̅(s⁻¹) |
|---|---|---|
| 100 | 14.9 ± 0.5 | 0.54 ± 0.11 |
| 200 | 15.1 ± 0.2 | 0.82 ± 0.05 |
| 300 | 13.8 ± 0.4 | 1.03 ± 0.11 |
| 400 | 14.6 ± 1.1 | 1.19 ± 0.08 |
| 500 | 14.4 ± 1.8 | 1.39 ± 0.07 |

TABLE 11
Statistics of τ_{off} and τₒₙ measured with different GMP concentrations.
All measurements were performed as described in Methods in Example
2. GMP was added to the cis chamber with a desired concentration.
A +200 mV voltage was continually applied during the measurements.
1̅/̅τ̅_̅{̅o̅f̅f̅}̅ and 1̅/̅τ̅ₒ̅ₙ̅ were mean values of 1/τ_{off} and 1/τₒₙ from three
independent measurements, respectively.

| Concentration(μM) | 1̅/̅τ̅_̅{̅o̅f̅f̅}̅(s⁻¹) | 1̅/̅τ̅ₒ̅ₙ̅(s⁻¹) |
|---|---|---|
| 100 | 11.5 ± 0.9 | 0.02 ± 0.07 |
| 200 | 10.6 ± 1.4 | 0.50 ± 0.12 |
| 300 | 10.5 ± 1.1 | 0.68 ± 0.02 |
| 400 | 10.2 ± 0.2 | 0.83 ± 0.04 |
| 500 | 11.4 ± 1.3 | 1.00 ± 0.04 |

TABLE 12
Statistics of τ_{off} and τₒₙ of AMP binding events at different voltages.
All measurements were performed as described in Methods in
Example 2. AMP was added to the cis chamber with a final
concentration of 500 μM. 1̅/̅τ̅_̅{̅o̅f̅f̅}̅ and 1̅/̅τ̅ₒ̅ₙ̅ were mean values of
1/τ_{off} and 1/τₒₙ from three independent measurements, respectively.

| Voltage(mV) | 1̅/̅τ̅_̅{̅o̅f̅f̅}̅(s⁻¹) | 1̅/̅τ̅ₒ̅ₙ̅(s⁻¹) |
|---|---|---|
| +40 | 30.5 ± 1.7 | 0.45 ± 0.03 |
| +80 | 27.6 ± 1.9 | 0.71 ± 0.12 |
| +120 | 24.2 ± 2.1 | 1.13 ± 0.08 |
| +160 | 18.0 ± 1.4 | 1.23 ± 0.11 |
| +200 | 14.5 ± 1.8 | 1.38 ± 0.08 |

TABLE 13
Characteristic parameters of binding events from different NMPs.
All measurements were performed as described in Methods in
Example 2. Each NMP was added to the cis chamber with a final
concentration of 300 μM. A +200 mV voltage was continually
applied during the measurements. All statistical results were
derived from results of three independent measurements.

| NMP class | %̅ I̅_̅{̅b̅}̅ (%) | S̅.̅D̅.̅(pA) | τ̅_̅{̅o̅f̅f̅}̅(ms) | τ̅ₒ̅ₙ̅(ms) |
|---|---|---|---|---|
| C | 7.1 ± 0.2 | 3.23 ± 0.09 | 47.6 ± 0.9 | 1511.6 ± 108.3 |
| U | 8.64 ± 0.09 | 2.14 ± 0.06 | 130.3 ± 14.8 | 2097.9 ± 474.7 |
| A | 10.89 ± 0.14 | 3.14 ± 0.14 | 72.76 ± 2.07 | 979.5 ± 106.1 |
| G | 11.8 ± 0.2 | 3.02 ± 0.13 | 96.3 ± 10.4 | 1473.1 ± 44.4 |
| m⁵C | 8.46 ± 0.07 | 4.98 ± 0.15 | 32.7 ± 4.4 | 1399.0 ± 143.8 |
| m⁶A | 9.48 ± 0.04 | 2.6 ± 0.2 | 38.4 ± 1.9 | 652.9 ± 27.7 |
| Ψ | 10.2 ± 0.2 | 2.40 ± 0.05 | 36.9 ± 3.4 | 2047.7 ± 283.8 |
| D | 11.49 ± 0.04 | 2.44 ± 0.05 | 66.8 ± 12.6 | 2417.9 ± 505.9 |
| I | 12.36 ± 0.04 | 2.48 ± 0.01 | 103.2 ± 0.9 | 1419.0 ± 155.9 |
| m⁷G | 16.58 ± 0.07 | 6.08 ± 0.02 | 86.5 ± 13.6 | 1053.3 ± 82.7 |
| m¹A | 22.09 ± 0.03 | 6.47 ± 0.04 | 37.72 ± 7.09 | 2629.7 ± 703.1 |

TABLE 14
Validation and testing accuracies of different models. 400 events for each NMP type
were used as the training set and 100 events for each NMP type were used as the testing
set. All models were trained using the Classification Learner toolbox in MATLAB. The
validation accuracies were derived from the 10-fold cross-validation results (Valid. Acc).
Considering both validation and testing accuracies (Test. Acc), the linear SVM model
reported the best score, which is marked with red characters. The linear SVM model was
selected for further use.

| | | Valid. | Test. | | | Valid. | Test. |
|---|---|---|---|---|---|---|---|
| Model | | Acc | Acc | Model | | Acc | Acc |
| Decision | Fine Tree | 99.5 | 99.4 | K | Coarse KNN | 98.2 | 98.7 |
| Trees | Medium Tree | 99.5 | 99.4 | Nearest | Cosine KNN | 75.9 | 76.5 |
| | Coarse Tree | 45.4 | 45.5 | Neighbor | Cubic KNN | 99.2 | 99.5 |
| | | | | (KNN) | | | |
| Discriminant | Linear | 99.4 | 99.2 | | Weighted | 99.4 | 99.4 |
| Analysis | Discriminant | | | | KNN | | |
| | Quadratic | 98.9 | 98.6 | Ensemble | Boosted | 99.4 | 99.3 |
| | Discriminant | | | | Trees | | |
| Naïve | Gaussian | 98.5 | 98.3 | | Bagged Trees | 99.4 | 99.7 |
| Bayes | Naïve | | | | | | |
| | Bayes | | | | | | |
| | Kernel | 99.6 | 99.5 | | Subspace | 98.4 | 97.4 |
| | Naïve | | | | Discriminant | | |
| | Bayes | | | | | | |
| Support | Linear | 99.6 | 99.7 | | Subspace | 71.3 | 93.2 |
| Vector | SVM | | | | KNN | | |
| Machine | Cubic | 99.5 | 99.5 | | RUSBoost | 99.5 | 99.4 |
| (SVM) | SVM | | | | Trees | | |
| | Fine | 99.5 | 99.3 | Neural | Narrow | 99.5 | 99.6 |
| | Gaussian | | | Network | Neural | | |
| | SVM | | | | Network | | |
| | Medium | 99.6 | 99.5 | | Medium | 99.5 | 99.5 |
| | Gaussian | | | | Neural | | |
| | SVM | | | | Network | | |
| | Coarse | 99.5 | 99.3 | | Wide Neural | 99.4 | 99.5 |
| | Gaussian | | | | Network | | |
| | SVM | | | | | | |
| K Nearest | Fine KNN | 99.5 | 99.5 | | Bilayered | 99.4 | 99.8 |
| Neighbor | | | | | Neural | | |
| (KNN) | | | | | Network | | |
| | Medium | 99.2 | 99.3 | | Trilayered | 99.5 | 99.6 |
| | KNN | | | | Neural | | |
| | | | | | Network | | |

TABLE 15
MicroRNA sequences.

| abbreviations | sequences (5′-3′) |
|---|---|
| hsa-miR-21 | UAGCUUAU(m⁵C)AGACUGAUGUUGA |
| | (SEQ ID NO: 4) |
| hsa-miR-17 | CAAAGUGCUUAC(m⁶A)GUGCAGGUAG |
| | (SEQ ID NO: 5) |

TABLE 16
Calibration coefficients of different NMPs. The calibration
coefficient (δ) is defined as the number of NMP binding
events occurring per unit concentration per min. The value
was acquired during measurements with a sole NMP. The final
concentration of each NMP in cis was 300 μM. A +200
mV voltage was continually applied during the measurements.
δ̅ is the mean value of δ from three independent measurements.

| NMP class | δ̅ (μM⁻¹ *min⁻¹) |
|---|---|
| C | 0.133 ± 0.010 |
| U | 0.097 ± 0.014 |
| A | 0.21 ± 0.02 |
| G | 0.136 ± 0.004 |
| m⁵C | 0.144 ± 0.014 |
| m⁶A | 0.307 ± 0.013 |
| Ψ | 0.099 ± 0.014 |
| D | 0.085 ± 0.018 |
| I | 0.142 ± 0.015 |
| m⁷G | 0.191 ± 0.015 |
| m¹A | 0.08 ± 0.02 |

### Supplementary Movie 1: Simultaneous Sensing of Eleven Types of NMPs.

Electrophysiology measurements were performed as described in Methods in Example 2 in a 1.5 M KCl buffer (1.5 M KCl, 10 mM MOPS, pH 7.0). A transmembrane potential of +200 mV was continually applied. NMPs were simultaneously added to cis with a final concentration of 100 μM for each analyte. Characteristic events of different NMPs were clearly observed from the trace. Assisted by the machine learning algorithm, each event was automatically identified and labelled with C, U, A, G, m⁵C, m⁶C, ψ, I, D, m⁷G or m¹A, respectively. For demonstration, the movie was played back at a 1.0× speed of the actual data acquisition.

### REFERENCES

1. Wang, Y. et al. Osmosis-driven motion-type modulation of biological nanopores for parallel optical nucleic acid sensing. ACS applied materials & interfaces 10, 7788-7797 (2018).
2. Wang, S. et al. Single molecule observation of hard-soft-acid-base (HSAB) interaction in engineered Mycobacterium smegmatis porin A (MspA) nanopores. Chemical Science 11, 879-887 (2020).

### Example 3: Nanopore Identification of Alditol Epimers and its Application in Rapid Analysis of “Zero-Sugar” Drinks

Alditols, which have a sweet taste but produce much lower calories than natural sugars, are widely used as artificial sweeteners. Alditols are the reduced forms of monosaccharide aldoses and different alditols are diastereomers or epimers of each other and direct and rapid identification by conventional methods is difficult. Nanopores, which are emerging single molecule sensors with exceptional resolution when engineered appropriately, are useful for the recognition of diastereomers and epimers. In this work, direct distinguishing of alditols corresponding to all fifteen monosaccharide aldoses was achieved by a boronic acid appended hetero-octameric Mycobacterium smegmatis porin A (MspA) nanopore (MspA-PBA). Thirteen alditols including glycerol, erythritol, threitol, adonitol, arabitol, xylitol, mannitol, sorbitol, allitol, dulcitol, iditol, talitol and gulitol (L-sorbitol) could be fully distinguished and their sensing features constitute a complete nanopore alditol database. To automate event classification, a custom machine learning algorithm was developed and delivered a 99.9% validation accuracy. This strategy was also used to identify alditol components in commercially available “zero-sugar” drinks, suggesting its use in rapid and sensitive quality control for the food and medical industry.

### INTRODUCTION

A main cause of obesity and diabetes in humans is excessive sugar consumption. Sugar substitutes, which preserve the taste of sweetness and reduce caloric intake¹, are widely used as food additives. Alditols, also known as sugar alcohols are obtained from the reduction of an aldose, and are one type of commonly used sugar substitute. Chemically, the aldehyde group at the reducing end of an aldose is reduced to the hydroxyl group, producing the acyclic polyol structure of an alditol². Alditols are absorbed slowly and incompletely in the human small intestine, and provide fewer calories per gram than sugars. They can thus cause less variation in the blood glucose levels than other carbohydrates.

Different alditols vary considerably in their sweetness and physiological metabolism. For example, the sweetness of xylitol is significantly higher than that of arabitol or adonitol³, although they are diastereomers. Erythritol and xylitol inhibit the growth of mutans streptococci but with different mechanisms⁴. The analysis and detection of alditols are necessary in the medical and food industries, but the similarities in their chemical structures pose significant technical challenges to the design of sensing strategies.

Conventionally, gas chromatography (GC)⁵, high-performance liquid chromatography (HPLC)^{5, 6, 7, 8} and liquid chromatography-mass spectrometry (LC-MS)⁹ are widely used in analysis of alditols but quantification is recommended only for alditols with different molecular weights or polarities, such as sorbitol, erythritol, xylitol or mannitol. This may be due to the inability to discriminate chromatographically between epimers. In GC, it is usually necessary to increase the vaporization rate¹⁰ by the derivatization of alditols as, for example the acetates, which might not be conducive in the discrimination of alditol epimers. Recent analytical strategies including chemiluminescence^{11, 12, 13}, ion mobility spectrometry (IMS)¹⁴, enzymatic fluorometric¹⁵ and colorimetric sensor arrays¹⁶ promise to provide a simpler and faster solution but due to the existence of alditol epimers, there still is a need for a strategy which is rapid, label free and capable of simultaneously discriminating all alditols.

Nanopore, an emerging single molecule sensor which provides rapid and sensitive profiling of nucleotides^{17, 18}, amino acids^{19, 20, 21}, biothiols^{22, 23}, neurotransmitter^{24, 25, 26}, nucleic acids^{27, 28}, peptides^{29, 30} and proteins^{31, 32, 33, 34, 35}, has a great potential to achieve this task. By introducing chemical reactivity into the nanopore lumen, its sensitivity and selectivity could be further improved, disclosing information that is not easily accessed by other means^{18, 36, 37} Phenylboronic acid (PBA), which is known to form covalent bonds with 1,2 or 1,3-diols in aqueous solution³⁸, can bind with polyols including sugars^{39, 40, 41, 42} and sugar alcohols^{13, 43}. Recent reports have shown that PBA can serve as a chemically specific adapter of a heterogeneous α-hemolysin (α-HL)⁴⁴ permitting the detection of saccharides. However, the cylindrical lumen of α-HL fails to provide a sufficient resolution to distinguish between chemically similar molecules, including epimeric monosaccharides. To the best of our knowledge, discrimination of alditol epimers using nanopore has not been reported.

The MspA nanopore is conically shaped and has demonstrated superior resolution in the discrimination of epigenetic modifications⁴⁵, DNA lesions^{46, 47}, RNA structures²⁷ and protein structures^{31, 48}. Engineered MspA has also directly observed the coordination chemistry of a single metal ion at high resolution^{22, 49, 50, 51}. However, the octameric symmetry of MspA has posed a technical challenge to the introduction of a sole reactive site for sensing. A hetero-octameric MspA nanopore sensor has not been reported previously. In this paper, a hetero-octameric MspA nanopore containing a single phenylboronic acid (MspA-PBA) was designed, prepared and used as an alditol sensor. Thirteen types of alditols including glycerol, tetritols, pentitols and hexitols were detected by this nanopore, forming a complete database of nanopore sensing data for alditol epimers. Direct identification of such a large variety of alditols has not been reported previously. Assisted by an artificial intelligence classification model, identification of alditols in 4 kinds of “zero-sugar” beverages was also performed.

### Results and Discussion

### Identification of Alditols Using a PBA Appended MspA

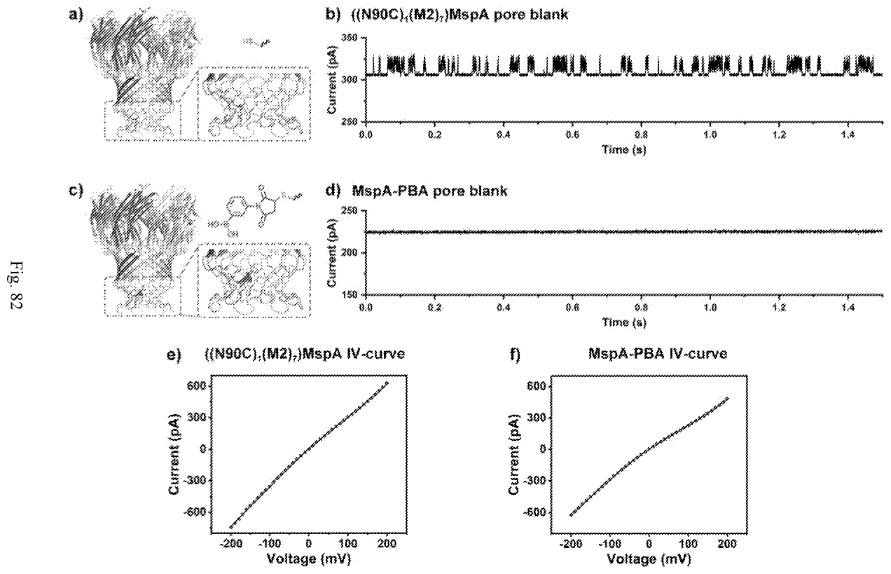

A specially engineered MspA, which contains a PBA appended to its pore constriction, was designed and is termed MspA-PBA (Method 2 in Example 3) in this paper. MspA-PBA was prepared by mixing the hetero-octameric MspA ((N90C)₁(M2)₇) with 3-(maleimide) phenylboronic acid (MPBA) (FIG. 1a, Method 2 in Example 3). The success of MspA-PBA preparation can be confirmed by the value and the noise characteristics of its open pore current during nanopore measurements (FIG. 82).

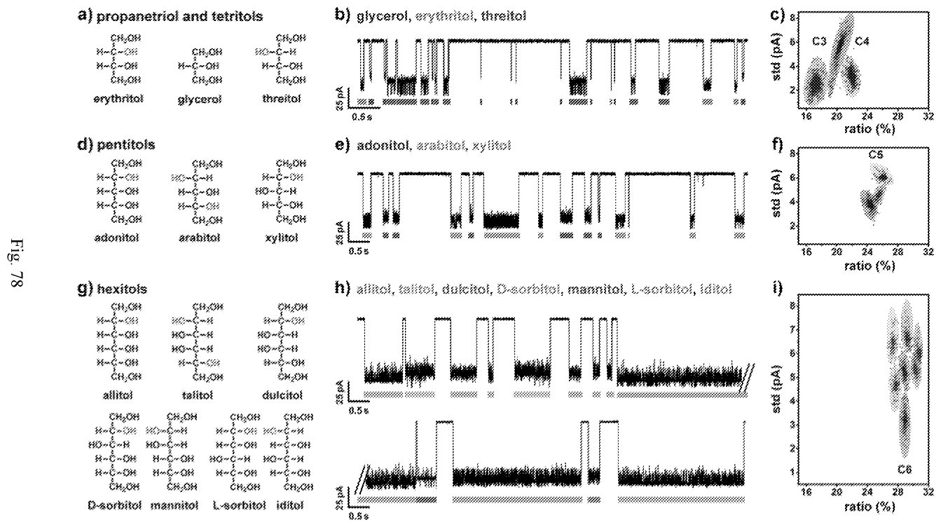

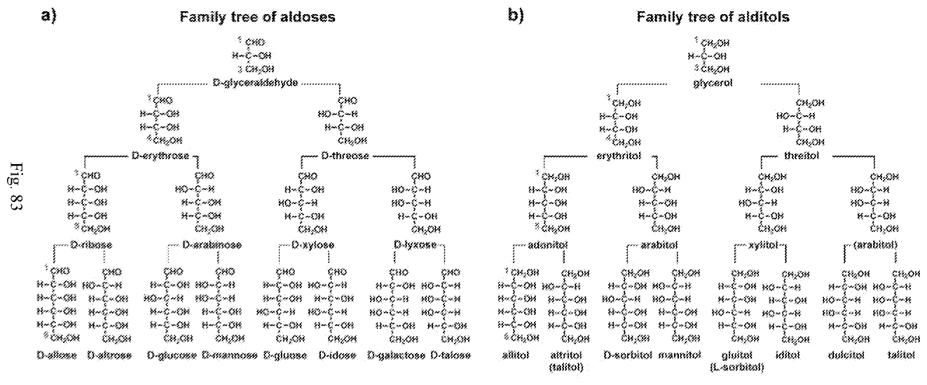

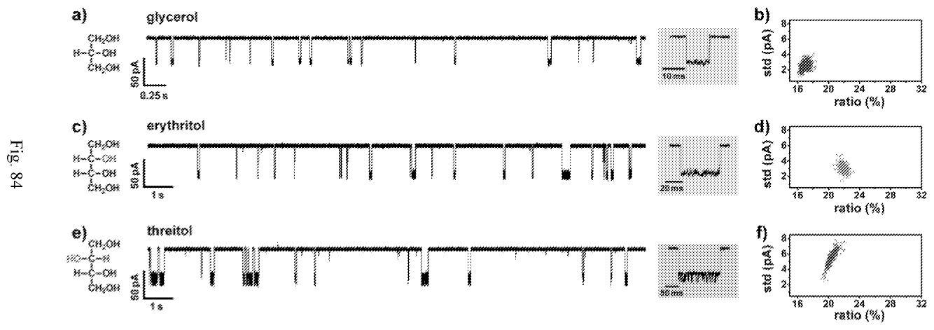

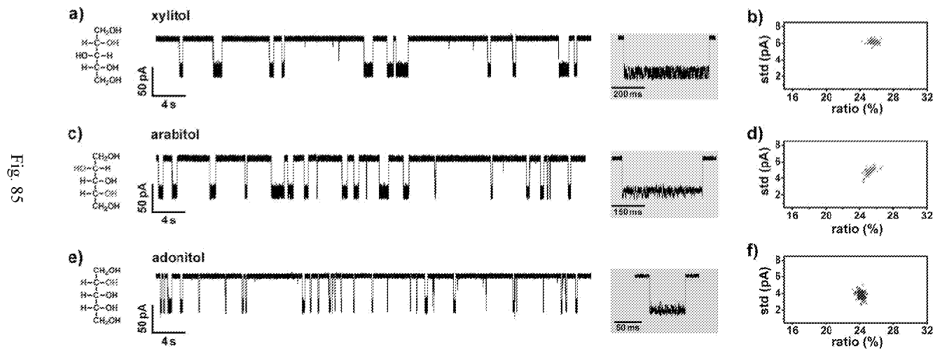

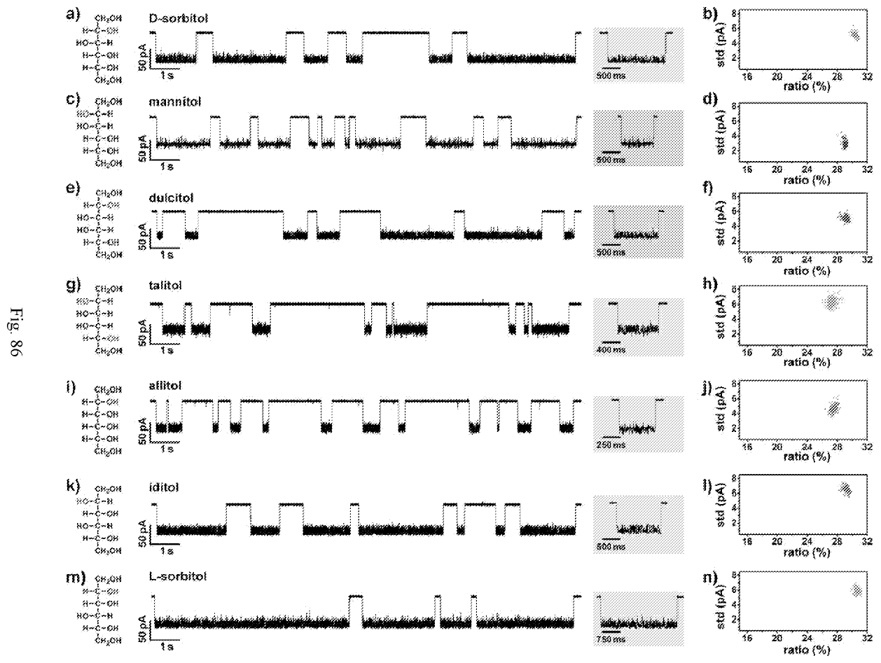

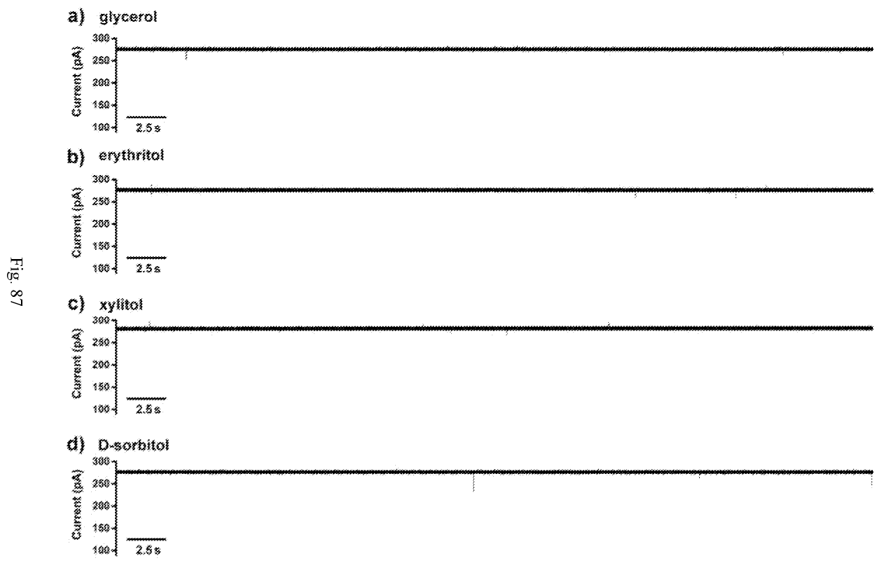

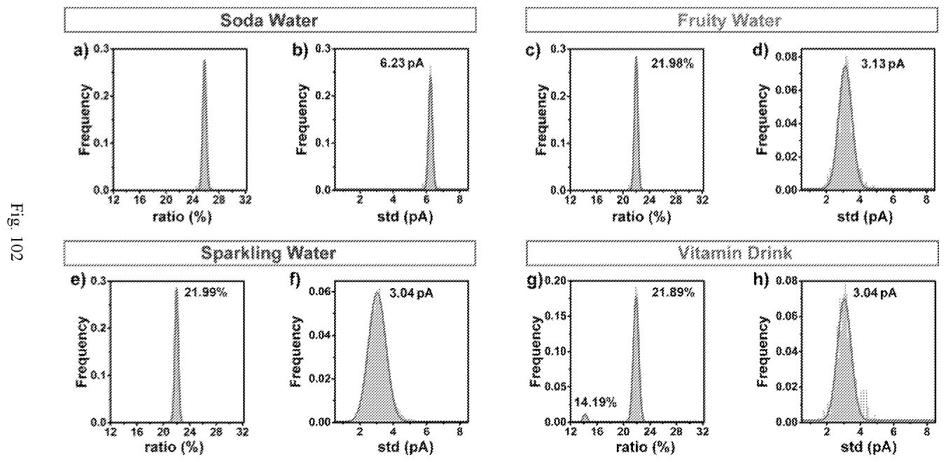

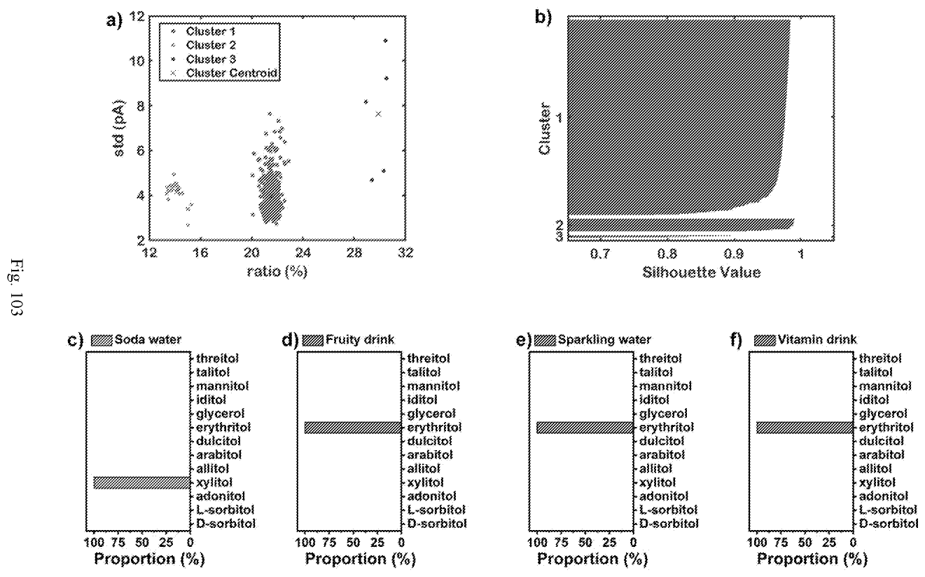

All subsequent nanopore measurements were carried out with a 1.5 M KCl buffer (1.5 M KCl, 10 mM 3-(N-Morpholino) propanesulfonic acid (MOPS), pH 7.0) and a +100 m V continually applied potential (Method 1 in Example 3). 13 types of alditols, which were derived from the reduction of the carbonyl group of C₃-C₆ monoaldoses were treated as model polyols (FIGS. 77b and 83). The PBA of MspA-PBA can complex with a polyol, forming a cyclic ester at the pore constriction and resulting in the corresponding nanopore event being reported. All alditols were added to the cis side of the pore and the final concentration of alditols was set at 4 mM with the exception of glycerol, which was set at 8 mM instead. Characteristic nanopore events corresponding to different alditol types were immediately detected (FIGS. 77b and 84-86) but no alditol events were observed with M2 MspA, again confirming that the placement of the PBA is critical in the generation of alditol sensing events (FIG. 87). In addition, single molecule sensing of glycerol and D-Sorbitol by MspA-PBA are as shown in FIGS. 104 and 105, respectively.

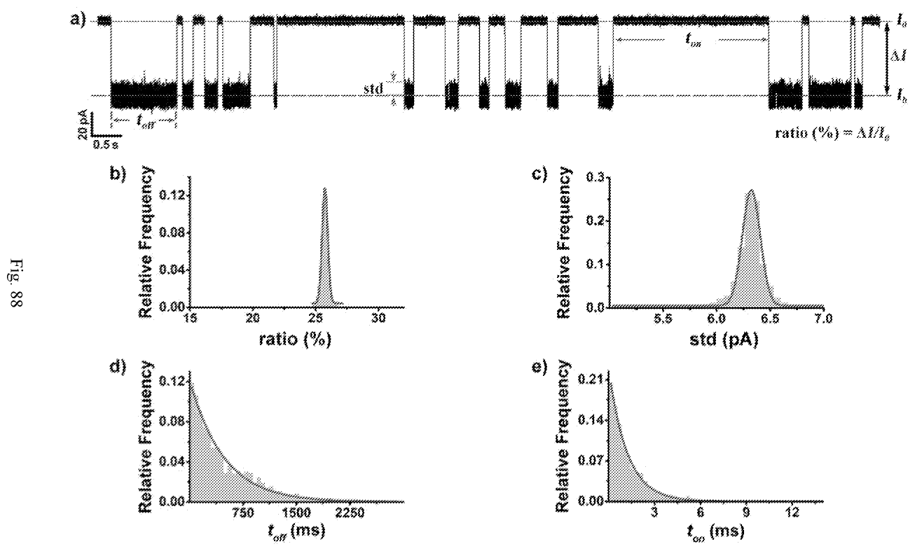

To describe the sensing events quantitatively, event parameters such as the open-pore current (I₀), the current blockade (I_{b}), the percentage blockage (ratio), the event dwell time (τ_{off}), the inter-event intervals (τₒₙ) and the standard deviation value of the blockage level (std) were defined as described in FIG. 88. The percentage blockage, also referred to for simplicity as ratio, is defined as (I₀−I_{b})/I₀.

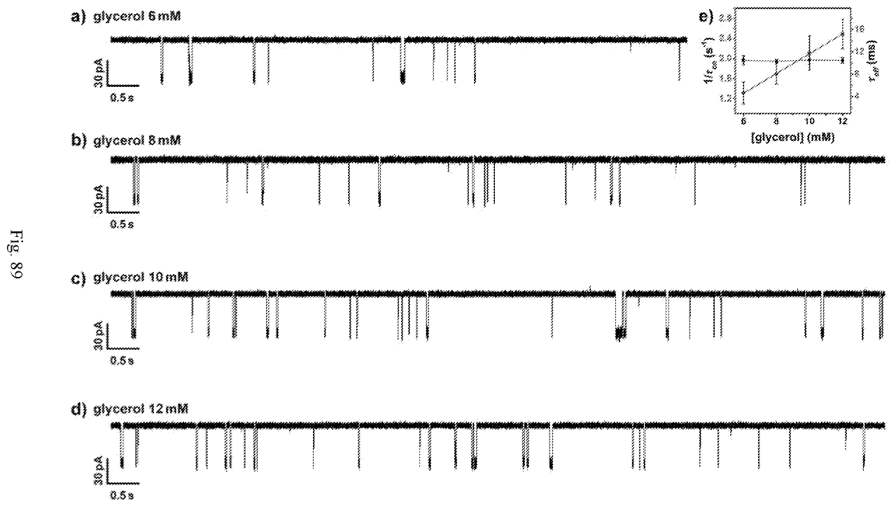

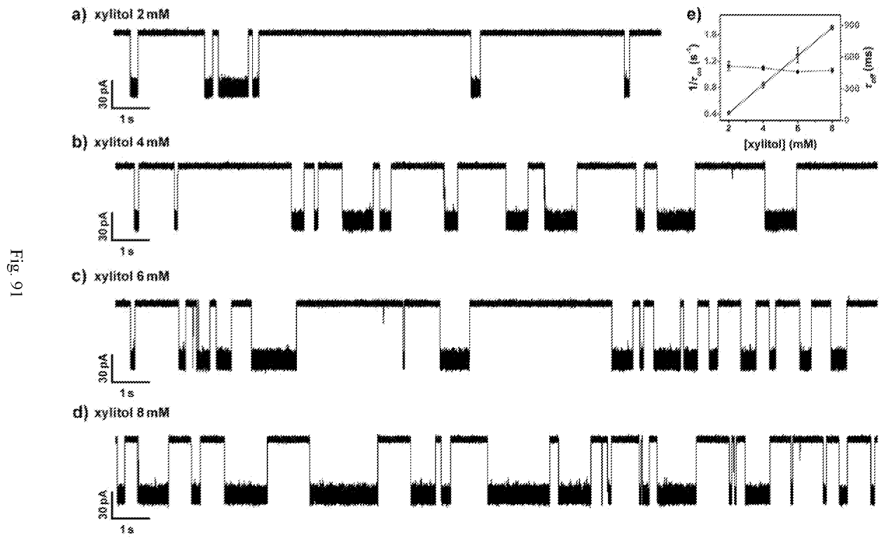

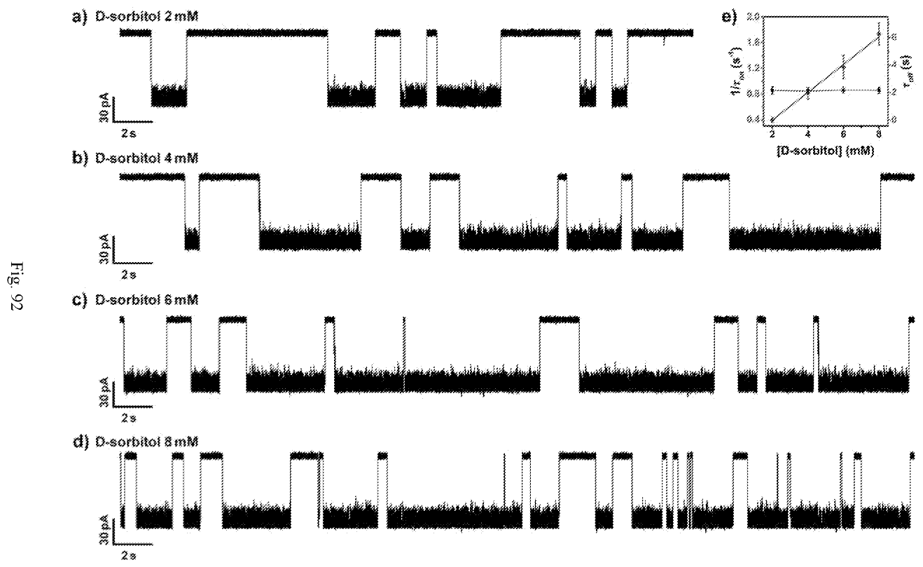

For each type of alditol added, the rate of event appearance was proportionally increased to the final concentrations of glycerol, tetritol, pentitol and hexitol (FIGS. 89-92), confirming that the events were generated by the added alditol. Quantitatively, the reciprocal of the mean inter-event interval (1̅/̅τ̅ₒ̅ₙ̅, N=3) is linearly correlated with the alditol concentration, consistent with a bimolecular model. The mean event dwell time τ̅_̅{̅o̅f̅f̅}̅ (N=3) however is independent of the alditol concentration, and this is consistent with a unimolecular dissociation mechanism (FIGS. 89e-92e, Table 18). The equilibrium binding constants (K_{b}) of glycerol, tetritol, pentitol and hexitol were calculated and compared according to the equation K_{b}=kₒₙ/k_{off}, from which the association rate kₒₙ=1/(τₒₙ*c) and the dissociation rate k_{off}=1/τ_{off} respectively, were derived. The binding constant for borate cyclic ester formation for alditols increases as the number of hydroxyl groups in the alditol increases (FIG. 77c and Table 18), consistent with conclusions drawn previously from the corresponding NMR measurements.⁵² Nanopore measurements were also performed with different applied voltages. As shown in FIG. 93, both 1/τ_{off} and 1/τₒₙ of glycerol, erythritol, xylitol and D-sorbitol stay almost unchanged at a higher applied potential. This is expected because all alditols tested in this paper are electrically neutral and the formation of a borate ester is independent of the local electric field.

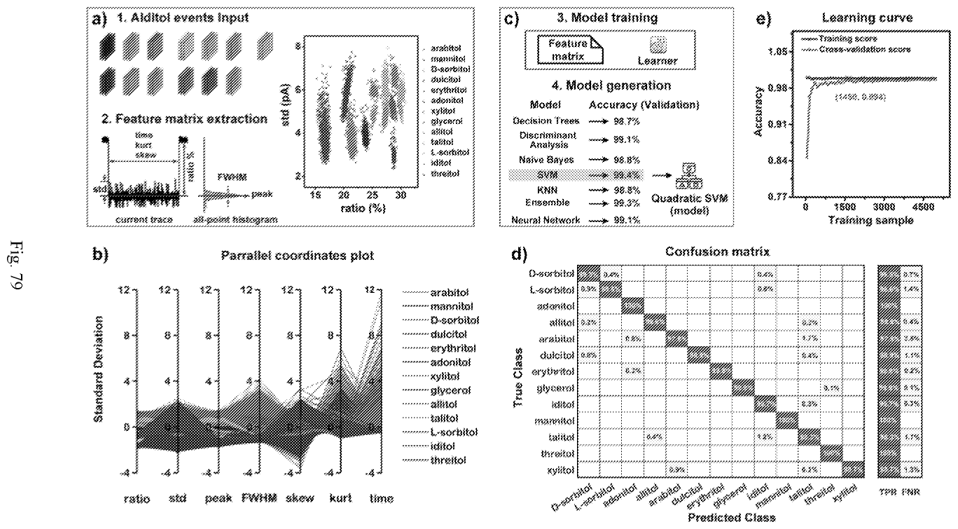

Generally, based on three independent measurements for each alditol (N=3), the ratio of glycerol (<17%), tetritols (20˜22%), pentitols (24˜26%) and hexitols (27˜31%) increased in proportion to their molecular size (Table 17). By simultaneously considering two event features, the ratio and the std, events corresponding to different alditols could be fully resolved, as shown in the scatter plots of ratio versus std formed by events acquired from the independent measurements of 13 different alditol types (n=5129) (FIG. 77d). Here, std describes the overall noise fluctuations within the blockage level of a nanopore event. Thirteen separated populations of events were clearly observed in the scatter plot, again confirming that the MspA-PBA has an excellent resolution and can distinguish between minor structural differences amongst the different alditol types. We further grouped alditols according to their carbon numbers to discuss the resolution of MspA-PBA in the discrimination between alditol epimers. In the first group, as shown in FIG. 78a, threitol and erythritol are epimers that have opposite configurations at only one stereogenic center, C-2. They also both have an extra pair of —CHOH— units compared to glycerol. These three alditols were sensed simultaneously and the glycerol events were easily identified by the feature of ratio, while erythritol and threitol events could be distinguished by their std (FIG. 78b). This difference might be related to the different binding mechanisms to a PBA of erythro-diol and threo-diol.⁵³ A total of 2147 events were recorded and plotted as a scatter plot of ratio vs. std (FIG. 78c). The result shown in FIG. 77d is consistent with those acquired from independent measurements.

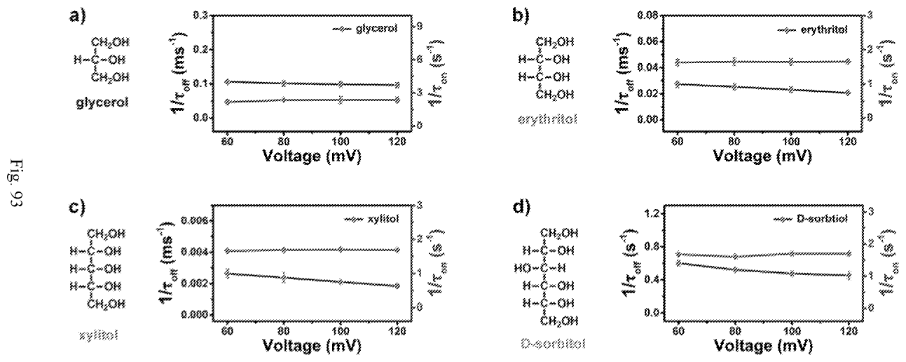

Following the same principle, pentitols and hexitols were also respectively evaluated in the second and the third group. Since arabitol is the reduction product of both arabinose and lyxose, it is an epimer of adonitol and xylitol, differing only stereochemically at C-2 or C-4, respectively (FIG. 78d). Simultaneous sensing of all three pentitols using MspA-PBA was performed and each type of pentitol can be directly identified based on the distinct blockage characteristics (FIGS. 78e, 78f). Seven types of hexitols, containing four pairs of epimers (FIG. 78g), were also simultaneously sensed. Highly discriminatory current blockage features were demonstrated (FIGS. 78h, 78i). A raw continuous current trace of FIG. 78h and a zoomed-in demonstration of each hexitol event was supplementary in FIG. 94. These results successfully demonstrate the feasibility of alditol sensing by MspA-PBA. To the best of our knowledge, the complete differentiation of all 15 D-aldose derived alditols in ensembles or single-molecules has never been reported, and this is evidence of the superior resolution of MspA-PBA.

### Rapid Identification of Alditols by Machine Learning

Although events caused by different alditols are visually identifiable, in order to automate data analysis and avoid misjudgment caused by human bias, a custom machine learning algorithm was developed based on the results described above. Generally, the machine learning based classification model for alditols could be trained by learning the characteristics of the input alditol events. The optimum classifier could be evaluated by the accuracy and the cost of cross-validation.

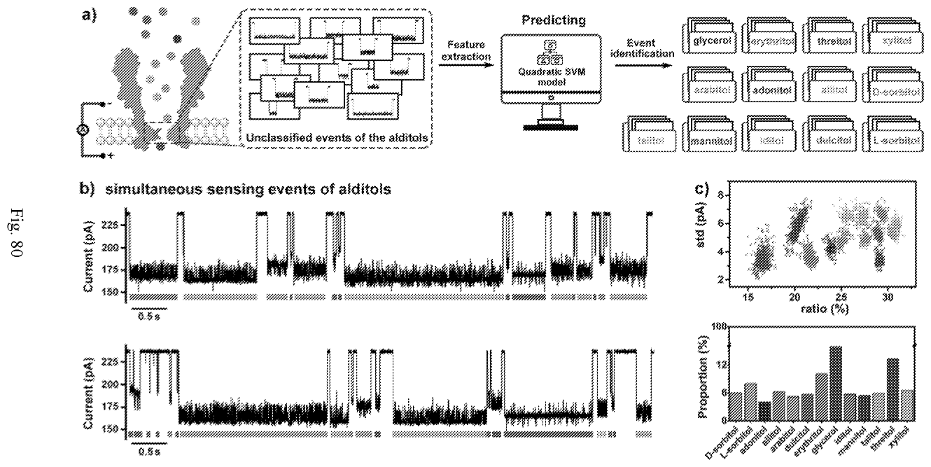

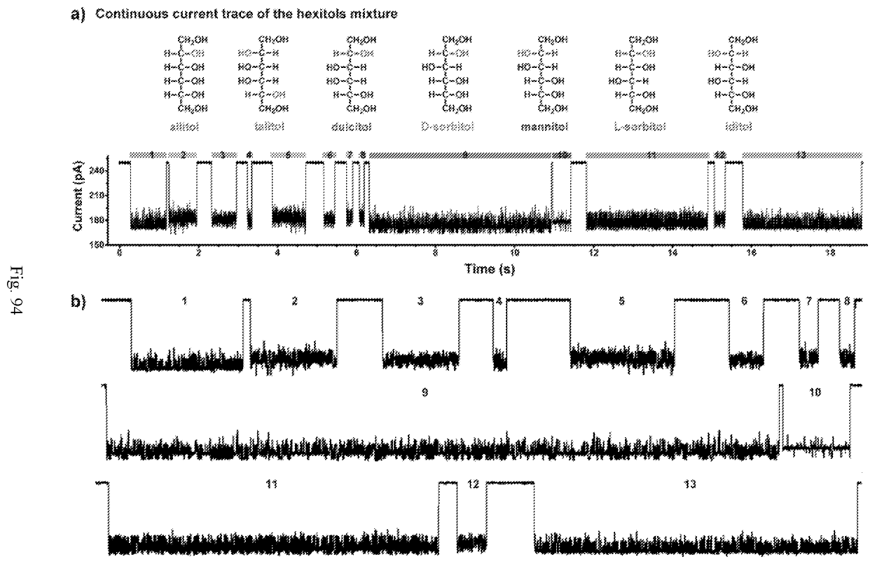

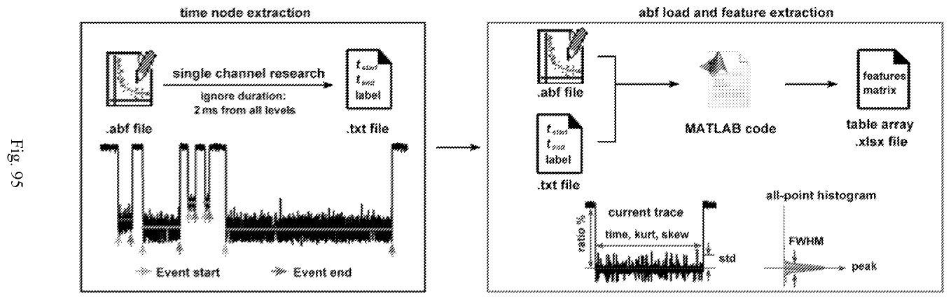

Existing sensing events in the independent measurements of alditols were first extracted from the raw time current traces. Seven features, the percentage blockage (ratio), standard deviation of the blocking current (std), kurtosis (kurt), skewness (skew), dwell time (time), the central value of the distribution (peak) and noise (FWHM) were automatically extracted by MATLAB to form a feature matrix (n=5129) (FIG. 95). The feature matrices of glycerol, erythritol, threitol, adonitol, arabitol, xylitol, mannitol, iditol, allitol, D-sorbitol, L-sorbitol, dulcitol and talitol were generated from measurements with a sole and known analyte and therefore have known labels. This feature matrix was used as a training dataset (FIG. 79a). The parallel coordinate plots of features showed that all 7 features have a narrowly defined distribution and all play important roles in the event classification (FIG. 79b). 10-fold cross-validation, which randomly splits the training data into a training subset and a validation subset for model training and validation, was performed. A set of classifiers including Decision Trees, Discriminant Analysis, Support Vector Machine (SVM), Naïve Bayes, K Nearest Neighbor (KNN), Ensemble and Neural Network were estimated with default settings of parameters (FIG. 96). All the models demonstrated satisfactory validation accuracies of >98.7%. The Quadratic SVM of SVM classifier reported the highest score of 99.4% and the lowest total cost (30/5129) (FIG. 79c). The confusion matrix results using Quadratic SVM are shown in FIG. 79d, in which most alditol events can be seen to report a true positive rate (TPR) of over 97%. A TPR of 100% was achieved for threitol, adonitol and mannitol. To evaluate the efficiency of the training, a learning curve generated by 10-fold cross-validation was plotted and clearly showed no overfitting because the classification model trained with more than 1450 training samples has the same predictive ability for the training and the testing dataset (FIG. 79e).

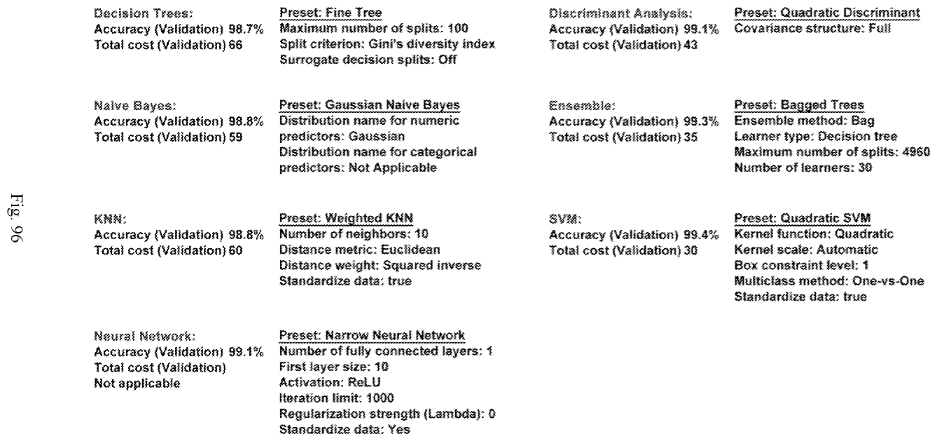

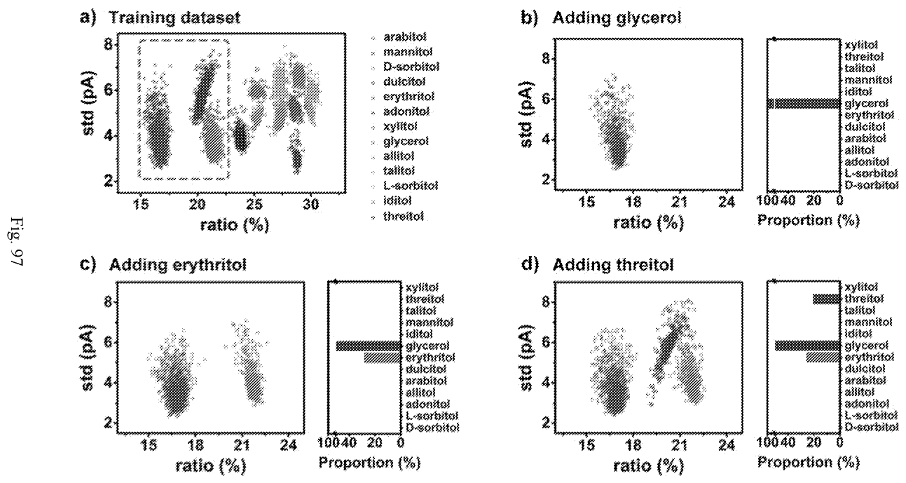

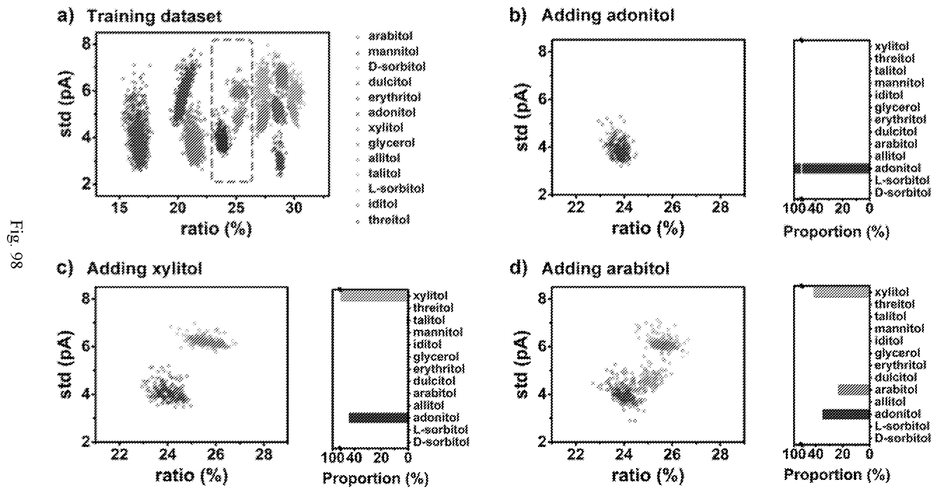

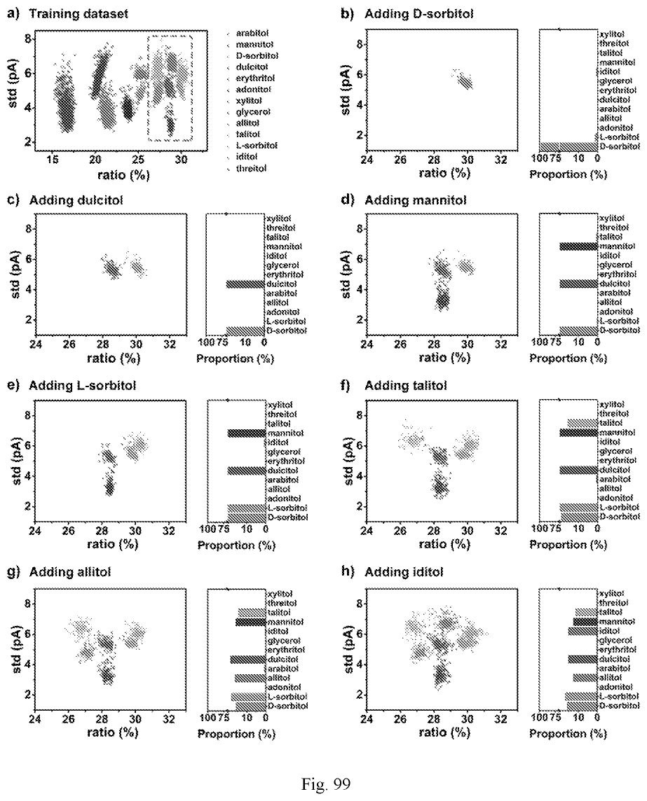

The trained Quadratic SVM model was then employed to predict events with unknown identities during simultaneous sensing of alditol mixtures (FIG. 80a). Glycerol, erythritol and threitol were sequentially added to cis with final concentrations of 8 mM, 4 mM and 4 mM, respectively. With the Quadratic SVM model, a newly added alditol could be accurately identified (FIG. 97). The color-coded scatter plot drawn according to the prediction label has a population consistent with that in the training dataset. The same performance was also observed when pentitols (adonitol, xylitol and arabitol) or hexitols (D-sorbitol, dulcitol, mannitol, L-sorbitol, talitol, allitol and iditol) were sequentially added to cis with each component at a final concentration of 4 mM (FIGS. 98 and 99). We further expanded the complexity of the prediction dataset and evaluated the capability of the Quadratic SVM model in the classification of all 13 types of alditol in a mixture. The alditols were added to cis in the order: glycerol, tetritols, pentitols and hexitols. The final concentration of glycerol is 6 mM. The final concentration of erythritol and threitol are both 4 mM and that of each other alditols is 2 mM. To show event identification from the mixture after each addition, representative raw current traces and the corresponding labels predicted by machine learning are demonstrated in FIGS. 80b and 100a-100d. Generally, each time a new group of alditols were added, the prediction results report the appearance of the corresponding alditol type (FIGS. 80c and 100e-100h). Thus, an alditol classifier that can automatically identify all kinds of alditols in a mixture during nanopore sensing has been successfully constructed, and can effectively reduce the workload and subjective bias from human interference.

### Rapid Identification of Alditols in “Zero-Sugar” Drinks

The trained classifier and the MspA-PBA sensor were further applied to the identification of alditol ingredients in commercial “zero-sugar” drinks. The consumption of sweetened beverages has been shown to be associated with an increased risk of obesity, type 2 diabetes and cardiovascular disease. A sugar substitute is an alternative for people who are at risk or suffering from these diseases. It is thus important to ensure truly zero addition of sugars in the corresponding food. As has been reported in the press, to obtain better taste or higher profits, trace amounts of sugar are added to sugar substitute foods without being specified in the ingredient list. Moreover, the type of sugar substitutes in “zero-sugar” foods and drinks is also a critical parameter. Alditols, such as xylitol, have an energy of only ˜2.4 kcal/g, and the human body obtains essentially zero calories from it, compared to sugar, which has approximately 4 kcal/g.³ However, arabitol and adonitol which are diastereomers of xylitol, have lower sweetness and are thus used less in the health-food industry. Though xylitol, sorbitol and mannitol are all commonly used alditols in food, the consumption of sorbitol and mannitol generates more severe gastrointestinal disturbances than xylitol.⁵⁴ For this reason, the content of sorbitol or mannitol in food should be restricted, and the label of the food could include a warning that “excess consumption may have a laxative effect”.

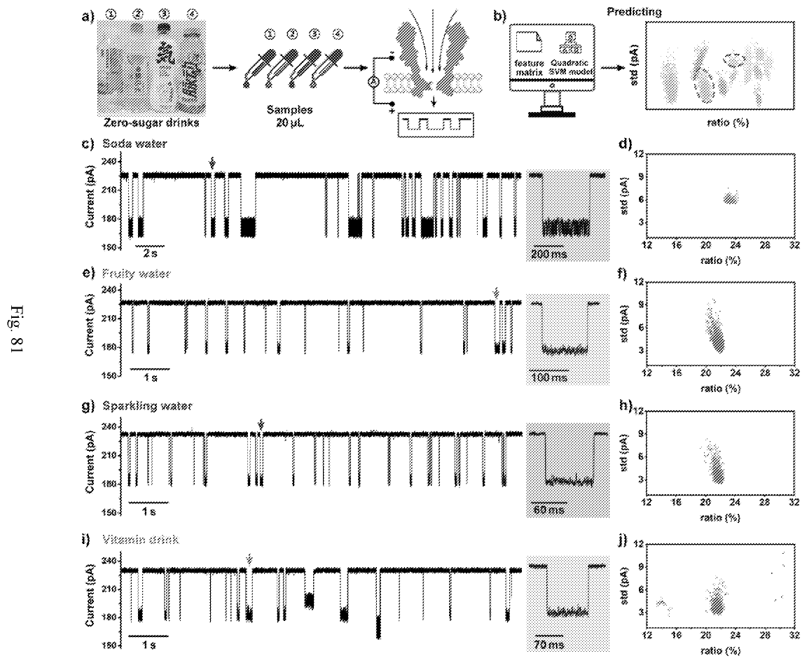

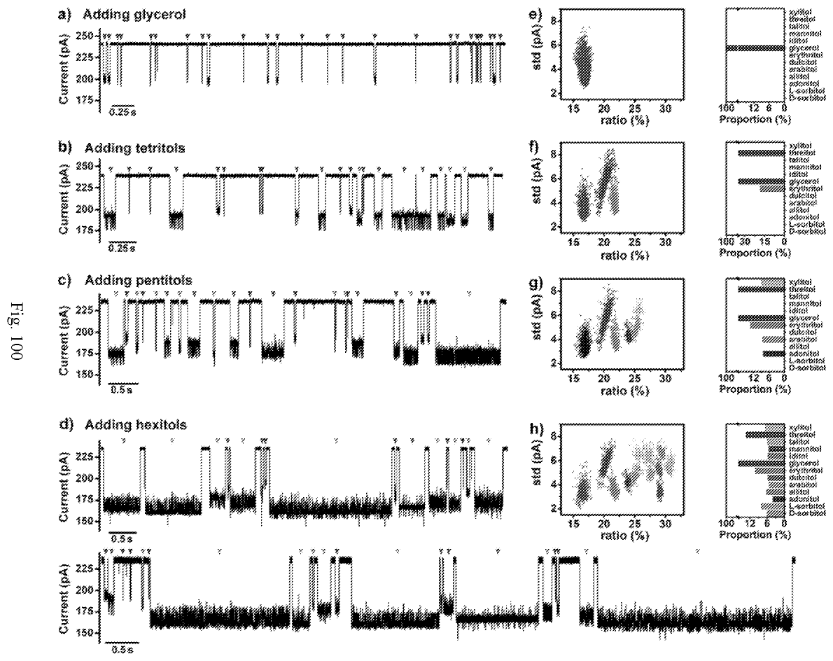

Four kinds of commonly accessible “zero-sugar” drinks including Soda Water (NongFu Spring®), Fruity Water (Coca-Cola Ice-Dew®), Sparkling Water (Genki Forest®) and Vitamin Drink (Danone Mizone®) were purchased at a local supermarket and tested in follow-up measurements (FIG. 101). Experimentally, nanopore measurements were carried out under a continuous +100 mV transmembrane potential with MspA-PBA in a 1.5 M KCl buffer (Method 1 in Example 3). A sample (20 μL) of each beverage were directly added to cis during separate measurements (FIG. 81a). Successive resistive pulses from alditols were immediately observed in the current-time trace. As shown in FIG. 81c, the addition of Soda Water resulted in the consecutive appearance of noise abundant blockage events, which have the same current fluctuation feature as xylitol. The characteristics of the event were highly uniform, as described in the scatter plot of ratio vs. std (automatically extracted using MATLAB) where only a single cluster was observed (FIG. 81d, n=614). The histogram of ratio and std of Soda Water events show a Gaussian distribution and the derived ratio and std were 25.79% and 6.23 pA, respectively (FIGS. 102a, 102b), which are consistent with the statistics of xylitol (ratio=25.70±0.10% and std=6.28±0.07 pA, Table 17).

Different from Soda Water, events acquired from Fruity Water and Sparkling Water have a relatively short residing resistive pulse and lower current fluctuation (FIGS. 81e and 81g) and the event characteristics were also highly consistent during each separate measurement (FIGS. 81f, 81h, n=950 and 781 respectively). According to the statistics, the ratio from Fruity Water and Sparkling Water were 21.98% and 21.99%, and the std values were 3.13 pA and 3.04 pA, respectively (FIGS. 102c-102f). We speculate that the sweetener in these two kinds of “zero-sugar” drinks is erythritol (ratio=22.00±0.17% and std=3.24±0.18 pA, Table 17). When sensed by MspA-PBA, the Vitamin Drink demonstrated three distinct event populations (FIGS. 81i and 81j, n=423). The statistics corresponding to the main population of events were ratio=21.89% and std=3.04 pA (FIGS. 102g, 102h), which may also be from erythritol. The secondary population of events has a ratio of 14.19%, as estimated according to the size exclusion mechanism obtained in the measurements of the thirteen alditols. This may be from other polyol ingredients which have a molecular weight lower than that of glycerol (ratio=17.07±0.15%, Table 17).

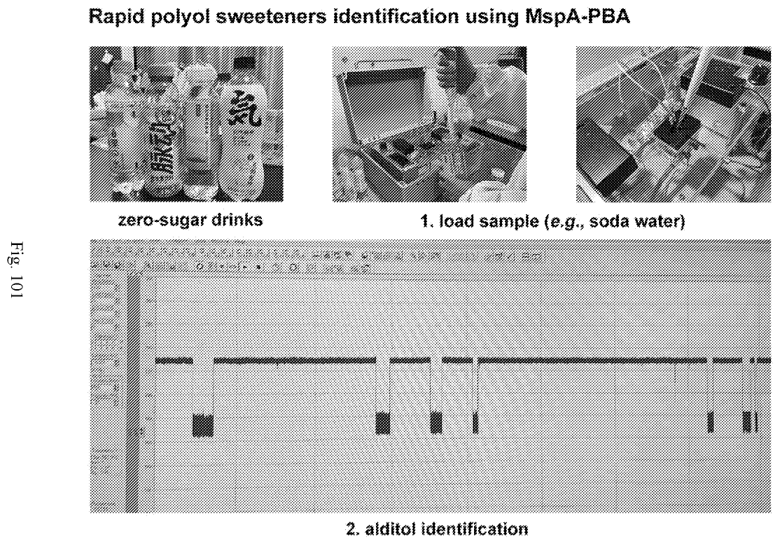

To verify the above results of visual identification of alditol types in the “zero-sugar” drinks, seven features were extracted from the events and predicted using the trained Quadratic SVM model. Since Vitamin Drink has three distinguishable populations, a k-means cluster analysis of events was performed using a custom algorithm of MATLAB to extract the events from major components in Vitamin Drink (FIG. 103a). As shown in the silhouette plot of clusters (FIG. 103b), most points in both clusters have a silhouette value >0.8, showing that those points are well-separated from neighboring clusters. Events in cluster 1 (n=393) were screened out as the predictive datasets of Vitamin Drink, while all events acquired from soda water (n=614), fruity water (n=950) and sparkling water (n=781) were included as their respective predictive datasets. Statistics of the alditol proportion in the machine learning predicting results were consistent with the expectation that all events acquired from the soda water dataset were from xylitol (FIG. 103c, 100%), while the alditol in Fruity Water, Vitamin Drink and Sparkling Water is 100% erythritol (FIGS. 103d-103f). All the above results demonstrate that the MspA-PBA could serve as a superior sensor for rapid alditol identification in drinks, a conclusion that has not been reported previously. Requiring only 30 s of measurement, each alditol molecule in drinks can be immediately analyzed according to their characteristic resistive pulses of different alditols and the measurement requires no sample pretreatment.

### Conclusion

In summary, we have presented here a strategy to identify polyol sweeteners using a phenylboronic acid appended hetero-octameric MspA. The sole PBA in the pore constriction serves as an adapter for alditols by its reversible formation of a boric acid ester. As a result of this characteristic chemical reactivity and the superior resolution of the conical shaped MspA lumen, thirteen alditols: glycerol, erythritol, threitol, arabitol, adonitol, xylitol, talitol, mannitol, allitol, iditol, dulcitol, sorbitol and gulitol (L-sorbitol) can be fully distinguished. According to the characteristics of corresponding events, a complete feature matrix of alditol sensing using nanopore has been established. To the best of our knowledge, a complete sugar alcohol database which contains alditols corresponding to all fifteen monosaccharide aldoses, has not been reported previously. A machine learning based alditol classifier has also been developed to automate alditol identification without any human bias. Multiple event features were simultaneously considered to discriminate between different alditols and a general validation accuracy of 99.4% was achieved. The trained classifier could be employed to predict events during simultaneous sensing of alditol in a mixture. This strategy is further applied in rapid identification of alditols in “zero-sugar” drinks. Four types of commercial beverages were tested, only microliters of samples are needed and no pretreatment is necessary. The whole measurement takes less than a minute, which is useful in rapid and high-resolution analysis of natural products containing polyol structures in the nutrition and medical industry. In future, engineered MspA sensors may be integrated into an array^{55, 56, 57} to boost their sensitivity and when engineered into our personal electronics, may be used in daily life.

### Author Contributions

Y. L., S. Y. Z. and S. H. conceived the project. Y. L., Y. Q. W, S. Y. Z. and P. P. F. prepared the MspA nanopores. Y. L., Y. Q. W, S. Y. Z., P. P. F. and Y. L. W. performed the measurements. Y. L., Y. Q. W and P. P. F. designed the machine-learning algorithms. P. K. Z. set up the instruments. S. H. and Y. L. wrote the paper. S. H. and H. Y. C. supervised the project.

### Data Availability Statement

All data presented in this work can be requested from the corresponding author upon reasonable request.

### Code Availability Statement

The custom machine learning code is shared as a supplementary material named as “AlditolClassifier”.

### Competing Interest Statement

Y. L., S. Y. Z., Y. Q. W. and S. H. have filed patents describing the preparation of heterogeneous MspA and its applications thereof.

### Acknowledgments

The authors acknowledge Prof. Zijian Guo, Prof. Shaolin Zhu, Prof. Congqing Zhu, Prof. Jie Li and Prof. Ran Xie in Nanjing University for valuable discussions.

This project was funded by National Natural Science Foundation of China (Grant No. 31972917, No. 91753108, No. 21675083), Supported by the Fundamental Research Funds for the Central Universities (Grant No. 020514380257, No. 020514380261), Programs for high-level entrepreneurial and innovative talents introduction of Jiangsu Province (individual and group program), Natural Science Foundation of Jiangsu Province (Grant No. BK20200009), Excellent Research Program of Nanjing University (Grant No. ZYJH004), Shanghai Municipal Science and Technology Major Project, State Key Laboratory of Analytical Chemistry for Life Science (Grant No. 5431ZZXM1902), Technology innovation fund program of Nanjing University, China Postdoctoral Science Foundation (Grant No. 2021M691508).

### REFERENCES

1. Grembecka M. Sugar alcohols—their role in the modern world of sweeteners: a review. Eur Food Res Technol 2015, 241 (1): 1-14.
2. Schiweck H, Bar A, Vogel R, Schwarz E, Kunz M, Dusautois C, et al. Sugar Alcohols. Ullmann's Encyclopedia of Industrial Chemistry, 2012.
3. Makinen K K. The Latest on Sugar Substitutes of the Alditol Type with Special Consideration of Erythritol and Xylitol-Rectifications and Recommendations. J Food Microbiol Saf Hyg 2016, 1 (3): 1000115.
4. de Cock P, Makinen K, Honkala E, Saag M, Kennepohl E, Eapen A. Erythritol Is More Effective Than Xylitol and Sorbitol in Managing Oral Health Endpoints. Int J Dent 2016, 2016:9868421.
5. Mechri B, Tekaya M, Cheheb H, Hammami M. Determination of Mannitol Sorbitol and Myo-Inositol in Olive Tree Roots and Rhizospheric Soil by Gas Chromatography and Effect of Severe Drought Conditions on Their Profiles. J Chromatogr Sci 2015, 53 (10): 1631-1638.
6. Sim H-J, Jeong J-S, Kwon H-J, Kang T H, Park H M, Lee Y-M, et al. HPLC with pulsed amperometric detection for sorbitol as a biomarker for diabetic neuropathy. J Chromatogr B 2009, 877 (14): 1607-1611.
7. Miwa I, Kanbara M, Wakazono H, Okuda J. Analysis of sorbitol, galactitol, and myo-inositol in lens and sciatic nerve by high-performance liquid chromatography. Anal Biochem 1988, 173 (1): 39-44.
8. Schimpf K J, Meek C C, Leff R D, Phelps D L, Schmitz D J, Cordle C T. Quantification of myo-inositol, 1,5-anhydro-D-sorbitol, and D-chiro-inositol using high-performance liquid chromatography with electrochemical detection in very small volume clinical samples. Biomedical chromatography: BMC 2015, 29 (11): 1629-1636.
9. Li Y, Liang J, Gao J-N, Shen Y, Kuang H-X, Xia Y-G. A novel LC-MS/MS method for complete composition analysis of polysaccharides by aldononitrile acetate and multiple reaction monitoring. Carbohydr Polym 2021, 272:118478.
10. Melton L D, Smith B G. Determination of Neutral Sugars by Gas Chromatography of their Alditol Acetates. Curr Protoc Food Anal Chem 2001, 00 (1): E3.2.1-E3.2.13.
11. Hosseinzadeh R, Mohadjerani M, Pooryousef M. A new selective fluorene-based fluorescent internal charge transfer (ICT) sensor for sugar alcohols in aqueous solution. Anal Bioanal Chem 2016, 408 (7): 1901-1908.
12. Niu W, Kong H, Wang H, Zhang Y, Zhang S, Zhang X. A chemiluminescence sensor array for discriminating natural sugars and artificial sweeteners. Anal Bioanal Chem 2012, 402 (1): 389-395.
13. Resendez A, Panescu P, Zuniga R, Banda I, Joseph J, Webb D-L, et al. Multiwell Assay for the Analysis of Sugar Gut Permeability Markers: Discrimination of Sugar Alcohols with a Fluorescent Probe Array Based on Boronic Acid Appended Viologens. Anal Chem 2016, 88 (10): 5444-5452.
14. Browne C A, Forbes T P, Sisco E. Detection and identification of sugar alcohol sweeteners by ion mobility spectrometry. Anal Methods 2016, 8 (28): 5611-5618.
15. Zhang X, Lomora M, Einfalt T, Meier W, Klein N, Schneider D, et al. Active surfaces engineered by immobilizing protein-polymer nanoreactors for selectively detecting sugar alcohols. Biomaterials 2016, 89:79-88.
16. Musto C J, Lim S H, Suslick K S. Colorimetric Detection and Identification of Natural and Artificial Sweeteners. Anal Chem 2009, 81 (15): 6526-6533.
17. Ayub M, Hardwick S W, Luisi B F, Bayley H. Nanopore-Based Identification of Individual Nucleotides for Direct RNA Sequencing. Nano Lett 2013, 13 (12): 6144-6150.
18. Clarke J, Wu H-C, Jayasinghe L, Patel A, Reid S, Bayley H. Continuous base identification for single-molecule nanopore DNA sequencing. Nat Nanotechnol 2009, 4 (4): 265-270.
19. Yuan B, Li S, Ying Y-L, Long Y-T. The analysis of single cysteine molecules with an aerolysin nanopore. Analyst 2020, 145 (4): 1179-1183.
20. Wei X, Ma D, Zhang Z, Wang L Y, Gray J L, Zhang L, et al. N-Terminal Derivatization-Assisted Identification of Individual Amino Acids Using a Biological Nanopore Sensor. ACS Sens 2020, 5 (6): 1707-1716.
21. Boersma A J, Bayley H. Continuous stochastic detection of amino acid enantiomers with a protein nanopore. Angew Chem Int Ed Engl 2012, 51 (38): 9606-9609.
22. Cao J, Jia W, Zhang J, Xu X, Yan S, Wang Y, et al. Giant single molecule chemistry events observed from a tetrachloroaurate (III) embedded Mycobacterium smegmatis porin A nanopore. Nat Commun 2019, 10 (1): 5668.
23. Hu P, Zhang Y, Wang D, Qi G, Jin Y. Glutathione Content Detection of Single Cells under Ingested Doxorubicin by Functionalized Glass Nanopores. Anal Chem 2021, 93 (9): 4240-4245.
24. Jia W, Hu C, Wang Y, Gu Y, Qian G, Du X, et al. Programmable nano-reactors for stochastic sensing. Nat Commun 2021, 12 (1): 5811.
25. Boersma A J, Brain K L, Bayley H. Real-Time Stochastic Detection of Multiple Neurotransmitters with a Protein Nanopore. ACS Nano 2012, 6 (6): 5304-5308.
26. Zhang X, Dou L, Zhang M, Wang Y, Jiang X, Li X, et al. Real-time sensing of neurotransmitters by functionalized nanopores embedded in a single live cell. Mol Biomed 2021, 2 (1): 6.
27. Wang Y, Guan X, Zhang S, Liu Y, Wang S, Fan P, et al. Structural-profiling of low molecular weight RNAs by nanopore trapping/translocation using Mycobacterium smegmatis porin A. Nat Commun 2021, 12 (1): 3368.
28. Sheng Y, Zhou K, Liu Q, Liu L, Wu H-C. Probing Conformational Polymorphism of DNA Assemblies with Nanopores. Anal Chem 2020, 92 (11): 7485-7492.
29. Zhang L, Gardner M L, Jayasinghe L, Jordan M, Aldana J, Burns N, et al. Detection of single peptide with only one amino acid modification via electronic fingerprinting using reengineered durable channel of Phi29 DNA packaging motor. Biomaterials 2021, 276:121022.
30. Ji Z, Wang S, Zhao Z, Zhou Z, Haque F, Guo P. Fingerprinting of Peptides with a Large Channel of Bacteriophage Phi29 DNA Packaging Motor. Small 2016, 12 (33): 4572-4578.
31. Liu Y, Pan T, Wang K, Wang Y, Yan S, Wang L, et al. Allosteric Switching of Calmodulin in a Mycobacterium smegmatis porin A (MspA) Nanopore-Trap. Angew Chem Int Ed 2021, 60 (44): 23863-23870.
32. Tripathi P, Benabbas A, Mehrafrooz B, Yamazaki H, Aksimentiev A, Champion P M, et al. Electrical unfolding of cytochrome c during translocation through a nanopore constriction. Proc Natl Acad Sci USA 2021, 118 (17): e2016262118.
33. Wloka C, Galenkamp N S, van der Heide N J, Lucas F L R, Maglia G. Chapter Nineteen-Strategies for enzymological studies and measurements of biological molecules with the cytolysin A nanopore. In: Heuck A P (ed). Methods in Enzymology, vol. 649. Academic Press, 2021, pp 567-585.
34. Schmid S, Stömmer P, Dietz H, Dekker C. Nanopore electro-osmotic trap for the label-free study of single proteins and their conformations. Nat Nanotechnol 2021, 16 (11): 1244-1250.
35. Schmid S, Dekker C. Nanopores: a versatile tool to study protein dynamics. Essays Biochem 2021, 65 (1): 93-107.
36. Roozbahani G M, Chen X, Zhang Y, Wang L, Guan X. Nanopore Detection of Metal Ions: Current Status and Future Directions. Small Methods 2020, 4 (10): 2000266.
37. Bétermier F, Cressiot B, Di Muccio G, Jarroux N, Bacri L, Morozzo della Rocca B, et al. Single-sulfur atom discrimination of polysulfides with a protein nanopore for improved batteries. Commun Mater 2020, 1 (1): 59.
38. Lorand J P, Edwards J O. Polyol Complexes and Structure of the Benzeneboronate Ion. J Org Chem 1959, 24 (6): 769-774.
39. James T D, Sandanayake KRAS, Shinkai S. A Glucose-Selective Molecular Fluorescence Sensor. Angew Chem Int Ed 1994, 33 (21): 2207-2209.
40. Cappuccio F E, Suri J T, Cordes D B, Wessling R A, Singaram B. Evaluation of Pyranine Derivatives in Boronic Acid Based Saccharide Sensing: Significance of Charge Interaction Between Dye and Quencher in Solution and Hydrogel. J Fluoresc 2004, 14 (5): 521-533.
41. Resendez A, Malhotra S V. Boronic Acid Appended Naphthyl-Pyridinium Receptors as Chemosensors for Sugars. Sci Rep 2019, 9 (1): 6651.
42. Yang W, Lin L, Wang B. A new type of boronic acid fluorescent reporter compound for sugar recognition. Tetrahedron Lett 2005, 46 (46): 7981-7984.
43. Liang X, James T D, Zhao J. 6,6′-Bis-substituted BINOL boronic acids as enantioselective and chemoselective fluorescent chemosensors for d-sorbitol. Tetrahedron 2008, 64 (7): 1309-1315.
44. Ramsay W J, Bayley H. Single-Molecule Determination of the Isomers of d-Glucose and d-Fructose that Bind to Boronic Acids. Angew Chem Int Ed Engl 2018, 57 (11): 2841-2845.
45. Laszlo A H, Derrington I M, Brinkerhoff H, Langford K W, Nova I C, Samson J M, et al. Detection and mapping of 5-methylcytosine and 5-hydroxymethylcytosine with nanopore MspA. Proc Natl Acad Sci USA 2013, 110 (47): 18904-18909.
46. Ma F, Yan S, Zhang J, Wang Y, Wang L, Wang Y, et al. Nanopore Sequencing Accurately Identifies the Cisplatin Adduct on DNA. ACS Sens 2021, 6 (8): 3082-3092.
47. Wang Y, Patil K M, Yan S, Zhang P, Guo W, Wang Y, et al. Nanopore Sequencing Accurately Identifies the Mutagenic DNA Lesion 06-Carboxymethyl Guanine and Reveals Its Behavior in Replication. Angew Chem Int Ed 2019, 58 (25): 8432-8436.
48. Liu Y, Wang K, Wang Y, Wang L, Yan S, Du X, et al. Machine Learning Assisted Simultaneous Structural Profiling of Differently Charged Proteins in a Mycobacterium smegmatis Porin A (MspA) Electroosmotic Trap. J Am Chem Soc 2022, 144 (2): 757-768.
49. Wang S, Cao J, Jia W, Guo W, Yan S, Wang Y, et al. Single molecule observation of hard-soft-acid-base (HSAB) interaction in engineered Mycobacterium smegmatis porin A (MspA) nanopores. Chem Sci 2020, 11 (3): 879-887.
50. Cao J, Zhang S, Zhang J, Wang S, Jia W, Yan S, et al. A Single-Molecule Observation of Dichloroaurate(I) Binding to an Engineered Mycobacterium smegmatis porin A (MspA) Nanopore. Anal Chem 2021, 93 (3): 1529-1536.
51. Zhang J, Cao J, Jia W, Zhang S, Yan S, Wang Y, et al. Mapping Potential Engineering Sites of Mycobacterium smegmatis porin A (MspA) to Form a Nanoreactor. ACS Sens 2021, 6 (6): 2449-2456.
52. Van Duin M, Peters J A, Kieboom A P G, Van Bekkum H. Studies on borate esters II11For part I see reference 7: Structure and stability of borate esters of polyhydroxycarboxylates and related polyols in aqueous alkaline media as studied by 11B NMR. Tetrahedron 1985, 41 (16): 3411-3421.
53. Peters J A. Interactions between boric acid derivatives and saccharides in aqueous media: Structures and stabilities of resulting esters. Coord Chem Rev 2014, 268:1-22.
54. Makinen K K. Gastrointestinal Disturbances Associated with the Consumption of Sugar Alcohols with Special Consideration of Xylitol: Scientific Review and Instructions for Dentists and Other Health-Care Professionals. Int J Dent 2016, 2016:5967907-5967907.
55. Kamiya K, Osaki T, Nakao K, Kawano R, Fujii S, Misawa N, et al. Electrophysiological measurement of ion channels on plasma/organelle membranes using an on-chip lipid bilayer system. Sci Rep 2018, 8 (1): 17498.
56. Yamada T, Sugiura H, Mimura H, Kamiya K, Osaki T, Takeuchi S. Highly sensitive VOC detectors using insect olfactory receptors reconstituted into lipid bilayers. Sci Adv 2021, 7 (3): eabd2013.
57. Quick J, Loman N J, Duraffour S, Simpson J T, Severi E, Cowley L, et al. Real-time, portable genome sequencing for Ebola surveillance. Nature 2016, 530 (7589): 228-232.

### Supporting Information

### Materials

Hexadecane, pentane, threitol and Genapol X-80 were purchased from Sigma-Aldrich. Arabitol was purchased from Tokyo Chemical Industry Co., Ltd. (TCI). Glycerol, dioxane-free isopropyl-β-D-thiogalactopyranoside (IPTG), kanamycin sulfate, imidazole and tris (hydroxymethyl)aminomethane (Tris) were from Solarbio. Potassium chloride (KCl), mannitol, D-sorbitol, talitol and 3-(N-Morpholino)propane sulfonic acid (MOPS) were from Aladdin (China). Xylitol, adonitol, iditol and dulcitol were from Shanghai Yuanye Biotechnology. DS-PAGE electrophoresis buffer powder was from Beyotime. Precision Plus Protein™ Dual color Standards, TGX™ FastCast™ Acylamide Kit (4-15%), stacking gel buffer (0.5M Tris-HCl buffer, pH 6.8) and resolving gel buffer (1.5M Tris-HCl buffer, pH 8.8) were obtained from Bio-Rad. 1,2-diphytanoyl-sn-glycero-3-phosphocholine (DPhPC) was from Avanti Polar Lipids. L-sorbitol, allitol and erythritol were from Macklin (China). E. coli BL21 (DE3) was from TransGen Biotech, E. coli BL21 (DE3) pLysS was from Sangon Biotech. Luria-Bertani (LB) agar and LB broth were from Hopebio. 3-(maleimide) phenylboronic acid (MPBA, Cat. #sc-352346) was from Santa Cruz Biotechnology (Shanghai) Co., Ltd.

The potassium chloride buffer (1.5 M KCl, 10 mM MOPS, pH 7.0) was prepared with Milli-Q water and membrane (0.2 μm, Whatman) filtered prior to use. The stock solutions of erythritol, threitol, adonitol, arabitol, xylitol, allitol, talitol, D-sorbitol, mannitol, L-sorbitol, iditol and dulcitol were prepared with a 400 mM concentration in the KCl buffer for subsequent measurements. The stock solution of glycerol with a concentration of 2 M in the KCl buffer was prepared for subsequent measurements. Fruity water was purchased from Coca-Cola Ice-Dew®, soda water from NongFu Spring®, vitamin drink from Danone Mizone®, and sparkling water from Genki Forest®.

### Methods

### 1. Nanopore Measurements and Data Analysis

All nanopore measurements were performed as described previously.^{1,2} Briefly, the measurement device has two custom chambers separated by a thick Teflon film containing a drilled (˜100 μm) aperture. Before the measurement, the aperture was first treated with 0.5% (v/v) hexadecane in pentane and set for pentane evaporation. Electrolyte buffer (500 μL) was added to the electrically grounded chamber (cis chamber) and the opposing chamber (trans chamber). All nanopore measurements in this paper were performed with a 1.5 M KCl buffer (1.5 M KCl, 10 mM MOPS, pH 7.0). Two custom-made Ag/AgCl electrodes were placed in both chambers in contact with the buffers and the patch-clamp amplifier to form a closed circuit. A pentane solution of DPhPC (100 μL, 5 mg/mL) was added to both chambers to form a lipid bilayer. MspA was then added to cis to initiate spontaneous pore insertion. Excess nanopores are removed by exchanging the buffer in the cis chamber upon single nanopore insertion.

A custom Faraday cage mounted on a floating optical table (Jiangxi Liansheng Technology) was employed to avoid interference from external electromagnetic and vibration noises. All electrophysiology results were acquired with an Axonpatch 200B patch-clamp amplifier paired with a Digidata 1550B digitizer (Molecular Devices). Unless otherwise stated, the voltage applied during all measurements was +100 mV and all measurements were carried out at room temperature (rt) (25° C.). All single-channel recordings were sampled at 25 kHz and low-pass filtered with a corner frequency of 1 kHz.

All protein trapping events were detected by the “single channel search” function in Clampfit 10.7. All Axon abf files were imported into MATLAB using a ‘abfload’ algorithm (Harald Hentschke (2021). abfload (https://www.mathworks.com/matlabcentral/fileexchange/6190-abfload, MATLAB Central File Exchange. Retrieved Sep. 1, 2021) to extract the features of nanopore events. The machine learning model training were performed using the Classification Learner toolbox of MATLAB. The prediction process, learning curve plotting and cluster analysis was performed using a custom algorithm in MATLAB. For validation and technology exchange, the machine learning code and sample data “AlditolClassifier” were also submitted. Subsequent analyses, including histogram plotting, scatter plot generation and curve fitting were performed by Origin 9.2 (Origin Lab).

### 2. Nanopore Preparations.

Unless stated otherwise, all measurements in this work were performed with a boronic acid appended hetero-octameric MspA. Briefly, the hetero-octameric MspA was composed of M2 MspA-D16H6 and N90C MspA-H6. M2 MspA-D16H6 is a variant of M2 MspA (D90N/D91N/D93N/D118R/D134R/E139K) with a hexahistidine tag and a 16 consecutive aspartic acid tags at its C-terminus to enhance the discrimination between hetero-octameric MspAs during gel electrophoresis. N90C MspA-H6 is another variant of M2 MspA however with a mutation of asparagine to cysteine and a hexahistidine tag at its C-terminus. Both genes were introduced in a co-expression vector pETDuet-1³ and expressed with E. coli BL21 (DE3) pLysS competent cells (Genscript, New Jersey). Experimentally, the E. coli BL21 (DE3) pLysS containing the recombinant plasmids (Genscript, New Jersey) was first recovered by streaking on LB agar containing ampicillin (50 μg/mL) and chloramphenicol (34 μg/mL). After incubation at 37° C. for about 15 h, a single colony was inoculated and added to the LB broth containing 50 μg/mL ampicillin and 34 μg/mL chloramphenicol. The mixture was shaken overnight at 37° C., and then transferred to the same LB broth medium (1 L) with a ratio of 1:100 (v/v). The culture was shaken at 37° C. and 175 rpm until the optical density at 600 nm (OD₆₀₀) reached 0.7. After cooling the medium to 16° C., IPTG with a final concentration of 0.1 mM was added to induce protein expression, and the culture was shaken at 175 rpm at 16° C. for 24 h. Finally, the medium was centrifuged at 4000 rpm for 20 min at 4° C. The bacterial pellet was stored at −80° C.

The bacterial pellet was resuspended in a 150 mL lysis buffer (100 mM Na₂HPO₄/NaH₂PO₄, 0.1 mM EDTA, 150 mM NaCl, 0.5% (w/v) Genapol X-80, pH-6.5) and heated at 60° C. for 50 min. The lytic cell was then centrifuged at 13,000 rpm for 40 min at 4° C. and the supernatant, which contain the target protein, was collected. The protein mixture was purified using nickel affinity chromatography and eluted with a linear gradient of imidazole (5 mM-500 mM) by mixing buffer A (0.5 M NaCl, 20 mM HEPES, 5 mM imidazole, 2 mM TCEP, 0.5% (w/v) Genapol X-80, pH 8.0) with buffer B (0.5 M NaCl, 20 mM HEPES, 500 mM imidazole, 2 mM TCEP, 0.5% (w/v) Genapol X-80, pH 8.0). The eluent fractions were characterized by 4-15% SDS-PAGE gel to identify the heterogeneously-assembled MspAs in the fractions. The mixed MspAs were separated by electrophoresis for 16 h with a 10% SDS-PAGE and a tris-Gly buffer at rt. The gel fragment containing the band which corresponds to the MspA (N90C)₁(M2)₇ pore type was extracted after stained with coomassie brilliant blue and rehydrated in the extraction solution (150 mM NaCl, 15 mM Tris-HCl, pH 7.5, 0.2% DDM, 0.5% Genapol X-80, 5 mM TCEP, 10 mM EDTA) for 12 h.

The freshly prepared MspA (N90C)₁(M2)₇ was modified in ensemble with 3-(maleimide) phenylboronic acid (MPBA, 500 mM in DMSO) with a ratio of 2:1 (v/v) to form a boronic acid appended hetero-octameric MspA. For simplicity, this boronic acid appended hetero-octameric MspA is referred to as MspA-PBA all through this manuscript. The prepared MspA-PBA is immediately used in all subsequent electrophysiology measurements.

The octameric M2 MspA was used as a control in FIG. 87. It was expressed with E. coli BL21 (DE3) and purified by nickel affinity chromatography as reported previously.¹

TABLE 17
Mean current percentage blockage (ratio, %) and standard deviations
of the blocking level (std, pA) of alditol trapping events
derived from “single channel search” function in Clampfit
10.7. All measurements were carried out as described in Methods
1 in Example 3. All statistical results were derived from
results of three independent measurements (N = 3).

| Alditols | ratio (%) | std (pA) |
|---|---|---|
| glycerol | 17.07 ± 0.15 | 2.57 ± 0.07 |
| erythritol | 22.00 ± 0.17 | 3.24 ± 0.18 |
| threitol | 20.3 ± 0.2 | 5.84 ± 0.18 |
| arabitol | 25.13 ± 0.15 | 4.71 ± 0.03 |
| xylitol | 25.70 ± 0.10 | 6.28 ± 0.07 |
| adonitol | 24.13 ± 0.15 | 3.83 ± 0.07 |
| dulcitol | 28.9 ± 0.3 | 5.22 ± 0.06 |
| D-sorbitol | 30.43 ± 0.06 | 5.30 ± 0.17 |
| mannitol | 29.2 ± 0.3 | 3.09 ± 0.17 |
| allitol | 27.57 ± 0.06 | 4.64 ± 0.03 |
| talitol | 27.3 ± 0.2 | 6.33 ± 0.06 |
| iditol | 29.3 ± 0.2 | 6.64 ± 0.03 |
| L-sorbitol | 30.77 ± 0.06 | 5.97 ± 0.04 |

TABLE 18
1̅/̅τ̅ₒ̅ₙ̅ and τ̅_̅{̅o̅f̅f̅}̅ of alditols with different number of hydroxyl groups
measured at various concentrations. The mean inter-event interval (τₒₙ)
and the mean dwell time (τ_{off}) were derived from single-exponential
fitting results as described in FIG. 82. All measurements were carried
out as described in Methods 1 in Example 3. The value of 1̅/̅τ̅ₒ̅ₙ̅ was
positively correlated with the concentration of the alditols and
the τ̅_̅{̅o̅f̅f̅}̅ was independent of the alditol concentration as shown in
FIGS. 88e-91e. The equilibrium binding constant (K_{b}) was
calculated according to the equation K_{b} = kₒₙ/k_{off}, in which the
association rate kₒₙ = 1/(τₒₙ * c) and the dissociation rate k_{off} = 1/τ_{off}
were derived accordingly. All statistical results were derived from
results of three independent measurements (N = 3).

| | Concen- | | | | |
|---|---|---|---|---|---|
| Typical | tration | | | | |
| alditols | (mM) | 1̅/̅τ̅ₒ̅ₙ̅ (s⁻¹) | τ̅_̅{̅o̅f̅f̅}̅ | | K_{b} (M⁻¹) |
| glycerol | 6 | 1.3 ± 0.2 | 10.5 ± 0.8 | ms | 2.21 ± 0.05 |
| | 8 | 1.7 ± 0.2 | 10.3 ± 0.4 | ms | |
| | 10 | 2.1 ± 0.3 | 10.5 ± 0.7 | ms | |
| | 12 | 2.5 ± 0.3 | 10.4 ± 0.5 | ms | |
| erythritol | 2 | 0.42 ± 0.05 | 42.4 ± 0.5 | ms | 8.6 ± 0.3 |
| | 4 | 0.80 ± 0.05 | 43.4 ± 3.2 | ms | |
| | 6 | 1.16 ± 0.03 | 42.3 ± 2.3 | ms | |
| | 8 | 1.59 ± 0.09 | 44.2 ± 1.5 | ms | |
| xylitol | 2 | 0.42 ± 0.02 | 515.3 ± 43.5 | ms | 103.0 ± 3.5 |
| | 4 | 0.84 ± 0.04 | 496.3 ± 18.6 | ms | |
| | 6 | 1.3 ± 0.1 | 461.1 ± 13.4 | ms | |
| | 8 | 1.71 ± 0.03 | 474.6 ± 21.2 | ms | |
| sorbitol | 2 | 0.40 ± 0.04 | 2.1 ± 0.3 | s | 441.2 ± 19.1 |
| (D) | 4 | 0.81 ± 0.09 | 2.11 ± 0.12 | s | |
| | 6 | 1.2 ± 0.2 | 2.2 ± 0.2 | s | |
| | 8 | 1.7 ± 0.1 | 2.2 ± 0.3 | s | |

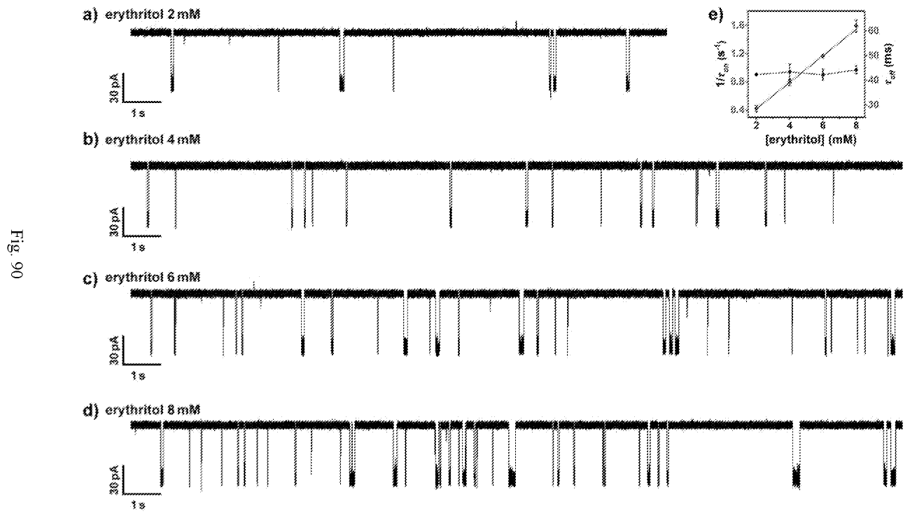

TABLE 19
Mean dwell time (τ̅_̅{̅o̅f̅f̅}̅) of alditols measured at different voltages. The
mean dwell time (τ_{off}) was derived from single-exponential fitting
results as described in FIG. 82. All measurements were carried out as
described in Methods 1 in Example 3. The value of τ̅_̅{̅o̅f̅f̅}̅ was positively
correlated with the voltages as shown in FIG. 91. All statistical results
were derived from results of three independent measurements (N = 3).

| Typical | Voltage | | |
|---|---|---|---|
| alditols | (mV) | [symbol] | 1̅/̅τ̅ₒ̅ₙ̅ (s⁻¹) |
| glycerol | +60 | (106.2 ± 6.9) *10⁻³ | 2.2 ± 0.2 |
| | +80 | (101.2 ± 7.9) *10⁻³ | 2.35 ± 0.14 |
| | +100 | (98.8 ± 8.3) *10⁻³ ms⁻¹ | 2.4 ± 0.3 |
| | +120 | (96.1 ± 7.5) *10⁻³ ms⁻¹ | 2.4 ± 0.2 |
| erythritol | +60 | (27.4 ± 2.5) *10⁻³ ms⁻¹ | 1.62 ± 0.10 |
| | +80 | (25.4 ± 2.5) *10⁻³ ms⁻¹ | 1.65 ± 0.11 |
| | +100 | (23.1 ± 2.2) *10⁻³ ms⁻¹ | 1.64 ± 0.10 |
| | +120 | (20.7 ± 0.2) *10⁻³ ms⁻¹ | 1.65 ± 0.06 |
| xylitol | +60 | (26.3 ± 2.8) *10⁻⁴ ms⁻¹ | 1.66 ± 0.02 |
| | +80 | (23.8 ± 3.2) *10⁻⁴ | 1.69 ± 0.02 |
| | +100 | (21.1 ± 9.6) *10⁻⁴ ms⁻¹ | 1.70 ± 0.03 |
| | +120 | (18.4 ± 1.4) *10⁻⁴ ms⁻¹ | 1.68 ± 0.04 |
| sorbitol (D) | +60 | (60.0 ± 3.4) *10⁻² s⁻¹ | 1.67 ± 0.03 |
| | +80 | (52.1 ± 1.8) *10⁻² s⁻¹ | 1.60 ± 0.06 |
| | +100 | (47.5 ± 2.6) *10⁻² s⁻¹ | 1.70 ± 0.06 |
| | +120 | (45.3 ± 4.5) *10⁻² s⁻¹ | 1.70 ± 0.05 |

[symbol]

indicates data missing or illegible when filed

Movie S1. Simultaneous sensing of pentitols. The electrophysiology recording was carried out as described in Methods 1 in Example 3. All nanopore measurements were performed with a 1.5 M KCl buffer (1.5 M KCl, 10 mM MOPS, pH 7.0). Arabitol, adonitol and xylitol were added to cis with a final concentration of 4 mM for each component. A transmembrane potential of +100 mV was continuously applied, during which highly consistent resistive pulses caused by arabitol, adonitol and xylitol were observed. Event identification was carried out by machine learning prediction. The identified events were labeled as Ar (arabitol, pink), Ad (adonitol, royal) and Xy (xylitol, green), respectively. For demonstration purpose, the movie is played back with the actual data acquisition speed.

Movie S2. Simultaneous sensing of propanetriol, tetritols, pentitols and hexitols. The electrophysiology recording was carried out as described in Methods 1 in Example 3. All nanopore measurements were performed with a 1.5 M KCl buffer (1.5 M KCl, 10 mM MOPS, pH 7.0) and under a transmembrane potential of +100 mV. Glycerol, tetritols mixture (erythritol and threitol), pentitols mixture (xylitol, adonitol, arabitol) and hexitols mixture (D-/L-sorbitol, talitol, allitol, iditol, dulcitol, mannitol) were added to cis. The final concentration of glycerol is 6 mM. The final concentration of erythritol and threitol are both 4 mM and that of other alditols is 2 mM each. Event identification was carried out by machine learning prediction. The identified events were labeled as G (glycerol, dark gray), E (erythritol, red), Th (threitol, blue), Ar (arabitol, pink), Ad (adonitol, royal), X (xylitol, green), D-S (D-sorbitol, sky-blue), D (dulcitol, purple), M (mannitol, wine), L-S (L-sorbitol, brown), Ta (talitol, orange), Al (allitol, dark yellow) and I (iditol, dark cyan), respectively. For demonstration purpose, the movie is played back with the actual data acquisition speed.

### REFERENCE

1. Yan, S. et al. Direct sequencing of 2′-deoxy-2′-fluoroarabinonucleic acid (FANA) using nanopore-induced phase-shift sequencing (NIPSS). Chemical Science 10, 3110-3117 (2019).
2. Wang, Y. et al. Osmosis-Driven Motion-Type Modulation of Biological Nanopores for Parallel Optical Nucleic Acid Sensing. ACS Applied Materials & Interfaces 10, 7788-7797 (2018).
3. Pavlenok, M. & Niederweis, M. Hetero-oligomeric MspA pores in Mycobacterium smegmatis. FEMS Microbiol Lett 363 (2016).

### Example 4: Single Molecule Identification of Disaccharides and Oligosaccharides with a Mycobacterium smegmatis Porin A Nanopore Modified with Boronic Acid

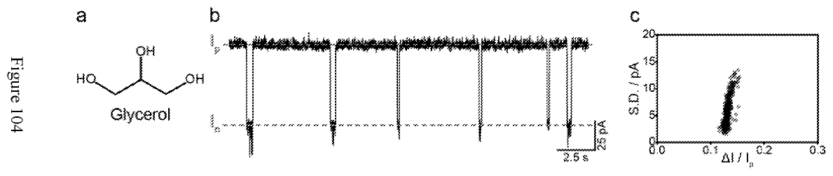

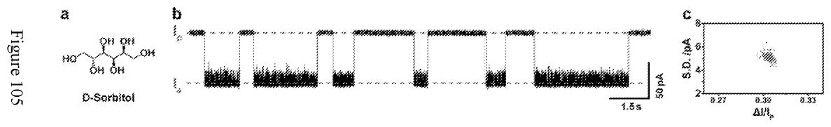

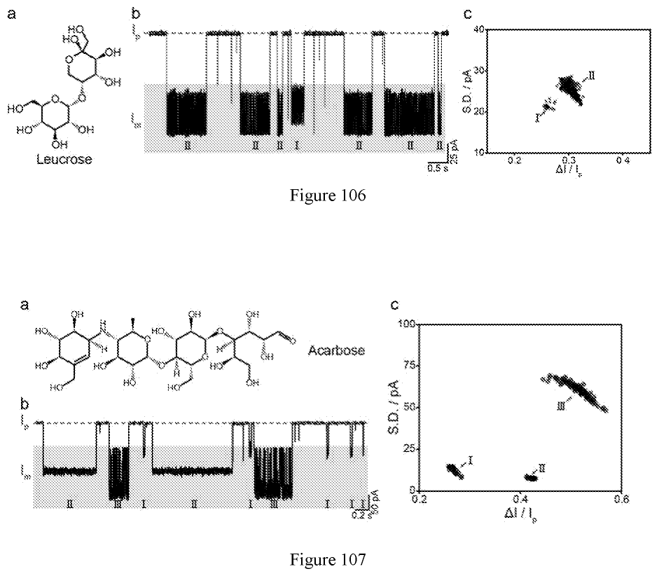

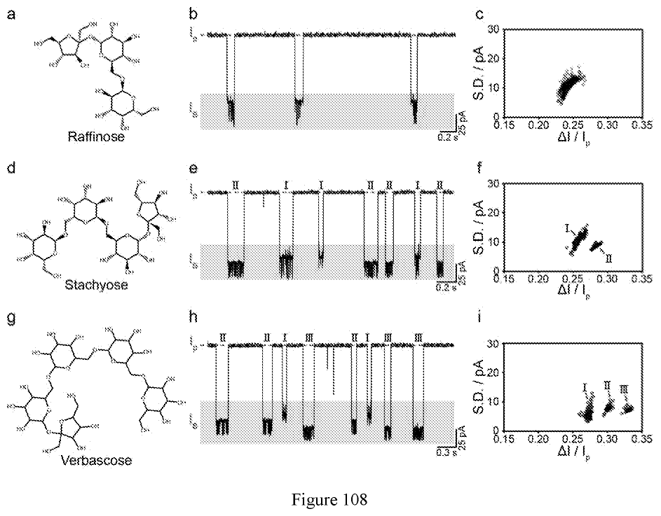

Disaccharides are composed of two monosaccharides joined by a glycosidic linkage. And oligosaccharides are carbohydrate chains containing 3-10 sugar units. They are extremely stable, naturally abundant, and have important biological functions. All polysaccharides can be sequenced by detecting disaccharide or oligosaccharide fragments produced by their hydrolysis. Mycobacterium smegmatis porin A nanopore modified with boronic acid are suitable for the detection of disaccharides or oligosaccharides. Here, MspA-PBA was used to sense disaccharides of leucrose (FIG. 106a) and soybean oligosaccharides (FIGS. 108a, d, g) as examples. MspA-PBA is prepared by the same method in Example 1. The measurement was performed with a single MspA-PBA and a 1.5 M KCl, 10 mM MOPS, pH 7.0 buffer with the continuous application of a +160 mV bias. Leucrose was added to cis with a 20 mM final concentration. Leucrose report two type of events, respectively denoted with roman numerals (FIGS. 106b, c). Oligosaccharide (raffinose, stachyose or verbascose) was added to cis with a 20 mM final concentration. Raffinose report one type of event (FIGS. 108b, c). Stachyose report two type of events, respectively denoted with roman numerals (FIGS. 108e, f). And verbascose report three type of events, respectively denoted with roman numerals (FIGS. 108h, i). The result are shown in FIG. 106 and FIG. 108, indicating that both disaccharides and oligosaccharides can be perfectly identified using MspA-PBA. Demonstrated the potential of a nanopore-based polysaccharides sequencing scheme.

### Example 5: Single Molecule Identification of Carbohydrate-Base Drug with a Mycobacterium smegmatis Porin A Nanopore Modified with Boronic Acid

The essential roles of carbohydrates in various physiological processes suggest that carbohydrate-based drugs can demonstrate high efficacy and specificity as novel therapeutic approaches. Common carbohydrate-base drugs include polysaccharides/oligosaccharides, small molecule glycosides and glycomimetics, glycopeptides and glycoproteins. Mycobacterium smegmatis porin A nanopore modified with boronic acid may be an excellent single molecule sensor for carbohydrate-base drug. Acarbose, an α-glucosidase inhibitor, is a complex oligosaccharide whose structure is similar to that of oligosaccharides. And acarbose is widely used to treat diabetes mellitus type 2. Here, acarbose is sensed by MspA-PBA as a proof-of-concept for the analysis of carbohydrate-base drugs in nanopores (FIG. 107a). MspA-PBA is prepared by the same method in Example 1. The measurement was performed with a single MspA-PBA and a 1.5 M KCl, 10 mM MOPS, pH 7.0 buffer with the continuous application of a +160 mV bias. Acarbose was added to cis with a 20 mM final concentration. Acarbose report three type of events, respectively denoted with roman numerals (FIGS. 107b, c). The results in FIG. 107 demonstrate that acarbose can be recognized by MspA-PBA, which provides new insights into the fields of carbohydrate-based drug quality control and carbohydrate-based drug development based on nanopore analytical methods.

### Example 6: Single Molecule Identification of Cis-Diols in Fruits with a Mycobacterium smegmatis Porin A Nanopore Modified with Boronic Acid

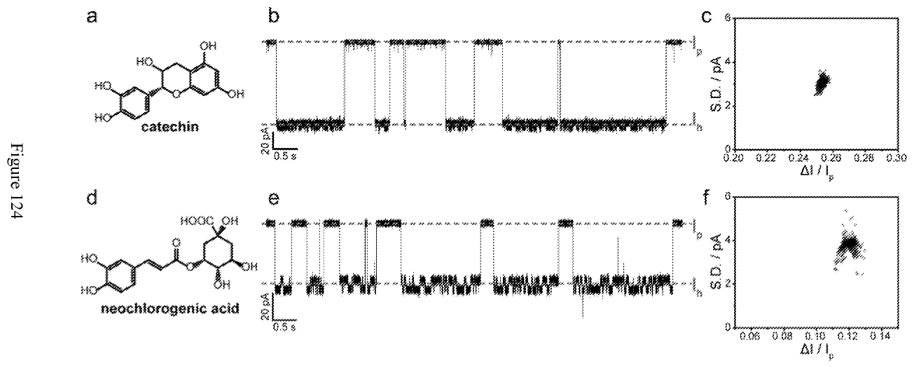

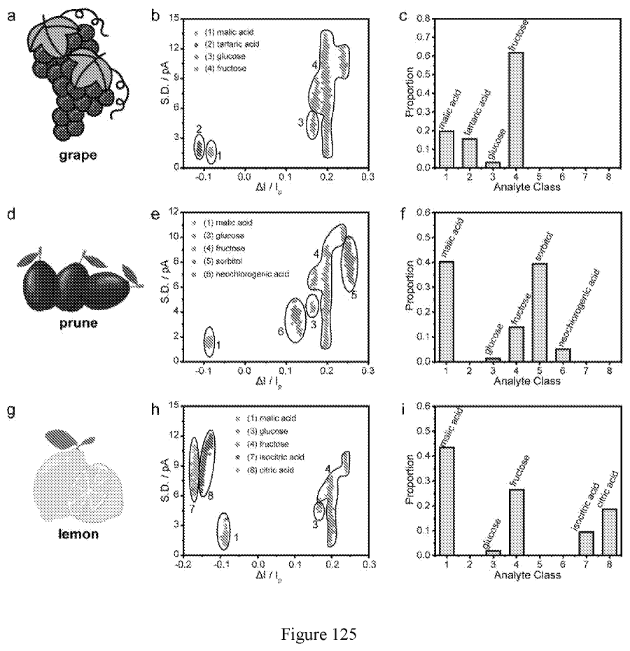

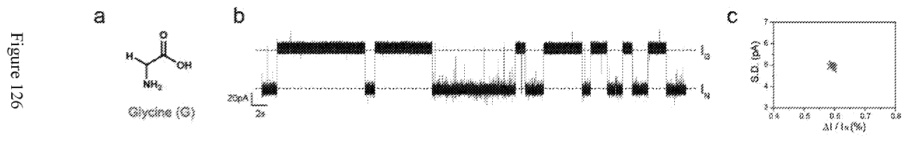

Fruits are rich in cis-diols, which can reversibly bind with phenylboronic acid. Cis-diols in fruits mainly include saccharides, alditols, 1,2-diphenols and α-hydroxy acids. Mycobacterium smegmatis porin A nanopore modified with boronic acid (MspA-PBA) can detect cis-diols in fruit. We first detected ten cis-diols that may be present in fruit. Two types of saccharides including glucose and fructose (FIG. 2), two types of alditols including sorbitol and xylitol (FIG. 78), four types of α-hydroxy acids including malic acid, tartaric acid, citric acid and isocitrate acid (FIG. 123) and two types of 1,2-diphenols including catechin and neochlorogenic acid (FIG. 124) were sensed using MspA-PBA. All measurements were performed with a single MspA-PBA and a 1.5 M KCl, 100 mM MOPS, pH 7.0 buffer with the continuous application of a +160 mV bias. All analytes were added to both cis and trans. Malic acid was added to 0.2 mM (FIG. 123b). Tartaric acid was added to 0.4 mM (FIG. 123e). Citric acid was added to 6 mM (FIG. 123h). Isocitric acid was added to 1 mM (FIG. 123k). Catechin was added to 0.8 mM (FIG. 124b). Neochlorogenic acid was added to 0.5 mM (FIG. 124e). Iₚ stands for the open pore current of the MspA-PBA. Iₕ stands for the blockage level when analyte was bound to the pore. ΔI=Iₚ−Iₕ. All signals are highly distinguishable. Then grape juice, prune juice and lemon juice were sensed using MspA-PBA (FIG. 109 and FIG. 125). 5 μL fruit juice was added to cis and trans, respectively. Other experimental conditions were the same as the above. The analyte identity was predicted by a trained machine learning model. Four populations respectively from events of malic acid, tartaric acid, glucose and fructose were detected in grape juice (FIGS. 125b and c). Five populations respectively from events of malic acid, glucose, fructose, sorbitol and neochlorogenic acid were detected in lemon juice (FIGS. 125e and f). Five populations respectively from events of malic acid, glucose, fructose, isocitric acid and citric acid were detected (FIGS. 125h and i). These results demonstrated that MspA-PBA can be used for single molecule sensing of natural fruit juices.

### Example 7: Discrimination of Nucleoside Diphosphates and Triphosphates Using MspA-PBA

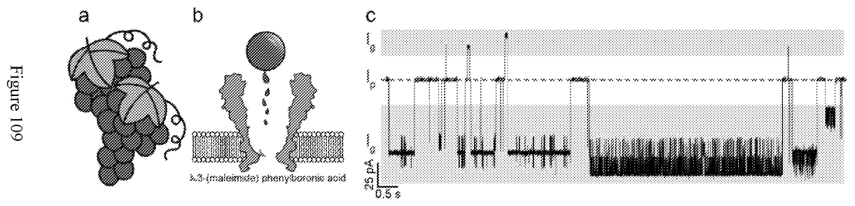

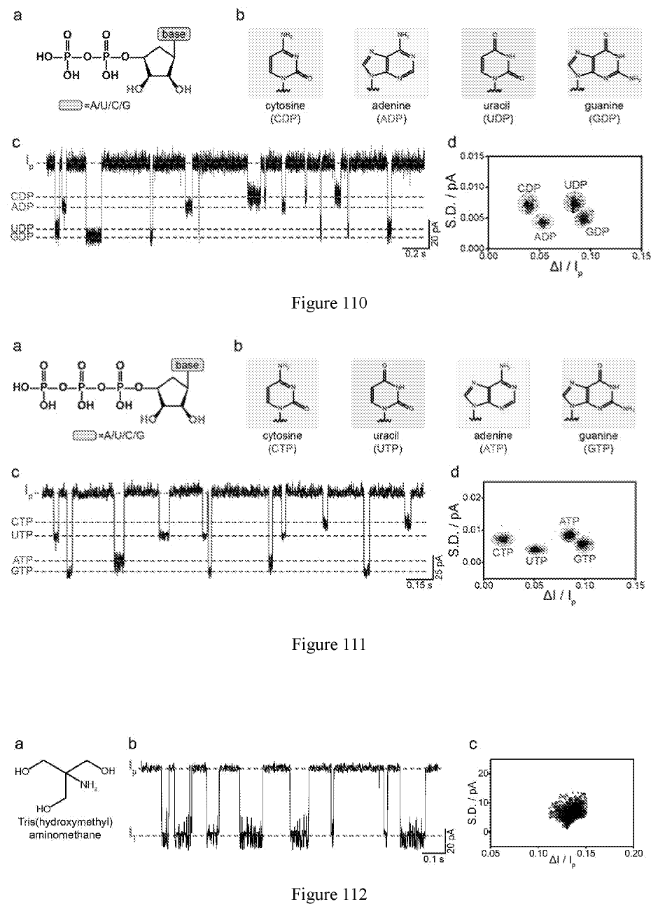

Nucleotides can exist in various phosphorylated forms, including nucleoside monophosphate (NMP), nucleoside diphosphate (NDP), or nucleoside triphosphate (NTP). The structure of NDP and NTP comprise a nitrogen base (C, U,A or G) linked to a five-carbon sugar and two or three phosphate groups, respectively (FIG. 110a and FIG. 111a). MspA-PBA is also suitable for the detection of NDPs and NTPs. Here, four classical NDPs (CDP, UDP, ADP and GDP) and four classical NTPs (CTP, UTP, ATP and GTP) were tested with MspA-PBA (FIG. 110b and FIG. 111b). The measurements with these nucleotides were performed in a 1.5 M KCl buffer (1.5 M KCl, 10 mM MOPS, pH 7.0). A transmembrane potential of +200 mV was continuously applied. Four types of NDPs were simultaneously added to cis with a final concentration of 300 μM respectively. Four types of NTPs were simultaneously added to cis with a final concentration of 300 μM respectively. Events of CDP, UDP, ADP and GDP differ significantly in their blockade amplitude, in which CDP results in the shallowest blockade, followed by ADP, UDP and GDP (FIG. 110c). By simultaneously considering two event features, the % Iₚ and the S.D., the four types of NDP could be clearly distinguished, as demonstrate in FIG. 110d. Four types of NTPs were also well distinguished with each other, and the % Iₚ is CTP <UTP <ATP <GTP (FIGS. 111b and c). These results suggest that both NDP and NTPs can be fully identified using MspA-PBA.

### Example 8: MspA-PBA is Used to Sense Tris (hydroxymethyl) aminomethane

The measurement was performed with a single MspA-PBA and a 1.5 M KCl, 10 mM MOPS, pH 7.0 buffer with the continuous application of a +140 mV bias. The addition of Tris (hydroxymethyl) aminomethane to cis to a 1 mM final concentration. The chemical structure of Tris (hydroxymethyl) aminomethane was shown (FIG. 112, a). Tris (hydroxymethyl) aminomethane report single type of events (FIG. 112, b). The scatter plot of % I_{b} versus τ_{off} for Tris (hydroxymethyl) aminomethane sensing events report single population (FIG. 112, c).

### Example 9: MspA-PBA is Used to Sense Noradrenaline

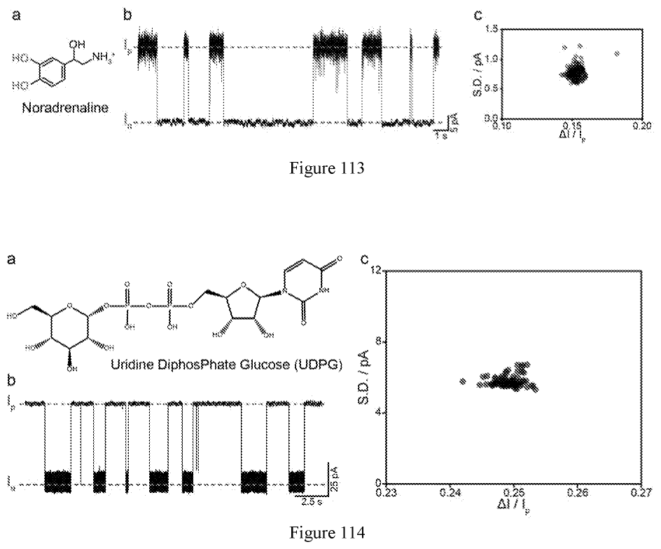

The measurement was performed with a single MspA-PBA and a 1.5 M KCl, 10 mM HEPES, pH 8.0 buffer with the continuous application of a +140 m V bias. The addition of noradrenaline to cis to a 0.3 mM final concentration. The chemical structure of noradrenaline was shown (FIG. 113, a). Noradrenaline report single type of events (FIG. 113, b). The scatter plot of % I_{b} versus τ_{off} for noradrenaline sensing events report single population (FIG. 113, c).

### Example 10: Single Molecule Sensing of Nucleotide Sugars with a Mycobacterium smegmatis Porin A Nanopore Modified with Boronic Acid

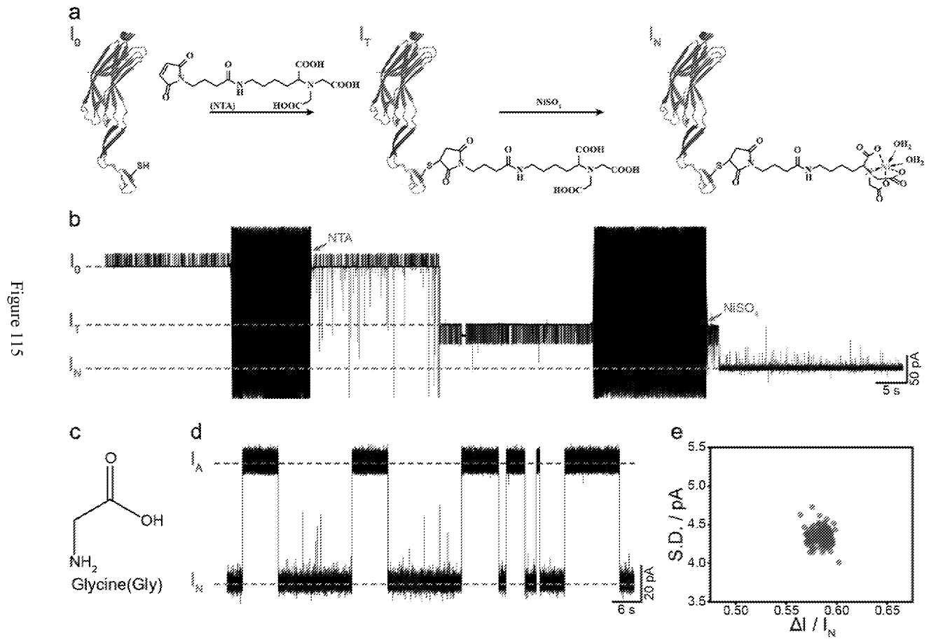

Nucleotide sugars are glycosyl donors in the biosynthesis of carbohydrates and their conjugates in all living organisms, consisting of a monosaccharide and a nucleoside mono- or diphosphate moiety. Here, uridine diphosphate glucose (UDPG) was chosen as an example detected by MspA-PBA (FIG. 114a). MspA-PBA is prepared in the same method as described in Example 1. The measurement was performed with a single MspA-PBA and a 1.5 M KCl, 10 mM MOPS, pH 7.0 buffer with the application of a +100 m V bias. When UDPG was added to cis with a final concentration of 10 mM, continuous current blockages were observed (FIG. 114b), demonstrating a single population in the scatter plot (FIG. 114c). The results suggest that MspA-PBA can efficiently detect nucleotide sugars.

### Example 11: Single Molecule Discrimination of Twenty Proteinogenic Amino Acids and their Post-Translational Modification with a Mycobacterium smegmatis Porin A Nanopore Modified with a Nickel Ion

Protein is the major workhorse of life, built from twenty amino acids. Protein sequencing is a tremendous challenge, hampered by the lack of techniques with sufficient resolution to discriminate the subtle molecular differences among all twenty amino acids. Moreover, post-translational modifications (PTMs), which alter the properties of proteins and allow proteins to perform their primary biological functions, also lacking suitable analysis methods. Here, we present evidence that a nickel-modified MspA nanopore can detect and discrimination of all twenty proteinogenic amino acids, as well as their modifications, which may pave the way to nanopore protein sequencing.

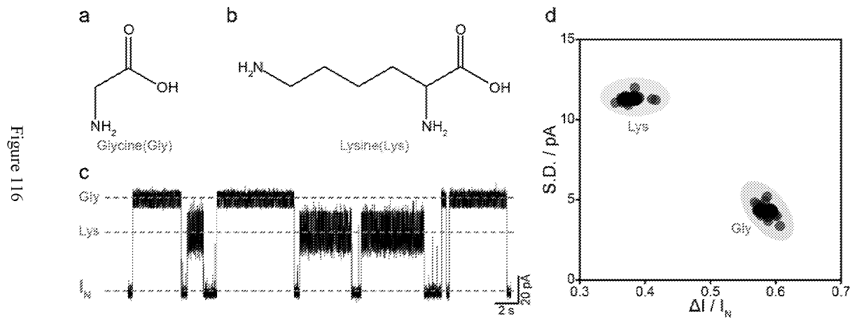

To construct nickel-modified nanopore, maleimide-C3-NTA was employed as a bridge between nanopore and nickel (FIG. 115a). (N90C)₁(M2)₇ was prepared as described in Example 1. Nanopore measurements were carried out in a 1.5 M KCl, 10 mM MOPS, pH 7.0 buffer. A +100 mV bias was continuously applied. Real time observation of NTA-Ni modification was demonstrated in FIG. 115b. After a single (N90C)₁(M2)₇ was inserted in the lipid bilayer, it reported a stable open current with upwards noise. When maleimide-C3-NTA was added to cis with a final concentration of 0.5 mM, an irreversible current drop of about 110 pA was observed, with downwards resistance pulses, indicating that NTA was covalently linked to (N90C)₁(M2)₇. Subsequently, nickel was added to trans with a final concentration of 50 μM, and then an irreversible current drop of about 80 pA was observed, representing the successful chelation of Ni by NTA.

Mycobacterium smegmatis porin A nanopore modified with nickel is suitable for the detection of amino acids by coordination interactions. Here, glycine was used as a typical example to demonstrating the concept (FIG. 115c). The measurements were performed in a 1.5 M KCl, 10 mM MOPS, pH 7.0 buffer with the application of +100 mV bias. With the addition of glycine to cis with a final concentration of 10 mM, continuous upwards current blockages appeared (FIG. 115d), demonstrating a single distribution in the scatter plot (FIG. 115e). Simultaneous sensing of glycine and lysine was further performed (FIGS. 116a, b). Amino acids were simultaneously added to cis with a final concentration of 10 mM for each analyte. Characteristic events from two amino acids are clearly recognized from the trace (FIG. 116c). The scatter plot also demonstrated two distinct distributions without any overlaps.

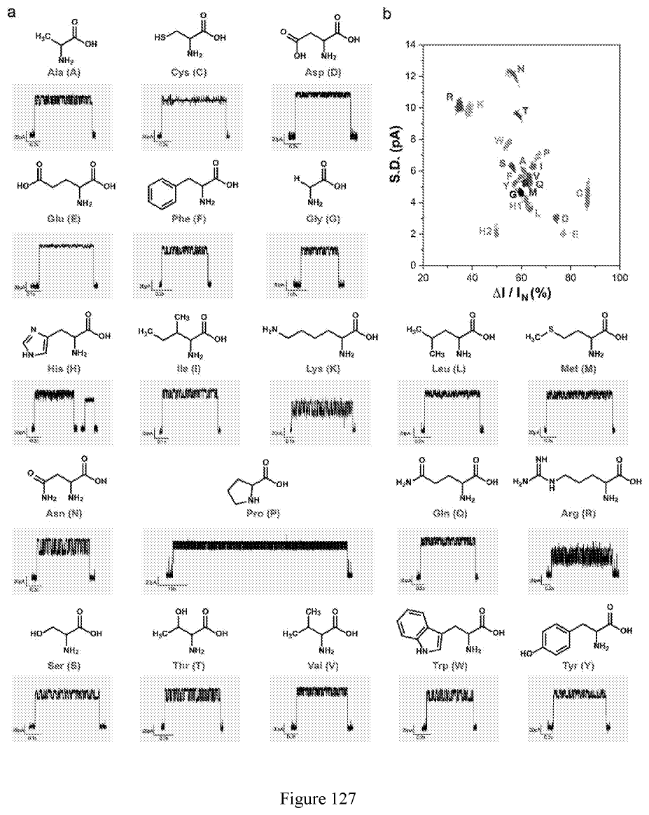

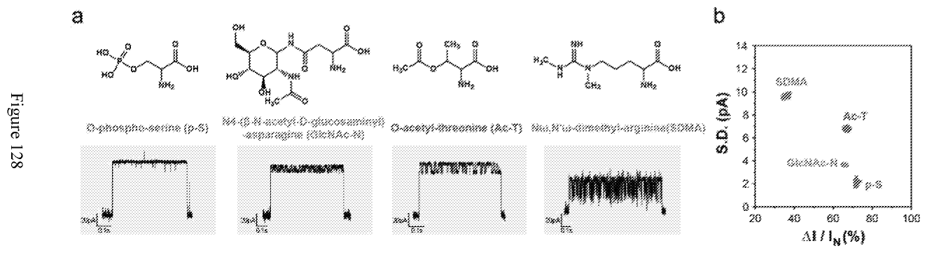

In order to improve the detection efficiency, we adjusted the pH of buffer from 7 to 9 (1.5 M KCl, 10 mM CHES, pH 9.0). Because the amount of amino acid in fully deprotonated form is higher under alkaline conditions, which is conducive to the coordination. The sensing performance was firstly evaluated with glycine (FIG. 126). A transmembrane potential of +100 mV was continuously applied. After a single nickel modified nanopore was inserted in the membrane, glycine was added to cis with a final concentration of 1 mM. Glycine also reported one type of event (FIG. 126c). Then, we performed similar measurements for all twenty amino acids using nickel-modified nanopore independently (FIG. 127a). The final concentration of amino acids was 2 mM except for proline (40 mM), and characteristic current blockade events were immediately observed with unique event characteristics. Only histidine demonstrated two types of events while the rest reported one type of event. Events from twenty proteinogenic amino acids are clearly distinguishable in the scatter plot (FIG. 127b).

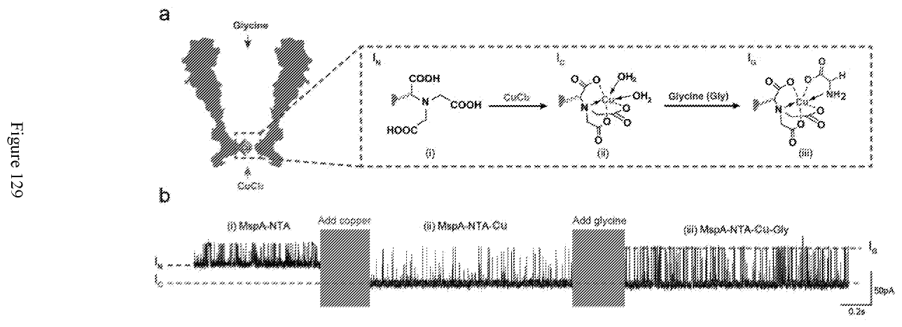

Amino acids with post-translational modifications are also detectable using nickel-modified MspA. Here, we demonstrated the detection of four common modifications, phosphorylation, glycosylation, acetylation and methylation (FIG. 128a). Nanopore measurements was performed with a 1.5 M KCl, 10 mM CHES, pH 9.0 buffer and a +100 m V bias. Each amino acid was added to cis with a final concentration of 2 mM individually. As shown in FIG. 128b, the blockage events of amino acids with four different modifications could be clearly distinguished from each other according to the characteristics.

### Example 12: Saccharides Sensing by Mycobacterium smegmatis Porin A Nanopore Modified with Boronic Acid at Site 91

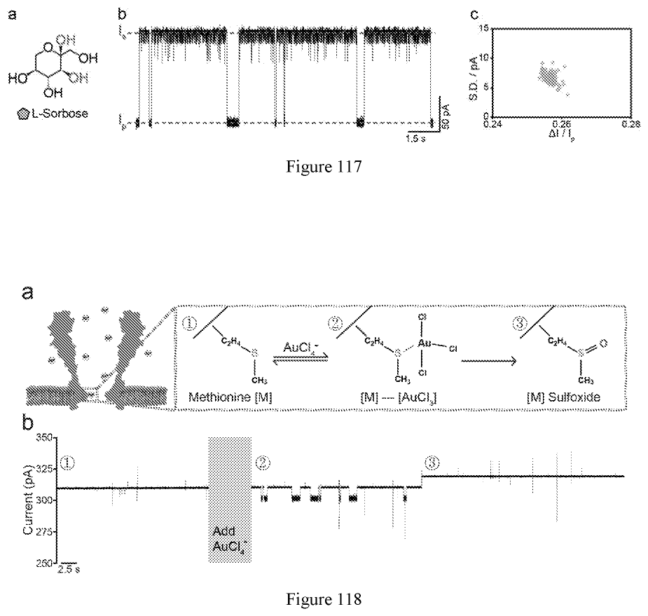

In order to expand the versatility of this method, we prepared the heterogeneously assembled MspA octamer with a mutation at position 91 to cysteine. The MspA hetero-octamer which is referred to as (N91C)₁(M2)₇ is prepared by the same method as for the preparation of (N90C)₁(M2)₇ in Example 1. (N91C)₁(M2)₇ contains a single cysteine at site 91 of the N91C MspA-H6 component, at the pore constriction. To chemically modify the (N91C)₁(M2)₇, 5 μL of freshly prepared (N91C)₁(M2)₇ and 2.5 μL DMSO solution of 3-(maleimide) phenylboronic acid (500 mM) were mixed and added to a 42.5 μL 1.5 M KCl buffer (1.5 M KCl, 10 mM MOPS, pH 7.0). The mixture was set at rt for 10 min. This PBA conjugated (N91C)₁(M2)₇ hetero-octamer is referred to as MspA-91PBA. MspA-91PBA is used to sense L-Sorbose (FIG. 117a). The measurement was performed with a single MspA-91PBA and a 1.5 M KCl, 10 mM MOPS, pH 7.0 buffer with the continuous application of a −160 mV bias. The addition of L-Sorbose to cis to a 10 mM final concentration. Iₛ stands for the blockage level when a saccharide was bound to the pore (FIG. 117b). The scatter plot of the ΔI/Iₚ vs the standard deviation (S.D.) in FIG. 117c shows the statistical population of L-Sorbose sensing events. FIG. 117 illustrates that the introduction of modifications at any site in the nanopore can achieve the corresponding function.

### Example 13: Nanopore-Based Real-Time Monitoring of Chemical Reactions

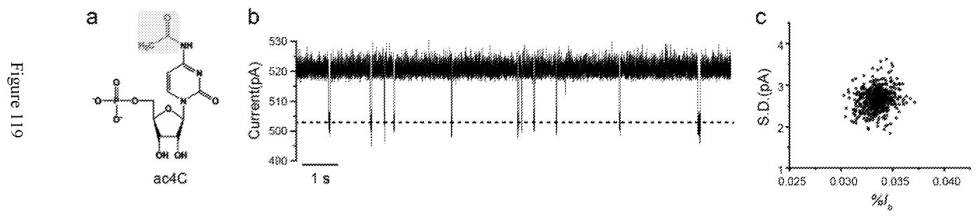

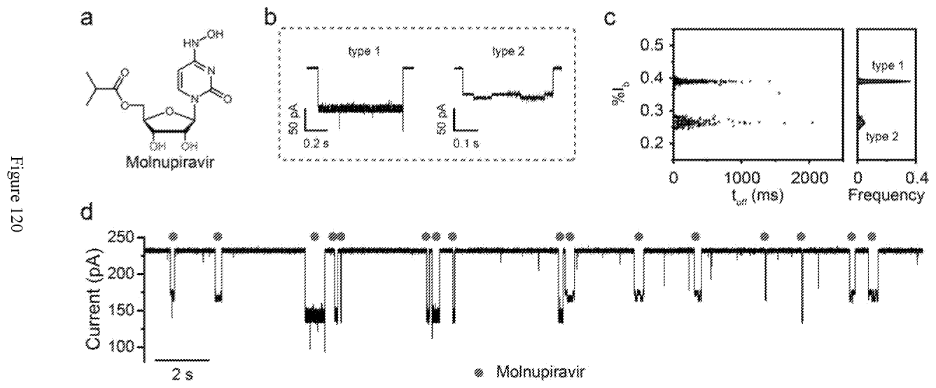

The MspA hetero-octamer which is referred to as (N91M) (M2)₇ is prepared by the same method as for the preparation of (N90C)₁(M2)₇ in Example 1. (N91M)₁(M2)₇ contains a single methionine at site 91 of the N91M MspA-H6 component, at the pore constriction, and capable of binding an [AuCl₄]⁻ ion. Subsequently, [AuCl₄]⁻ oxidizes methionine residues to sulfoxides (FIG. 118a). The measurement was performed with a single (N90C)₁(M2)₇ and a 1.5 M KCl, 10 mM HEPES, pH 7.0 buffer with the continuous application of a +100 m V bias. The tetrachloroaurate(III) was added to cis with a 1 mM final concentration. Stage 1 for the open pore current of (N91M)₁(M2)₇. Stage 2 for the blockage events when an [AuCl₄]⁻ ion was bound to the pore. Stage 3 for the methionine residues were oxidized to sulfoxide in the pore (FIG. 118b). FIG. 118 shows how we can use nanopores to monitor chemical reactions in real time.

### Example 14: Single Molecule Sensing of N-Acetylcytidine-5-Monophosphate (ac4C) with MspA-PBA

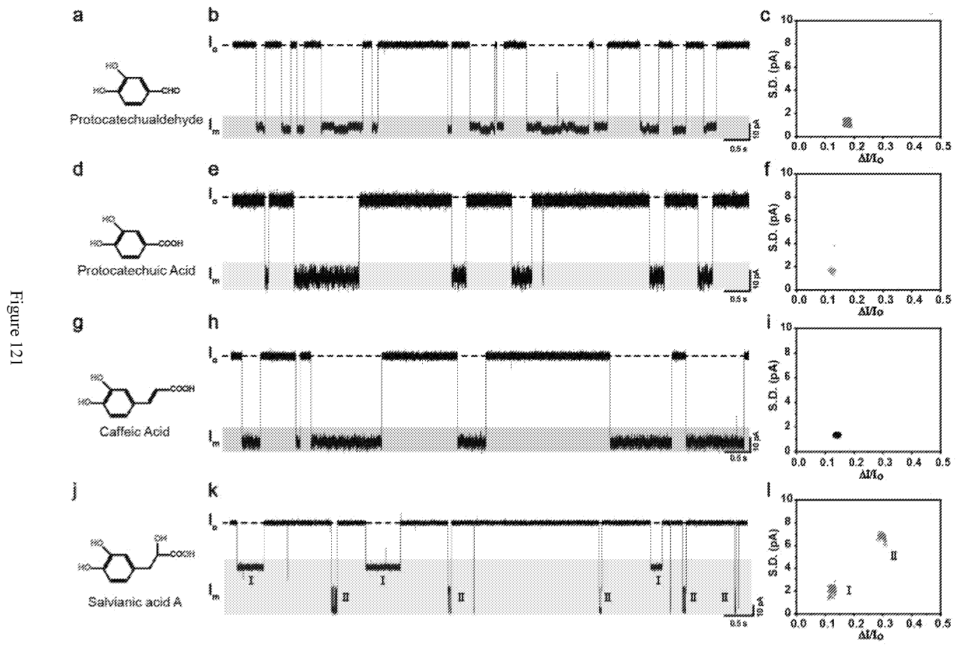

Ac4C is a modified CMP in which one of the exocyclic amino hydrogens is substituted by an acetyl group (FIG. 119a). Acknowledging the existence of a cis-diol, ac4C can bind with the phenylboronic acid of MspA-PBA. The measurements were carried out in a 1.5 M KCl buffer (1.5 M KCl, 10 mM MOPS, pH 7.0). A transmembrane potential of +200 mV was continually applied. With the addition of ac4C to a final concentration of 300 μM in cis, successive resistive pulses immediately appeared, confirming the feasibility of direct ac4C sensing with MspA-PBA (FIG. 119b). Only a single distribution could be clearly observed in the scatter plot of of % Iₚ versus S.D., demonstrating the high homogeneity of ac4C binding events (FIG. 119c).

### Example 15: MspA-PBA is Used to Sense Nucleoside Analogs Molnupiravir

The measurement was performed with a single MspA-PBA and a 1.5 M KCl, 10 mM MOPS, pH 7.0 buffer with the continuous application of a +100 mV bias. The addition of molnupiravir to cis to a 0.5 mM final concentration. The chemical structure of molnupiravir was shown (FIG. 120, a). Molnupiravir reports two types of events, respectively denoted with type 1 and 2 (FIG. 120, b). The scatter plot of % I_{b} versus τ_{off} for molnupiravir sensing events report two main distributions (FIG. 120, c). A representative trace containing molnupiravir binding events was shown (FIG. 120, d). The results were shown in FIG. 120, indicating that nucleoside analogs can be perfectly identified using MspA-PBA.

### Example 16: Single Molecule Identification of Chinese Herb—Salvia Miltiorrhiza with a Mycobacterium smegmatis Porin A Nanopore Modified with Boronic Acid

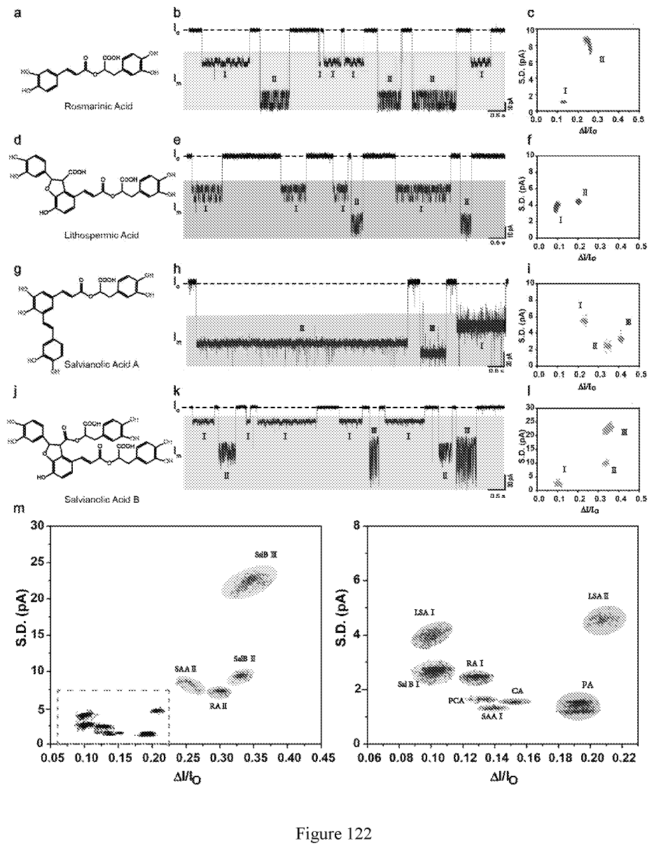

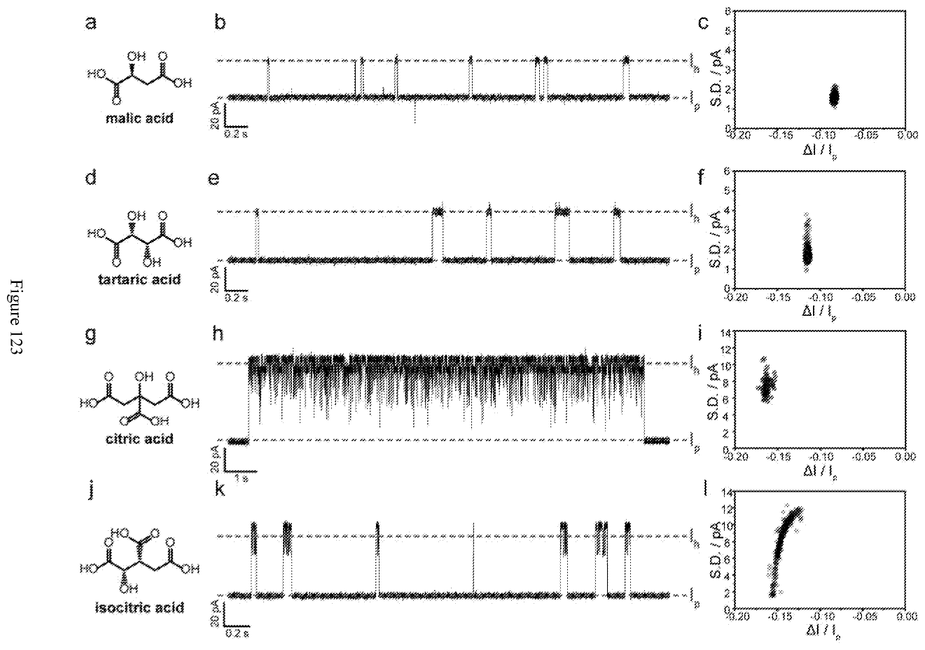

Danshen (Salvia Miltiorrhiza) is a commonly used Chinese materia medica for treating cardiovascular diseases for many years. Among the water-soluble components, salvianolic acids are the main substances that have real therapeutic effects. Because of the complex composition of salvia miltiorrhiza aqueous solution, it is very important for the detection and quality control of salvianolic acids injection and other medicines. Here, MspA-PBA is used to sense salvianolic acids which are the main active component of water-soluble part of Danshen. The measurement was performed with a single MspA-PBA and a 1.5 M KCL, 100 mM MOPS, pH 7.0 buffer with the continuous application of the +100m V bias. The addition of each salvianolic acids to cis to 1 mM final concentration resulted the different types of signals. The results showed in FIG. 121-122 illustrates that the salvianolic acids with similarity structures could be entirely distinguished by single MspA-PBA under conditions mentioned above. And the Salvianolic Acid B, an active ingredient relatively stable and the most deeply studied, has very characteristic signals which is helpful for rapid qualitative and quantitative analysis in chinese herbal products' quality control.

### Example 17: The Construction of Copper-Modified Mycobacterium smegmatis Porin A Nanopore

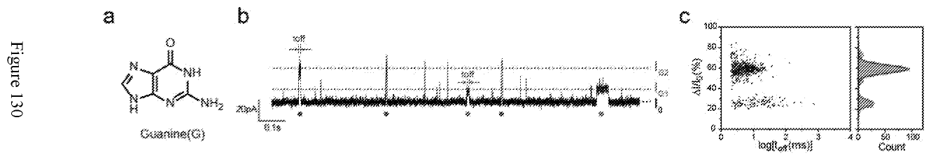

Since NTA is a universal metal chelating agent, the scope of metal-modified nanopore could be further expanded. Here, copper was employed as an example to demonstrate this concept (FIG. 129). The construction mechanism of copper-modified nanopores was similar to nickel-modified nanopores (FIG. 129a). Electrophysical measurements were carried out in a 1.5 M KCl, 10 mM CHES, pH 9.0 buffer using NTA-modified MspA (1 μL prepared (N90C)₁(M2)₇ and 8 μL 20 mM maleimide-C₃-NTA incubated for 12 h). A transmembrane potential of +100 mV was continually applied. After a single NTA-modified MspA was inserted in the membrane, copper ions were added to trans with a final concentration of 100 μM (FIG. 129b). An irreversible current drop of about 30 pA was immediately observed, suggesting the coordination of a single copper ion. Glycine was further added to cis with a final concentration of 100 μM, and frequent upwards current events began to appear (FIG. 129b). These results illustrated the successful construction of copper-modified nanopores and their application in sensing amino acids.

### Example 18: Single-Molecule Sensing of Guanine with Nickel-Modified Mycobacterium smegmatis Porin A Nanopore

Nucleobases are important building blocks of nucleic acid. Studies on the coordination chemistry of nucleobases haven been employed as models for exploring the metal-DNA interactions. Here, we demonstrated the application of nickel-modified MspA in sensing of guanine (FIG. 130a). The measurements were performed with a 1.5 M KCl, 10 mM CHES, pH 9.0 buffer. A +100 mV transmembrane potential was continuously applied. After the addition of guanine with a 20 mM final concentration, two types of events were immediately observed (FIG. 130b, c), possibly due to different coordination modes. The result demonstrated the potential of nickel-modified nanopore in nucleobase detection.

1. A protein nanopore comprising at least one sensing moiety, wherein the sensing moiety is a metal ion which is attached to a reactive amino acid residue in the nanopore and is capable of interacting with a target analyte.

2. The protein nanopore according to claim 1, wherein the metal ion is attached to the reactive amino acid residue via a ligand, and the metal ion and the ligand form a coordination complex.

3. The protein nanopore according to claim 2, wherein the ligand is nitrilotriacetic acid (NTA).

4. The protein nanopore according to claim 1, wherein the metal ion is selected from Ni²⁺, Cu²⁺, Co²⁺, Zn²⁺, Cd²⁺, Ag²⁺ Pb²⁺, Fe²⁺ or Fe³⁺.

5. The protein nanopore according to claim 1, wherein the reactive amino acid residue is selected from the group consisting of cysteine, methionine and lysine.

6. The protein nanopore according to claim 1, wherein the protein nanopore is a heterogeneous protein nanopore in which one or more but not all monomers comprise the sensing moiety and the other monomers do not comprise the sensing moiety.

7. The protein nanopore according to claim 6, wherein the heterogeneous protein nanopore is a variant of the nanopore selected from the group consisting of MspA, α-HL, Aerolysin, ClyA, FhuA, FraC, PlyA/B, CsgG and Phi 29 connector.

8. The protein nanopore according to claim 7, wherein the heterogeneous protein nanopore is a variant of MspA.

9. The protein nanopore according to claim 6, wherein the protein nanopore is a heterogeneous MspA nanopore that comprises Ni²⁺ attached to the reactive amino acid residue via a ligand.

10. The protein nanopore according to claim 9, wherein Ni²⁺ is attached to the reactive amino acid residue via NTA.

11. The protein nanopore according to claim 9, wherein the reactive amino acid residue is located at a position selected from 83-111, or is located at 90, 91, 92 and 93.

12. The protein nanopore according to claim 11, wherein the heterogeneous protein nanopore has a mutation of N90C, N90M or N91C on one or more monomers compared to M2 MspA.

13. A protein nanopore comprising at least one sensing module, wherein the protein nanopore is a heterogeneous MspA in which one or more but not all monomers comprise the sensing module and the other monomers do not comprise the sensing module, wherein the sensing module is capable of interacting with a target analyte.

14. The protein nanopore according to claim 13, wherein the sensing module consists of one or more reactive amino acid residues that are comprised in one or more monomers of the heterogeneous MspA.

15. The protein nanopore according to claim 14, wherein the reactive amino acid residue is selected from methionine, histidine, cysteine or lysine or their combination thereof.

16. The protein nanopore according to claim 12, wherein the sensing module consists of one or more sensing moieties that are attached to one or more reactive amino acid residues comprised in one or more monomers of the heterogeneous protein nanopore, and the other monomers of the heterogeneous protein nanopore do not comprise the reactive amino acid residue.

17. The protein nanopore according to claim 16, wherein the reactive amino acid residue is selected from the group consisting of cysteine, methionine, lysine.

18. The protein nanopore according to claim 16, wherein the sensing moiety is a moiety comprising boronic acid.

19. The protein nanopore according to claim 18, wherein the moiety comprising boronic acid is phenylboronic acid (PBA).

20. The protein nanopore according to claim 13, wherein the reactive amino acid residue is located at one or more positions selected from 83-111, or is located at 90, 91, 92 and/or 93.

21. The protein nanopore according to claim 13, wherein the heterogeneous protein nanopore has a mutation of N90C, N90M and/or N91C on one or more monomers compared to M2 MspA.

22. A method for characterizing a target analyte, comprising:
(i) providing the protein nanopore according to claim 1;
(ii) applying a voltage between the two sides of the protein nanopore reactor;
(iii) allowing the target analyte to pass through the nanopore; and
(iv) measuring an ionic current through the nanopore to provide a current pattern, and characterizing the target analyte based on the current pattern.

23.-27. (canceled)

28. The method according to claim 22, wherein the target analyte can interact with boronic acid, metal ion, methionine, histidine, cysteine, lysine or any combination thereof.

29. The method according to claim 28, wherein:
the analyte that can interact with boronic acid is selected from a chemical compound comprising 1,2-diol or 1,3-diol, an ion comprising metal element, hydrogen peroxide and any combination thereof;
the analyte that can interact with metal ion is a molecule that can interact with the metal ion by coordination; and
the analyte that can interact with methionine, histidine, cysteine or lysine is an ion comprising metal element.

30. The method according to claim 29, wherein:
the ion comprising metal element is selected from alkaline-earth metal ion, transition metal ion and any combination thereof, or selected from AuCl₄⁻, Mg²⁺, Ca²⁺, Ba²⁺, Ni²⁺, Cu²⁺, Co²⁺, Zn²⁺, Cd²⁺, Ag²⁺, Pb²⁺ and any combination thereof;
the chemical compound comprising 1,2-diol or 1,3-diol is selected from saccharide or a derivative thereof, α-hydroxy acid, a chemical compound comprising a ribose, nucleotide sugar, alditol, polyphenol, catecholamine or catecholamine derivative, tris(hydroxymethyl)methyl aminomethane (Tris), protocatechualdehyde, protocatechuic acid, caffeic acid, rosmarinic acid, lithospermic acid, salvianic acid A, salvianolic acid B and any combination thereof; and
the molecule that can interact with the metal ion by coordination contains nitrogen, oxygen, sulfur, phosphorus or carbon atom that can coordinate with the metal ion.

31. (canceled)

32. The method according to claim 30, wherein:
the saccharide is selected from monosaccharide, oligosaccharide, polysaccharide and any combination thereof, or selected from disaccharide, trisaccharide, tetrasccharide, complex oligosaccharide, pentasaccharide and any combination thereof;
the derivative of saccharide is selected from N-acetylneuraminic acid (sialic acid), N-Acetyl-D-Galactosamine and any combination thereof;
α-hydroxy acid is selected from tartaric acid, malic acid, citric acid, isocitric acid and any combination thereof;
the chemical compound comprising a ribose is selected from nucleotide or modified nucleotide, derivative of nucleotide or modified nucleotide, nucleoside or nucleoside analogue, and any combination thereof;
the nucleotide sugar is selected from uridine diphosphate glucose (UDPG), uridine diphosphate N-acetylglucosamine, uridine diphosphate glucuronic acid, adenosine diphosphate glucose, uridine diphosphate galactose, uridine diphosphate xylose, guanosine diphosphate mannose, guanosine diphosphate fucose, cytidine monophosphate N-acetylneuraminic acid, uridine diphosphate N-acetylgalactosamine and any combination thereof;
the alditol is selected from glycerin, propanetriol, tetritol, pentitol, hexitol, erythritol, threitol, arabitol, xylitol, adonitol, fucitol, sorbitol such as L-sorbitol or D-sorbitol, mannitol, dulcitol, iditol, talitol, allitol, maltitol, lactitol, isomalt and any combination thereof;
the polyphenol is selected from catechin, neochlorogenic acid, anthocyanin, proanthocyanidin, catechol or derivative thereof, such as catechol, 3-fluorocatechol, 3-chlorocatechol, 3-bromocatechol, 4-fluorocatechol, 4-chlorocatechol, 4-bromocatechol, 3-methylcatechol, 4-methylcatechol, 3-methoxycatechol, 3-propylcatechol, 3-isopropylcatechol, 3,6-dibromocatechol, 4,5-dibromocatechol, 3,6-dichlorocatechol, and any combination thereof;
the catecholamine or catecholamine derivative is selected from epinephrine, norepinephrine, isoprenaline and any combination thereof; and
the molecule that can interact with the metal ion by coordination is a compound contains at least one carboxylic acid group or at least one amine group, an amino acid, modified amino acid, polymer of amino acids or modified amino acids, a chemical compound comprising guanine, adenine, thymine, cytosine or uracil, and any combination thereof.

33. The method according to claim 32, wherein:
the monosaccharide is selected from D-glyceraldehyde, D-erythrose, D-ribose, 2′-deoxy-D-ribose, D-xylose, L-arabinose, D-lyxose, D-glucose, D-galactose, D-mannose, D-fructose, L-sorbose, L-fucose, D-allose, D-tagatose, L-rhamnose, D-galactose and any combination thereof;
the disaccharide is selected from sucrose, isomaltulose, maltulose, turanose, leucrose, trehalulose, lactulose, maltose, and any combination thereof;
the trisaccharide is selected from raffinose;
the tetrasccharide is selected from stachyose;
the complex oligosaccharide is selected from acarbose;
the pentasaccharide is selected from, verbascose;
the nucleotide is selected from adenine nucleotide, cytosine nucleotide, uracil nucleotide, guanine nucleotide and any combination thereof;
the modified nucleotide is selected from a nucleotide containing 5-methylcytidine (m⁵C), N6-methyladenosine (m⁶A), pseudouridine (Ψ), inosine (I), N7-methylguanosine (m⁷G), N1-methyladenosine (m¹A), dihydrouridine (D), N2-methylguanosine (m²G), N2,N2-dimethylguanosine (m₂²G), wybutosine (Y), 5-methyluridine (T), N-acetylcytidine (ac4C) and any combination thereof;
the derivative of nucleotide or modified nucleotide is selected from monophosphate derivative, diphosphate derivative, triphosphate derivative and tetraphosphate derivative of a nucleotide or a modified nucleotide and any combination thereof, or selected from ADP, UDP, GDP, CDP, ATP, UTP, GTP, CTP and any combination thereof; and
the nucleoside analogue is selected from galidesvir, ribavirin, molnupiravir, remdesivir, loxoribine, mizoribine, 5-azacytidine, capecitabine, doxifluridine, 5-fluorouridine, forodesine, clitocine, pyrazofurin, sangivamycin, pseudouridimycin and any combination thereof;
the sorbitol is selected from L-sorbitol or D-sorbitol and any combination thereof;
the catechol or derivative thereof is selected from catechol, 3-fluorocatechol, 3-chlorocatechol, 3-bromocatechol, 4-fluorocatechol, 4-chlorocatechol, 4-bromocatechol, 3-methylcatechol, 4-methylcatechol, 3-methoxycatechol, 3-propylcatechol, 3-isopropylcatechol, 3,6-dibromocatechol, 4,5-dibromocatechol, 3,6-dichlorocatechol, and any combination thereof;
the amino acid is selected from alanine, cysteine, aspartic acid, glutamic acid, phenylalanine, glycine, histidine, isoleucine, lysine, leucine, methionine, asparagine, proline, glutamine, arginine, serine, threonine, valine, tryptophan, tyrosine, pyrolysine, selenocysteine and any combination thereof;
the modified amino acid is selected from phosphorylate amino acid, glycosylated amino acid, acetylated amino acid, methylated amino acid and any combination thereof, or selected from O-phospho-serine (p-S), N4-(β-N-acetyl-D-glucosaminyl)-asparagine (GlcNAc-N), O-acetyl-threonine (Ac-T), Nω, N′ω-dimethyl-arginine (SDMA) and any combination thereof; and
the chemical compound comprising guanine, adenine, thymine, cytosine or uracil is selected from guanine, adenine, thymine, cytosine or uracil, or a nucleoside comprising any one of them, or a nucleotide comprising any one of them, wherein the nucleotide is a ribonucleotide or a deoxyribonucleotide.

34.-36. (canceled)

37. A method for characterizing a target analyte, comprising:
(i) providing the protein nanopore according to claim 13;
(ii) applying a voltage between the two sides of the protein nanopore reactor;
(iii) allowing the target analyte to pass through the nanopore; and
(iv) measuring an ionic current through the nanopore to provide a current pattern, and characterizing the target analyte based on the current pattern.

38. The method according to claim 37, wherein the target analyte can interact with boronic acid, metal ion, methionine, histidine, cysteine, lysine or any combination thereof.

39. The method according to claim 38, wherein:
the analyte that can interact with boronic acid is selected from a chemical compound comprising 1,2-diol or 1,3-diol, an ion comprising metal element, hydrogen peroxide and any combination thereof;
the analyte that can interact with metal ion is a molecule that can interact with the metal ion by coordination; and
the analyte that can interact with methionine, histidine, cysteine or lysine is an ion comprising metal element.

40. The method according to claim 39, wherein:
the ion comprising metal element is selected from alkaline-earth metal ion, transition metal ion and any combination thereof, or selected from AuCl₄⁻, Mg²⁺, Ca²⁺, Ba²⁺, Ni²⁺, Cu²⁺, Co²⁺, Zn²⁺, Cd²⁺, Ag²⁺, Pb²⁺ and any combination thereof;
the chemical compound comprising 1,2-diol or 1,3-diol is selected from saccharide or a derivative thereof, α-hydroxy acid, a chemical compound comprising a ribose, nucleotide sugar, alditol, polyphenol, catecholamine or catecholamine derivative, tris(hydroxymethyl)methyl aminomethane (Tris), protocatechualdehyde, protocatechuic acid, caffeic acid, rosmarinic acid, lithospermic acid, salvianic acid A, salvianolic acid B and any combination thereof; and
the molecule that can interact with the metal ion by coordination contains nitrogen, oxygen, sulfur, phosphorus or carbon atom that can coordinate with the metal ion.

41. The method according to claim 40, wherein:
the saccharide is selected from monosaccharide, oligosaccharide, polysaccharide and any combination thereof, or selected from disaccharide, trisaccharide, tetrasccharide, complex oligosaccharide, pentasaccharide and any combination thereof;
the derivative of saccharide is selected from N-acetylneuraminic acid (sialic acid), N-Acetyl-D-Galactosamine and any combination thereof;
α-hydroxy acid is selected from tartaric acid, malic acid, citric acid, isocitric acid and any combination thereof;
the chemical compound comprising a ribose is selected from nucleotide or modified nucleotide, derivative of nucleotide or modified nucleotide, nucleoside or nucleoside analogue, and any combination thereof;
the nucleotide sugar is selected from uridine diphosphate glucose (UDPG), uridine diphosphate N-acetylglucosamine, uridine diphosphate glucuronic acid, adenosine diphosphate glucose, uridine diphosphate galactose, uridine diphosphate xylose, guanosine diphosphate mannose, guanosine diphosphate fucose, cytidine monophosphate N-acetylneuraminic acid, uridine diphosphate N-acetylgalactosamine and any combination thereof;
the alditol is selected from glycerin, propanetriol, tetritol, pentitol, hexitol, erythritol, threitol, arabitol, xylitol, adonitol, fucitol, sorbitol such as L-sorbitol or D-sorbitol, mannitol, dulcitol, iditol, talitol, allitol, maltitol, lactitol, isomalt and any combination thereof;
the polyphenol is selected from catechin, neochlorogenic acid, anthocyanin, proanthocyanidin, catechol or derivative thereof, such as catechol, 3-fluorocatechol, 3-chlorocatechol, 3-bromocatechol, 4-fluorocatechol, 4-chlorocatechol, 4-bromocatechol, 3-methylcatechol, 4-methylcatechol, 3-methoxycatechol, 3-propylcatechol, 3-isopropylcatechol, 3,6-dibromocatechol, 4,5-dibromocatechol, 3,6-dichlorocatechol, and any combination thereof;
the catecholamine or catecholamine derivative is selected from epinephrine, norepinephrine, isoprenaline and any combination thereof; and
the molecule that can interact with the metal ion by coordination is a compound contains at least one carboxylic acid group or at least one amine group, an amino acid, modified amino acid, polymer of amino acids or modified amino acids, a chemical compound comprising guanine, adenine, thymine, cytosine or uracil, and any combination thereof.

42. The method according to claim 41, wherein:
the monosaccharide is selected from D-glyceraldehyde, D-erythrose, D-ribose, 2′-deoxy-D-ribose, D-xylose, L-arabinose, D-lyxose, D-glucose, D-galactose, D-mannose, D-fructose, L-sorbose, L-fucose, D-allose, D-tagatose, L-rhamnose, D-galactose and any combination thereof;
the disaccharide is selected from sucrose, isomaltulose, maltulose, turanose, leucrose, trehalulose, lactulose, maltose and any combination thereof;
the trisaccharide is selected from raffinose;
the tetrasccharide is selected from stachyose;
the complex oligosaccharide is selected from acarbose;
the pentasaccharide is selected from verbascose;
the nucleotide is selected from adenine nucleotide, cytosine nucleotide, uracil nucleotide, guanine nucleotide and any combination thereof;
the modified nucleotide is selected from a nucleotide containing 5-methylcytidine (m⁵C), N6-methyladenosine (m⁶A), pseudouridine (Ψ), inosine (I), N7-methylguanosine (m⁷G), N1-methyladenosine (m¹A), dihydrouridine (D), N2-methylguanosine (m²G), N2,N2-dimethylguanosine (m₂²G), wybutosine (Y), 5-methyluridine (T), N-acetylcytidine (ac4C) and any combination thereof;
the derivative of nucleotide or modified nucleotide is selected from monophosphate derivative, diphosphate derivative, triphosphate derivative and tetraphosphate derivative of a nucleotide or a modified nucleotide and any combination thereof, or selected from ADP, UDP, GDP, CDP, ATP, UTP, GTP, CTP and any combination thereof; and
the nucleoside analogue is selected from galidesvir, ribavirin, molnupiravir, remdesivir, loxoribine, mizoribine, 5-azacytidine, capecitabine, doxifluridine, 5-fluorouridine, forodesine, clitocine, pyrazofurin, sangivamycin, pseudouridimycin and any combination thereof;
the sorbitol is selected from L-sorbitol or D-sorbitol and any combination thereof;
the catechol or derivative thereof is selected from catechol, 3-fluorocatechol, 3-chlorocatechol, 3-bromocatechol, 4-fluorocatechol, 4-chlorocatechol, 4-bromocatechol, 3-methylcatechol, 4-methylcatechol, 3-methoxycatechol, 3-propylcatechol, 3-isopropylcatechol, 3,6-dibromocatechol, 4,5-dibromocatechol, 3,6-dichlorocatechol, and any combination thereof;
the amino acid is selected from alanine, cysteine, aspartic acid, glutamic acid, phenylalanine, glycine, histidine, isoleucine, lysine, leucine, methionine, asparagine, proline, glutamine, arginine, serine, threonine, valine, tryptophan, tyrosine, pyrolysine, selenocysteine and any combination thereof;
the modified amino acid is selected from phosphorylate amino acid, glycosylated amino acid, acetylated amino acid, methylated amino acid and any combination thereof, or selected from O-phospho-serine (p-S), N4-(β-N-acetyl-D-glucosaminyl)-asparagine (GlcNAc-N), O-acetyl-threonine (Ac-T), Nω, N′ω-dimethyl-arginine (SDMA) and any combination thereof; and
the chemical compound comprising guanine, adenine, thymine, cytosine or uracil is selected from guanine, adenine, thymine, cytosine or uracil, or a nucleoside comprising any one of them, or a nucleotide comprising any one of them, wherein the nucleotide is a ribonucleotide or a deoxyribonucleotide.
